(19) United States
(12) Patent Application Publication
(10) Pub. No.: US 2022/0332800 A1
(43) Pub. Date: Oct. 20, 2022

(54) NOVEL ANTI-ZIKA VIRUS ANTIBODIES AND USES THEREOF

(71) Applicant: Takeda Vaccines, Inc., Cambridge, MA (US)

(72) Inventors: Subash C. DAS, Billerica, MA (US); Isamu TSUJI, Cambridge, MA (US); Jill A. LIVENGOOD, Fort Collins, CO (US); Hansi DEAN, Weston, MA (US)

(73) Assignee: Takeda Vaccines, Inc., Cambridge, MA (US)

(21) Appl. No.: 17/295,770

(22) PCT Filed: Sep. 20, 2019
(86) PCT No.: PCT/US2019/052189

§ 371 Date: May 20, 2021

Related U.S. Application Data
(60) Provisional application No. 62/770,148, filed on Nov. 20, 2018.

(51) Int. Cl. C07K 16/10; A61K 39/42; A61K 45/06; G01N 33/569

(52) U.S. Cl. CPC C07K 16/1081; A61K 39/42; A61K 45/06; G01N 33/56983; C07K 2317/21; C07K 2317/24; C07K 2317/92; C07K 2317/31; A61K 2039/545

(57) ABSTRACT

The present invention provides isolated monoclonal antibodies, or antigen-binding portions thereof, that bind to ZIKV glycoproteins, pharmaceutical compositions comprising the antibodies and methods of use. The antibodies, or antigen-binding portions thereof, according to the present invention are useful for inhibiting or neutralizing ZIKV activity, thus providing a means of treating or preventing ZIKV infection in humans. The antibodies, or antigen-binding portions thereof, according to the present invention may be used prophylactically or therapeutically and may be used alone or in combination with one or more other anti-viral agents or vaccines.

### RELATED APPLICATION INFORMATION

This application is a 35 U.S.C. § 371 National Stage filing of International Application No. PCT/US2019/052189 filed Sep. 20, 2019, which claims the benefit of priority to U.S. Provisional Application No. 62/770,148, filed Nov. 20, 2018. The entire contents of the aforementioned applications are incorporated herein by reference.

### STATEMENT REGARDING FEDERALLY-SPONSORED RESEARCH

This invention was made with government support under Contract No. HHSO100201600015C with the Department of Health and Human Services, Office of the Assistant Secretary for Preparedness and Response, Biomedical Advanced Research and Development Authority. This invention was created in the performance of a Cooperative Research and Development Agreement with the Centers for Disease Control and Prevention, an Agency of the Department of Health and Human Services. The Government of the United States has certain rights in the invention.

### SEQUENCE LISTING

The instant application contains a Sequence Listing which is submitted herewith in electronically readable ASCII format via EFS-Web, and is hereby incorporated by reference in its entirety. The electronica Sequence Listing file was created on May 18, 2021, is named “T08500WOUS_SL_ST25” and is 135 KB in size.

### BACKGROUND

A Zika virus (ZIKV) is a positive-stranded RNA arthropod-borne virus (arbovirus) in the genus Flavivirus, family Flaviviridae (Gubler, D J, et al., In: Knipe, D M, et al., eds., Fields Virology, 5^{th} edn., Philadelphia, Pa.: Lippincott Williams & Wilkins Publishers, 2007: 1155-227, incorporated herein by reference). It is thought to be principally transmitted to humans by the mosquito, Aedes aegypti. In addition to transmission by mosquitoes, ZIKV may be sexually (Foy, B D, et al., (2011), Emerg. Infect. Dis. 17:880-882, incorporated herein by reference) and vertically (Mlakar, J. et al., (2016), N. Engl. J. Med. 374:951-958, incorporated herein by reference) transmitted, or transmitted via blood products or tissue samples. ZIKV generally causes a mild disease, with a rash and mild febrile illness in the majority of symptomatic individuals. However, when pregnant women are infected with ZIKV, there is an increased risk of developing microcephaly in the fetus (Schuler-Faccini, L. et al., (2016), MMWR Morb. Mortal. W ly Rep. 65:59-62, incorporated herein by reference) or other developmental abnormalities (Brasil et al., (2016) N. Engl. J. Med., March 4, incorporated herein by reference). There have also been reports that ZIKV is associated with

Guillain-Barre syndrome in patients infected with the virus (Cao-Lormeau, V M, et al., (2016), Lancet, April 9; 387(10027): 1531-9, incorporated herein by reference). In addition, there have also been reports of an association of ZIKV with brain ischemia, myelitis and meningoencephalitis (Carteaux, G. et al. (2016), N. Engl. J. Med. 374(16): 1595, incorporated herein by reference).

ZIKV belongs to the genus Flavivirus, which also includes the West Nile virus, dengue virus, tick-borne encephalitis virus, yellow fever virus, and several other viruses which may cause encephalitis. Flaviviruses are enveloped, with icosahedral and spherical geometries. The diameter is around 50 nm. Genomes are linear positive-sense RNA and non-segmented, around 10-11 kb bases complexed with multiple copies of the capsid protein (C), surrounded by an icosahedral shell consisting of 180 copies each of the envelope glycoprotein (E) (˜500 amino acids), and the membrane protein (M) (˜75 amino acids) or precursor membrane protein (prM) (˜165 amino acids), all anchored in a lipid membrane. The genome also codes for seven non-structural proteins (NS1, NS2A, NS2B, NS3, NS4A, NS4B and NS5) (WO2018010789, incorporated herein by reference).

A ZIKV was first isolated from macaques in 1947 in the Zika forest in Uganda (G. W. A. Dick, S. F. Kitchen, A. J. Haddow, Zika virus. I. Isolations and serological specificity. Trans. R. Soc. Trop. Med. Hyg. 46, 509-520 (1952), incorporated herein by reference) and the first human infection was reported in Nigeria in 1954 (F. N. Macnamara, Zika virus: a report on three cases of human infection during an epidemic of jaundice in Nigeria. Trans. R. Soc. Trop. Med. Hyg. 48, 139-145 (1954), incorporated herein by reference). Since then, ZIKV infections were sporadically reported in Africa and southeast Asia (D. Musso, Van Mai Cao-Lormeau, D. J. Gubler, Zika virus: following the path of dengue and chikungunya? The Lancet. 386, 243-244 (2015), incorporated herein by reference), but epidemics were reported in Micronesia in 2007 (M. R. Duffy et al., Zika virus outbreak on Yap Island, Federated States of Micronesia. N Engl J Med. 360, 2536-2543 (2009), incorporated herein by reference) and in French Polynesia in 2013-14, with the virus subsequently spreading to other countries in the Oceanian continent (V.-M. Cao-Lormeau, D. Musso, Emerging arboviruses in the Pacific. Lancet. 384, 1571-1572 (2014); D. Musso, E. J. Nilles, V.-M. Cao-Lormeau, Rapid spread of emerging Zika virus in the Pacific area. Clin. Microbiol. Infect. 20, 0595-6 (2014), each incorporated herein by reference). After its introduction into Brazil in 2015, ZIKV has spread rapidly and in February 2016, the World Health Organization (WHO) declared it a Public Health Emergency of International Concern (L. R. Baden, L. R. Petersen, D. J. Jamieson, A. M. Powers, M. A. Honein, Zika Virus. N. Engl. J. Med. 374, 1552-1563 (2016); A. S. Fauci, D. M. Morens, Zika Virus in the Americas -Yet Another Arbovirus Threat. N Engl J Med, 374(7):601-4 (2016); D. L. Heymann et al., Zika virus and microcephaly: why is this situation a PHEIC? Lancet 387, 719-721 (2016), each incorporated herein by reference).

A phenomenon that is characteristic of certain Flaviviruses is the disease-enhancing activity of cross-reactive antibodies elicited by previous infection by heterologous viruses. In the case of Dengue virus (DENV), for which 4 serotypes are known, there is epidemiological evidence that a primary infection protects from reinfection with the same serotype, but represents a risk factor for the development of severe disease upon reinfection with a different serotype (S. B. Haistead, Dengue Antibody-Dependent Enhancement: Knowns and Unknowns. Microbiol Spectr. 2, 249-271 (2014), incorporated herein by reference). The exacerbated disease is triggered by E and prM-specific antibodies that fail to neutralize the incoming virus but instead enhance its capture by Fc receptor-expressing (FcR⁺) cells, leading to enhanced viral replication and activation of cross-reactive memory T cells. The resulting cytokine storm is thought to be the basis of the most severe form of disease known as dengue hemorragic fever/dengue shock syndrome (S. B. Haistead, Neutralization and antibody-dependent enhancement of dengue viruses. Adv Virus Res. 60, 421-467 (2003); G. Screaton, J. Mongkolsapaya, S. Yacoub, C. Roberts, New insights into the immunopathology and control of dengue virus infection. Nat Rev Immunol. 15, 745-759 (2015), each incorporated herein by reference). The role of antibodies in severe dengue disease is supported by studies showing that waning levels of maternal antibodies in infants represent a higher risk for development of severe dengue disease (S. B. Halstead, Neutralization and antibody-dependent enhancement of dengue viruses. Adv Virus Res. 60, 421-467 (2003); S. B. Haistead et al., Dengue hemorrhagic fever in infants: research opportunities ignored. Emerging Infect Dis. 8, 1474-1479 (2002); T. H. Nguyen et al., Dengue hemorrhagic fever in infants: a study of clinical and cytokine profiles. J In feet Dis. 189, 221-232 (2004); A. L. Rothman, Dengue: defining protective versus pathologic immunity. J Clin Invest 113, 946-951 (2004), each incorporated herein by reference).

Moreover, according to the WHO, the recent increase in cases of microcephaly and other neurological disorders potentially associated with a ZIKV infection has prompted an increase in demand for laboratory testing to detect a ZIKV infection. In this context, high specificity of the antibodies is required in order to distinguish ZIKV infection from infection of other Flaviviruses. However, known anti-ZIKV antibodies are typically cross-reactive for other Flaviviruses and, thus, not useful to distinguish ZIKV infection from infection of other Flaviviruses.

In view of the above, it is an object of the present invention to provide novel antibodies, which specifically bind to ZIKV epitopes. It is also an object of the present invention to provide potently neutralizing anti-ZIKV antibodies. Such antibodies preferably do not contribute to antibody-dependent enhancement (ADE) of a ZIKV infection. It is also an object of the present invention to provide highly specific anti-ZIKV antibodies useful in diagnosis and testing of ZIKV infection and diagnostic methods using such antibodies. Additionally, these antibodies could be used for characterization of vaccine drug substance or vaccine drug product and immune responses to vaccine candidates.

### SUMMARY

The present invention provides anti-ZIKV antibodies, antigen-binding portions thereof.

In a first aspect, the present invention provides an isolated antibody, or antigen-binding portion thereof, that binds to a ZIKV, wherein the isolated antibody, or antigen-binding portion thereof, comprises a heavy chain variable region comprising a CDR3 comprising the amino acid sequence of SEQ ID NO: 5 and a light chain variable region comprising a CDR3 comprising the amino acid sequence of SEQ ID NO: 10.

In some embodiments, the isolated antibody, or antigen-binding portion thereof, further comprises a heavy chain variable region comprising a CDR2 comprising the amino acid sequence of SEQ ID NO: 4 and a light chain variable region comprising a CDR2 comprising the amino acid sequence of SEQ ID NO: 9. In other embodiments, the isolated antibody, or antigen-binding portion thereof, comprises a heavy chain variable region comprising a CDR1 comprising the amino acid sequence of SEQ ID NO: 3 and a light chain variable region comprising a CDR1 comprising the amino acid sequence of SEQ ID NO: 8.

In one aspect, the present invention provides an isolated antibody, or antigen-binding portion thereof, that binds to a ZIKV, wherein the isolated antibody, or antigen-binding portion thereof, comprises a heavy chain variable region comprising a CDR3 comprising the amino acid sequence of SEQ ID NO: 15 and a light chain variable region comprising a CDR3 comprising the amino acid sequence of SEQ ID NO: 20.

In some embodiments, or antigen-binding portion thereof, further comprises a heavy chain variable region comprising a CDR2 comprising the amino acid sequence of SEQ ID NO: 14 and a light chain variable region comprising a CDR2 comprising the amino acid sequence of SEQ ID NO: 19. In other embodiments, the isolated antibody, or antigen-binding portion thereof, comprises a heavy chain variable region comprising a CDR1 comprising the amino acid sequence of SEQ ID NO: 13 and a light chain variable region comprising a CDR1 comprising the amino acid sequence of SEQ ID NO: 18.

In one aspect, the present invention provides an isolated antibody, or antigen-binding portion thereof, that binds to a ZIKV, wherein the isolated antibody, or antigen-binding portion thereof, comprises a heavy chain variable region comprising a CDR3 comprising the amino acid sequence of SEQ ID NO: 25 and a light chain variable region comprising a CDR3 comprising the amino acid sequence of SEQ ID NO: 30.

In some embodiments, the isolated antibody, or antigen-binding portion thereof, further comprises a heavy chain variable region comprising a CDR2 comprising the amino acid sequence of SEQ ID NO: 24 and a light chain variable region comprising a CDR2 comprising the amino acid sequence of SEQ ID NO: 29. In other embodiments, the isolated antibody, or antigen-binding portion thereof, comprises a heavy chain variable region comprising a CDR1 comprising the amino acid sequence of SEQ ID NO: 23 and a light chain variable region comprising a CDR1 comprising the amino acid sequence of SEQ ID NO: 28.

In one aspect, the present invention provides an isolated antibody, or antigen-binding portion thereof, that binds to a ZIKV, wherein the isolated antibody, or antigen-binding portion thereof, comprises a heavy chain variable region comprising a CDR3 comprising the amino acid sequence of SEQ ID NO: 35 and a light chain variable region comprising a CDR3 comprising the amino acid sequence of SEQ ID NO: 40.

In some embodiments, the isolated antibody, or antigen-binding portion thereof, further comprises a heavy chain variable region comprising a CDR2 comprising the amino acid sequence of SEQ ID NO: 34 and a light chain variable region comprising a CDR2 comprising the amino acid sequence of SEQ ID NO: 39. In other embodiments, the isolated antibody, or antigen-binding portion thereof, comprises a heavy chain variable region comprising a CDR1 comprising the amino acid sequence of SEQ ID NO: 33 and a light chain variable region comprising a CDR1 comprising the amino acid sequence of SEQ ID NO: 38.

In one aspect, the present invention provides an isolated antibody, or antigen-binding portion thereof, that binds to a ZIKV, wherein the isolated antibody, or antigen-binding portion thereof, comprises a heavy chain variable region comprising a CDR3 comprising the amino acid sequence of SEQ ID NO: 45 and a light chain variable region comprising a CDR3 comprising the amino acid sequence of SEQ ID NO: 50.

In some embodiments, the isolated antibody, or antigen-binding portion thereof, further comprises a heavy chain variable region comprising a CDR2 comprising the amino acid sequence of SEQ ID NO: 44 and a light chain variable region comprising a CDR2 comprising the amino acid sequence of SEQ ID NO: 49. In other embodiments, the isolated antibody, or antigen-binding portion thereof, comprises a heavy chain variable region comprising a CDR1 comprising the amino acid sequence of SEQ ID NO: 43 and a light chain variable region comprising a CDR1 comprising the amino acid sequence of SEQ ID NO: 48.

In one aspect, the present invention provides an isolated antibody, or antigen-binding portion thereof, that binds to a ZIKV, wherein the isolated antibody, or antigen-binding portion thereof, comprises a heavy chain variable region comprising a CDR3 comprising the amino acid sequence of SEQ ID NO: 55 and a light chain variable region comprising a CDR3 comprising the amino acid sequence of SEQ ID NO: 60.

In some embodiments, the isolated antibody, or antigen-binding portion thereof, further comprises a heavy chain variable region comprising a CDR2 comprising the amino acid sequence of SEQ ID NO: 54 and a light chain variable region comprising a CDR2 comprising the amino acid sequence of SEQ ID NO: 59. In other embodiments, the isolated antibody, or antigen-binding portion thereof, comprises a heavy chain variable region comprising a CDR1 comprising the amino acid sequence of SEQ ID NO: 53 and a light chain variable region comprising a CDR1 comprising the amino acid sequence of SEQ ID NO: 58.

In one aspect, the present invention provides an isolated antibody, or antigen-binding portion thereof, that binds to a ZIKV, wherein the isolated antibody, or antigen-binding portion thereof, comprises a heavy chain variable region comprising a CDR3 comprising the amino acid sequence of SEQ ID NO: 89 and a light chain variable region comprising a CDR3 comprising the amino acid sequence of SEQ ID NO: 94.

In some embodiments, the isolated antibody, or antigen-binding portion thereof, further comprises a heavy chain variable region comprising a CDR2 comprising the amino acid sequence of SEQ ID NO: 88 and a light chain variable region comprising a CDR2 comprising the amino acid sequence of SEQ ID NO: 93. In other embodiments, the isolated antibody, or antigen-binding portion thereof, comprises a heavy chain variable region comprising a CDR1 comprising the amino acid sequence of SEQ ID NO: 87 and a light chain variable region comprising a CDR1 comprising the amino acid sequence of SEQ ID NO: 92.

In one aspect, the present invention provides an isolated antibody, or an antigen-binding portion thereof, that binds to a ZIKV, wherein the isolated antibody, or antigen-binding portion thereof, comprises a heavy chain variable region comprising a CDR3 comprising the amino acid sequence of SEQ ID NO: 132 and a light chain variable region comprising a CDR3 comprising the amino acid sequence of SEQ ID NO: 137.

In some embodiments, the isolated antibody, or antigen-binding portion thereof, further comprises a heavy chain variable region comprising a CDR2 comprising the amino acid sequence of SEQ ID NO: 131 and a light chain variable region comprising a CDR2 comprising the amino acid sequence of SEQ ID NO: 136. In other embodiments, the isolated antibody, or antigen-binding portion thereof, comprises a heavy chain variable region comprising a CDR1 comprising the amino acid sequence of SEQ ID NO: 130 and a light chain variable region comprising a CDR1 comprising the amino acid sequence of SEQ ID NO: 135.

In one aspect, the present invention provides an isolated antibody, or antigen-binding portion thereof, that binds to a ZIKV, wherein the isolated antibody, or antigen-binding portion thereof, comprises a heavy chain variable region comprising a CDR3 domain comprising the amino acid sequence of SEQ ID NO: 5, a CDR2 domain comprising the amino acid sequence of SEQ ID NO: 4, and a CDR1 domain comprising the amino acid sequence of SEQ ID NO: 3, and a light chain variable region comprising a CDR3 domain comprising the amino acid sequence of SEQ ID NO: 10, a CDR2 domain comprising the amino acid sequence of SEQ ID NO: 9, and a CDR1 domain comprising the amino acid sequence of SEQ ID NO: 8.

In one aspect, the present invention provides an isolated antibody, or antigen-binding portion thereof, that binds to a ZIKV, wherein the isolated antibody, or antigen-binding portion thereof, comprises a heavy chain variable region comprising a CDR3 domain comprising the amino acid sequence of SEQ ID NO: 15, a CDR2 domain comprising the amino acid sequence of SEQ ID NO: 14, and a CDR1 domain comprising the amino acid sequence of SEQ ID NO: 13, and a light chain variable region comprising a CDR3 domain comprising the amino acid sequence of SEQ ID NO: 20, a CDR2 domain comprising the amino acid sequence of SEQ ID NO: 19, and a CDR1 domain comprising the amino acid sequence of SEQ ID NO: 18.

In one aspect, the present invention provides an isolated antibody, or antigen-binding portion thereof, that binds to a ZIKV, wherein the isolated antibody, or antigen-binding portion thereof, comprises a heavy chain variable region comprising a CDR3 domain comprising the amino acid sequence of SEQ ID NO: 25, a CDR2 domain comprising the amino acid sequence of SEQ ID NO: 24, and a CDR1 domain comprising the amino acid sequence of SEQ ID NO: 23, and a light chain variable region comprising a CDR3 domain comprising the amino acid sequence of SEQ ID NO: 30, a CDR2 domain comprising the amino acid sequence of SEQ ID NO: 29, and a CDR1 domain comprising the amino acid sequence of SEQ ID NO: 28.

In one aspect, the present invention provides an isolated antibody, or antigen-binding portion thereof, that binds to a ZIKV, wherein the isolated antibody, or antigen-binding portion thereof, comprises a heavy chain variable region comprising a CDR3 domain comprising the amino acid sequence of SEQ ID NO: 35, a CDR2 domain comprising the amino acid sequence of SEQ ID NO: 34, and a CDR1 domain comprising the amino acid sequence of SEQ ID NO: 33, and a light chain variable region comprising a CDR3 domain comprising the amino acid sequence of SEQ ID NO: 40, a CDR2 domain comprising the amino acid sequence of SEQ ID NO: 39, and a CDR1 domain comprising the amino acid sequence of SEQ ID NO: 38.

In one aspect, the present invention provides an isolated antibody, or antigen-binding portion thereof, that binds to a ZIKV, wherein the isolated antibody, or antigen-binding portion thereof, comprises a heavy chain variable region comprising a CDR3 domain comprising the amino acid sequence of SEQ ID NO: 45, a CDR2 domain comprising the amino acid sequence of SEQ ID NO: 44, and a CDR1 domain comprising the amino acid sequence of SEQ ID NO: 43, and a light chain variable region comprising a CDR3 domain comprising the amino acid sequence of SEQ ID NO: 50, a CDR2 domain comprising the amino acid sequence of SEQ ID NO: 49, and a CDR1 domain comprising the amino acid sequence of SEQ ID NO: 48.

In one aspect, the present invention provides an isolated antibody, or antigen-binding portion thereof, that binds to a ZIKV, wherein the isolated antibody, or antigen-binding portion thereof, comprises a heavy chain variable region comprising a CDR3 domain comprising the amino acid sequence of SEQ ID NO: 55, a CDR2 domain comprising the amino acid sequence of SEQ ID NO: 54, and a CDR1 domain comprising the amino acid sequence of SEQ ID NO: 53, and a light chain variable region comprising a CDR3 domain comprising the amino acid sequence of SEQ ID NO: 60, a CDR2 domain comprising the amino acid sequence of SEQ ID NO: 59, and a CDR1 domain comprising the amino acid sequence of SEQ ID NO: 58.

In one aspect, the present invention provides an isolated antibody, or antigen-binding portion thereof, that binds to a ZIKV, wherein the isolated antibody, or antigen-binding portion thereof, comprises a heavy chain variable region comprising a CDR3 domain comprising the amino acid sequence of SEQ ID NO: 89, a CDR2 domain comprising the amino acid sequence of SEQ ID NO: 88, and a CDR1 domain comprising the amino acid sequence of SEQ ID NO: 87, and a light chain variable region comprising a CDR3 domain comprising the amino acid sequence of SEQ ID NO: 94, a CDR2 domain comprising the amino acid sequence of SEQ ID NO: 93, and a CDR1 domain comprising the amino acid sequence of SEQ ID NO: 92.

In one aspect, the present invention provides an isolated antibody, or an antigen-binding portion thereof, that binds to a ZIKV, comprising a heavy chain variable region comprising a CDR3 domain comprising the amino acid sequence of SEQ ID NO: 132, a CDR2 domain comprising the amino acid sequence of SEQ ID NO: 131, and a CDR1 domain comprising the amino acid sequence of SEQ ID NO: 130, and a light chain variable region comprising a CDR3 domain comprising the amino acid sequence of SEQ ID NO: 137, a CDR2 domain comprising the amino acid sequence of SEQ ID NO: 136, and a CDR1 domain comprising the amino acid sequence of SEQ ID NO: 135.

In one aspect, the present invention provides an isolated antibody, or an antigen-binding portion thereof, that binds to a ZIKV, comprising a heavy chain variable domain comprising an amino acid sequence set forth in SEQ ID NO: 2, or a sequence having at least 70%, 75%, 80%, 85%, 90%, 95%, 96%, 97%, 98%, or 99% identity to SEQ ID NO: 2, and a light chain variable domain comprising an amino acid sequence set forth in SEQ ID NO: 7, or a sequence having at least 70%, 75%, 80%, 85%, 90%, 92%, 94%, 95%, 96%, 97%, 98%, or 99% identity to SEQ ID NO: 7.

In one aspect, the present invention provides an isolated antibody, or an antigen-binding portion thereof, that binds to a ZIKV, comprising a heavy chain variable domain comprising an amino acid sequence set forth in SEQ ID NO: 2 and a light chain variable domain comprising an amino acid sequence set forth in SEQ ID NO: 7.

In one aspect, the present invention provides an isolated antibody, or an antigen-binding portion thereof, that binds to a ZIKV, comprising a heavy chain comprising an amino acid sequence set forth in SEQ ID NO: 1, or a sequence having at least 70%, 75%, 80%, 85%, 90%, 92%, 94%, 95%, 96%, 97%, 98%, or 99% identity to SEQ ID NO: 1, and a light chain comprising an amino acid sequence set forth in SEQ ID NO: 6, or a sequence having at least 70%, 75%, 80%, 85%, 90%, 92%, 94%, 95%, 96%, 97%, 98%, or 99% identity to SEQ ID NO: 6.

In one aspect, the present invention provides an isolated antibody, or an antigen-binding portion thereof, that binds to a ZIKV, comprising a heavy chain comprising an amino acid sequence set forth in SEQ ID NO: 1 and a light chain comprising an amino acid sequence set forth in SEQ ID NO: 6.

In one aspect, the present invention provides an isolated antibody, or an antigen-binding portion thereof, that binds to a ZIKV, comprising a heavy chain variable region comprising an amino acid sequence set forth in SEQ ID NO: 12, or a sequence having at least 70%, 75%, 80%, 85%, 90%, 92%, 94%, 95%, 96%, 97%, 98%, or 99% identity to SEQ ID NO: 12, and a light chain variable region comprising an amino acid sequence set forth in SEQ ID NO: 17, or a sequence having at least 70%, 75%, 80%, 85%, 90%, 92%, 94%, 95%, 96%, 97%, 98%, or 99% identity to SEQ ID NO: 17.

In one aspect, the present invention provides an isolated antibody, or an antigen-binding portion thereof, that binds to a ZIKV, comprising a heavy chain variable region comprising the amino acid sequence of SEQ ID NO: 12 and a light chain variable region comprising the amino acid sequence of SEQ ID NO: 17.

In one aspect, the present invention provides an isolated antibody, or an antigen-binding portion thereof, that binds to a ZIKV, comprising a heavy chain comprising an amino acid sequence set forth in SEQ ID NO: 11, or a sequence having at least 70%, 75%, 80%, 85%, 90%, 92%, 94%, 95%, 96%, 97%, 98%, or 99% identity to SEQ ID NO: 11, and a light chain comprising an amino acid sequence set forth in SEQ ID NO: 16, or a sequence having at least 70%, 75%, 80%, 85%, 90%, 92%, 94%, 95%, 96%, 97%, 98%, or 99% identity to SEQ ID NO: 16.

In one aspect, the present invention provides an isolated antibody, or an antigen-binding portion thereof, that binds to a ZIKV, comprising a heavy chain comprising the amino acid sequence of SEQ ID NO: 11 and a light chain comprising the amino acid sequence of SEQ ID NO: 16.

In one aspect, the present invention provides an isolated antibody, or an antigen-binding portion thereof, that binds to a ZIKV, comprising a heavy chain comprising the amino acid sequence of SEQ ID NO: 11 and a light chain comprising the amino acid sequence of SEQ ID NO: 16.

In one aspect, the present invention provides an isolated antibody, or an antigen-binding portion thereof, that binds to a ZIKV, comprising a heavy chain variable region comprising an amino acid sequence set forth in SEQ ID NO: 22, or a sequence having at least 70%, 75%, 80%, 85%, 90%, 92%, 94%, 95%, 96%, 97%, 98%, or 99% identity to SEQ ID NO: 22, and a light chain variable region comprising an amino acid sequence set forth in SEQ ID NO: 27, or a sequence having at least 70%, 75%, 80%, 85%, 90%, 92%, 94%, 95%, 96%, 97%, 98%, or 99% identity to SEQ ID NO: 27.

In one aspect, the present invention provides an isolated antibody, or an antigen-binding portion thereof, that binds to a ZIKV, comprising a heavy chain variable region comprising the amino acid sequence of SEQ ID NO: 22 and a light chain variable region comprising the amino acid sequence of SEQ ID NO: 27.

In one aspect, the present invention provides an isolated antibody, or an antigen-binding portion thereof, that binds to a ZIKV, comprising a heavy chain comprising an amino acid sequence set forth in SEQ ID NO: 21, or a sequence having at least 70%, 75%, 80%, 85%, 90%, 92%, 94%, 95%, 96%, 97%, 98%, or 99% identity to SEQ ID NO: 21, and a light chain comprising an amino acid sequence set forth in SEQ ID NO: 26, or a sequence having at least 70%, 75%, 80%, 85%, 90%, 92%, 94%, 95%, 96%, 97%, 98%, or 99% identity to SEQ ID NO: 26.

In one aspect, the present invention provides an isolated antibody, or an antigen-binding portion thereof, that binds to a ZIKV, comprising a heavy chain comprising the amino acid sequence of SEQ ID NO: 21 and a light chain comprising the amino acid sequence of SEQ ID NO: 26.

In one aspect, the present invention provides an isolated antibody, or an antigen-binding portion thereof, that binds to a ZIKV, comprising a heavy chain variable region comprising an amino acid sequence set forth in SEQ ID NO: 32, or a sequence having at least 70%, 75%, 80%, 85%, 90%, 92%, 94%, 95%, 96%, 97%, 98%, or 99% identity to SEQ ID NO: 32, and a light chain variable region comprising an amino acid sequence set forth in SEQ ID NO: 37, or a sequence having at least 70%, 75%, 80%, 85%, 90%, 92%, 94%, 95%, 96%, 97%, 98%, or 99% identity to SEQ ID NO: 37.

In one aspect, the present invention provides an isolated antibody, or an antigen-binding portion thereof, that binds to a ZIKV, comprising a heavy chain variable region comprising the amino acid sequence of SEQ ID NO: 32 and a light chain variable region comprising the amino acid sequence of SEQ ID NO: 37.

In one aspect, the present invention provides an isolated antibody, or an antigen-binding portion thereof, that binds to a ZIKV, comprising a heavy chain comprising an amino acid sequence set forth in SEQ ID NO: 31, or a sequence having at least 70%, 75%, 80%, 85%, 90%, 92%, 94%, 95%, 96%, 97%, 98%, or 99% identity to SEQ ID NO: 31, and a light chain comprising an amino acid sequence set forth in SEQ ID NO: 36, or a sequence having at least 70%, 75%, 80%, 85%, 90%, 92%, 94%, 95%, 96%, 97%, 98%, or 99% identity to SEQ ID NO: 36.

In one aspect, the present invention provides an isolated antibody, or an antigen-binding portion thereof, that binds to a ZIKV, comprising a heavy chain comprising the amino acid sequence of SEQ ID NO: 31 and a light chain comprising the amino acid sequence of SEQ ID NO: 36.

In one aspect, the present invention provides an isolated antibody, or an antigen-binding portion thereof, that binds to a ZIKV, comprising a heavy chain variable region having an amino acid sequence set forth in SEQ ID NO: 42, or a sequence comprising at least 70%, 75%, 80%, 85%, 90%, 92%, 94%, 95%, 96%, 97%, 98%, or 99% identity to SEQ ID NO: 42, and a light chain variable region having an amino acid sequence set forth in SEQ ID NO: 47, or a sequence comprising at least 70%, 75%, 80%, 85%, 90%, 92%, 94%, 95%, 96%, 97%, 98%, or 99% identity to SEQ ID NO: 47.

In one aspect, the present invention provides an isolated antibody, or an antigen-binding portion thereof, that binds to a ZIKV, comprising a heavy chain variable region comprising the amino acid sequence of SEQ ID NO: 42 and a light chain variable region comprising the amino acid sequence of SEQ ID NO: 47.

In one aspect, the present invention provides an isolated antibody, or an antigen-binding portion thereof, that binds to a ZIKV, comprising a heavy chain comprising an amino acid sequence set forth in SEQ ID NO: 41, or a sequence having at least 70%, 75%, 80%, 85%, 90%, 92%, 94%, 95%, 96%, 97%, 98%, or 99% identity to SEQ ID NO: 41, and a light chain comprising an amino acid sequence set forth in SEQ ID NO: 46, or a sequence having at least 70%, 75%, 80%, 85%, 90%, 92%, 94%, 95%, 96%, 97%, 98%, or 99% identity to SEQ ID NO: 46.

In one aspect, the present invention provides an isolated antibody, or an antigen-binding portion thereof, that binds to a ZIKV, comprising a heavy chain comprising the amino acid sequence of SEQ ID NO: 41 and a light chain comprising the amino acid sequence of SEQ ID NO: 46.

In one aspect, the present invention provides an isolated antibody, or an antigen-binding portion thereof, that binds to a ZIKV, comprising a heavy chain variable region comprising an amino acid sequence set forth in SEQ ID NO: 52, or a sequence having at least 70%, 75%, 80%, 85%, 90%, 92%, 94%, 95%, 96%, 97%, 98%, or 99% identity to SEQ ID NO: 52, and a light chain variable region comprising an amino acid sequence set forth in SEQ ID NO: 57, or a sequence having at least 70%, 75%, 80%, 85%, 90%, 92%, 94%, 95%, 96%, 97%, 98%, or 99% identity to SEQ ID NO: 57.

In one aspect, the present invention provides an isolated antibody, or an antigen-binding portion thereof, that binds to a ZIKV, comprising a heavy chain variable region comprising the amino acid sequence of SEQ ID NO: 52 and a light chain variable region comprising the amino acid sequence of SEQ ID NO: 57.

In one aspect, the present invention provides an isolated antibody, or an antigen-binding portion thereof, that binds to a ZIKV, comprising a heavy chain comprising an amino acid sequence set forth in SEQ ID NO: 51, or a sequence having at least 70%, 75%, 80%, 85%, 90%, 92%, 94%, 95%, 96%, 97%, 98%, or 99% identity to SEQ ID NO: 51, and a light chain comprising an amino acid sequence set forth in SEQ ID NO: 56, or a sequence having at least 70%, 75%, 80%, 85%, 90%, 92%, 94%, 95%, 96%, 97%, 98%, or 99% identity to SEQ ID NO: 56.

In one aspect, the present invention provides an isolated antibody, or an antigen-binding portion thereof, that binds to a ZIKV, comprising a heavy chain comprising the amino acid sequence of SEQ ID NO: 51 and a light chain comprising the amino acid sequence of SEQ ID NO: 56.

In one aspect, the present invention provides an isolated antibody, or an antigen-binding portion thereof, that binds to a ZIKV, comprising a heavy chain variable domain comprising an amino acid sequence set forth in SEQ ID NO: 86, or a sequence having at least 70%, 75%, 80%, 85%, 90%, 95%, 96%, 97%, 98%, or 99% identity to SEQ ID NO: 86, and a light chain variable domain comprising an amino acid sequence set forth in SEQ ID NO: 91, or a sequence having at least 70%, 75%, 80%, 85%, 90%, 92%, 94%, 95%, 96%, 97%, 98%, or 99% identity to SEQ ID NO: 91.

In one aspect, the present invention provides an isolated antibody, or an antigen-binding portion thereof, that binds to a ZIKV, comprising a heavy chain variable domain comprising an amino acid sequence set forth in SEQ ID NO: 86 and a light chain variable domain comprising an amino acid sequence set forth in SEQ ID NO: 91.

In one aspect, the present invention provides an isolated antibody, or an antigen-binding portion thereof, that binds to a ZIKV, comprising a heavy chain comprising an amino acid sequence set forth in SEQ ID NO: 85, or a sequence having at least 70%, 75%, 80%, 85%, 90%, 92%, 94%, 95%, 96%, 97%, 98%, or 99% identity to SEQ ID NO: 85, and a light chain comprising an amino acid sequence set forth in SEQ ID NO: 90, or a sequence having at least 70%, 75%, 80%, 85%, 90%, 92%, 94%, 95%, 96%, 97%, 98%, or 99% identity to SEQ ID NO: 90.

In one aspect, the present invention provides an isolated antibody, or an antigen-binding portion thereof, that binds to a ZIKV, comprising a heavy chain comprising an amino acid sequence set forth in SEQ ID NO: 85 and a light chain comprising an amino acid sequence set forth in SEQ ID NO: 90.

In one aspect, the present invention provides an isolated antibody, or an antigen-binding portion thereof, that binds to a ZIKV, comprising a heavy chain variable domain comprising an amino acid sequence set forth in SEQ ID NO: 96, or a sequence having at least 70%, 75%, 80%, 85%, 90%, 95%, 96%, 97%, 98%, or 99% identity to SEQ ID NO: 96, and a light chain variable domain comprising an amino acid sequence set forth in SEQ ID NO: 101, or a sequence having at least 70%, 75%, 80%, 85%, 90%, 92%, 94%, 95%, 96%, 97%, 98%, or 99% identity to SEQ ID NO: 101.

In one aspect, the present invention provides an isolated antibody, or an antigen-binding portion thereof, that binds to a ZIKV, comprising a heavy chain variable domain comprising an amino acid sequence set forth in SEQ ID NO: 96 and a light chain variable domain comprising an amino acid sequence set forth in SEQ ID NO: 101.

In one aspect, the present invention provides an isolated antibody, or an antigen-binding portion thereof, that binds to a ZIKV, comprising a heavy chain comprising an amino acid sequence set forth in SEQ ID NO: 95, or a sequence having at least 70%, 75%, 80%, 85%, 90%, 92%, 94%, 95%, 96%, 97%, 98%, or 99% identity to SEQ ID NO: 95, and a light chain comprising an amino acid sequence set forth in SEQ ID NO: 100, or a sequence having at least 70%, 75%, 80%, 85%, 90%, 92%, 94%, 95%, 96%, 97%, 98%, or 99% identity to SEQ ID NO: 100.

In one aspect, the present invention provides an isolated antibody, or an antigen-binding portion thereof, that binds to a ZIKV, comprising a heavy chain comprising an amino acid sequence set forth in SEQ ID NO: 95 and a light chain comprising an amino acid sequence set forth in SEQ ID NO: 100.

In one aspect, the present invention provides an isolated antibody, or an antigen-binding portion thereof, that binds to a ZIKV, comprising a heavy chain variable domain comprising an amino acid sequence set forth in SEQ ID NO: 106, or a sequence having at least 70%, 75%, 80%, 85%, 90%, 95%, 96%, 97%, 98%, or 99% identity to SEQ ID NO: 106, and a light chain variable domain comprising an amino acid sequence set forth in SEQ ID NO: 111, or a sequence having at least 70%, 75%, 80%, 85%, 90%, 92%, 94%, 95%, 96%, 97%, 98%, or 99% identity to SEQ ID NO: 111.

In one aspect, the present invention provides an isolated antibody, or an antigen-binding portion thereof, that binds to a ZIKV, comprising a heavy chain variable domain comprising an amino acid sequence set forth in SEQ ID NO: 106 and a light chain variable domain comprising an amino acid sequence set forth in SEQ ID NO: 111.

In one aspect, the present invention provides an isolated antibody, or an antigen-binding portion thereof, that binds to a ZIKV, comprising a heavy chain comprising an amino acid sequence set forth in SEQ ID NO: 105, or a sequence having at least 70%, 75%, 80%, 85%, 90%, 92%, 94%, 95%, 96%, 97%, 98%, or 99% identity to SEQ ID NO: 105, and a light chain comprising an amino acid sequence set forth in SEQ ID NO: 110, or a sequence having at least 70%, 75%, 80%, 85%, 90%, 92%, 94%, 95%, 96%, 97%, 98%, or 99% identity to SEQ ID NO: 110.

In one aspect, the present invention provides an isolated antibody, or an antigen-binding portion thereof, that binds to a ZIKV, comprising a heavy chain comprising an amino acid sequence set forth in SEQ ID NO: 105 and a light chain comprising an amino acid sequence set forth in SEQ ID NO: 110.

In one aspect, the present invention provides an isolated antibody, or an antigen-binding portion thereof, that binds to a ZIKV, comprising a heavy chain variable domain comprising an amino acid sequence set forth in SEQ ID NO: 129, or a sequence having at least 70%, 75%, 80%, 85%, 90%, 95%, 96%, 97%, 98%, or 99% identity to SEQ ID NO: 129, and a light chain variable domain comprising an amino acid sequence set forth in SEQ ID NO: 134, or a sequence having at least 70%, 75%, 80%, 85%, 90%, 92%, 94%, 95%, 96%, 97%, 98%, or 99% identity to SEQ ID NO: 134.

In one aspect, the present invention provides an isolated antibody, or an antigen-binding portion thereof, that binds to a ZIKV, comprising a heavy chain variable domain comprising an amino acid sequence set forth in SEQ ID NO: 129 and a light chain variable domain comprising an amino acid sequence set forth in SEQ ID NO: 134.

In one aspect, the present invention provides an isolated antibody, or an antigen-binding portion thereof, that binds to a ZIKV, comprising a heavy chain comprising an amino acid sequence set forth in SEQ ID NO: 128, or a sequence having at least 70%, 75%, 80%, 85%, 90%, 92%, 94%, 95%, 96%, 97%, 98%, or 99% identity to SEQ ID NO: 128, and a light chain comprising an amino acid sequence set forth in SEQ ID NO: 133, or a sequence having at least 70%, 75%, 80%, 85%, 90%, 92%, 94%, 95%, 96%, 97%, 98%, or 99% identity to SEQ ID NO: 133.

In one aspect, the present invention provides an isolated antibody, or an antigen-binding portion thereof, that binds to a ZIKV, comprising a heavy chain comprising an amino acid sequence set forth in SEQ ID NO: 128 and a light chain comprising an amino acid sequence set forth in SEQ ID NO: 133.

In one aspect, the present invention provides an antibody, or antigen-binding portion thereof, that binds an epitope on E-protein of a ZIKV as the isolated antibody, or antigen-binding portion thereof, as described herein.

In one aspect, the present invention provides an antibody, or antigen-binding portion thereof, that binds an epitope on E-protein of a ZIKV comprising an amino acid sequence of SEQ ID NO: 127, wherein the epitope comprises amino acids of T309, T335, G337 and S368 present in SEQ ID NO: 127.

In one aspect, the present invention provides an antibody, or antigen-binding portion thereof, that binds an epitope on E-protein of a ZIKV comprising an amino acid sequence of

SEQ ID NO: 127, wherein the epitope comprises amino acids of T369 and E370 present in SEQ ID NO: 127.

In one aspect, the present invention provides an antibody, or antigen-binding portion thereof, that binds an epitope on E-protein of a ZIKV comprising an amino acid sequence of SEQ ID NO: 127, wherein the epitope comprises amino acids of T335, S368, T369 and E370 present in SEQ ID NO: 127.

In one aspect, the present invention provides an antibody, or antigen-binding portion thereof, that binds an epitope on E-protein of a ZIKV comprising an amino acid sequence of SEQ ID NO: 127, wherein the epitope comprises amino acids of E162, G181, G182, K301 and G302 present in SEQ ID NO: 127.

In one aspect, the present invention provides an antibody, or antigen-binding portion thereof, that binds an epitope on E-protein of a ZIKV comprising an amino acid sequence of SEQ ID NO: 127, wherein the epitope comprises amino acids of 1317, T397, H398 and H399 present in SEQ ID NO: 127.

In certain aspects, an isolated antibody, or antigen-binding portion thereof, as described herein, neutralizes a viral infection caused by one or more ZIKV strains. In some embodiments, the one or more ZIKV strains are selected from the group consisting of ZikaSPH (Brazil 2015, KU321639.1), Brazil-ZKV (Brazil 2015, KU497555.1), PRVABC59 (Puerto Rico 2015, KU501215.1), Haiti1225 (Haiti 2014, KU509998.1), Natal RGN (Brazil, KU527068.1), SV0127-14 (Thailand 2014, KU681081.3), CPC-0740 (Philippine 2012, KU681082.3), SSABR1 (Brazil, KU707826.1), VE Ganxian (China, KU744693.1), MR766-NIID (Uganda, LC002520.1), MR 766 (Uganda 1947, AY632535.2), and H/PF (French Polynesia 2013, KJ776791.1) (WO 2017/109225, incorporated by reference). In some embodiments, the isolated antibody, or antigen-binding portion thereof, neutralizes a ZIKV in vitro with an IC₅₀ or an EC₅₀ less than or equal to about 10⁻⁹ M. In some embodiments, the isolated antibody, or antigen-binding portion thereof, demonstrates a protective effect in vivo in a ZIKV infected animal. In some embodiments, the isolated antibody, or antigen-binding portion thereof, does not contribute to ADE of a ZIKV infection.

In certain aspects, an isolated antibody, or antigen-binding portion thereof, as described herein, does not bind to a dengue virus.

In certain aspects, an isolated antibody, or antigen-binding portion thereof, as described herein, is a monoclonal antibody, or antigen-binding portion thereof. In some embodiments, wherein the isolated antibody, or antigen-binding portion thereof, is a recombinant monoclonal antibody, or antigen-binding portion thereof.

In certain aspects, an isolated antibody, or antigen-binding portion thereof, as described herein, is a human antibody, or antigen-binding portion thereof.

In certain aspects, an isolated antibody, or antigen-binding portion thereof, as described herein, is a humanized antibody, or antigen-binding portion thereof

In certain aspects, an isolated antibody, or antigen-binding portion thereof, as described herein, is of an IgG isotype. In some embodiments, the IgG isotype is IgG₁, IgG₂, IgG₃, or IgG₄. In some embodiments, the isolated antibody, or antigen-binding portion thereof, is an IgG₁ isotype. In some embodiments, the isolated antibody, or antigen-binding portion thereof, is an IgG₂ isotype. In some embodiments, the isolated antibody, or antigen-binding portion thereof, is an IgG₃ isotype. In some embodiments, the isolated antibody, or antigen-binding portion thereof, is an IgG₄ isotype.

In certain aspects, an isolated antibody, or antigen-binding portion thereof, as described herein, binds to a ZIKV with a dissociation constant (KD) of 100 nM or less.

In certain aspects, an isolated antibody, or antigen-binding portion thereof, as described herein, is a purified antibody, a single chain antibody, Fab, Fab′, F(ab′)₂, Fv or scFv.

In certain aspects, an isolated antibody, or antigen-binding portion thereof, as described herein, is labelled with a label. In some embodiments, the label is selected from the group consisting of biotin, horseradish peroxidase (HRP), alkaline phosphatase (AP), glucose oxidase, β-galactosidase, an Alexa dye, FITC, TRITC, a DyLight fluor, Green fluorescent protein (GFP), and R-phycoerythrin. In some embodiments, the label is biotin or horseradish peroxidase (HRP). In certain aspects, an isolated antibody, or antigen-binding portion thereof, as described herein, is a multi-specific antibody. In some embodiments, the isolated antibody, or antigen-binding portion thereof is a bispecific antibody.

In one aspect, the present invention provides a pharmaceutical composition comprising an isolated antibody, or antigen-binding portion thereof, as described herein, and a pharmaceutically acceptable excipient, carrier or diluent.

In some embodiments, the pharmaceutical composition is a lyophilized composition. In some embodiments, the pharmaceutical composition is a liquid composition. In some embodiments, the pharmaceutical composition is frozen. In some embodiments, the pharmaceutical composition is frozen at a temperature less than or equal to −65° C. In some embodiments, the pharmaceutical composition is provided as a single-dose product.

In one aspect, the present invention provides a syringe comprising an pharmaceutical composition, as described herein.

In one aspect, the present invention provides an isolated nucleic acid molecule encoding an isolated antibody, or antigen-binding portion thereof, as described herein. In one aspect, the present invention provides an isolated nucleic acid molecule encoding an antibody comprising a heavy chain variable region comprising a nucleic acid sequence of SEQ ID NO: 62, or a sequence having at least 70%, 75%, 80%, 85%, 90%, 92%, 94%, 95%, 96%, 97%, 98%, or 99% identity to SEQ ID NO: 62, and a light chain variable region comprising a nucleic acid sequence of SEQ ID NO: 64, or a sequence having at least 70%, 75%, 80%, 85%, 90%, 92%, 94%, 95%, 96%, 97%, 98%, or 99% identity to SEQ ID NO: 64.

In one aspect, the present invention provides an isolated nucleic acid molecule encoding an antibody comprising a heavy chain comprising a nucleic acid sequence of SEQ ID NO: 61, or a sequence having at least 70%, 75%, 80%, 85%, 90%, 92%, 94%, 95%, 96%, 97%, 98%, or 99% identity to SEQ ID NO: 61, and a light chain comprising a nucleic acid sequence of SEQ ID NO: 63, or a sequence having at least 70%, 75%, 80%, 85%, 90%, 92%, 94%, 95%, 96%, 97%, 98%, or 99% identity to SEQ ID NO: 63.

In one aspect, the present invention provides an isolated nucleic acid molecule encoding an antibody comprising a heavy chain variable region comprising a nucleic acid sequence of SEQ ID NO: 66, or a sequence having at least 70%, 75%, 80%, 85%, 90%, 92%, 94%, 95%, 96%, 97%, 98%, or 99% identity to SEQ ID NO: 66, and a light chain variable region comprising a nucleic acid sequence of SEQ ID NO: 68, or a sequence having at least 70%, 75%, 80%, 85%, 90%, 92%, 94%, 95%, 96%, 97%, 98%, or 99% identity to SEQ ID NO: 68.

In one aspect, the present invention provides an isolated nucleic acid molecule encoding an antibody comprising a heavy chain comprising a nucleic acid sequence of SEQ ID NO: 65, or a sequence having at least 70%, 75%, 80%, 85%, 90%, 92%, 94%, 95%, 96%, 97%, 98%, or 99% identity to SEQ ID NO: 65, and a light chain comprising a nucleic acid sequence of SEQ ID NO: 67, or a sequence having at least 70%, 75%, 80%, 85%, 90%, 92%, 94%, 95%, 96%, 97%, 98%, or 99% identity to SEQ ID NO: 67.

In one aspect, the present invention provides an isolated nucleic acid molecule encoding an antibody comprising a heavy chain variable region comprising a nucleic acid sequence of SEQ ID NO: 70, or a sequence having at least 70%, 75%, 80%, 85%, 90%, 92%, 94%, 95%, 96%, 97%, 98%, or 99% identity to SEQ ID NO: 70, and a light chain variable region comprising a nucleic acid sequence of SEQ ID NO: 72, or a sequence having at least 70%, 75%, 80%, 85%, 90%, 92%, 94%, 95%, 96%, 97%, 98%, or 99% identity to SEQ ID NO: 72.

In one aspect, the present invention provides an isolated nucleic acid molecule encoding an antibody comprising a heavy chain comprising a nucleic acid sequence of SEQ ID NO: 69, or a sequence having at least 70%, 75%, 80%, 85%, 90%, 92%, 94%, 95%, 96%, 97%, 98%, or 99% identity to SEQ ID NO: 69, and a light chain comprising a nucleic acid sequence of SEQ ID NO: 71, or a sequence having at least 70%, 75%, 80%, 85%, 90%, 92%, 94%, 95%, 96%, 97%, 98%, or 99% identity to SEQ ID NO: 71.

In one aspect, the present invention provides an isolated nucleic acid molecule encoding an antibody comprising a heavy chain variable region comprising a nucleic acid sequence of SEQ ID NO: 74, or a sequence having at least 70%, 75%, 80%, 85%, 90%, 92%, 94%, 95%, 96%, 97%, 98%, or 99% identity to SEQ ID NO: 74, and a light chain variable region comprising a nucleic acid sequence of SEQ ID NO: 76, or a sequence having at least 70%, 75%, 80%, 85%, 90%, 92%, 94%, 95%, 96%, 97%, 98%, or 99% identity to SEQ ID NO: 76.

In one aspect, the present invention provides an isolated nucleic acid molecule encoding an antibody comprising a heavy chain comprising a nucleic acid sequence of SEQ ID NO: 73, or a sequence having at least 70%, 75%, 80%, 85%, 90%, 92%, 94%, 95%, 96%, 97%, 98%, or 99% identity to SEQ ID NO: 73, and a light chain comprising a nucleic acid sequence of SEQ ID NO: 75, or a sequence having at least 70%, 75%, 80%, 85%, 90%, 92%, 94%, 95%, 96%, 97%, 98%, or 99% identity to SEQ ID NO: 75.

In one aspect, the present invention provides an isolated nucleic acid molecule encoding an antibody comprising a heavy chain variable region comprising a nucleic acid sequence of SEQ ID NO: 78, or a sequence having at least 70%, 75%, 80%, 85%, 90%, 92%, 94%, 95%, 96%, 97%, 98%, or 99% identity to SEQ ID NO: 78, and a light chain variable region comprising a nucleic acid sequence of SEQ ID NO: 80, or a sequence having at least 70%, 75%, 80%, 85%, 90%, 92%, 94%, 95%, 96%, 97%, 98%, or 99% identity to SEQ ID NO: 80.

In one aspect, the present invention provides an isolated nucleic acid molecule encoding an antibody comprising a heavy chain comprising a nucleic acid sequence of SEQ ID NO: 77, or a sequence having at least 70%, 75%, 80%, 85%, 90%, 92%, 94%, 95%, 96%, 97%, 98%, or 99% identity to SEQ ID NO: 77, and a light chain comprising a nucleic acid sequence of SEQ ID NO: 79, or a sequence having at least 70%, 75%, 80%, 85%, 90%, 92%, 94%, 95%, 96%, 97%, 98%, or 99% identity to SEQ ID NO: 79.

In one aspect, the present invention provides an isolated nucleic acid molecule encoding an antibody comprising a heavy chain variable region comprising a nucleic acid sequence of SEQ ID NO: 82, or a sequence having at least 70%, 75%, 80%, 85%, 90%, 92%, 94%, 95%, 96%, 97%, 98%, or 99% identity to SEQ ID NO: 82, and a light chain variable region comprising a nucleic acid sequence of SEQ ID NO: 84, or a sequence having at least 70%, 75%, 80%, 85%, 90%, 92%, 94%, 95%, 96%, 97%, 98%, or 99% identity to SEQ ID NO: 84.

In one aspect, the present invention provides an isolated nucleic acid molecule encoding an antibody comprising a heavy chain comprising a nucleic acid sequence of SEQ ID NO: 81, or a sequence having at least 70%, 75%, 80%, 85%, 90%, 92%, 94%, 95%, 96%, 97%, 98%, or 99% identity to SEQ ID NO: 81, and a light chain comprising a nucleic acid sequence of SEQ ID NO: 83, or a sequence having at least 70%, 75%, 80%, 85%, 90%, 92%, 94%, 95%, 96%, 97%, 98%, or 99% identity to SEQ ID NO: 83.

In one aspect, the present invention provides an isolated nucleic acid molecule encoding an antibody comprising a heavy chain variable region comprising a nucleic acid sequence of SEQ ID NO: 116, or a sequence having at least 70%, 75%, 80%, 85%, 90%, 92%, 94%, 95%, 96%, 97%, 98%, or 99% identity to SEQ ID NO: 116, and a light chain variable region comprising a nucleic acid sequence of SEQ ID NO: 118, or a sequence having at least 70%, 75%, 80%, 85%, 90%, 92%, 94%, 95%, 96%, 97%, 98%, or 99% identity to SEQ ID NO: 118.

In one aspect, the present invention provides an isolated nucleic acid molecule encoding an antibody comprising a heavy chain comprising a nucleic acid sequence of SEQ ID NO: 115, or a sequence having at least 70%, 75%, 80%, 85%, 90%, 92%, 94%, 95%, 96%, 97%, 98%, or 99% identity to SEQ ID NO: 115, and a light chain comprising a nucleic acid sequence of SEQ ID NO: 117, or a sequence having at least 70%, 75%, 80%, 85%, 90%, 92%, 94%, 95%, 96%, 97%, 98%, or 99% identity to SEQ ID NO: 117.

In one aspect, the present invention provides an isolated nucleic acid molecule encoding an antibody comprising a heavy chain variable region comprising a nucleic acid sequence of SEQ ID NO:120, or a sequence having at least 70%, 75%, 80%, 85%, 90%, 92%, 94%, 95%, 96%, 97%, 98%, or 99% identity to SEQ ID NO: 120, and a light chain variable region comprising a nucleic acid sequence of SEQ ID NO: 122, or a sequence having at least 70%, 75%, 80%, 85%, 90%, 92%, 94%, 95%, 96%, 97%, 98%, or 99% identity to SEQ ID NO: 122.

In one aspect, the present invention provides an isolated nucleic acid molecule encoding an antibody comprising a heavy chain comprising a nucleic acid sequence of SEQ ID NO: 119, or a sequence having at least 70%, 75%, 80%, 85%, 90%, 92%, 94%, 95%, 96%, 97%, 98%, or 99% identity to SEQ ID NO: 119, and a light chain comprising a nucleic acid sequence of SEQ ID NO: 121, or a sequence having at least 70%, 75%, 80%, 85%, 90%, 92%, 94%, 95%, 96%, 97%, 98%, or 99% identity to SEQ ID NO: 121.

In one aspect, the present invention provides an isolated nucleic acid molecule encoding an antibody comprising a heavy chain variable region comprising a nucleic acid sequence of SEQ ID NO: 124, or a sequence having at least 70%, 75%, 80%, 85%, 90%, 92%, 94%, 95%, 96%, 97%, 98%, or 99% identity to SEQ ID NO: 124, and a light chain variable region comprising a nucleic acid sequence of SEQ ID NO: 126, or a sequence having at least 70%, 75%, 80%, 85%, 90%, 92%, 94%, 95%, 96%, 97%, 98%, or 99% identity to SEQ ID NO: 126.

In one aspect, the present invention provides an isolated nucleic acid molecule encoding an antibody comprising a heavy chain comprising a nucleic acid sequence of SEQ ID NO: 123, or a sequence having at least 70%, 75%, 80%, 85%, 90%, 92%, 94%, 95%, 96%, 97%, 98%, or 99% identity to SEQ ID NO: 123, and a light chain comprising a nucleic acid sequence of SEQ ID NO: 125, or a sequence having at least 70%, 75%, 80%, 85%, 90%, 92%, 94%, 95%, 96%, 97%, 98%, or 99% identity to SEQ ID NO: 125.

In one aspect, the present invention provides an isolated nucleic acid molecule encoding an antibody comprising a heavy chain variable region comprising a nucleic acid sequence of SEQ ID NO: 139, or a sequence having at least 70%, 75%, 80%, 85%, 90%, 92%, 94%, 95%, 96%, 97%, 98%, or 99% identity to SEQ ID NO: 139, and a light chain variable region comprising a nucleic acid sequence of SEQ ID NO: 141, or a sequence having at least 70%, 75%, 80%, 85%, 90%, 92%, 94%, 95%, 96%, 97%, 98%, or 99% identity to SEQ ID NO: 141.

In one aspect, the present invention provides an isolated nucleic acid molecule encoding an antibody comprising a heavy chain comprising a nucleic acid sequence of SEQ ID NO: 138, or a sequence having at least 70%, 75%, 80%, 85%, 90%, 92%, 94%, 95%, 96%, 97%, 98%, or 99% identity to SEQ ID NO: 138, and a light chain comprising a nucleic acid sequence of SEQ ID NO: 140, or a sequence having at least 70%, 75%, 80%, 85%, 90%, 92%, 94%, 95%, 96%, 97%, 98%, or 99% identity to SEQ ID NO: 140. In some embodiments, any one of the nucleic acid sequences described herein may or may not include a leader sequence, which encodes a signal peptide.

In one aspect, the present invention provides a vector comprising a nucleic acid molecule, as described herein.

In one aspect, the present invention provides an isolated cell expressing the nucleic acid molecule or an vector, as described herein.

In one aspect, the present invention provides a kit comprising an isolated antibody, or antigen-binding portion thereof, as described herein, and instructions for use.

In one aspect, the present invention provides a method of neutralizing an infectious ZIKV, the method comprising exposing a cell infected with an infectious ZIKV to an isolated antibody, or antigen-binding portion thereof, as described herein, wherein the exposing results in enhanced protection of the cell from viral infection, or from cell death.

In some embodiments, the enhanced protection is observed when the isolated antibody is used alone, or when it is used in combination with at least one additional therapeutic agent or anti-ZIKV treatment modality. In some embodiments, the additional therapeutic agent is selected from the group consisting of an anti-viral drug, an anti-inflammatory drug, a different antibody to the infectious ZIKV, a vaccine for the infectious ZIKV, an immunomodulator and an interferon. In some embodiments, the additional therapeutic agent is selected from the group consisting of corticosteroids and non-steroidal anti-inflammatory drugs.

In one aspect, the present invention provides a method of preventing, treating or ameliorating at least one symptom of a ZIKV infection, or of decreasing the frequency or severity of at least one symptom of a ZIKV infection, the method comprising administering an effective amount of at least one isolated antibody, or antigen-binding portion thereof, as described herein, or a pharmaceutical composition, as described herein, to a subject in need thereof.

In some embodiments, the at least one symptom is selected from the group consisting of fever, headache, arthralgia, myalgia and a maculopapular rash. In some embodiments, the subject in need thereof is at risk for exposure to, or for acquiring a ZIKV infection and the subject is selected from the group consisting of a pregnant woman who has been exposed to a ZIKV, or who has been bitten by a mosquito suspected of harboring a ZIKV, a woman who lives in an area that has an outbreak of a ZIKV, or is visiting an area that has an outbreak of a ZIKV and who is considering conceiving a child, an immunocompromised individual, a person who is suspected of having been exposed to a person harboring a ZIKV, a person who comes into physical contact or close physical proximity with a ZIKV-infected individual, a hospital employee, a pharmaceutical researcher, maintenance personnel responsible for cleaning a hospital facility or institution where a ZIKV-infected patient has been treated, individuals who have visited or are planning to visit an area or country known to have or suspected to have an outbreak of a ZIKV, or a country known to have mosquitoes harboring a ZIKV.

In one aspect, the present invention provides a method of decreasing the likelihood of transmitting a ZIKV infection to the fetus of a pregnant female, and/or prevention of transmission to the male reproductive organs, the method comprising administering to a subject in need thereof an effective amount of at least one isolated antibody, or antigen-binding portion, as described herein, or a pharmaceutical composition as described herein, to a subject in need thereof.

In some embodiments, the isolated antibody, or antigen-binding portion thereof, or the pharmaceutical composition, is administered prophylactically or therapeutically to the subject in need thereof. In some embodiments, the isolated antibody, or antigen-binding portion thereof, or the pharmaceutical composition, is administered in combination with a second therapeutic agent, wherein the second therapeutic agent is selected from the group consisting of an anti-viral drug, an anti-inflammatory drug, a different antibody to a ZIKV, a vaccine for a ZIKV, an immunomodulator, and an interferon. In some embodiments, the anti-inflammatory drug is selected from corticosteroids and non-steroidal anti-inflammatory drugs. In some embodiments, the isolated antibody or antigen-binding portion thereof, or the pharmaceutical composition, is administered subcutaneously, intravenously, intradermally, intramuscularly, intranasally, or orally. In some embodiments, the effective amount of the isolated antibody, or antigen-binding portion thereof, does not exceed 750 mg, preferably does not exceed 500 mg, more preferably does not exceed 250 mg, and even more preferably does not exceed 100 mg. In some embodiments, the isolated antibody, or antigen-binding portion thereof, is administered at a (single) dose of about 0.001 to about 60 mg/kg body weight, preferably about 0.001 to about 1, about 1 to about 5, about 5 to about 10, about 10 to about 20, or about 20 to about 60 mg/kg body weight. The average body weight of a human subject is 60 to 80 kg. All the embodiments disclosed herein can be further combined with one another.

In one aspect, the present invention provides a method of in vitro diagnosing a ZIKV infection in a subject, comprising: (i) contacting a sample isolated from a subject with a plate coated with a ZIKV E protein, VLP, virus or inactivated virus and incubating the sample on the plate for a period of time; (ii) contacting the sample having been incubated on the plate in step (i) with an isolated antibody, or antigen-binding portion thereof, as described herein, and further incubating for a period of time; and (iii) determining inhibition of binding of the isolated antibody, or antigen-binding portion thereof, to the plate, wherein the inhibition of the isolated antibody, or antigen-binding portion thereof, to the plate reflects the presence of an anti-ZIKV antibody in the sample; and wherein the presence of the anti-ZIKV antibody in the sample indicates a ZIKV infection in the subject, thereby diagnosing a ZIKV infection in the subject.

In some embodiments, the sample is selected from the group consisting of blood, saliva and urine; preferably the sample is blood, such as whole blood, plasma or serum. In some embodiments, the isolated antibody, or antigen-binding portion thereof, added in step (ii), is labelled with a label. In some embodiments, the label is selected from the group consisting of biotin, horseradish peroxidase (HRP), alkaline phosphatase (AP), glucose oxidase, β-galactosidase, an Alexa dye, FITC, TRITC, a DyLight fluor, Green fluorescent protein (GFP), and R-phycoerythrin. In some embodiments, the label is biotin or horseradish peroxidase (HRP). In some embodiments, the sample isolated from the subject is diluted, for example 1:5-1:50, preferably, 1:5-1:25, more preferably, 1:10. In some embodiments, the period of time for incubation in step (i) is at least 5 minutes, preferably at least 15 minutes, more preferably at least 30 minutes, even more preferably at least 45 minutes and most preferably at least 60 minutes. In some embodiments, the period of time for further incubation in step (iii) is at least 1 minute, preferably, at least 3 minutes, more preferably at least 5 minutes, even more preferably at least 10 minutes and most preferably at least 15 minutes.

In one aspect, the present invention provides a method of determining the potency/concentration of a test anti-ZIKV antibody, comprising: (a) coating a well of a plate with a purified inactivated Zika virus (PIZV); (b) incubating the PIZV coated on the well with one of a plurality of solutions of a test antibody, wherein the plurality of solutions of the test antibody are prepared by a serial dilution of a concentrated solution of the test antibody; (c) incubating the test antibody, which binds to the PIZV, with a secondary antibody, wherein the binding of the secondary antibody to the isolated antibody produces a signal; (d) detecting the signal; (e) repeating steps (a)-(d) with the remaining of the plurality of solutions prepared in step (b); and (f) ploting a dilution curve with the signal of the secondary antibody against the concentration of each of the plurality of solutions prepared in step (b), wherein an IC₅₀ of the dilution curve indicates the potency of the test antibody.

In one aspect, the present invention provides a method of determining the potency/concentration of a test PIZV, comprising: (a) coating a well of a plate with a first anti-ZIKV antibody which binds to a first epitope on a test PIZV and captures the test PIZV; (b) incubating the coated well with the test PIZV; (c) further incubating the coated well with one of a plurality of solutions of a second anti-ZIKV antibody which binds to a second epitope on the test PIVZ, wherein the plurality of solutions of the second anti-ZIKV antibody are prepared by a serial dilution of a concentrated solution of the second anti-ZIKV antibody; (d) further incubating the coated well with a secondary antibody conjugated to an enzyme or a reporter, wherein the binding of the secondary antibody to the second anti-ZIKV antibody produces a signal; (e) detecting the signal; (f) repeating steps (a)-(e) with the remaining of the plurality of solutions of the second anti-ZIKV antibody prepared in step (c); and (g) ploting a dilution curve with the signal of the secondary anti-ZIKV antibody against the concentration of each of the plurality of solutions of the second anti-ZIKV antibody prepared in step (c), wherein an IC₅₀ of the dilution curve indicates the potency of the test PIZV, wherein at least one of the first anti-ZIKV antibody and the second anti-ZIKV antibody is any one of the isolated neutralizing antibodies described herein; and wherein the first epitope does not overlap with the second epitope. In one embodiment, the second anti-ZIKV antibody is any one of the isolated neutralizing antibodies described herein.

### BRIEF DESCRIPTION OF THE DRAWINGS

FIG. 1. Comparison of inhouse Mabs with 4G2 and C10 at Normalized Concentration by MNT. 10 μg/ml of each antibody was serially diluted and subjected to MNT testing against live Zika Virus. Relative infectivity was plotted against antibody dilutions. IC₅₀ values were calculated from the curve data.

FIGS. 2A and 2B. Comparison of inhouse Mabs with 4G2 and C10 at Normalized Concentration by Indirect ELISA. 10 μg/ml of each antibody was serially diluted and tested for binding against either ZIKV-VLP (FIG. 2A) or PIZV (FIG. 2B). Absorbances were plotted against antibody dilutions and a sigmoidal curve was generated. IC₅₀ values were calculated from the plot.

FIG. 3. Epitope binning of hybridoma subclone supernatants with schematics of binning steps. Red circle with blue spikes indicate VLP. 1st and 2nd mabs are represented with different colors. Simultaneous binding of two antibodies results in increased signal as shown by a plot (top right), competition with each other does not result in signal increase as shown by a flat line (bottom right).

FIG. 4. Competition binning of hybridoma supernatant set with sensorgrams representing binding or nonbinding of 2nd antibody after the 1st antibody is bound. Increased in signal indicates binding at different epitopes, flat lines or no increase in signal indicate competition for the same epitope.

FIG. 5. Ideal sandwich ELISA antibody pairs based on competition assay of binning with three-dimensional structure of Zika virus as shown. Small empty circles indicate different epitopes. Larger empty circles represent the clones binding to same epitope. The right panel shows the sandwich ELISA pairs as either a capture or a detection mab.

FIG. 6. Hierarchical analysis of clustering for binning experiment of top 5 clones. Respond units are indicated crossed between the 1^{st} mAb and 2^{nd} mAb.

FIG. 7. Constellation plot of epitope binning from the 5 clones.

FIG. 8. Hierarchical clustering of epitope binning from the 5 clones.

FIG. 9. Hierarchical analysis of clustering for binning experiment of top 10 clones. Respond units are indicated crossed between the Pt mAb and 2nd mAb.

FIG. 10. Constellation plot of epitope binning from the 10 clones.

FIG. 11. Western blot screening of top 6 subclones. Numbers in the left indicate the position of the molecular weight markers. Arrows indicate the position of E or PrM protein. Lane 1 and 2 are positive controls (anti-E protein mabs DAK or IBT) for identification of E protein. Lanes 3-11 show the mabs that were screened. All the antibodies were tested against ZIKV-VLP.

FIGS. 12A-G. SDS-PAGE (12% ClearPage gel) analysis of antibody stocks after purification. A) 102-1, B) 306-2, C) 270-12, D) 289-3, E) 242-3, F) 11-3, and G) 78-2.

FIGS. 13A and B. Western blot analysis under nonreducing and denaturing conditions. Numbers in the left indicate the position of molecular weight markers. Bands in each lane show the size of E protein. Except 289-3, all other clones show binding to E protein. Purified mAbs binding activity to Zika VLP protein (FIG. 13A) and purified mAbs binding activity to Zika PRVABC59 live virus (FIG. 13B).

FIG. 14. Visualization of critical residues for Fab binding in crystal structure of E protein dimers of 5 clones (102-1, 289-3, 242-3, 270-12 and 306-2). Critical residues (light spheres, i.e., all the spheres except for 5368) were visualized on a crystal structure of the target protein (PDB ID: SIRE, Sirohi et al, 2016). Secondary residue (dark sphere, i.e., S368) that may contribute to binding are also shown.

### DETAILED DESCRIPTION

Various aspects of the disclosure relate to anti-ZIKV antibodies, or antigen-binding portions thereof, and pharmaceutical compositions thereof, as well as nucleic acids, recombinant expression vectors and host cells for making such antibodies, or antigen-binding portion thereof. Further aspects of the disclosure relate to methods of using the isolated antibodies as described herein to treat a ZIKV infection and diagnose a ZIKV infection.

### I. Definitions

In order that the present invention may be more readily understood, certain terms are first defined. In addition, it should be noted that whenever a value or range of values of a parameter are recited, it is intended that values and ranges intermediate to the recited values are also intended to be part of this present invention.

“Zika virus”, or “ZIKV” is a member of the Flaviviridae family, and has been associated with microcephaly and other developmental abnormalities in the fetuses of pregnant women exposed to the virus (Schuler-Faccini, L. et al., (2016), MMWR Morb. Mortal. W ly Rep. 65:59-62, incorporated herein by reference) and has also been associated with Guillian-Barre syndrome in adults (Cao-Lormeau, VM, et al., (2016), Lancet, April 9; 387(10027):1531-9, incorporated herein by reference). The term “ZIKV” also includes variants of ZIKV isolated from different ZIKV isolates, including ZikaSPH (Brazil 2015, KU321639.1), Brazil-ZKV (Brazil 2015, KU497555.1), PRVABC59 (Puerto Rico 2015, KU501215.1), Haiti1225 (Haiti 2014, KU509998.1), Natal RGN (Brazil, KU527068.1), SV0127-14 (Thailand 2014, KU681081.3), CPC-0740 (Philippine 2012, KU681082.3), SSABR1 (Brazil, KU707826.1), VE Ganxian (China, KU744693.1), MR766-NIID (Uganda, LC002520.1), MR 766 (Uganda 1947, AY632535.2), and H/PF (French Polynesia 2013, KJ776791.1) (WO 2017/109225, incorporated by reference). The term “ZIKV” also includes Cambodia 2010 (JN860885) or Micronesia 2007 (EU545988) (Mlakar et al., N Engl J Med. 2016 Mar 10; 374(10):951-8, incorporated by reference). Further, the term “ZIKV” includes FLR (Colombia 2015) strain (WO 2018/017497, incorporated by reference).

The amino acid sequence of ZIKV envelope glycoprotein (E), noted herein as “ZIKV E protein” can be found in Gen Bank as accession number AWH65849.1 as part of viral protein and corresponds to amino acids 291-794 and provided below as SEQ ID NO: 127.

(SEQ ID NO: 127)

IRCIGVSNRD FVEGMSGGTW VDVVLEHGGC VTVMAQDKPT
VDIELVTTTV SNMAEVRSYC YEASISDMAS DSRCPTQGEA
YLDKQSDTQY VCKRTLVDRG WGNGCGLFGK GSLVTCAKFA
CSKKMTGKSI QPENLEYRIM LSVHGSQHSG MIVNDTGHET
DENRAKVEIT PNSPRAEATL GGFGSLGLDC EPRTGLDFSD
LYYLTMNNKH WLVHKEWFHD IPLPWHAGAD TGTPHWNNKE
ALVEFKDAHA KRQTVVVLGS QEGAVHTALA GALEAEMDGA
KGRLSSGHLK CRLKMDKLRL KGVSYSLCTA AFTFTKIPAE
TLHGTVTVEV QYAGTDGPCK VPAQMAVDMQ TLTPVGRLIT
ANPVITESTE NSKMMLELDP PFGDSYIVIG VGEKKITHHW
HRSGSTIGKA FEATVRGAKR MAVLGDTAWD FGSVGGALNS
LGKGIHQIFG AAFKSLFGGM SWFSQILIGT LLMWLGLNTK
NGSISLMCLA LGGVLIFLST AVSA

The term “ZIKV infection”, as used herein refers to the disease or condition resulting from exposure to the virus (e.g. after being bitten by a mosquito harboring the virus), or to an infected animal, or to an infected human patient, or contact with the bodily fluids or tissues from an animal or human patient having a ZIKV infection. The “symptoms associated with a ZIKV infection” include fever, headache, arthralgia, myalgia and a maculopapular rash. The “condition resulting from exposure to the virus”, or “the condition associated with exposure to the virus” also includes microcephaly (or developmental abnormalities) of a fetus in a pregnant woman who was infected with the virus after being bitten by a mosquito harboring the virus. Another “condition resulting from exposure to the virus”, or “condition associated with exposure to the virus” includes Guillain-Barre Syndrome.

An antibody “which binds” an antigen of interest, e.g., a ZIKV E protein, is one capable of binding that antigen with sufficient affinity such that the antibody is useful in targeting a cell expressing the antigen. In a preferred embodiment, the antibody specifically binds to a ZIKV E protein. Examples of anti-ZIKV antibodies are disclosed in the Examples, below. Unless otherwise indicated, the term “anti-ZIKV antibody”, “antibody of the present invention”, or “antibody of the invention” used herein is meant to refer to an antibody which binds to a ZIKV.

The terms “specific binding”, “specifically binding”, “specifically binds”, or “binds specifically to” as used herein, means that an antibody or antigen-binding portion thereof forms a complex with an antigen that is relatively stable under physiologic conditions. The interaction of an anti-ZIKV antibody with the antigen is dependent upon the presence of a particular structure (e.g., an antigenic determinant or epitope) on the antigen; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound thereto.

In one embodiment, the phrase “binds to a ZIKV”, “specifically binds to a ZIKV” or “specific binding to a ZIKV”, as used herein, refers to the ability of an anti-ZIKV antibody interact with a ZIKV with a dissociation constant (KD) of between about 1 pM (0.001 nM) to 1,000 nM, between about 500 pM (0.5 nM) to 1,000 nM, between about 500 pM (0.5 nM) to 500 nM, between about 1 nM to 200 nM, between about 1 nM to 100 nM, between about 1 nM to 50 nM, between about 1 nM to 20 nM, or between about 1 nM to 5 nM as measured by surface plasmon resonance, e.g., BIACORE™ or solution-affinity ELISA. The term “high affinity” antibody refers to those mAbs having a binding affinity to ZIKV, expressed as KD, of at least 10⁻⁷ M; preferably 10 nM; more preferably 1 nM, even more preferably 0.1 nM, even more preferably 0.01 nM, even more preferably 0.001 nM.

The term “KD”, as used herein, is intended to refer to the equilibrium dissociation constant of a particular antibody-antigen interaction. KD is calculated by kₐ/k_{d}. In one embodiment, the antibodies, or antigen-binding portions thereof, according to the present invention have a KD of about 1,000 nM or less, about 1,000 nM or less, about 500 nM or less, about 200 nM or less, about 100 nM or less, about 75 nM or less, about 50 nM or less, about 25 nM or less, about 21 nM or less, about 12 nM or less, about 11 nM or less, about 10 nM or less, about 9 nM or less, about 8 nM or less, about 7 nM or less, about 6 nM or less, about 5 nM or less, about 4 nM or less, about 3 nM or less, about 2 nM or less, about 1 nM or less, about 0.5 nM or less, about 0.3 nM or less, about 0.1 nM or less, about 0.01 nM or less, or about 0.001 nM or less.

The term “kₐ” or “kₒₙ”, as used herein, is intended to refer to the on rate constant for association of an antibody to the antigen to form the antibody/antigen complex. The term “ k_{d}” or “k_{off}”, as used herein, is intended to refer to the off rate constant for dissociation of an antibody from the antibody/antigen complex. By the term “slow off rate”, “ k_{d}” or “k_{off}” is meant an antibody that dissociates from a ZIKV, or a ZIKV-VLP expressing a ZIKV E protein, with a rate constant of 1×10⁻³ s⁻¹ or less, preferably 1×10⁻⁴ s⁻¹ or less, as determined by surface plasmon resonance, e.g., BIACORE™ or Bio-Layer Interferometry.

In one embodiment, K_{d} is determined by surface plasmon resonance or Bio-Layer Interferometry, or by any other method known in the art. The term “surface plasmon resonance”, as used herein, refers to an optical phenomenon that allows for the analysis of real-time biomolecular interactions by detection of alterations in protein concentrations within a biosensor matrix, for example using the BIACORE™ system (Pharmacia Biosensor AB, Uppsala, Sweden and Piscataway, N.J.). Bio-Layer Interferometry refers to an optical phenomenon that allows for the analysis of real-time biospecific interactions by measuring the interference patterns of reflected white light, for example using the Octet™ system (ForteBio, Pall Corp. Fremont, Calif.). For further description of the Octet™ system, see Li, B et al. (2011) J. Pharm. Biomed. Anal. 54(2):286-294 and Abdiche, Y.N., et al. (2009) Anal. Biochem. 386(2):172-180, each incorporated herein by reference.

The antibodies, or antigen-binding portions, according to the present invention may be mono-specific, bi-specific, or multi-specific. Multi-specific antibodies may contain antigen-binding domains specific for different epitopes of one antigen or may contain antigen-binding domains specific for two or more unrelated antigens. See, e.g., Tutt et al., 1991, J. Immunol. 147:60-69; Kufer et al., 2004, Trends Biotechnol. 22:238-244. Any of the multi-specific antigen-binding molecules of the present invention, or variants thereof, may be constructed using standard molecular biological techniques (e.g., recombinant DNA and protein expression technology), as will be known to a person of ordinary skill in the art.

The term “multivalent antibody” is used herein to denote an antibody comprising two or more antigen binding sites. In certain embodiments, the multivalent antibody may be engineered to have the three or more antigen binding sites, and is generally not a naturally occurring antibody.

The term “dual variable domain” or “DVD,” as used interchangeably herein, are antigen binding proteins that comprise two or more antigen binding sites and are tetravalent or multivalent binding proteins. Such DVDs may be monospecific, i.e., capable of binding one antigen, or multispecific, i.e. capable of binding two or more antigens. DVD binding proteins comprising two heavy chain DVD polypeptides and two light chain DVD polypeptides are referred to a DVD Ig. Each half of a DVD Ig comprises a heavy chain DVD polypeptide, and a light chain DVD polypeptide, and two antigen binding sites. Each binding site comprises a heavy chain variable domain and a light chain variable domain with a total of 6 CDRs involved in antigen binding per antigen binding site. In one embodiment, the CDRs described herein are used in an anti-ZIKV DVD.

In some embodiments, ZIKV-specific antibodies are generated in a bi-specific format (a “bi-specific”) in which variable regions binding to distinct domains of ZIKV are linked together to confer dual-domain specificity within a single binding molecule. Appropriately designed bi-specifics may enhance overall ZIKV-infection inhibitory efficacy through increasing both specificity and binding avidity. Variable regions with specificity for individual domains (e.g., segments of the N-terminal domain), or that can bind to different regions within one domain, are paired on a structural scaffold that allows each region to bind simultaneously to the separate epitopes, or to different regions within one domain. In one example for a bi-specific, heavy chain variable regions (VH) from a binder with specificity for one domain are recombined with light chain variable regions (VL) from a series of binders with specificity for a second domain to identify non-cognate VL partners that can be paired with an original VH without disrupting the original specificity for that VH. In this way, a single VL segment (e.g., VL1) can be combined with two different VH domains (e.g., VH1 and VH2) to generate a bi-specific comprised of two binding “arms” (VH1-VL1 and VH2-VL1). Use of a single VL segment reduces the complexity of the system and thereby simplifies and increases efficiency in cloning, expression, and purification processes used to generate the bi-specific (see, for example, US 2011/0195454 and US2010/0331527, each incorporated herein by reference).

Alternatively, antibodies that bind more than one domain and a second target, such as, but not limited to, for example, a second different anti-ZIKV antibody, may be prepared in a bi-specific format using techniques described herein, or other techniques known to those skilled in the art. Antibody variable regions binding to distinct regions may be linked together with variable regions that bind to relevant sites on, for example, ZIKV, to confer dual-antigen specificity within a single binding molecule. Appropriately designed bi-specifics of this nature serve a dual function. Variable regions with specificity for one domain are combined with a variable region with specificity for another domain and are paired on a structural scaffold that allows each variable region to bind to the separate antigens.

The term “antibody” broadly refers to an immunoglobulin (Ig) molecule, generally comprised of four polypeptide chains, two heavy (H) chains and two light (L) chains interconnected by disulfide bonds (i.e., “full antibody molecules”), or any functional fragment, mutant, variant, or derivative thereof, that retains the essential antigen-binding features of an Ig molecule. Such mutant, variant, or derivative antibody formats are known in the art. Non-limiting embodiments of which are discussed below.

In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. In certain embodiments of the present invention, the FRs of the antibody (or antigen binding fragment thereof) may be identical to the human germline sequences, or may be naturally or artificially modified.

Substitution of one or more CDR residues or omission of one or more CDRs is also possible. Antibodies have been described in the scientific literature in which one or two CDRs can be dispensed with for binding. Padlan et al. (1995 FASEB J. 9: 133-139, incorporated herein by reference) analyzed the contact regions between antibodies and their antigens, based on published crystal structures, and concluded that only about one fifth to one third of CDR residues actually contact the antigen. Padlan also found many antibodies in which one or two CDRs had no amino acids in contact with an antigen (see also, Vajdos et al. 2002 J Mol Biol 320:415-428, incorporated herein by reference). CDR residues not contacting antigen can be identified based on previous studies (for example residues H60-H65 in CDRH2 are often not required), from regions of Kabat CDRs lying outside Chothia CDRs, by molecular modeling and/or empirically. If a CDR or residue(s) thereof is omitted, it is usually substituted with an amino acid occupying the corresponding position in another human antibody sequence or a consensus of such sequences. Positions for substitution within CDRs and amino acids to substitute can also be selected empirically. Empirical substitutions can be conservative or non-conservative substitutions.

Immunoglobulin molecules can be of any type (e.g., IgG, IgE, IgM, IgD, IgA and IgY) and class (e.g., IgG₁, IgG₂, IgG₃, IgG₄, IgA₁ and IgA₂) or subclass.

The term “antigen-binding portion” of an antibody (or simply “antibody portion”) or “antigen-binding fragment” of an antibody, as used herein, refers to one or more fragments of an antibody that retain the ability to specifically bind to an antigen (e.g., a ZIKV). The terms “antigen-binding portion” of an antibody and “antigen-binding fragment” of an antibody, as used herein, include any naturally occurring, enzymatically obtainable, synthetic, or genetically engineered polypeptide or glycoprotein that specifically binds an antigen to form a complex. Such antibody embodiments may also be bispecific, dual specific, or multi-specific formats; specifically binding to two or more different antigens. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546, Winter et al., PCT publication WO 90/05144 Al, each incorporated herein by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883, incorporated herein by reference). Such single chain antibodies are also intended to be encompassed within the term “antigen-binding portion” of an antibody. In certain embodiments, scFv molecules may be incorporated into a fusion protein. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123, each incorporated herein by reference). Such antibody binding portions are known in the art (Kontermann and Dubel eds., Antibody Engineering (2001) Springer-Verlag. New York. 790 pp. (ISBN 3-540-41354-5) incorporated herein by reference).

The present invention encompasses an anti-ZIKV monoclonal antibody conjugated to a therapeutic moiety (“immunoconjugate”), such as an anti-viral drug to treat ZIKV infection. As used herein, the term “immunoconjugate” refers to an antibody, which is chemically or biologically linked to a radioactive agent, a cytokine, an interferon, a target or reporter moiety, an enzyme, a peptide o r protein or a therapeutic agent. The antibody may be linked to the radioactive agent, cytokine, interferon, target or reporter moiety, enzyme, peptide or therapeutic agent at any location along the molecule so long as it is able to bind its target. Examples of immunoconjugates include antibody drug conjugates and antibody-toxin fusion proteins. In one embodiment, the agent may be a second different antibody to a ZIKV, or a ZIKV E protein. In certain embodiments, the antibody may be conjugated to an agent specific for a virally infected cell. The type of therapeutic moiety that may be conjugated to the anti-ZIKV antibody and will take into account the condition to be treated and the desired therapeutic effect to be achieved. Examples of suitable agents for forming immunoconjugates are known in the art; see for example, WO 05/103081, incorporated herein by reference.

The term “antibody construct” as used herein refers to a polypeptide comprising one or more of the antigen-binding portions disclosed herein linked to a linker polypeptide or an immunoglobulin constant domain. Linker polypeptides comprise two or more amino acid residues joined by peptide bonds and are used to link one or more antigen-binding portions. Such linker polypeptides are well known in the art (see e.g., Holliger, P., et al. (1993) Proc. Natl. Acad. Sci. USA 90:6444-6448; Poljak, R. J., et al. (1994) Structure 2:1121-1123, each incorporated herein by reference). An immunoglobulin constant domain refers to a heavy or light chain constant domain. Antibody fragments, such as Fab and F(ab′)₂ fragments, can be prepared from whole antibodies using conventional techniques, such as papain or pepsin digestion, respectively, of whole antibodies. Moreover, antibodies and the antigen-binding portions thereof can be obtained using standard recombinant DNA techniques, as described herein.

An “isolated antibody”, as used herein, is intended to refer to an antibody that is substantially free of other antibodies having different antigenic specificities (e.g., an isolated antibody that specifically binds a ZIKV is substantially free of antibodies that specifically bind antigens other than a ZIKV). Moreover, an isolated antibody may be substantially free of other cellular material and/or chemicals.

In one embodiment, an isolated antibody that specifically binds a ZIKV does not have cross-reactivity to other viruses, for example, other Flaviviruses, such as a dengue virus. Preferably, the antibody, or an antigen binding portion thereof, according to the present invention does not bind to any dengue virus, Dengue virus-like particles and/or to Dengue virus envelope protein (DENV E protein). More preferably, the antibody, or an antigen-binding portion thereof, according to the present invention does not bind to the four DENV serotypes DENV1, DENV2, DENV3 and DENV4, to Dengue virus-like particles (DENV-VLP) and/or to DENV E protein of any of the four DENV serotypes DENV1, DENV2, DENV3 and DENV4. Thereby “not binding” means that, for the antibody, or an antigen binding portion thereof, no IC₅₀ or EC₅₀ value under 10² ng/ml, preferably under 10³ ng/ml, more preferably under 5*10³ng/ml, even more preferably under 8*10³ng/ml, and most preferably under 10⁴ ng/ml can be determined in a standard ELISA to DENV-VLP and/or to DENV E protein. In other words, the concentration of the antibody, or an antigen binding portion thereof, required to achieve 50% maximal binding at saturation (IC₅₀ or EC₅₀) to DENV-VLP and/or to DENV E protein in a standard ELISA is typically more than 10² ng/ml, preferably more than 10³ ng/ml, more preferably more than 5*10³ng/ml, even more preferably more than 8*10³ng/ml, and most preferably more than 10⁴ ng/ml. In certain embodiment, DENV-VLP is a Dengue reporter virus particle (RVP).

A “neutralizing antibody”, as used herein (or an “blocking antibody” or an “antibody that neutralizes ZIKV activity” or “antagonist antibody”), is intended to refer to an antibody whose binding to ZIKV results in inhibition of at least one biological activity of ZIKV. For example, an anti-ZIKV antibody, or antigen-binding portion thereof, according to the present invention may prevent or block ZIKV attachment to, fusion with, and/or entry into a host cell. In addition, a “neutralizing antibody” is one that can neutralize, i.e., prevent, inhibit, reduce, impede or interfere with, the ability of a pathogen to initiate and/or perpetuate an infection in a host. The terms “neutralizing antibody” and “an antibody that neutralizes” or “antibodies that neutralize” are used interchangeably herein. These antibodies can be used, alone or in combination, as prophylactic or therapeutic agents with other anti-viral agents upon appropriate formulation, or in association with active vaccination, or as a diagnostic tool. They can also be used to ensure that an inactivated antigen (vaccine) retains these neutralizing epitopes important for stimulating an immune response. In some embodiments, the antibodies described herein exhibit a neutralization potency against ZIKV with an IC₅₀ or EC₅₀ ranging from about 10⁻¹¹ M to about 10⁻⁹M, including about 10⁻¹¹ M, 5×10⁻¹¹ M, 10⁻¹⁰ M, 5×10⁻¹⁰ M and 10⁻⁹M.

The term “humanized antibody” refers to antibodies which comprise heavy and light chain variable region sequences from a nonhuman species (e.g., a mouse) but in which at least a portion of the VH and/or VL sequence has been altered to be more “human-like”, i.e., more similar to human germline variable sequences. In particular, the term “humanized antibody” is an antibody or a variant, derivative, analog or fragment thereof which immunospecifically binds to an antigen of interest and which comprises a framework region (FR) having substantially the amino acid sequence of a human antibody and a complementary determining region (CDR) having substantially the amino acid sequence of a non-human antibody. As used herein, the term “substantially” in the context of FR refers to a FR having an amino acid sequence at least 80%, preferably at least 85%, at least 90%, at least 95%, at least 98% or at least 99% identical to the amino acid sequence of a human FR. As used herein, the term “substantially” in the context of a CDR refers to a CDR having an amino acid sequence at least 70%, preferably at least 75%, at least 80%, at least 85%, at least 90%, at least 95%, at least 98% or at least 99% identical to the amino acid sequence of a non-human antibody CDR. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor antibody) and all or substantially all of the framework regions are those of a human immunoglobulin consensus sequence. Preferably, a humanized antibody also comprises at least a portion of an immunoglobulin constant region (Fc), typically that of a human immunoglobulin. In some embodiments, a humanized antibody contains both the light chain as well as at least the variable domain of a heavy chain. The isolated antibody also may include the CH1, hinge, CH2, CH3, and CH4 regions of the heavy chain. In some embodiments, a humanized antibody only contains a humanized light chain. In other embodiments, a humanized antibody only contains a humanized heavy chain. In specific embodiments, a humanized antibody only contains a humanized variable domain of a light chain and/or humanized heavy chain.

The humanized antibody can be selected from any class of immunoglobulins, including IgM, IgG, IgD, IgA, and IgE, and any isotype, including without limitation IgG₁, IgG₂, IgG₃, and IgG₄. The humanized antibody may comprise sequences from more than one class or isotype, and particular constant domains may be selected to optimize desired effector functions using techniques well-known in the art.

The present invention also includes fully human anti-ZIKV monoclonal antibodies comprising variants of any of the HCVR, LCVR, and/or CDR amino acid sequences disclosed herein having one or more conservative substitutions. For example, the present invention includes anti-ZIKV antibodies having HCVR, LCVR, and/or CDR amino acid sequences with, e.g., 10 or fewer, 8 or fewer, 6 or fewer, 4 or fewer, 3 or fewer, etc. conservative amino acid substitutions relative to any of the HCVR, LCVR, and/or CDR amino acid sequences disclosed herein.

The fully human anti-ZIKV monoclonal antibodies disclosed herein may comprise one or more amino acid substitutions, insertions and/or deletions in the framework and/or CDR regions of the heavy and light chain variable domains as compared to the corresponding germline sequences. Such mutations can be readily ascertained by comparing the amino acid sequences disclosed herein to germline sequences available from, for example, public antibody sequence databases. The present invention includes antibodies, and antigen-binding portions thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding portions which comprise one or more individual germline mutations or combinations thereof. In certain embodiments, all of the framework and/or CDR residues within the VH and/or VL domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies, or antigen-binding portions, according to the present invention may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding portions that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding portions obtained in this general manner are encompassed within the present invention.

The term “human antibody”, as used herein, is intended to include antibodies having variable and constant regions derived from human germline immunoglobulin sequences. The human mAbs of the present invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include mAbs in which CDR sequences derived from the germline of another mammalian species (e.g., mouse), have been grafted onto human FR sequences. The term includes antibodies recombinantly produced in a non-human mammal, or in cells of a non-human mammal. The term is not intended to include antibodies isolated from or generated in a human subject.

The term “recombinant”, as used herein, refers to antibodies, or antigen-binding portions thereof, according to the present invention created, expressed, isolated or obtained by technologies or methods known in the art as recombinant DNA technology which include, e.g., DNA splicing and transgenic expression. The term refers to antibodies expressed in a non-human mammal (including transgenic non-human mammals, e.g., transgenic mice), or a cell (e.g., CHO cells) expression system or isolated from a recombinant combinatorial human antibody library.

The terms “Kabat numbering,” “Kabat definitions,” and “Kabat labeling” are used interchangeably herein. These terms, which are recognized in the art, refer to a system of numbering amino acid residues which are more variable (i.e., hypervariable) than other amino acid residues in the heavy and light chain variable regions of an antibody, or an antigen-binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242, each incorporated herein by reference). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

As used herein, the term “CDR” refers to the complementarity determining region within antibody variable sequences. There are three CDRs in each of the variable regions of the heavy chain (HC) and the light chain (LC), which are designated CDR1, CDR2 and CDR3 (or specifically HC CDR1, HC CDR2, HC CDR3, LC CDR1, LC CDR2, and LC CDR3), for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991), incorporated herein by reference) not only provides an unambiguous residue numbering system applicable to any variable region of an antibody, but also provides precise residue boundaries defining the three CDRs. These CDRs may be referred to as Kabat CDRs. Chothia and coworkers (Chothia &Lesk, J. Mol. Biol. 196:901-917 (1987) and Chothia et al., Nature 342:877-883 (1989), each incorporated herein by reference) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995), incorporated herein by reference) and MacCallum (J Mol Biol 262(5):732-45 (1996), incorporated herein by reference). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3, or FR4, a framework region, as referred by others, represents the combined FR's within the variable region of a single, naturally occurring immunoglobulin chain. As used herein, a FR represents one of the four sub-regions, and FRs represents two or more of the four sub-regions constituting a framework region.

The framework and CDR regions of a humanized antibody need not correspond precisely to the parental sequences, e.g., the donor antibody CDR or the consensus framework may be mutagenized by substitution, insertion and/or deletion of at least one amino acid residue so that the CDR or framework residue at that site does not correspond to either the donor antibody or the consensus framework. In a preferred embodiment, such mutations, however, will not be extensive. Usually, at least 80+, preferably at least 85%, more preferably at least 90%, and most preferably at least 95% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (See e.g., Winnaker, From Genes to Clones (Verlagsgesellschaft, Weinheim, Germany 1987). In a family of immunoglobulins, each position in the consensus sequence is occupied by the amino acid occurring most frequently at that position in the family. If two amino acids occur equally frequently, either can be included in the consensus sequence.

“Percent (%) amino acid sequence identity” with respect to a peptide or polypeptide sequence is defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, GAP, BESTFIT or

Megalign (DNASTAR) software. Those skilled in the art can determine appropriate parameters for measuring alignment, including any algorithms needed to achieve maximal alignment over the full length of the sequences being compared. In one embodiment, the disclosure includes an amino acid sequence having at least 70%, at least 75%, at least 80%, at least 85%, at least 90%, at least 92%, at least 94%, at least 95%, at least 96%, at least 97%, at least 98%, or at least 99% identity to an amino acid sequence set forth in any one of SEQ ID NOs: 1 to 60, 85 to 114 and 128-137. Preferably, residue positions, which are not identical, differ by conservative amino acid substitutions.

A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24: 307-331, incorporated herein by reference. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443 45, incorporated herein by reference. A “moderately conservative” replacement is any change having a nonnegative value in the PAM250 log-likelihood matrix.

Sequence similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the present invention to a database containing a large number of sequences from different organisms is the computer program BLAST, especially BLASTP or TBLASTN, using default parameters. See, e.g., Altschul et al. (1990) J. Mol. Biol. 215: 403-410 and (1997) Nucleic Acids Res. 25:3389-3402, each incorporated herein by reference.

“Percent (%) nucleic acid sequence identity” with respect to a nucleic acid sequence is defined as the percentage of nucleotides in a candidate sequence that are identical with nucleotides in the specific nucleic acid sequence, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as FASTA, BLAST or GAP software. A nucleic acid molecule having substantial identity to a reference nucleic acid molecule may, in certain instances, encode a polypeptide having the same or substantially similar amino acid sequence as the polypeptide encoded by the reference nucleic acid molecule. Those skilled in the art can determine appropriate parameters for measuring alignment, including any algorithms needed to achieve maximal alignment over the full length of the sequences being compared. In one embodiment, the disclosure includes a nucleic acid sequence having at least 70%, at least 75%, at least 80%, at least 85%, at least 90%, at least 92%, at least 94%, at least 95%, at least 96%, at least 97%, at least 98%, or at least 99% identity to a nucleic acid sequence set forth in any one of SEQ ID NOs: 61 to 84, 115-126 and 138-141.

“Antibody-dependent-enhancement (ADE)” is a mechanism by which a virus, when bound to antiviral antibodies, enters cells having Fc receptors, leading to increased infectivity in the cells. ADE has been demonstrated in vitro for many viruses, but the only compelling data for ADE in man comes from dengue virus infections. This virus can use this mechanism to infect macrophages, causing a normally mild viral infection to become life-threatening. For example, an initial dengue virus infection is clinically manifested for most of the cases by dengue fever (DF), which is a self-limited febrile illness. Although rarely fatal, DF is characterized by often-severe disseminated body pain, headache, fever, rash, lymphadenopathy and leukopenia. Subsequent infection with a heterologous Dengue virus can lead to the much more severe to fatal disease of dengue hemorrhagic fever (DHF) or dengue shock syndrome (DSS). It is hypothesized that the presence of antibodies to the serotype causing the primary infection enhances the infection by a heterologous serotype in secondary infections. During such secondary infection, with a different serotype of dengue virus, cross-reactive antibodies that are not neutralizing form virus-antibody complexes that are taken into monocytes and Langerhans cells (dendritic cells) and increase the number of infected cells. This leads to the activation of cytotoxic lymphocytes, which can result in plasma leakage and the hemorrhagic features characteristic of DHF and DSS. This antibody-dependent enhancement of infection is one reason why the development of a successful vaccine against dengue virus has proven to be so difficult. Although less frequent, DHF/DSS can occur after primary infection, so virus virulence and immune activation are also believed to contribute to the pathogenesis of the disease.

The term “activity” includes activities such as the binding specificity/affinity of an antibody for an antigen, for example, an anti-ZIKV antibody that binds to a ZIKV antigen. In one embodiment, an anti-ZIKV antibody activity includes, but it not limited to, binding to a ZIKV in vitro; binding to a ZIKV in cells infected with a ZIKV in vivo; and neutralizing a ZIKV in vitro or in vivo.

In one embodiment, the antibody, or antigen-binding portion thereof, is capable of neutralizing a ZIKV. In one embodiment, the antibody, or antigen-binding portion thereof, is not capable of neutralizing a ZIKV.

The term “epitope” refers to an antigenic determinant that interacts with a specific antigen-binding site in the variable region of an antibody molecule known as a paratope. Conversely, the “epitope” can also interact with a specific cellular receptor or binding site on a host. The fact that the antibody binds to this certain epitope is what makes this invention so important. A single antigen may have more than one epitope. Thus, different antibodies may bind to different areas on an antigen and may have different biological effects. For example, the term “epitope” also refers to a site on an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. The epitope to which the antibodies bind may consist of a single contiguous sequence of 2 or more (e.g., 2, 3, 4, 5, 6, 7, 8, 9, 10, 11, 12, 13, 14, 15, 16, 17, 18, 19, 20 or more) amino acids located within a ZIKV E protein (e.g. a linear epitope in a domain). Epitopes may also be conformational, that is, composed of a plurality of non-contiguous amino acids, i.e., non-linear amino acids. A conformational epitope typically includes at least 3 amino acids, and more commonly, at least 5 amino acids, e.g., 7-10 amino acids in a unique spatial conformation. In certain embodiments, epitopes may include determinants that are chemically active surface groupings of molecules such as amino acids, sugar side chains, phosphoryl groups, or sulfonyl groups, and, in certain embodiments, may have specific charge characteristics. In certain embodiments, an antibody is said to specifically bind a ZIKV when it preferentially recognizes an epitope on an intact ZIKV, including a live or inactivated ZIKV.

Epitopes formed from contiguous amino acids are typically retained on exposure to denaturing solvents, whereas epitopes formed by tertiary folding are typically lost on treatment with denaturing solvents.

Various techniques known to persons of ordinary skill in the art can be used to determine whether an antibody “interacts with one or more amino acids” within a polypeptide or protein. Exemplary techniques include, for example, site-directed mutagenesis (e.g., alanine scanning mutational analysis). Other methods include routine cross-blocking assays (such as that described in Antibodies, Harlow and Lane, Cold Spring Harbor Press, Cold Spring Harbor, NY, incorporated herein by reference), peptide blot analysis (Reineke (2004) Methods Mol. Biol. 248: 443-63, incorporated herein by reference), peptide cleavage analysis crystallographic studies and NMR analysis. In addition, methods such as epitope excision, epitope extraction and chemical modification of antigens can be employed (Tomer (2000) Prot. Sci. 9: 487-496, incorporated herein by reference). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues that correspond to the specific amino acids with which the antibody interacts. See, e.g., Ehring (1999) Analytical Biochemistry 267: 252-259; Engen and Smith (2001) Anal. Chem. 73: 256A-265A, each incorporated herein by reference.

Modification-Assisted Profiling (MAP), also known as Antigen Structure-based Antibody Profiling (ASAP) is a method that categorizes large numbers of monoclonal antibodies (mAbs) directed against the same antigen according to the similarities of the binding profile of each antibody to chemically or enzymatically modified antigen surfaces (see US 2004/0101920, incorporated herein by reference). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP may facilitate identification of rare hybridoma clones that produce mAbs having the desired characteristics. MAP may be used to sort the antibodies of the present invention into groups of antibodies binding different epitopes.

The term “competitive binding” or “epitope binning”, as used herein, refers to a situation in which a first antibody competes with a second antibody, for a binding site on a third molecule, e.g., an antigen. The term includes competition between two antibodies in both orientations, i.e., the first antibody, or an antigen-binding portion thereof, that binds and blocks binding of the second antibody, or an antigen-binding portion thereof, and vice versa. Specifically, in a first orientation, the first antibody is allowed to bind to a ZIKV E protein under saturating conditions followed by assessment of binding of the second antibody to the ZIKV E protein. In a second orientation, the second antibody is allowed to bind to a ZIKV E protein under saturating conditions followed by assessment of binding of the first antibody to the ZIKV E protein. If, in both orientations, only the saturating antibody is capable of binding to the ZIKV E protein, then it is concluded that the first antibody and the second antibody compete for binding to the ZIKV E protein. As will be appreciated by a person of ordinary skill in the art, the first antibody that competes for binding with the second antibody may not necessarily bind to the same epitope as the second antibody, but may sterically block binding of the second antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. 1990 50: 1495-1502, incorporated herein by reference). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

The term “competitive binding assay” is an assay used to determine whether two or more antibodies bind to the same epitope. The assay includes a real-time, label-free bio-layer interferometry assay, enzyme linked immunosorbent assays (ELISA), an radioimmunoassay (RIA), surface plasmon resonance, flow cytometry or any other quantitative or qualitative antibody-binding assay available in the art. In one embodiment, competitive binding between two antibodies may be measured by a real-time, label-free bio-layer interferometry assay. For example, to determine if a test antibody cross-competes with a reference anti-ZIKV antibody of the present invention, the reference antibody is allowed to bind to a ZIKV-VLP under saturating conditions. Next, the ability of a test antibody to bind to the ZIKV-VLP is assessed. If the test antibody is able to bind to the ZIKV-VLP following saturation binding with the reference anti-ZIKV antibody, it can be concluded that the test antibody binds to a different epitope than the reference anti-ZIKV antibody. On the other hand, if the test antibody is not able to bind to the ZIKV-VLP following saturation binding with the reference anti-ZIKV antibody, then the test antibody may bind to the same epitope as the epitope bound by the reference anti-ZIKV antibody. However, it is also possible that an antibody that competes for binding with a reference antibody may not bind to the identical epitope as the reference antibody, but may sterically block binding of the reference antibody by binding an overlapping or adjacent epitope. In one embodiment, a competitive binding assay is a competition fluorescent activated cell sorting (FACS) assay which is used to determine whether two or more antibodies bind to the same epitope by determining whether the fluorescent signal of a labeled antibody is reduced due to the introduction of a non-labeled antibody, where competition for the same epitope will lower the level of fluorescence.

The term “labeled antibody” as used herein, refers to an antibody, or an antigen-binding portion thereof, with a label incorporated that provides for the identification of the binding protein, e.g., an antibody. Preferably, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). Examples of labels for polypeptides include, but are not limited to, the following: radioisotopes or radionuclides (e.g., ³H, ¹⁴C, ₃₅S, ₉₀Y, ₉₉Tc, ¹¹¹In, ₁₂₅I, ¹³¹I, ¹⁷⁷Lu, ¹⁶⁶Ho, or ¹⁵³Sm); fluorescent labels (e.g., FITC, rhodamine, lanthanide phosphors), enzymatic labels (e.g., horseradish peroxidase, luciferase, alkaline phosphatase); chemiluminescent markers;

biotinyl groups; predetermined polypeptide epitopes recognized by a secondary reporter (e.g., leucine zipper pair sequences, binding sites for secondary antibodies, metal binding domains, epitope tags); and magnetic agents, such as gadolinium chelates.

The antibodies specific for a ZIKV-VLP may contain no additional labels or moieties, or they may contain an N-terminal or C-terminal label or moiety. In one embodiment, the label or moiety is biotin. In a binding assay, the location of a label (if any) may determine the orientation of the peptide relative to the surface upon which the peptide is bound. For example, if a surface is coated with avidin, a peptide containing an N-terminal biotin will be oriented such that the C-terminal portion of the peptide will be distal to the surface. In one embodiment, the label may be a radionuclide, a fluorescent dye or a MRI-detectable label. In certain embodiments, such labeled antibodies may be used in diagnostic assays including imaging assays.

Various aspects of the present invention are described in further detail in the following subsections.

### II. Anti-ZIKV Antibodies

One aspect disclosed herein provides anti-ZIKV antibodies, or antigen-binding portions thereof. Collectively, the novel antibodies are referred to herein as “anti-ZIKV antibodies.” While the term “antibody” is used throughout, it should be noted that antibody fragments (i.e., antigen-binding portions of an anti-ZIKV antibody) are also included in the disclosure and may be included in the embodiments (methods and compositions) described throughout. In certain embodiments, the antigen-binding portion of an anti-ZIKV antibody is a Fab, a Fab′, a F(ab′)₂, a Fv, a disulfide linked Fv, an scFv, a single domain antibody, or a diabody.

The anti-ZIKV antibodies and antigen-binding portions thereof described herein encompass proteins having amino acid sequences that vary from those of the described antibodies, but that retain the ability to react with ZIKV. Such variant antibodies and antibody fragments comprise one or more additions, deletions, or substitutions of amino acids when compared to parent sequence, but exhibit biological activity that is essentially equivalent to that of the described antibodies. The percent amino acid identity is at least 70%. Likewise, the antibody-encoding DNA sequences of the present invention encompass sequences that comprise one or more additions, deletions, or substitutions of nucleotides when compared to the disclosed sequence, but that encode an antibody or antibody fragment that is essentially bioequivalent to an antibody, or an antigen-binding portion thereof, according to the present invention. The percent nucleic acid identity is at least 70%.

In certain embodiments of the present invention, the anti-ZIKV antibodies comprise modifications to the Fc region of the antibodies to allow for reduced binding to Fc receptors on macrophages and other cells bearing Fc receptors, while at the same time maintaining the ability to neutralize the virus, and in so doing act to prevent ADE from occurring while at the same time allowing for a decrease in viral infectivity through the normal viral attachment and/or neutralization of fusion.

The antibodies of the present invention may be mono-specific, bi-specific, or multi-specific. Multi-specific antibodies may be specific for different epitopes of one target polypeptide or may contain antigen-binding domains specific for more than one target polypeptide. See, e.g., Tuft et al., 1991, J. Immunol. 147:60-69; Kufer et al., 2004, Trends Biotechnol. 22:238-244.

In certain embodiments of the present invention, the anti-ZIKV antibodies are able to bind to and neutralize the activity of a ZIKV, as determined by in vitro or in vivo assays. The ability of the antibodies of the present invention to bind to and neutralize the activity of a ZIKV and thus the attachment and/or entry of the virus into a host cell followed by the ensuing viral infection, may be measured using any standard method known to those skilled in the art, including binding assays (e.g., indirect ELISA), or activity assays (e.g., a TCID50-based micro-neutralization titers (MNT) assay and a reporter virus particle (RVP) assay, as described in Example 3. TCID50 represents tissue culture infectious dose 50%. In some embodiments, the antibodies exhibit a neutralization potency against ZIKV with an IC₅₀ ranging from about 10⁻¹¹ M to about 10⁻⁹ M.

In a MNT assay, the determination of the neutralization potency of a test antibody is based on enhanced protection of cells from viral infection by a live ZIKV. In brief, a serially diluted test sample, e.g., hybridoma supernatant, is mixed with 100 TCID50/well of Zika live viruses and then added to monolayer of cells (e.g., Vero cells) in 96-well plates and plates are observed after at least 5 days post-infection for presence or absence of cytopathic effect (CPE) for endpoint titer. In a Zika RVP assay, the determination of the neutralization potency of a test antibody is based on reduction in expression of a reporter gene, such as Renilla luciferase, horseradish peroxidase (HRP), alkaline phosphatase (AP), glucose oxidase, (β-galactosidase, Green fluorescent protein (GFP) and R-phycoerythrin. In this assay, Zika RVPs are used in place of Zika live viruses to determine neutralizing antibody titers. Zika RVPs retain the antigenic determinants of wild type virions including capsid, envelope, pre-membrane and membrane proteins CprME. The Zika RVPs express a reporter gene upon infection of permissive cells. If the reporter gene is Renilla luciferase, the half maximal effective concentration EC₅₀ titer of antibodies was determined using a bioluminescent reaction which generates a glow-type luminescent signal by the interaction of the Renilla luciferase and coelenterazine substrate. The luminescent signal was measured using a luminescence enabled plate reader. Reduction in luminescent signal in the presence of serum indicates neutralization.

In certain embodiments of the present invention, the anti-ZIKV antibodies cross react with ZIKV strains, including MR766 (Uganda 1947), PRVABC59 (Puerto Rico 2015), FLR (Colombia 2015), SV0127-14 (Thailand, 2014), CPC-0740 (Philippine 2012), and Fortaleza (Brazil 2015). In certain embodiments of the present invention, the anti-ZIKV antibodies also bind to ZIKV-VLPs derived from cells expressing ZIKV prM/E with an IC₅₀ or EC₅₀ ranging from about 80 pM to about 150 nM.

In one embodiment, anti-ZIKV antibodies are disclosed which have the ability to bind to ZIKV, as described in the Examples below. In one embodiment, the anti-ZIKV antibodies, or antigen-binding portions thereof, are able to neutralize a ZIKV. The ZIKV is selected from the group consisting of MR766 (Uganda 1947), PRVABC59 (Puerto Rico 2015), FLR (Colombia 2015), SV0127-14 (Thailand, 2014), CPC-0740 (Philippine 2012), and Fortaleza (Brazil 2015).

Example 1 describes the generation of anti-ZIKV antibodies. In certain embodiments, the antibodies, or antigen-binding portions thereof, according to the present invention are obtained from rabbits immunized with immunogens, such as a purified inactivated Zika virus (PIZV) and/or ZIKV-VLPs comprised of prM and E proteins.

Non-limiting, exemplary in vitro assays for measuring binding activity are illustrated in Example 3 as described herein. In Example 3, the binding of anti-ZIKV E was determined by evaluating binding to ZIKV-VLPs produced in cells expressing prM/E. The k_{off} were determined by Bio-layer interferometry (BLI) using an Octet-96 device. Neutralization assays were used to determine the effect of anti-ZIKV antibodies on infectivity of ZIKV. Indirect ELISA assay was conducted to determine cross reactivity of anti-ZIKV antibodies.

The heavy and light chain amino acid sequences for these anti-ZIKV antibodies are set forth in Table 6. The heavy and light chain nucleotide sequences for these anti-ZIKV antibodies are set forth in Table 7.

Thus, in one embodiment, the disclosure includes anti-ZIKV antibodies, or antigen-binding portions thereof, comprising a heavy chain variable region comprising an amino acid sequence selected from the group consisting of SEQ ID NOs: 2, 12, 22, 32, 42, 52, 86, 96, 106 and 129; and a light chain variable region comprising an amino acid sequence selected from the group consisting of 7, 17, 27, 37, 47, 57, 91, 101, 111 and 134. In one embodiment, the disclosure includes anti-ZIKV antibodies, or antigen-binding portions thereof, comprising a heavy chain comprising an amino acid sequence selected from the group consisting of SEQ ID NOs: 1, 11, 21, 31, 41, 51, 85, 95, 105, and 128; and a light chain comprising an amino acid sequence selected from the group consisting of 6, 16, 26, 37, 46, 56, 90, 100, 110 and 133.

In one embodiment, the disclosure includes an anti-ZIKV antibody, or antigen-binding portion thereof, comprising an HC CDR set (CDR1, CDR2, and CDR3) selected from those set forth in Table 6; and an LC CDR set (CDR1, CDR2, and CDR3) selected from those set forth in Table 6.

In one embodiment, an anti-ZIKV antibody, or antigen-binding portion thereof, is the 102-1 antibody. Specifically, the 102-1 antibody comprises a heavy chain variable region comprising a CDR3 domain comprising the amino acid sequence of SEQ ID NO: 5, a CDR2 domain comprising the amino acid sequence of SEQ ID NO: 4, and a CDR1 domain comprising the amino acid sequence of SEQ ID NO: 3; and a light chain variable region comprising a CDR3 domain comprising the amino acid sequence of SEQ ID NO: 10, a CDR2 domain comprising the amino acid sequence of SEQ ID NO: 9, and a CDR1 domain comprising the amino acid sequence of SEQ ID NO: 8. More specifically, the 102-1 antibody comprises a heavy chain variable region comprising the amino acid sequence of SEQ ID NO: 2 and a light chain variable region comprising the amino acid sequence of SEQ ID NO: 7. Even more specifically, the 102-1 antibody consists of a heavy chain comprising the amino acid sequence of SEQ ID NO: 1 and a light chain comprising the amino acid sequence of SEQ ID NO: 6.

In one embodiment, an anti-ZIKV antibody, or antigen-binding portion thereof, comprises a heavy chain variable domain comprising an amino acid sequence set forth in SEQ ID NO: 2, or a sequence having at least 70%, 75%, 80%, 85%, 90%, 95%, 96%, 97%, 98%, or 99% identity to SEQ ID NO: 2; and a light chain variable domain comprising an amino acid sequence set forth in SEQ ID NO: 7, or a sequence having at least 70%, 75%, 80%, 85%, 90%, 92%, 94%, 95%, 96%, 97%, 98%, or 99% identity to SEQ ID NO: 7. In one embodiment, an anti-ZIKV antibody, or antigen-binding portion thereof, comprises a heavy chain comprising an amino acid sequence set forth in SEQ ID NO: 1, or a sequence having at least 70%, 75%, 80%, 85%, 90%, 92%, 94%, 95%, 96%, 97%, 98%, or 99% identity to SEQ ID NO: 1; and/or a light chain comprising an amino acid sequence set forth in SEQ ID NO: 6, or a sequence having at least 70%, 75%, 80%, 85%, 90%, 92%, 94%, 95%, 96%, 97%, 98%, or 99% identity to SEQ ID NO: 6.

In one embodiment, an anti-ZIKV antibody, or antigen-binding portion thereof, is the 242-3 antibody. Specifically, the 242-3 antibody comprises a heavy chain variable region comprising a CDR3 domain comprising the amino acid sequence of SEQ ID NO: 15, a CDR2 domain comprising the amino acid sequence of SEQ ID NO: 14, and a CDR1 domain comprising the amino acid sequence of SEQ ID NO: 13; and a light chain variable region comprising a CDR3 domain comprising the amino acid sequence of SEQ ID NO: 20, a CDR2 domain comprising the amino acid sequence of SEQ ID NO: 19, and a CDR1 domain comprising the amino acid sequence of SEQ ID NO: 18. More specifically, the 242-3 antibody comprises a heavy chain variable region comprising the amino acid sequence of SEQ ID NO: 12 and a light chain variable region comprising the amino acid sequence of SEQ ID NO: 17. Even more specifically, the 242-3 antibody consists of a heavy chain comprising the amino acid sequence of SEQ ID NO: 11 and a light chain comprising the amino acid sequence of SEQ ID NO: 16.

In one embodiment, an anti-ZIKV antibody, or antigen-binding portion thereof, comprises a heavy chain variable domain comprising an amino acid sequence set forth in SEQ ID NO: 12, or a sequence having at least 70%, 75%, 80%, 85%, 90%, 95%, 96%, 97%, 98%, or 99% identity to SEQ ID NO: 12; and a light chain variable domain comprising an amino acid sequence set forth in SEQ ID NO: 17, or a sequence having at least 70%, 75%, 80%, 85%, 90%, 92%, 94%, 95%, 96%, 97%, 98%, or 99% identity to SEQ ID NO: 17. In one embodiment, an anti-ZIKV antibody, or antigen-binding portion thereof, comprises a heavy chain comprising an amino acid sequence set forth in SEQ ID NO: 11, or a sequence having at least 70%, 75%, 80%, 85%, 90%, 92%, 94%, 95%, 96%, 97%, 98%, or 99% identity to SEQ ID NO: 11; and a light chain comprising an amino acid sequence set forth in SEQ ID NO: 16, or a sequence having at least 70%, 75%, 80%, 85%, 90%, 92%, 94%, 95%, 96%, 97%, 98%, or 99% identity to SEQ ID NO: 16.

In one embodiment, an anti-ZIKV antibody, or antigen-binding portion thereof, is the 289-3 antibody. Specifically, the 289-3 antibody comprises a heavy chain variable region comprising a CDR3 domain comprising the amino acid sequence of SEQ ID NO: 25, a CDR2 domain comprising the amino acid sequence of SEQ ID NO: 24, and a CDR1 domain comprising the amino acid sequence of SEQ ID NO: 23; and a light chain variable region comprising a CDR3 domain comprising the amino acid sequence of SEQ ID NO: 30, a CDR2 domain comprising the amino acid sequence of SEQ ID NO: 29, and a CDR1 domain comprising the amino acid sequence of SEQ ID NO: 28. More specifically, the 289-3 antibody comprises a heavy chain variable region comprising the amino acid sequence of SEQ ID NO: 22 and a light chain variable region comprising the amino acid sequence of SEQ ID NO: 27. Even more specifically, the 289-3 antibody consists of a heavy chain comprising the amino acid sequence of SEQ ID NO: 21 and a light chain comprising the amino acid sequence of SEQ ID NO: 26.

In one embodiment, an anti-ZIKV antibody, or antigen-binding portion thereof, comprises a heavy chain variable domain comprising an amino acid sequence set forth in SEQ ID NO: 22, or a sequence having at least 70%, 75%, 80%, 85%, 90%, 95%, 96%, 97%, 98%, or 99% identity to SEQ ID NO: 22; and a light chain variable domain comprising an amino acid sequence set forth in SEQ ID NO: 27, or a sequence having at least 70%, 75%, 80%, 85%, 90%, 92%, 94%, 95%, 96%, 97%, 98%, or 99% identity to SEQ ID NO: 27. In one embodiment, an anti-ZIKV antibody, or antigen-binding portion thereof, comprises a heavy chain comprising an amino acid sequence set forth in SEQ ID NO: 21, or a sequence having at least 70%, 75%, 80%, 85%, 90%, 92%, 94%, 95%, 96%, 97%, 98%, or 99% identity to SEQ ID NO: 21; and a light chain comprising an amino acid sequence set forth in SEQ ID NO: 26, or a sequence having at least 70%, 75%, 80%, 85%, 90%, 92%, 94%, 95%, 96%, 97%, 98%, or 99% identity to SEQ ID NO: 26.

In one embodiment, an anti-ZIKV antibody, or antigen-binding portion thereof, is the 306-2 antibody. Specifically, the 306-2 antibody comprises a heavy chain variable region comprising a CDR3 domain comprising the amino acid sequence of SEQ ID NO: 35, a CDR2 domain comprising the amino acid sequence of SEQ ID NO: 34, and a CDR1 domain comprising the amino acid sequence of SEQ ID NO: 33; and a light chain variable region comprising a CDR3 domain comprising the amino acid sequence of SEQ ID NO: 40, a CDR2 domain comprising the amino acid sequence of SEQ ID NO: 39, and a CDR1 domain comprising the amino acid sequence of SEQ ID NO: 38. More specifically, the 306-2 antibody comprises a heavy chain variable region comprising the amino acid sequence of SEQ ID NO: 32 and a light chain variable region comprising the amino acid sequence of SEQ ID NO: 37. Even more specifically, the 306-2 antibody consists of a heavy chain comprising the amino acid sequence of SEQ ID NO: 31 and a light chain comprising the amino acid sequence of SEQ ID NO: 36.

In one embodiment, an anti-ZIKV antibody, or antigen-binding portion thereof, comprises a heavy chain variable domain comprising an amino acid sequence set forth in SEQ ID NO: 32, or a sequence having at least 70%, 75%, 80%, 85%, 90%, 95%, 96%, 97%, 98%, or 99% identity to SEQ ID NO: 32; and a light chain variable domain comprising an amino acid sequence set forth in SEQ ID NO: 37, or a sequence having at least 70%, 75%, 80%, 85%, 90%, 92%, 94%, 95%, 96%, 97%, 98%, or 99% identity to SEQ ID NO: 37. In one embodiment, an anti-ZIKV antibody, or antigen-binding portion thereof, comprises a heavy chain comprising an amino acid sequence set forth in SEQ ID NO: 31, or a sequence having at least 70%, 75%, 80%, 85%, 90%, 92%, 94%, 95%, 96%, 97%, 98%, or 99% identity to SEQ ID NO: 31; and a light chain comprising an amino acid sequence set forth in SEQ ID NO: 36, or a sequence having at least 70%, 75%, 80%, 85%, 90%, 92%, 94%, 95%, 96%, 97%, 98%, or 99% identity to SEQ ID NO: 36.

In one embodiment, an anti-ZIKV antibody, or antigen-binding portion thereof, is the 11-3 antibody. Specifically, the 11-3 antibody comprises a heavy chain variable region comprising a CDR3 domain comprising the amino acid sequence of SEQ ID NO: 45, a CDR2 domain comprising the amino acid sequence of SEQ ID NO: 44, and a CDR1 domain comprising the amino acid sequence of SEQ ID NO: 43; and a light chain variable region comprising a CDR3 domain comprising the amino acid sequence of SEQ ID NO: 50, a CDR2 domain comprising the amino acid sequence of SEQ ID NO: 49, and a CDR1 domain comprising the amino acid sequence of SEQ ID NO: 48. More specifically, the 11-3 antibody comprises a heavy chain variable region comprising the amino acid sequence of SEQ ID NO: 42 and a light chain variable region comprising the amino acid sequence of SEQ ID NO: 47. Even more specifically, the 11-3 antibody consists of a heavy chain comprising the amino acid sequence of SEQ ID NO: 41 and a light chain comprising the amino acid sequence of SEQ ID NO: 46.

In one embodiment, an anti-ZIKV antibody, or antigen-binding portion thereof, comprises a heavy chain variable domain comprising an amino acid sequence set forth in SEQ ID NO: 42, or a sequence having at least 70%, 75%, 80%, 85%, 90%, 95%, 96%, 97%, 98%, or 99% identity to SEQ ID NO: 42; and a light chain variable domain comprising an amino acid sequence set forth in SEQ ID NO: 47, or a sequence having at least 70%, 75%, 80%, 85%, 90%, 92%, 94%, 95%, 96%, 97%, 98%, or 99% identity to SEQ ID NO: 47. In one embodiment, an anti-ZIKV antibody, or antigen-binding portion thereof, comprises a heavy chain comprising an amino acid sequence set forth in SEQ ID NO: 41, or a sequence having at least 70%, 75%, 80%, 85%, 90%, 92%, 94%, 95%, 96%, 97%, 98%, or 99% identity to SEQ ID NO: 41; and a light chain comprising an amino acid sequence set forth in SEQ ID NO: 46, or a sequence having at least 70%, 75%, 80%, 85%, 90%, 92%, 94%, 95%, 96%, 97%, 98%, or 99% identity to SEQ ID NO: 46.

In one embodiment, an anti-ZIKV antibody, or antigen-binding portion thereof, is the 78-2 antibody. Specifically, the 78-2 antibody comprises a heavy chain variable region comprising a CDR3 domain comprising the amino acid sequence of SEQ ID NO: 55, a CDR2 domain comprising the amino acid sequence of SEQ ID NO: 54, and a CDR1 domain comprising the amino acid sequence of SEQ ID NO: 53; and a light chain variable region comprising a CDR3 domain comprising the amino acid sequence of SEQ ID NO: 60, a CDR2 domain comprising the amino acid sequence of SEQ ID NO: 59, and a CDR1 domain comprising the amino acid sequence of SEQ ID NO: 58. More specifically, the 78-2 antibody comprises a heavy chain variable region comprising the amino acid sequence of SEQ ID NO: 52 and a light chain variable region comprising the amino acid sequence of SEQ ID NO: 57. Even more specifically, the 78-2 antibody consists of a heavy chain comprising the amino acid sequence of SEQ ID NO: 51 and a light chain comprising the amino acid sequence of SEQ ID NO: 56.

In one embodiment, an anti-ZIKV antibody, or antigen-binding portion thereof, comprises a heavy chain variable domain comprising an amino acid sequence set forth in SEQ ID NO: 52, or a sequence having at least 70%, 75%, 80%, 85%, 90%, 95%, 96%, 97%, 98%, or 99% identity to SEQ ID NO: 52; and a light chain variable domain comprising an amino acid sequence set forth in SEQ ID NO: 57, or a sequence having at least 70%, 75%, 80%, 85%, 90%, 92%, 94%, 95%, 96%, 97%, 98%, or 99% identity to SEQ ID NO: 57. In one embodiment, an anti-ZIKV antibody, or antigen-binding portion thereof, comprises a heavy chain comprising an amino acid sequence set forth in SEQ ID NO: 51, or a sequence having at least 70%, 75%, 80%, 85%, 90%, 92%, 94%, 95%, 96%, 97%, 98%, or 99% identity to SEQ ID NO: 51; and a light chain comprising an amino acid sequence set forth in SEQ ID NO: 56, or a sequence having at least 70%, 75%, 80%, 85%, 90%, 92%, 94%, 95%, 96%, 97%, 98%, or 99% identity to SEQ ID NO: 56.

In one embodiment, an anti-ZIKV antibody, or antigen-binding portion thereof, is the 270-12 antibody. Specifically, the 270-12 antibody comprises a heavy chain variable region comprising a CDR3 domain comprising the amino acid sequence of SEQ ID NO: 89, a CDR2 domain comprising the amino acid sequence of SEQ ID NO: 88, and a CDR1 domain comprising the amino acid sequence of SEQ ID NO: 87; and a light chain variable region comprising a CDR3 domain comprising the amino acid sequence of SEQ ID NO: 94, a CDR2 domain comprising the amino acid sequence of SEQ ID NO: 93, and a CDR1 domain comprising the amino acid sequence of SEQ ID NO: 92. More specifically, the 270-12 antibody comprises a heavy chain variable region comprising the amino acid sequence of SEQ ID NO: 86 and a light chain variable region comprising the amino acid sequence of SEQ ID NO: 91. Even more specifically, the 270-12 antibody consists of a heavy chain comprising the amino acid sequence of SEQ ID NO: 85 and a light chain comprising the amino acid sequence of SEQ ID NO: 90.

In one embodiment, an anti-ZIKV antibody, or antigen-binding portion thereof, comprises a heavy chain variable domain comprising an amino acid sequence set forth in SEQ ID NO: 86, or a sequence having at least 70%, 75%, 80%, 85%, 90%, 95%, 96%, 97%, 98%, or 99% identity to SEQ ID NO: 86; and a light chain variable domain comprising an amino acid sequence set forth in SEQ ID NO: 91, or a sequence having at least 70%, 75%, 80%, 85%, 90%, 92%, 94%, 95%, 96%, 97%, 98%, or 99% identity to SEQ ID NO: 91. In one embodiment, an anti-ZIKV antibody, or antigen-binding portion thereof, comprises a heavy chain comprising an amino acid sequence set forth in SEQ ID NO: 85, or a sequence having at least 70%, 75%, 80%, 85%, 90%, 92%, 94%, 95%, 96%, 97%, 98%, or 99% identity to SEQ ID NO: 85; and/or a light chain comprising an amino acid sequence set forth in SEQ ID NO: 90, or a sequence having at least 70%, 75%, 80%, 85%, 90%, 92%, 94%, 95%, 96%, 97%, 98%, or 99% identity to SEQ ID NO: 90.

In one embodiment, an anti-ZIKV antibody, or antigen-binding portion thereof, is the 181-4/329-2 antibody. Specifically, the 181-4/329-2 antibody comprises a heavy chain variable region comprising a CDR3 domain comprising the amino acid sequence of SEQ ID NO: 99, a CDR2 domain comprising the amino acid sequence of SEQ ID NO: 98, and a CDR1 domain comprising the amino acid sequence of SEQ ID NO: 97; and a light chain variable region comprising a CDR3 domain comprising the amino acid sequence of SEQ ID NO: 104, a CDR2 domain comprising the amino acid sequence of SEQ ID NO: 103, and a CDR1 domain comprising the amino acid sequence of SEQ ID NO: 102. More specifically, the 181-4/329-2 antibody comprises a heavy chain variable region comprising the amino acid sequence of SEQ ID NO: 96 and a light chain variable region comprising the amino acid sequence of SEQ ID NO: 101. Even more specifically, the 181-4/329-2 antibody consists of a heavy chain comprising the amino acid sequence of SEQ ID NO: 95 and a light chain comprising the amino acid sequence of SEQ ID NO: 100.

In one embodiment, an anti-ZIKV antibody, or antigen-binding portion thereof, comprises a heavy chain variable domain comprising an amino acid sequence set forth in SEQ ID NO: 96, or a sequence having at least 70%, 75%, 80%, 85%, 90%, 95%, 96%, 97%, 98%, or 99% identity to SEQ ID NO: 96; and a light chain variable domain comprising an amino acid sequence set forth in SEQ ID NO: 101, or a sequence having at least 70%, 75%, 80%, 85%, 90%, 92%, 94%, 95%, 96%, 97%, 98%, or 99% identity to SEQ ID NO: 101. In one embodiment, an anti-ZIKV antibody, or antigen-binding portion thereof, comprises a heavy chain comprising an amino acid sequence set forth in SEQ ID NO: 95, or a sequence having at least 70%, 75%, 80%, 85%, 90%, 92%, 94%, 95%, 96%, 97%, 98%, or 99% identity to SEQ ID NO: 95; and/or a light chain comprising an amino acid sequence set forth in SEQ ID NO: 100, or a sequence having at least 70%, 75%, 80%, 85%, 90%, 92%, 94%, 95%, 96%, 97%, 98%, or 99% identity to SEQ ID NO: 100.

In one embodiment, an anti-ZIKV antibody, or antigen-binding portion thereof, is the 260-3 antibody. Specifically, the 260-3 antibody comprises a heavy chain variable region comprising a CDR3 domain comprising the amino acid sequence of SEQ ID NO: 109, a CDR2 domain comprising the amino acid sequence of SEQ ID NO: 108, and a CDR1 domain comprising the amino acid sequence of SEQ ID NO: 107; and a light chain variable region comprising a CDR3 domain comprising the amino acid sequence of SEQ ID NO: 114, a CDR2 domain comprising the amino acid sequence of SEQ ID NO: 113, and a CDR1 domain comprising the amino acid sequence of SEQ ID NO: 112. More specifically, the 260-3 antibody comprises a heavy chain variable region comprising the amino acid sequence of SEQ ID NO: 106 and a light chain variable region comprising the amino acid sequence of SEQ ID NO: 111. Even more specifically, the 260-3 antibody consists of a heavy chain comprising the amino acid sequence of SEQ ID NO: 105 and a light chain comprising the amino acid sequence of SEQ ID NO: 110.

In one embodiment, an anti-ZIKV antibody, or antigen-binding portion thereof, comprises a heavy chain variable domain comprising an amino acid sequence set forth in SEQ ID NO: 106, or a sequence having at least 70%, 75%, 80%, 85%, 90%, 95%, 96%, 97%, 98%, or 99% identity to SEQ ID NO: 106; and a light chain variable domain comprising an amino acid sequence set forth in SEQ ID NO: 111, or a sequence having at least 70%, 75%, 80%, 85%, 90%, 92%, 94%, 95%, 96%, 97%, 98%, or 99% identity to SEQ ID NO: 111. In one embodiment, an anti-ZIKV antibody, or antigen-binding portion thereof, comprises a heavy chain comprising an amino acid sequence set forth in SEQ ID NO: 105, or a sequence having at least 70%, 75%, 80%, 85%, 90%, 92%, 94%, 95%, 96%, 97%, 98%, or 99% identity to SEQ ID NO: 105; and/or a light chain comprising an amino acid sequence set forth in SEQ ID NO: 110, or a sequence having at least 70%, 75%, 80%, 85%, 90%, 92%, 94%, 95%, 96%, 97%, 98%, or 99% identity to SEQ ID NO: 110.

In one embodiment, an anti-ZIKV antibody, or antigen-binding portion thereof, is the 278-11 antibody. Specifically, the 278-11 antibody comprises a heavy chain variable region comprising a CDR3 domain comprising the amino acid sequence of SEQ ID NO: 132, a CDR2 domain comprising the amino acid sequence of SEQ ID NO: 131, and a CDR1 domain comprising the amino acid sequence of SEQ ID NO: 130; and a light chain variable region comprising a CDR3 domain comprising the amino acid sequence of SEQ ID NO: 137, a CDR2 domain comprising the amino acid sequence of SEQ ID NO: 136, and a CDR1 domain comprising the amino acid sequence of SEQ ID NO: 135. More specifically, the 278-11 antibody comprises a heavy chain variable region comprising the amino acid sequence of SEQ ID NO: 129 and a light chain variable region comprising the amino acid sequence of SEQ ID NO: 134. Even more specifically, the 278-11 antibody consists of a heavy chain comprising the amino acid sequence of SEQ ID NO: 128 and a light chain comprising the amino acid sequence of SEQ ID NO: 133.

In one embodiment, an anti-ZIKV antibody, or antigen-binding portion thereof, comprises a heavy chain variable domain comprising an amino acid sequence set forth in SEQ ID NO: 129, or a sequence having at least 70%, 75%, 80%, 85%, 90%, 95%, 96%, 97%, 98%, or 99% identity to SEQ ID NO: 129; and a light chain variable domain comprising an amino acid sequence set forth in SEQ ID NO: 134, or a sequence having at least 70%, 75%, 80%, 85%, 90%, 92%, 94%, 95%, 96%, 97%, 98%, or 99% identity to SEQ ID NO: 134. In one embodiment, an anti-ZIKV antibody, or antigen-binding portion thereof, comprises a heavy chain comprising an amino acid sequence set forth in SEQ ID NO: 128, or a sequence having at least 70%, 75%, 80%, 85%, 90%, 92%, 94%, 95%, 96%, 97%, 98%, or 99% identity to SEQ ID NO: 128; and/or a light chain comprising an amino acid sequence set forth in SEQ ID NO: 133, or a sequence having at least 70%, 75%, 80%, 85%, 90%, 92%, 94%, 95%, 96%, 97%, 98%, or 99% identity to SEQ ID NO: 133.

The foregoing anti-ZIKV antibody CDR sequences establish a novel family of ZIKV binding proteins, isolated in accordance with this disclosure, and comprising antigen-binding polypeptides that include the CDR sequences listed in Table 6.

To generate and to select CDRs having preferred ZIKV binding and/or neutralizing activity with respect to a ZIKV, standard methods known in the art for generating antibodies, or antigen-binding portions thereof, and assessing the ZIKV binding and/or neutralizing characteristics of those antibodies, or antigen-binding portions thereof, may be used, including but not limited to those specifically described herein.

In certain embodiments, the antibody comprises a heavy chain constant region, such as an IgG1, IgG2, IgG3, IgG4, IgA, IgE, IgM, or IgD constant region. In certain embodiments, the anti-ZIKV antibody, or antigen-binding portion thereof, comprises a heavy chain immunoglobulin constant domain selected from the group consisting of a human IgG constant domain, a human IgM constant domain, a human IgE constant domain, and a human IgA constant domain. In further embodiments, the antibody, or antigen-binding portion thereof, has an IgG1 heavy chain constant region, an IgG2 heavy chain constant region, an IgG3 constant region, or an IgG4 heavy chain constant region. Preferably, the heavy chain constant region is an IgG1 heavy chain constant region or an IgG4 heavy chain constant region. In one embodiment, the antibody, or antigen-binding portion thereof, is an IgG4 isotype.

Furthermore, the antibody can comprise a light chain constant region, either a kappa light chain constant region or a lambda light chain constant region. Preferably, the antibody comprises a kappa light chain constant region.

In certain embodiments, the anti-ZIKV antibody binding portion is a Fab, a Fab′, a F(ab′)2, a Fv, a disulfide linked Fv, an scFv, a single domain antibody, or a diabody.

In certain embodiments, the anti-ZIKV antibody, or antigen-binding portion thereof, is a multi-specific antibody, e.g. a bispecific antibody. Replacements of amino acid residues in the Fc portion to alter antibody effector function are have been described (Winter, et al. U.S. Pat. Nos. 5,648,260 and 5,624,821, incorporated herein by reference). The Fc portion of an antibody mediates several important effector functions e.g. cytokine induction, antibody-dependent cell-mediated cytotoxicity (ADCC), phagocytosis, complement dependent cytotoxicity (CDC) and half-life/clearance rate of antibody and antigen-antibody complexes. In some cases these effector functions are desirable for therapeutic antibody but in other cases might be unnecessary or even deleterious, depending on the therapeutic objectives. Certain human IgG isotypes, particularly IgG1 and IgG3, mediate ADCC and CDC via binding to FcRs and complement C1q, respectively. Neonatal Fc receptors (FcRn) are the critical components determining the circulating half-life of antibodies. In still another embodiment at least one amino acid residue is replaced in the constant region of the antibody, for example the Fc region of the antibody, such that effector functions of the antibody are altered.

One embodiment includes a labeled anti-ZIKV antibody, or antibody portion thereof, where the antibody is derivatized or linked to one or more functional molecule(s) (e.g., another peptide or protein). For example, a labeled antibody can be derived by functionally linking an antibody or antibody portion of the disclosure (by chemical coupling, genetic fusion, noncovalent association or otherwise) to one or more other molecular entities, such as another antibody (e.g., a bispecific antibody or a diabody), a detectable agent, a protein or peptide that can mediate the association of the antibody or antibody portion with another molecule (such as a streptavidin core region or a polyhistidine tag).

Useful detectable agents with which an isolated antibody, or antigen-binding portion thereof, may be derivatized include fluorescent compounds. Exemplary fluorescent detectable agents include fluorescein, fluorescein isothiocyanate, rhodamine, 5-dimethylamine-1-napthalenesulfonyl chloride, phycoerythrin and the like. An isolated antibody, or antigen-binding portion thereof, may also be derivatized with detectable enzymes, such as alkaline phosphatase, β-galactosidase, horseradish peroxidase, luciferase, glucose oxidase and the like. When an isolated antibody, or antigen-binding portion thereof, is derivatized with a detectable enzyme, it is detected by adding additional reagents that the enzyme uses to produce a detectable reaction product. For example, when the detectable agent is horseradish peroxidase, the isolated antibody, or antigen-binding portion thereof, is detected by an addition of hydrogen peroxide and diaminobenzidine, which leads to a colored reaction product. An isolated antibody, or antigen-binding portion thereof, may also be derivatized with biotin, and detected through indirect measurement of avidin or streptavidin binding. In one embodiment, the antibody is conjugated to an imaging agent. Examples of imaging agents that may be used in the compositions and methods as described herein include, but are not limited to, a radiolabel (e.g., indium), an enzyme, a fluorescent label, a luminescent label, a bioluminescent label, a magnetic label, and biotin.

In one embodiment, the antibodies are linked to a radiolabel, such as, but not limited to, indium (¹¹¹In). ¹¹¹In may be used to label the antibodies as described herein for use in diagnosing infection of a ZIKV. In a certain embodiment, anti-ZIKV antibodies as described herein are labeled with In via a bifunctional chelator which is a bifunctional cyclohexyl diethylenetriaminepentaacetic acid (DTPA) chelate (see U.S. Pat. Nos. 5,124,471;

5,434,287; and 5,286,850, each incorporated herein by reference).

In general, the antibody according to the present invention may be glycosylated. N-linked glycans attached to the CH2 domain of a heavy chain, for instance, can influence C1q and FcR binding, with aglycosylated antibodies having lower affinity for these receptors. Accordingly, the CH2 domain of the Fc moiety of the antibody according to the present invention may comprise one or more mutations, in which a glycosylated residue is substituted by a non-glycosylated residue. The glycan structure can also affect activity, e.g., differences in complement-mediated cell death may be seen depending on the number of galactose sugars (0, 1 or 2) at the terminus of a glycan's biantennary chain. Preferably, the antibody's glycans do not lead to a human immunogenic response after administration.

### III. Preparation of Anti-ZIKV Antibodies

### A. Generation of Rabbit Monoclonal Anti-ZIKV Antibodies

Monoclonal antibodies can be prepared using hybridoma methods, such as those described by Kohler and Milstein (1975) Nature 256:495, incorporated herein by reference. Using the hybridoma method, a mouse, hamster, rabbit or other appropriate host animal is immunized to elicit the production by lymphocytes of antibodies that will specifically bind to a ZIKV. Lymphocytes can also be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a ZIKV as determined by immunoprecipitation, immunoblotting, or by an in vitro binding assay (e.g., radioimmunoassay (RIA); enzyme-linked immunosorbent assay (ELISA)) can then be propagated either in vitro culture using standard methods (Goding, Monoclonal Antibodies: Principles and Practice, Academic Press, 1986, incorporated herein by reference) or in vivo as ascites tumors in an animal. The monoclonal antibodies can then be purified from the culture medium or ascites fluid as described for polyclonal antibodies.

In certain embodiments, the antibodies of the present invention are obtained from a rabbit immunized with a PIZV and/or a ZIKV-VLP. The PIZV may be produced by culturing cells, e.g., Vero cells, infected with a ZIKV strain, such as PRVABC59, harvesting/isolating live ZIKV particles, purifying isolated live ZIKV particles; and chemically inactivating ZIKV particles. The ZIKV-VLP may be obtained from a commercial source. In certain embodiments, one or more booster injections may be administered. In certain embodiments, the booster injections may comprise antigens that are different from the one for the initial immunization.

Generally, a rabbit is immunized with a PIZV and/or a ZIKV-VLP, and lymphatic cells (such as B-cells) are recovered from the spleen of the rabbit that produces antibodies.

The lymphatic cells may be fused with a myeloma cell line to prepare immortal hybridoma cell lines, and such hybridoma cell lines are screened and selected to identify hybridoma cell lines that produce antibodies specific to a ZIKV. DNAs encoding the variable regions of the heavy chain and light chain may be isolated and linked to desirable isotypic constant regions of the heavy chain and light chain. Alternatively, DNAs encoding the antigen-specific light and heavy chains may be isolated directly from antigen-specific lymphocytes. The DNAs are then cloned into an expression vector(s).

As in the experimental section below, the antibodies are characterized and selected for desirable characteristics, including affinity, selectivity, epitope, etc. While the constant region selected may vary according to specific use, high affinity antigen-binding and target specificity characteristics reside in the variable region.

### B. Generation of Fully Human Monoclonal Anti-ZIKV Antibodies

Methods for generating human anti-ZIKV antibodies can be directly prepared using various techniques known in the art. Immortalized human B lymphocytes immunized in vitro or isolated from an immunized individual that produce an antibody directed against a target antigen can be generated (see, e.g., Cole et al., Monoclonal Antibodies and Cancer Therapy, Alan R. Liss, p. 77 (1985); Boemer et al., 1991, J. Immunol, 147 (l):86-95; and U.S. Pat. 5,750,373, each incorporated herein by reference). Also, the human antibody can be selected from a phage library, where that phage library expresses human antibodies, as described, for example, in Vaughan et al., Nat. Biotech. 14:309-314, 1996; Sheets et al., Proc. Nat'l. Acad. Sci. 95:6157-6162, 1998; Hoogenboom and Winter, J. Mol. Biol. 227:381, 1991; and Marks et al., J. Mol. Biol. 222:581, 1991, each incorporated herein by reference. Techniques for the generation and use of antibody phage libraries are also described in U.S. Pat. Nos. 5,969,108, 6,172,197, 5,885,793, 6,521,404; 6,544,731; 6,555,313; 6,582,915; 6,593,081; 6,300,064; 6,653,068; 6,706,484; and 7,264,963; and Rothe et al., J. Mol. Bio. doi: 10.1016/j.jmb.2007.12.018, 2007, each incorporated herein by reference. Affinity maturation strategies and chain shuffling strategies (Marks et al., Bio/Technology 10:779-783, 1992, incorporated herein by reference) are known in the art and can be employed to generate high affinity human antibodies.

Human anti-ZIKV antibodies can also be made in a transgenic mouse containing human immunoglobulin loci that is capable upon immunization of producing the full repertoire of human antibodies in the absence of endogenous immunoglobulin production. This approach is described in U.S. Pat. Nos. 5,545,807; 5,545,806; 5,569,825; 5,625,126; 5,633,425; and 5,661,016, each incorporated herein by reference. In certain embodiments, the antibodies of the present invention are obtained from the transgenic mouse immunized with a PIZV and/or a ZIKV-VLP. The mouse receives at least 2 rounds, e.g., 5 rounds, of the PIZV and/or the ZIKV-VLP by intraperitoneal injection (IP) and allows to rest for one month. Then, the mouse is boosted 4 and 2 days prior to fusion of the spleen with the PIZV and/or the ZIKV-VLP. Lymphatic cells (such as B-cells) are recovered from the spleen of the mouse that produces antibodies. The lymphatic cells may be fused with a myeloma cell line to prepare immortal hybridoma cell lines, and such hybridoma cell lines are screened and selected to identify hybridoma cell lines that produce antibodies specific to a ZIKV. DNAs encoding the variable regions of the heavy chain and light chain may be isolated and linked to desirable isotypic constant regions of the heavy chain and light chain. The DNAs are then cloned into an expression vector(s).

C. Cloning of Anti-ZIKV Antibodies from Hybridomas

To isolate a DNA encoding a heavy chain, a light chain or a fragment thereof, a total RNA is isolated from hybridoma cells using an RNeasy® kit (Qiagen, Hilden, Germany). First and second-strand cDNA synthesis may be performed using a OneTaq® One-Step RT-PCR kit (New England BioLabs, Ipswich, Mass.). PCR products may be separated by agarose electrophoresis and fragments may be excised and purified by a QIAquick® gel extraction kit (Qiagen, Hilden, Germany). Fragments may be cloned directly into expression vectors with BspQI (New England BioLabs, Ipswich, Mass.) by Golden Gate cloning techniques. At least four colonies from each reaction are scaled up for miniprep-scale plasmid purification, e.g., by SequeMid® DNA Purification Kit (Aline Biosciences, Woburn, Mass.). Each plasmid is subjected to Sanger Sequencing. After analysis, unique recombinant heavy chains are paired with unique recombinant light chains. These plasmid pairs are transfected into mammalian cells. Eight to twelve days later conditioned medium from each pairing are screened by FLOW™ or Octet™ for binding to a ZIKV or a ZIKV E protein.

### D. Engineering of Humanized Anti-ZIKV Antibodies

Methods for engineering, humanizing antibodies can also be used and are well known in the art. A humanized antibody can have one or more amino acid residues from a source that is non-human, e.g., but not limited to, mouse, rat, rabbit, non-human primate or other mammal. These non-human amino acid residues are replaced by residues that are often referred to as “import” residues, which are typically taken from an “import” variable, constant or other domain of a known human sequence.

Such imported sequences can be used to reduce immunogenicity or reduce, enhance or modify binding, affinity, on-rate, off-rate, avidity, specificity, half-life, or any other suitable characteristic, as known in the art. In general, the CDR residues are directly and most substantially involved in ZIKV binding. Accordingly, part or all of the non-human CDR sequences are maintained while the non-human sequences of the variable and constant regions can be replaced with human or other amino acids.

Antibodies can also optionally be humanized with retention of high affinity for the ZIKV. To achieve this goal, humanized anti-ZIKV antibodies can be optionally prepared by a process of analysis of the parental sequences and various conceptual humanized products using three-dimensional models of the parental, engineered, and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind ZIKV. In this way, framework (FR) residues can be selected and combined from the consensus and import sequences so that the desired antibody characteristic, such as increased affinity for the target antigen(s), is achieved.

Humanization of antibodies of the present invention can be performed using any known method, such as but not limited to those described in, Jones et al., Nature 321:522, 1986; Riechmann et al., Nature 332:323, 1988; Verhoeyen et al., Science 239:1534, 1988, Sims et al., J. Immunol. 151:2296, 1993; Chothia and Lesk, J. Mol. Biol. 196:901, 1987, Carter et al., Proc. Natl. Acad. Sci. U.S.A. 89:4285, 1992; Presta et al., J. Immunol. 151:2623, 1993; Raguska et al., Proc. Natl. Acad. Sci. U.S.A. 91(3):969-973, 1994; U.S. Pat. Nos. 5,639,641; 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; 4,816,567; 7,557,189; 7,538,195; and 7,342,110; PCT/U598/16280; US96/18978; US91/09630; US91/05939; US94/01234; GB89/01334; GB91/01134; GB92/01755; WO90/14443; WO90/14424; WO90/14430; and EP 229246, each incorporated herein by reference, including the references cited therein.

### E. Production of Recombinant Anti-ZIKV Antibodies

Antibodies may be produced by expression from host cells, wherein an expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express antibodies in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

Preferred mammalian host cells for expressing the recombinant antibodies disclosed herein include 293-6E cells, Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621, each incorporated herein by reference), NSO myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Standard molecular biology techniques are used to prepare the recombinant expression vector, transfect the host cells, select for transformants, culture the host cells and recover the antibody from the culture medium. The recombinant expression vector encoding antibodies may be prepared using nucleic acid molecules corresponding to the amino acid sequences disclosed herein, e.g., SEQ ID NOs: 1-60, 85-114 and 128-137.

In one embodiment, an isolated nucleic acid molecule comprises a heavy chain variable region or a light chain variable region. The nucleic acid sequence of the heavy chain variable region is selected from the group consisting of SEQ ID NOs: 62, 66, 70, 74, 78, 82, 116, 120, 124 and 139. Alterntively, the heavy chain variable region has a sequence having at least 70%, 75%, 80%, 85%, 90%, 92%, 94%, 95%, 96%, 97%, 98%, or 99% identity to SEQ ID NO: 62, 66, 70, 74, 78, 82, 116, 120, 124 or 139. The nucleic acid sequence of the light chain variable region is selected from the group consisting of SEQ ID NOs: 64, 68, 72, 76, 80, 84, 118, 122, 126 and 141. Alterntively, the light chain variable region has a sequence having at least 70%, 75%, 80%, 85%, 90%, 92%, 94%, 95%, 96%, 97%, 98%, or 99% identity to SEQ ID NO: 64, 68, 72, 76, 80, 84, 118, 122, 126 or 141. A recombinant expression vector comprising a nucleic acid disclosed herein encoding a heavy chain variable region can be co-transfected with a recombinant expression vector comprising a nucleic acid also disclosed herein encoding a light chain variable region to produce a monospecific or bispecific anti-ZIKV antibody. Alternatively, a recombinant expression vector comprises a nucleic acid disclosed herein encoding a heavy chain variable region and a nucleic acid also disclosed herein encoding a light chain variable region.

In one embodiment, an isolated nucleic acid molecule comprises a heavy chain and/or a light chain. The nucleic acid sequence of the heavy chain is selected from the group consisting of SEQ ID NOs: 61, 65, 69, 73, 77, 81, 115, 119, 123, and 138. Alterntively, the heavy chain has a sequence having at least 70%, 75%, 80%, 85%, 90%, 92%, 94%, 95%, 96%, 97%, 98%, or 99% identity to SEQ ID NO: 61, 65, 69, 73, 77, 81, 115, 119, 123 or 138. The nucleic acid sequence of the light chain is selected from the group consisting of SEQ ID NOs: 63, 67, 71, 75, 79, 83, 117, 121, 125 and 140. Alternatively, the light chain has a sequence having at least 70%, 75%, 80%, 85%, 90%, 92%, 94%, 95%, 96%, 97%, 98%, or 99% identity to SEQ ID NO: 63, 67, 71, 75, 79, 83, 117, 121, 125 or 140. A recombinant expression vector comprising a nucleic acid disclosed herein encoding a heavy chain can be co-transfected with a recombinant expression vector comprising a nucleic acid also disclosed herein encoding a light chain to produce an anti-ZIKV antibody. Alternatively, a recombinant expression vector comprises a nucleic acid disclosed herein encoding a heavy chain and a nucleic acid also disclosed herein encoding a light chain.

Host cells can also be used to produce functional antibody fragments, such as Fab fragments or scFv molecules. It will be understood that variations on the above procedure are within the scope of the disclosure. For example, it may be desirable to transfect a host cell with DNA encoding functional fragments of either the light chain and/or the heavy chain of an antibody. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies of the disclosure. In addition, bifunctional antibodies may be produced in which one heavy and one light chain are an antibody of the disclosure and the other heavy and light chain are specific for an antigen other than the antigens of interest by crosslinking an antibody of the disclosure to a second antibody by standard chemical crosslinking methods.

In a preferred system for recombinant expression of an antibody, or antigen-binding portion thereof, the host cells for a recombinant expression vector encoding both the antibody heavy chain and the antibody light chain is 293-6E cells.

Further, the present invention provides for a method of isolating the recombinant antibody from the culture medium. For example, harvested culture medium is centrifuged to remove cell debris and the clear supernatant containing secreted monoclonal antibodies is purified through MabSelect SuRe protein A column chromatography. The eluted antibody is dialyzed in PBS buffer, sterile filtered, and adjusted to pH 7.4. Purified antibody is serially diluted and tested in ELISA against a ZIKV-VLP and a ZIKV Envelope Recombinant Protein. Concentration determination is performed by ELISA at OD405nm using goat anti-rabbit IgG. The purity and integrity of recombinant antibodies is verified by SDS-PAGE under non-reduced and reduced conditions.

### IV. Pharmaceutical Compositions

The disclosure also provides pharmaceutical compositions comprising an antibody, or antigen-binding portion thereof, and a pharmaceutically acceptable carrier, diluent or excipient. The pharmaceutical compositions comprising antibodies are for use, but not limited to, in preventing, treating, managing, or ameliorating of a condition or one or more symptoms thereof, caused by a ZIKV infection. In a specific embodiment, the pharmaceutical composition comprises one or more antibodies, or antigen-binding portions thereof, according to the present invention. In another embodiment, the pharmaceutical composition comprises one or more antibodies, or antigen-binding portions thereof, according to the present invention and one or more prophylactic or therapeutic agents other than antibodies for treating an infection of a ZIKV that is detrimental.

Pharmaceutical compositions in accordance with the present invention will be prepared with suitable carriers, excipients, diluents, and other agents that are incorporated into formulations to provide improved transfer, delivery, tolerance, and the like.

Typically, the pharmaceutical composition comprises an antibody or antibody portion and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol, oil, and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody or antigen-binding portion thereof, according to the present invention.

Various delivery systems are known and can be used to administer one or more antibodies or the combination of one or more antibodies and a prophylactic agent or therapeutic agent useful for preventing, managing, treating, or ameliorating a ZIKV infection or one or more symptoms thereof, e.g., encapsulation in liposomes, microparticles, microcapsules, recombinant cells capable of expressing the antibody or antibody fragment, receptor-mediated endocytosis (see, e.g., Wu and Wu, J. Biol. Chem. 262:4429-4432 (1987), incorporated herein by reference), construction of a nucleic acid as part of a retroviral or other vector, etc.

A pharmaceutical composition is formulated to be compatible with its intended route of administration. Examples of routes of administration include, but are not limited to, parenteral (e.g., intravenous, intramuscular, intradermal, and subcutaneous), oral, intranasal (e.g., inhalation), transdermal (e.g., topical), transmucosal, and rectal administration. Alternatively, the routes of administration also include a pump. In a specific embodiment, the composition is formulated in accordance with routine procedures as a pharmaceutical composition adapted for intravenous, subcutaneous, intramuscular, oral, intranasal, or topical administration to a subject.

Typically, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (PEG-50 Hydrogenated Castor W)], etc. As the oily medium, there are, e.g., sesame oil, soybean oil, etc., which may be used in combination with a solubilizing agent such as benzyl benzoate, benzyl alcohol, etc. Where necessary, the composition may also include a local anesthetic such as lignocaine to ease pain at the site of the injection.

Formulations for injection may be presented in unit dosage form (e.g., in ampoules or in multi-dose containers) with an added preservative. The pharmaceutical compositions may take such forms as suspensions, solutions or emulsions in oily or aqueous vehicles, and may contain formulatory agents such as suspending, stabilizing and/or dispersing agents. Alternatively, the pharmaceutical compositions may be in powder form for constitution with a suitable vehicle (e.g., sterile pyrogen-free water) before use. Generally, the ingredients of compositions are supplied either separately or mixed together in unit dosage form, for example, as a dry lyophilized powder or water free concentrate in a hermetically sealed container such as an ampoule or sachette indicating the quantity of active agent. Where the mode of administration is infusion, composition can be dispensed with an infusion bottle containing sterile pharmaceutical grade water or saline. Where the mode of administration is by injection, an ampoule of sterile water for injection or saline can be provided so that the ingredients may be mixed prior to administration.

In particular, the disclosure also provides that one or more of the prophylactic or therapeutic agents, or pharmaceutical compositions are packaged in a hermetically sealed container such as an ampoule or sachette indicating the quantity of the agent. In one embodiment, one or more of the prophylactic or therapeutic agents, or pharmaceutical compositions is supplied as a dry sterilized lyophilized powder or water free concentrate in a hermetically sealed container and can be reconstituted (e.g., with water or saline) to the appropriate concentration for administration to a subject. The antibodies, or antigen-binding portions thereof, according to the present invention can be administered by a variety of methods known in the art, although for many therapeutic applications, the preferred route/mode of administration is subcutaneous injection, intravenous injection or infusion.

As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, polylactic acid, and poly(lactic-co-glycolic acid). Many methods for the preparation of such formulations are patented or generally known to those skilled in the art. See, e.g., Sustained and Controlled Release Drug Delivery Systems, J. R. Robinson, ed., Marcel Dekker, Inc., New York, 1978, incorporated herein by reference.

Formulations comprising nanoparticles to deliver the antibodies, or antigen-binding portions thereof, according to the present invention may be used both for therapeutic and diagnostic applications. Antibody-conjugated nanoparticles and methods of preparation and use are described in detail by Arruebo, M., et al. 2009 (“Antibody-conjugated nanoparticles for biomedical applications” in J. Nanomat. Volume 2009, Article ID 439389, 24 pages, doi: 10.1 155/2009/439389), incorporated herein by reference. Nanoparticles may be developed and conjugated to antibodies contained in pharmaceutical compositions to target virally infected cells. Nanoparticles for drug delivery have also been described in, for example, U.S. Pat. Nos. 8,257,740, or 8,246,995, each of which is incorporated herein by reference.

In certain embodiments, the formulations comprising nanoparticles can be used in a controlled release system. In certain embodiment, a controlled release system can be placed in proximity of the composition's target, thus requiring only a fraction of the systemic dose. In another embodiment, biodegradable, biocompatible polymers can be used in the formulations comprising nanoparticles.

If the method of the disclosure comprises oral administration, compositions can be formulated orally in the form of tablets, capsules, cachets, gel caps, solutions, suspensions, and the like. Tablets or capsules can be prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets may be coated by methods well-known in the art. Liquid preparations for oral administration may take the form of, but not limited to, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives, or hydrogenated edible fats);

emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of a prophylactic or therapeutic agent(s).

The present invention includes pharmaceutical compositions in which an anti-ZIKV antibody, or an antigen-binding portion thereof, according to the present invention is co-formulated with one or more of the additional therapeutically active component(s) as described herein.

### V. Therapeutic Uses of Anti-ZIKV Antibodies

The isolated antibodies and antigen-binding portions as described herein preferably are capable of neutralizing a ZIKV both in vivo and in vitro. Accordingly, such antibodies and antigen-binding portions can be used to inhibit infection of a ZIKV, e.g., in a cell culture exposed to a ZIKV, in human subjects or in other mammalian subjects infected with a ZIKV.

The anti-ZIKV antibodies or antigen-binding portions thereof, according to the present invention are useful for the treatment of a disease or disorder or condition associated with ZIKV infection and/or for ameliorating at least one symptom associated with such disease, disorder or condition. The symptom includes, but not limited to, fever, headache, arthralgia, myalgia, and maculopapular rash.

In some embodiments, the anti-ZIKV antibodies, or antigen-binding portions thereof, according to the present invention are useful in decreasing viral titers or reducing viral load in the host. In one embodiment, the anti-ZIKV antibody, or antigen-binding portion thereof, according to the present invention may be administered at a therapeutic dose to a subject with ZIKV infection.

In some embodiments, the anti-ZIKV antibodies may be useful for treating a subject who is at risk for acquiring a ZIKV infection (e.g. a pregnant female who is living in, or visiting, a country that has a ZIKV outbreak). The antibodies when administered to a subject in need thereof may reduce the ZIKV infection in the subject. They may be used to decrease viral loads in a subject. They may be used alone or as a combination therapy with other therapeutic moieties or modalities known in the art for treating a viral infection. The identified antibodies can be used prophylactically (before infection) to protect a mammal from infection, or can be used therapeutically (after infection is established) to ameliorate a previously established infection, or to ameliorate at least one symptom associated with the infection.

It is also contemplated herein to use one or more anti-ZIKV antibodies of the present invention prophylactically to subjects at risk for developing a ZIKV infection such as an immunocompromised individual, a person who has been bitten by a mosquito believed to harbor the ZIKV, a pregnant woman, who lives in, or is visiting a country known to have a ZIKV outbreak, or a woman who lives in, or is visiting a country known to have a ZIKV outbreak and who is considering conceiving a child, an individual visiting, or living in an area known to harbor mosquitoes suspected of carrying the ZIKV, an individuals who has visited or is planning to visit an area or country known to have or suspected to have an outbreak of ZIKV.

The term “administering” as used herein is meant to refer to the delivery of a substance (e.g., an anti-ZIKV antibody) to achieve a therapeutic objective (e.g., the treatment of a ZIKV infection). Methods of administration may be parenteral, enteral and topical. Parenteral administration is usually by injection, and includes, without limitation, intravenous, intramuscular, intraarterial, intrathecal, intracapsular, intraorbital, intracardiac, intradermal, intraperitoneal, transtracheal, subcutaneous, subcutcular, intraarticular, subcapsular, subarachnoid, intraspinal, intracranial and intrastemal injection and infusion. Topical administration includes transdermal and intranasal routes through epithelial or mucocutaneous linings (e.g., oral mucosa, rectal and intestinal mucosa, etc.). Administration can be systemic or local.

In addition, pulmonary administration can be employed, e.g., by use of an inhaler or nebulizer, and formulation with an aerosolizing agent. See, e.g., U.S. Pat. Nos. 6,019,968, 5,985, 320, 5,985,309, 5,934, 272, 5,874,064, 5,855,913, 5,290, 540, and 4,880,078; and PCT Publication Nos. WO 92/19244, WO 97/32572, WO 97/44013, WO 98/31346, and WO 99/66903, each of which is incorporated herein by reference in its entirety. In one embodiment, an antibody, combination therapy, or a composition is administered using Alkermes AIR® pulmonary drug delivery technology (Alkermes, Inc., Cambridge, Mass.). In a specific embodiment, prophylactic or therapeutic agents are administered intramuscularly, intravenously, intracranially, orally, intranasally, pulmonary, or subcutaneously. The prophylactic or therapeutic agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In one embodiment, a pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but certainly are not limited to AUTOPEN™ (Owen Mumford, Inc., Woodstock, UK), DISETRONIC™ pen (Disetronic Medical Systems, Burghdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition of the present invention include, but certainly are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L.P.) and the HUMIRA™ Pen (Abbott Labs, Abbott Park, Ill.), to name only a few.

As used herein, the term “effective amount” or “therapeutically effective amount” refers to an amount that produces the desired effect for which it is administered. The exact amount will depend on the purpose of the treatment, and will be ascertainable by one skilled in the art using known techniques (see, for example, Lloyd (1999) The Art, Science and Technology of Pharmaceutical Compounding). For example, the amount of an antibody is sufficient to inhibit worsening of a ZIKV infection, reduce ZIKV titers, or ameliorate the severity of at least one symptom or indication of a ZIKV infection.

As used herein, the term “subject” refers to an animal, preferably a mammal, more preferably a human, in need of amelioration, prevention and/or treatment of a disease or disorder such as a ZIKV infection. The subject may have a ZIKV infection or is predisposed to developing a ZIKV infection. Subjects “predisposed to developing a ZIKV infection”, or subjects “who may be at elevated risk for contracting a ZIKV infection”, are those subjects with compromised immune systems because of autoimmune disease, those persons receiving immunosuppressive therapy (for example, following organ transplant), those persons afflicted with human immunodeficiency syndrome (HIV) or acquired immune deficiency syndrome (AIDS), pregnant females who have been exposed to, or who may become exposed to the ZIKV if living in, or visiting an area that has an outbreak of ZIKV, in addition to a woman who lives in, or is visiting an area known to have a ZIKV outbreak and is considering conceiving a child, certain forms of anemia that deplete or destroy white blood cells, those persons receiving radiation or chemotherapy, or those persons afflicted with an inflammatory disorder.

Additionally, subjects of extreme young or old age are at increased risk. Any person who comes into physical contact or close physical proximity with an infected animal, or human patient, or is exposed to bodily fluids or tissues from an infected animal or human patient, has an increased risk of developing a ZIKV infection. Moreover, a subject is at risk of contracting an ZIKV infection due to proximity to an outbreak of the disease, e.g. subject resides in a densely-populated city or in close proximity to subjects having confirmed or suspected infections of ZIKV, or choice of employment, e.g. hospital worker, pharmaceutical researcher, or an individual who has visited or who is planning to visit an area or country known to have or suspected to have an outbreak of ZIKV. There is also an increased risk of severe outcomes in a subject if they contract a ZIKV infection. When a pregnant woman is infected with ZIKV, there is an increased risk that the baby may be born with microcephaly or other developmental abnormalities. Accordingly, if a woman is pregnant, or is considering conceiving a child, and she is living in an area where there is a ZIKV outbreak, or visiting an area where there is a ZIKV outbreak, or is in an area that is known to have mosquitoes that harbor the ZIKV, she is at risk for contracting a ZIKV infection. There is also an increased risk of developing Guillain-Barre syndrome after exposure of a subject to ZIKV.

As used herein, the terms “treat”, “treating”, or “treatment” refer to the reduction or amelioration of the severity of at least one symptom or indication of ZIKV infection due to the administration of a therapeutic agent such as an antibody of the present invention to a subject in need thereof. The terms include inhibition of progression of disease or of worsening of infection. The terms also include positive prognosis of disease, i.e., the subject may be free of infection or may have reduced or no viral titers upon administration of a therapeutic agent such as an antibody of the present invention. The therapeutic agent may be administered at a therapeutic dose to the subject.

The terms “prevent”, “preventing” or “prevention” refer to inhibition of manifestation of a ZIKV infection or any symptoms or indications of a ZIKV infection upon administration of an antibody, or antigen-binding portion, according to the present invention. The term includes prevention of spread of infection in a subject exposed to the virus or at risk of having a ZIKV infection.

The dose of antibody may vary depending upon the age and the size of a subject to be administered, route of administration, and the like. When an antibody, or an antigen-binding portion thereof, according to the present invention is used for treating a ZIKV infection in an adult patient, or for preventing such a ZIKV infection, it is advantageous to administer the antibody, or an antigen-binding portion thereof, according to the present invention normally at a single dose of about 0.001 to about 60 mg/kg body weight, more preferably about 0.001 to about 1, about 1 to about 5, about 5 to about 10, about 10 to about 20, or about 20 to about 60 mg/kg body weight.

Depending on the severity of the condition, the frequency and the duration of the treatment can be adjusted. In certain embodiments, the antibody, or antigen-binding portion thereof, according to the present invention can be administered as an initial dose of about 0.05 mg to about 4500 mg, about 0.1 mg to about 3500 mg, about 0.5 mg to about 2500 mg, about 1 mg to about 1500 mg, about 2.5 mg to about 750 mg, about 5 mg to about 500 mg, about 10 mg to about 300 mg, or about 20 mg to about 200 mg. For example, the initial dose is selected from the group consisting of 200, 160, 80, 40 and 20 mg. In one embodiment, subsequent doses is the same as the initial dose. In another embodiment, subsequent doses can be different from the initial dose. For example, the anti-ZIKV antibody is administered at a dose of160 mg initially on Day 1, followed by a second dose of 80 mg two weeks later (Day 15), and further followed by a subsequent maintenance dose of 40 mg beginning four weeks later (Day 29) and continuing every other week thereafter. The subsequent doses of administration may also be adjusted during the course of treatment by a physician depending on the needs of the subject following clinical examination.

In certain embodiments, the initial dose may be followed by administration of a second or a plurality of subsequent doses of the antibody or antigen-binding portion thereof in an amount that can be approximately the same or less than that of the initial dose, wherein the subsequent doses are separated by 1 to 24 hours (e.g., e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24), at least 2 days, at least 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks. In one embodiment, one or more (e.g., 1, 2, 3, 4, 5, 6, 7, 8, or more) subsequent doses are administered to a subject in need thereof. In certain embodiments of the present invention, the frequency at which subsequent doses are administered to a subject in need thereof can vary over the course of the treatment regimen. The frequency of administration may also be adjusted during the course of treatment by a physician depending on the needs of the subject following clinical examination.

The term “combination therapy”, as used herein, refers to the administration of two or more therapeutic substances, e.g., an anti-ZIKV antibody of the present invention and an additional therapeutic agent. The additional therapeutic agent may be administered concomitant with, prior to, or following the administration of the anti-ZIKV antibody.

As used herein, the term “additional therapeutic agent” or “second therapeutic agent” refers to any anti-infective agent or therapy, which can be a chemical moiety, or a biological therapy, used to treat, prevent, or ameliorate a ZIKV infection in a subject. For example, in the present invention, a second therapeutic agent may include, but not be limited to, an antibody to a ZIKV (in one embodiment the antibody to a ZIKV may be different than those described herein), a vaccine for a ZIKV, a direct-acting anti-viral agent, an immune modulator (e.g., an interferon), and an anti-inflammatory drug (such as corticosteroids, and non-steroidal anti-inflammatory drugs, such as anti-TNF).

In some embodiments, the anti-ZIKV antibodies, or antigen-binding portions thereof, according to the present invention may be combined with a second therapeutic agent to reduce the viral load in a patient with a ZIKV infection, or to ameliorate one or more symptoms of the infection and/or spread to fetus or male reproductive organs.

In certain embodiments, the second therapeutic agent is another different antibody, or antibody cocktail specific for a ZIKV E protein, wherein the different antibody or antibodies within the cocktail may or may not bind to the same epitope, or an overlapping epitope, as an antibody of the present invention. In certain embodiments, the second therapeutic agent is an anti-ZIKV antibody to a different ZIKV protein. The second antibody may be specific for one or more different ZIKV proteins from different strains of the virus. It is contemplated herein to use a combination (“cocktail”) of the antibodies of the present invention with neutralization or inhibitory activity against ZIKV. In some embodiments, non-competing antibodies may be combined and administered to a subject in need thereof, to reduce the ability of ZIKV to escape due to mutation. In some embodiments, the anti-ZIKV antibodies used in the combination bind to distinct non-overlapping epitopes on the E protein. The anti-ZIKV antibodies used in the combination may block the virus attachment to the cell, and/or may inhibit fusion of the virus with the cell membrane, and in so doing may block ZIKV entry into the host cells. The antibodies may interact with the E protein from a strain of a ZIKV selected from MR766 (Uganda 1947), PRVABC59 (Puerto Rico 2015), FLR (Colombia 2015), SV0127-14 (Thailand, 2014), CPC-0740 (Philippine 2012), and Fortaleza (Brazil 2015) strains, and when used alone, or in combination with any one or more of the agents noted above, may neutralize any one or more of the ZIKV strains noted, or variants thereof.

It is also contemplated herein to use a combination of anti-ZIKV antibodies, or antigen-binding portions thereof, according to the present invention, wherein the combination comprises one or more antibodies that do not cross-compete. In certain embodiments, the combination includes a cocktail comprising a mixture of at least two or at least three antibodies of the present invention. The anti-ZIKV antibodies within the cocktail may differ in their abilities to neutralize virus or virus infected cells, or in their abilities to block attachment of the virus to the cell, or block fusion of the virus to the cell membrane, or in their ability to bind a ZIKV E protein.

The additional therapeutically active component(s) may be administered to a subject prior to administration of an anti-ZIKV antibody, or an antigen-binding portion, according to the present invention. For example, a first component may be deemed to be administered “prior to” a second component if the first component is administered 1 week before, 72 hours before, 60 hours before, 48 hours before, 36 hours before, 24 hours before, 12 hours before, 6 hours before, 5 hours before, 4 hours before, 3 hours before, 2 hours before, 1 hour before, 30 minutes before, 15 minutes before, 10 minutes before, 5 minutes before, or less than 1 minute before administration of the second component. In other embodiments, the additional therapeutically active component(s) may be administered to a subject after administration of an anti-ZIKV antibody, or an antigen-binding portion thereof, according to the present invention. For example, a first component may be deemed to be administered “after” a second component if the first component is administered 1 minute after, 5 minutes after, 10 minutes after, 15 minutes after, 30 minutes after, 1 hour after, 2 hours after, 3 hours after, 4 hours after, 5 hours after, 6 hours after, 12 hours after, 24 hours after, 36 hours after, 48 hours after, 60 hours after, 72 hours after administration of the second component. In yet other embodiments, the additional therapeutically active component(s) may be administered to a subject concurrent with administration of an anti-ZIKV antibody, or an antigen-binding portion thereof, according to the present invention. “Concurrent” administration, for purposes of the present invention, includes, e.g., administration of an anti-ZIKV antibody and an additional therapeutically active component to a subject in a single dosage form, or in separate dosage forms administered to the subject within about 30 minutes or less of each other. If administered in separate dosage forms, each dosage form may be administered via the same route (e.g., both the anti-ZIKV antibody and the additional therapeutically active component may be administered intravenously, etc.); alternatively, each dosage form may be administered via a different route (e.g., the anti-ZIKV antibody may be administered intravenously, and the additional therapeutically active component may be administered orally). In any event, administering the components in a single dosage from, in separate dosage forms by the same route, or in separate dosage forms by different routes are all considered “concurrent administration,” for purposes of the present disclosure. For purposes of the present disclosure, administration of an anti-ZIKV antibody “prior to”, “concurrent with,” or “after” (as those terms are defined herein above) administration of an additional therapeutically active component is considered administration of an anti-ZIKV antibody “in combination with” an additional therapeutically active component.

As used herein, the term “in combination with” means that an additional therapeutically active component(s) may be administered prior to, concurrent with, or after the administration of at least one anti-ZIKV antibody of the present invention, or a cocktail comprising two or more of the antibodies the present invention. The term “in combination with” also includes sequential or concomitant administration of an anti-ZIKV antibody and a second therapeutic agent.

### VI. Non-Therapeutic Uses of Anti-ZIKV Antibodies

The anti-ZIKV antibodies or antigen-binding portions thereof, according to the present invention may also be used for non-pharmaceutical purposes, such as in diagnosis or detection of a ZIKV infection or for analytical purposes.

In one embodiment, the antibodies can be used in competition ELISA where antibody response to a vaccine can be tested to see if antibodies have been induced against certain immunogenic epitopes.

In one embodiment, the antibodies can be used in an adventituous agent testing where the viral infectivity is neutralized using the monoclonal antibodies.

In one embodiment, the antibodies can be used in immunoassays for diagnosis of ZIKV infection. Preferred examples of immunoassays include ELISA, immunofluorescence, immunohistochemistry and flow cytometry. More preferably, diagnosis includes ELISA. For example, a standard ELISA, a sandwich ELISA or a blockade of binding assay may be used. Other assays for which the antibodies could be used include western blot, PRNT, MNT, TCID50 based netralization assays.

A standard ELISA diagnostic assay for a ZIKV infection may comprise, e.g., contacting a sample, obtained from a subject, with an anti-ZIKV antibody, or antigen-binding portion thereof, according to the present invention, wherein the anti-ZIKV antibody is labeled with a detectable label or reporter molecule or used as a capture ligand to selectively isolate a ZIKV from the sample. Alternatively, an unlabeled anti-ZIKV antibody can be used in diagnostic applications in combination with a secondary antibody which is itself detectably labeled. The detectable label or reporter molecule can be a radioisotope, such as ³H, ¹⁴C, ³²P, ³⁵S, or ¹²⁵I; a fluorescent or chemiluminescent moiety such as fluorescein, fluorescein isothiocyanate, or rhodamine; or an enzyme such as alkaline phosphatase, β-galactosidase, horseradish peroxidase, or luciferase. Specific exemplary assays that can be used to detect or measure a ZIKV in a sample include enzyme-linked immunosorbent assay (ELISA), radioimmunoassay (RIA), and fluorescence-activated cell sorting (FACS). The sample is selected from tissues or bodily fluids that may be loaded with an infected ZIKV. The body fluids include blood (e.g. whole blood, plasma, serum), saliva and urine. Blood, in particular, plasma or serum, is most preferred. Generally, levels of ZIKV in a particular sample obtained from a healthy subject (e.g., a subject not afflicted with a disease associated with ZIKV will be measured to initially establish a baseline as a negative control. This baseline level of ZIKV can then be compared against the levels of ZIKV measured in samples obtained from individuals suspected of having a ZIKV-associated condition, or symptoms associated with such condition.

A blockade-of-binding assay for a ZIKV infection comprises at least three steps. Specifically, an isolated sample from a subject to be diagnosed (e.g., a sample of a body fluid, such as blood (e.g. whole blood, plasma, serum), saliva and urine) is added to an ELISA plate coated with a ZIKV E protein and incubated (for example, for at least about 30 minutes or at least about one hour) to allow for binding. Thereafter, the antibody, or antigen-binding portion thereof, according to the present invention is added (as “probe antibody”), wherein the antibody, or antigen-binding portion thereof, according to the present invention is preferably labelled, e.g. biotinylated or conjugated to horseradish peroxidase (HRP). After another incubation time (e.g., at least about 1 minute, preferably at least about 3 minutes, more preferably at least about 5 minutes, even more preferably at least about 10 minutes, most preferably at least about 15 minutes), inhibition of binding of the antibody, or antigen-binding portion thereof, according to the present invention can be determined. In general, inhibition of binding shows the presence of anti-ZIKV E protein antibodies in the sample of the subject, thus indicating ZIKV infection of the subject. In samples of non-infected subjects, in contrast, typically no inhibition of binding is expected. Importantly, such an assay using the anti-ZIKV antibodies of the present invention does not score positive in subjects that were already infected with other Flaviviruses. Flaviviruses typically induce a large number of antibodies that are cross-reactive with ZIKV. In other words this assay is highly specific and not affected by cross-reactive antibodies.

In a diagnosis assay, the anti-ZIKV antibody or the antigen-binding portion thereof is solubilized in a solution for direct use. The solution (vehicle) may be chosen according to the purpose, e.g. depending on the assay. Preferably, the solution comprises PBS (phosphate-buffered saline) or another buffer. Such buffers are preferably biological buffers, and the buffer can be any of MES, BIS-TRIS, ADA, PIPES, ACES, MOPSO, BIS-TRIS propane, BES, MOPS, TES, HEPES, DIPSO, TAPSO, Trizma, POPSO, HEPPS, TRICINE, Gly-Gly, BICINE, HEPBS, TAPS, AMPD, AMPSO, CHES, CAPSO, AMP, CAPS and CABS. It is also preferred that the solution is Ringer's solution. In addition, the solution may also comprise Tris, e.g., Tris-HCI.

The solution described above may also comprise a detergent e.g., a Tween (polysorbate), such as Tween 20 or Tween 80. Detergents are preferably present at low levels e.g., less than 0.01%. The solution may also include sodium salts (e.g., sodium chloride) to give tonicity. For example, a concentration of 10±2mg/ml NaCl is typical.

In addition, the solution described above may optionally comprise a protein stabilizer, such as BSA (bovine serum albumin) or HSA (human serum albumin). Further examples of protein stabilizers, which may optionally be included in the solution, include buffers, e.g. as described above; salts, such as sodium chloride; amino acids, such as histidine, glycine, and arginine; polyols/disaccharides/polysaccharides, such as trehalose and sucrose (disaccharides), mannitol and sorbitol (sugar alcohols); surfactants, such as polysorbate 20, polysorbate 80, and proteins like HSA or BSA; polymers, such as dextran and polyethylene glycol; and antioxidants.

Furthermore, the solution above may optionally comprise a preservative, such as sodium azide. Preservatives are typically used to prevent microbial contamination.

### VII. Kits For Use

The present disclosure also provides kits for use in neutralizing an infectious ZIKV, preventing, treating or ameliorating at least one symptom of a ZIKV infection, and decreasing the likelihood of transmitting a ZIKV infection to the fetus of a pregnant female, and/or prevention of transmission to the male reproductive organs. In addition, the present disclosure also provides kits for use in in vitro diagnosing a ZIKV infection.

In some embodiments, the kit can comprise instructions for use in accordance with any of the methods described herein. The included instructions can comprise a description of administration of the anti-ZIKV antibody to neutralize an infectious ZIKV, to prevent, treat or ameliorate at least one symptom of a ZIKV infection, to decrease the likelihood of transmitting a ZIKV infection to the fetus of a pregnant female, and/or to prevent transmission to the male reproductive organs as those described herein.

The instructions relating to the use of an anti-ZIKV antibody generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert (e.g., a paper sheet included in the kit), but machine-readable instructions (e.g., instructions carried on a magnetic or optical storage disk) are also acceptable.

The label or package insert indicates that the composition is used for neutralizing an infectious ZIKV, preventing, treating or ameliorating at least one symptom of a ZIKV infection, decreasing the likelihood of transmitting a ZIKV infection to the fetus of a pregnant female, and/or to preventing transmission to the male reproductive organs. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an anti-ZIKV antibody as those described herein.

In still other embodiments, the instructions comprise a description of using the anti-ZIKV antibody for in vitro diagnosing a ZIKV infection. Kits may optionally provide additional components such as buffers and interpretive information. Normally, the kit comprises a container and a label or package insert(s) on or associated with the container. In some embodiments, the disclosure provides articles of manufacture comprising contents of the kits described above.

It will be readily apparent to those skilled in the art that other suitable modifications and adaptations of the methods of the present invention described herein are obvious and may be made using suitable equivalents without departing from the scope of the present invention or the embodiments disclosed herein. Having now described the present invention in detail, the same will be more clearly understood by reference to the following examples, which are included for purposes of illustration only and are not intended to be limiting.

### EXAMPLES

The following examples are put forth so as to provide those of ordinary skill in the art with a complete disclosure and description of how to make and use the methods and compositions of the present invention, and are not intended to limit the scope of what the inventors regard as their invention.

### Example 1: Isolation of ZIKV-Specific Antibodies and Production of Anti-ZIKV Antibodies

### Animal and Immunization

A total of two (2) New Zealand White (NZW) female rabbits (TAK 466 and TAK 472), all obtained from Covance Research Products (Denver, PA) were selected due to their high anti-ZIKV ELISA antibody titer. The animal enrollment and treatment group assignment details are listed in Table 1.

TABLE 1
Animal Enrollment and Treatment Group Assignments

| | | Immunization | | Inoculation |
|---|---|---|---|---|
| Rabbit | Test Article | on Day | Dose Volume | Route |
| 1 | Lo-Zika Vax | 0, 14, 28 | 0.5 mL | IM |
| | (5 μg) | | | (single site) |
| | ZIKV-VLP | 109 (TAK 466) | 0.5 mL (5 μg) | IM |
| | | 130 (TAK 466) | 1.0 mL (50 μg) | IV |
| 2 | Lo-Zika Vax | 0, 28, 56, 95 | 0.5 mL | IM |
| | (5 μg) | | | (single site) |
| | ZIKV-VLP | 109 (TAK 472) | 0.5 mL (5 μg) | IM |
| | | 130 (TAK 472) | 1.0 mL (50 μg) | IV |

TABLE 2
Test Article Details

| | |
|---|---|
| Test Article: | Lo-Zika Vax |
| Lot Number: | 161205-8 |
| Storage Conditions: | 2-8° C. |
| Test Article: | ZIKV-VLP (Native Antigen Inc.) |
| Lot Number: | 17040712 |
| Concentration: | 0.12 mg/mL |
| Storage Conditions: | Short Term: +2-8° C. |
| | Long Term: −80 ± 15° C. |
| Purity: | >95% by SDS-PAGE |
| Buffer Constitution: | 20 mM Tris-HCI pH 7.5, 300 mM NaCl |

The Lo-Zika Vax vaccine is a PIZV formulated with alum and was handled aseptically in the biosafety cabinet. The vaccine was administered to the animals without preparation or formulations, as a ready to use vaccine. A ZIKV-VLP with adjuvant (Freund's incomplete adjuvant, 0.25 ml) (on Day 109) and the ZIKV-VLP without adjuvant (on Day 130) was administered to animals.

### Generation of Hybridoma

### Lymphocyte Isolation

Spleens from rabbits TAK466 and TAK472 were delivered to Abcam and splenocytes were isolated 4 days after last immunization following Abcam's protocols. Briefly, the spleen was transferred to a culture dish and excess fat was trimmed off using forceps and scissors.

The spleen was then punctured and flushed with culture medium (1:100 dilution) (RPMI medium with penicillin/streptomycin/fungizone, Fisher Cat # BW17745E) using a sterile needle and syringe and then crushing it into pieces with end of syringe. Suspension was then piptted few times and passed through a cell strainer collecting the filtrate into a Flacon tube. The cell debris was treated same way 3-5 times and passed through the strainer collecting all filtrate into the same Falcon tube. Then the cell suspension was spun at 1600rpm for 15 min and supernatant was discarded. Then red cell lysis buffer was added to the cell pellet and incubated for 4 min. More culture medium was added and cells spun at 1600 rpm. The cell pellet was finally resuspended in the medium and cell counts was done. Cells were aliquted into freezing medium (90% FBS and 10% DMSO) and frozen at −80° C. o/n, followed by transferring to liquid nitrogen storage. Details of the spleen and isolated splenocytes are described in Table 3.

TABLE 3
Rabbit spleen and splenocytes details for TAK466 and TAK472.

| Rabbit | | Tissue | Weight | Size | Viability | Total Cells |
|---|---|---|---|---|---|---|
| ID | Immunogen | type | (g) | (cm) | (%) | (in Millions) |
| TAK466 | PIZV and | spleen | 1.63 | 5.8 | 87.7 | 1180.4 |
| | VLP | | | | | |
| TAK472 | PIZV and | spleen | 1.13 | 4.7 | 87.9 | 1060.4 |
| | VLP | | | | | |

### Fusion

For each monoclonal fusion, 200 million splenocytes were fused with 100 million fusion partner cells (240E-W2 cells, developed and patented by Epitomics/Abcam) and plated into twenty 96-well plates. Four fusions were performed next day using TAK466 and TAK472 splenocytes and the resulting hybridomas were plated into a total of eighty 96-well plates.

After fusion, the hybridomas were cultured in tissue culture incubators under standard conditions. Cell growth was examined 2-3 weeks after fusion and fusion efficiency was analyzed. Fusion efficiency was calculated as the total number of wells containing hybridoma colonies divided by the total number of wells examined. A minimum of two plates were examined per fusion. The fusion information is listed in Table 4.

TABLE 4
Fusion details.

| | | Fusion |
|---|---|---|
| Rabbit ID | Fusion ID | Efficiency (%) |
| TAK466 | Fl | 94 |
| TAK466 | F2 | 82 |
| TAK472 | F3 | 77 |
| TAK472 | F4 | 68 |

### Screening

After fusion, multiclone supernatants were screened at Abcam by standard ELISA for reactivity with a ZIKV-VLP (Native Antigen, Inc.) and ZIKV E-Protein (Native Antigen, Inc.). For each ELISA screen, antigens were coated at 50 ng per well. TAK466 and TAK472 rabbit bleeds (pooled hyperimmune serum following 3 immunizations with PIZV as shown in Table 1) were used as positive controls at a 1:100 dilution for their corresponding plates in Table 5. Fresh media that had not been conditioned with cells was used as the negative control for all 80 plates.

One of the 96-well plates (plate #5) was pre-screened 9 days after the fusion, followed by a primary ELISA screen of all 80 plates with a ZIKV-VLP antigen 14 days after fusion. A confirming ELISA screen which included the primary antigen ZIKV-VLP and secondary antigen E-protein was performed 20 days after fusion. The screening process and ELISA results are shown in Table 5.

TABLE 5
Summary of screening process and results.

| | | | Number of | Number of | Number of |
|---|---|---|---|---|---|
| | Screening | Plates | Multiclones | Multiclones | Multiclones |
| Screen | antigen | screened | O.D. > 1 | 0.5 < O.D. < 1 | O.D. < 0.5 |
| Pre-screen | ZIKV- | 1 plate | 0 | 0 | 96 |
| | VLP | (hybridoma | | | |
| | | plate #5) | | | |
| Primary screen | ZIKV- | All 80 plates | 173 | 1,055 | 5,812 |
| | VLP | | | | |
| Confirm screen | ZIKV- | 348 strong | 194 | 114 | 40 |
| | VLP | positive | | | |
| | | clones from | | | |
| | | primary | | | |
| | | screen | | | |
| Confirm screen | ZIKV E- | 348 strong | 28 | 1 | 319 |
| | Protein | positive | | | |
| | | clones from | | | |
| | | primary | | | |
| | | screen | | | |

Supernatants from 315 positive multiclones were provided for testing.

Once multiclones were selected, subclones were generated by “limiting dilution” to ensure that most wells would only contain a single clone.

### Analysis of Hybridoma Clones

### Confirmation of Hybridoma Binding to ZIKV E-Protein and ZIKV-VLP:

All the clones received from Abcam were retested and confirmed for their binding to a ZIKV E protein and a ZIKV-VLP by indirect ELISA.

A total of 40×96-well plates were used to screen a total target of 3480 clones (data not shown). 311 clones were positive for a ZIKV-VLP binding while 26 clones were positive for a ZIKV E-protein binding. The ZIKV-VLP (Native Antigen) used here expresses PrM, M and E proteins.

### Example 2: Sequences of Anti-ZIKV Antibodies 102-1, 242-3, 289-3, 306-2, 11-3, 78-2, 270-12, 181-4/329-2, and 260-3

Clones 102-1, 242-3, 289-3, 306-2, 11-3, 78-2, 270-12, 181-4 and 260-3 were subjected to gene sequencing. Both the amino acid and nucleic acid sequences were determined. Table 6 sets forth the amino acid sequences of the heavy and light chains, variable regions and CDRs of anti-ZIKV antibodies of six clones. The nucleic acid sequences for the corresponding to the heavy and light chains and variable regions of the anti-ZIKV antibodies are set forth in Table 7.

TABLE 6
Amino acid sequences of anti-ZIKV antibodies
102-1, 242-3, 289-3, 306-2, 11-3, 78-2, 270-12,
181-4/329-2, 260-3 and 278-11.

| SEQ | | | |
|---|---|---|---|
| ID | Clone | Protein | |
| NO: | ID | domain | Sequence |
| 1 | 102-1 | Heavy | QSLEESGGDLVKPGASLTLTSKVSGFSFN |
| | | Chain | SNYWICWVRQAPGKGLEWIGCVFGGIHVT |
| | | | YYANWVKGRFTISKPSSTTVTLEMTSLTA |
| | | | ADTATYFCIISTGGSHRFNLWGPGTLVTV |
| | | | SSGQPKAPSVFPLAPCCGDTPSSTVTLGC |
| | | | LVKGYLPEPVTVTWNSGTLTNGVRTFPSV |
| | | | RQSSGLYSLSSVVSVTSSSQPVTCNVAHP |
| | | | ATNTKVDKTVAPSTCSKPTCPPPELLGGP |
| | | | SVFIFPPKPKDTLMISRTPEVTCVVVDVS |
| | | | QDDPEVQFTWYINNEQVRTARPPLREQQF |
| | | | NSTIRVVSTLPIAHQDWLRGKEFKCKVHN |
| | | | KALPAPIEKTISKARGQPLEPKVYTMGPP |
| | | | REELSSRSVSLTCMINGFYPSDISVEWEK |
| | | | NGKAEDNYKTTPAVLDSDGSYFLYSKLSV |
| | | | PTSEWQRGDVFTCSVMHEALHNHYTQKSI |
| | | | SRSPGK |
| 2 | 102-1 | VH | QSLEESGGDLVKPGASLTLTSKVSGFSFN |
| | | | SNYWICWVRQAPGKGLEWIGCVFGGIHVT |
| | | | YYANWVKGRFTISKPSSTTVTLEMTSLTA |
| | | | ADTATYFCIISTGGSHRFNLWGPGTLVTV |
| | | | SS |
| 3 | 102-1 | CDR-H1 | GFSFNSNYW |
| 4 | 102-1 | CDR-H2 | FGGIHVT |
| 5 | 102-1 | CDR-H3 | IISTGGSHRFNL |
| 6 | | Light | DVVMTQTPASVEAAVGGTVTIKCQASESI |
| | | Chain | YTYLSWYQQKPGQPPKLLIYRASTLDSGV |
| | | | PSRFSGSGSGTEFTLTISDLDCADAASYY |
| | | | CQATDVGGSGRGAFGGGTEVVVEGDPVAP |
| | | | TVLIFPPAADQVATGTVTIVCVANKYFPD |
| | | | VTVTWEVDGTTQTTGIENSKTPQNSADCT |
| | | | YNLSSTLTLTSTQYNSHKEYTCKVTQGTT |
| | | | SVVQSFNRGDC |
| 7 | 102-1 | VL | DVVMTQTPASVEAAVGGTVTIKCQASESI |
| | | | YTYLSWYQQKPGQPPKLLIYRASTLDSGV |
| | | | PSRFSGSGSGTEFTLTISDLDCADAASYY |
| | | | CQATDVGGSGRGAFGGGTEVVVE |
| 8 | 102-1 | CDR-L1 | ESIYTY |
| 9 | 102-1 | CDR-L2 | RAS |
| 10 | 102-1 | CDR-L3 | QATDVGGSGRGA |
| 11 | 242-3 | Heavy | QAQLEESGGDLVKPEGSLTLTCTASGFSF |
| | | Chain | TDRHYMCWIRQAPGKGLEWIACVYPGSSG |
| | | | STYYASWARGRFTISKTSSTTVTLRMTSL |
| | | | TAADTATYFCARSSYPDSSGYSYGMDLWG |
| | | | PGTLVTVSSGQPKAPSVFPLAPCCGDTPS |
| | | | STVTLGCLVKGYLPEPVTVTWNSGTLTNG |
| | | | VRTFPSVRQSSGLYSLSSVVSVTSSSQPV |
| | | | TCNVAHPATNTKVDKTVAPSTCSKPTCPP |
| | | | PELLGGPSVFIFPPKPKDTLMISRTPEVT |
| | | | CVVVDVSQDDPEVQFTWYINNEQVRTARP |
| | | | PLREQQFNSTIRVVSTLPIAHQDWLRGKE |
| | | | FKCKVHNKALPAPIEKTISKARGQPLEPK |
| | | | VYTMGPPREELSSRSVSLTCMINGFYPSD |
| | | | ISVEWEKNGKAEDNYKTTPAVLDSDGSYF |
| | | | LYSKLSVPTSEWQRGDVFTCSVMHEALHN |
| | | | HYTQKSISRSPGK |
| 12 | 242-3 | VH | QAQLEESGGDLVKPEGSLTLTCTASGFSF |
| | | | TDRHYMCWIRQAPGKGLEWIACVYPGSSG |
| | | | STYYASWARGRFTISKTSSTTVTLRMTSL |
| | | | TAADTATYFCARSSYPDSSGYSYGMDLWG |
| | | | PGTLVTVSS |
| 13 | 242-3 | CDR-H1 | GFSFTDRHY |
| 14 | 242-3 | CDR-H2 | YPGSSGST |
| 15 | 242-3 | CDR-H3 | ARSSYPDSSGYSYGMDL |
| 16 | 242-3 | Light | DIVMTQTPASVEAAVGGTVTIKCQASQNI |
| | | Chain | NSNLAWYQQKPGQRPKFLMYLTSKLASGV |
| | | | PSRFRGSGSGTQFTLTISDLECADAATYY |
| | | | CQTYYDISNYGYAFGGGTEVVVKGDPVAP |
| | | | TVLIFPPAADQVATGTVTIVCVANKYFPD |
| | | | VTVTWEVDGTTQTTGIENSKTPQNSADCT |
| | | | YNLSSTLTLTSTQYNSHKEYTCKVTQGTT |
| | | | SVVQSFNRGDC |
| 17 | 242-3 | VL | DIVMTQTPASVEAAVGGTVTIKCQASQNI |
| | | | NSNLAWYQQKPGQRPKFLMYLTSKLASGV |
| | | | PSRFRGSGSGTQFTLTISDLECADAATYY |
| | | | CQTYYDISNYGYAFGGGTEVVVK |
| 18 | 242-3 | CDR-L1 | QNINSN |
| 19 | 242-3 | CDR-L2 | LTS |
| 20 | 242-3 | CDR-L3 | QTYYDISNYGYA |
| 21 | 289-3 | Heavy | QEELEESGGDLVKPGASLTLTCKASGFSF |
| | | Chain | SSGAYICWVRQAPGKGPEWIACIYTGDGR |
| | | | TYYANWAKRRFTISSSSSTTVALEMPSLT |
| | | | DADTATYFCARAIAVGAGYGVGNYFTLWG |
| | | | PGTLVTVSSGQPKAPSVFPLAPCCGDTPS |
| | | | STVTLGCLVKGYLPEPVTVTWNSGTLTNG |
| | | | VRTFPSVRQSSGLYSLSSVVSVTSSSQPV |
| | | | TCNVAHPATNTKVDKTVAPSTCSKPTCPP |
| | | | PELLGGPSVFIFPPKPKDTLMISRTPEVT |
| | | | CVVVDVSQDDPEVQFTWYINNEQVRTARP |
| | | | PLREQQFNSTIRVVSTLPIAHQDWLRGKE |
| | | | FKCKVHNKALPAPIEKTISKARGQPLEPK |
| | | | VYTMGPPREELSSRSVSLTCMINGFYPSD |
| | | | ISVEWEKNGKAEDNYKTTPAVLDSDGSYF |
| | | | LYSKLSVPTSEWQRGDVFTCSVMHEALHN |
| | | | HYTQKSISRSPGK |
| 22 | 289-3 | VH | QEELEESGGDLVKPGASLTLTCKASGFSF |
| | | | SSGAYICWVRQAPGKGPEWIACIYTGDGR |
| | | | TYYANWAKRRFTISSSSSTTVALEMPSLT |
| | | | DADTATYFCARAIAVGAGYGVGNYFTLWG |
| | | | PGTLVTVSS |
| 23 | 289-3 | CDR-H1 | GFSFSSGAY |
| 24 | 289-3 | CDR-H2 | YTGDGRT |
| 25 | 289-3 | CDR-H3 | ARAIAVGAGYGVGNYFTL |
| 26 | 289-3 | Light | DIVMTQTPSSVSAAVGGTVTINCHASENI |
| | | Chain | YGYLSWYQQKPGQPPKLLIYKASTLASGV |
| | | | PSRFKGSGAGTQFTLTITALECADAATYY |
| | | | CQSYYTSSSNADGSENAFGGGTEVVVEGD |
| | | | PVAPTVLIFPPAADQVATGTVTIVCVANK |
| | | | YFPDVTVTWEVDGTTQTTGIENSKTPQNS |
| | | | ADCTYNLSSTLTLTSTQYNSHKEYTCKVT |
| | | | QGTTSVVQSFNRGDC |
| 27 | 289-3 | VL | DIVMTQTPSSVSAAVGGTVTINCHASENI |
| | | | YGYLSWYQQKPGQPPKLLIYKASTLASGV |
| | | | PSRFKGSGAGTQFTLTITALECADAATYY |
| | | | CQSYYTSSSNADGSENAFGGGTEVVVE |
| 28 | 289-3 | CDR-L1 | ENIYGY |
| 29 | 289-3 | CDR-L2 | KAS |
| 30 | 289-3 | CDR-L3 | QSYYTSSSNADGSENA |
| 31 | 306-2 | Heavy | QSLEESGGDLVKPGASLTLTCTASGFDFS |
| | | Chain | DRYYMCWVRQAPGKGLEWIGCIYVGSGDT |
| | | | YYASWAKGRFTISRTSSTTVTLQMTRLTA |
| | | | ADTATYFCARHPGTYFTLWGPGTLVTVSS |
| | | | GQPKAPSVFPLAPCCGDTPSSTVTLGCLV |
| | | | KGYLPEPVTVTWNSGTLTNGVRTFPSVRQ |
| | | | SSGLYSLSSVVSVTSSSQPVTCNVAHPAT |
| | | | NTKVDKTVAPSTCSKPTCPPPELLGGPSV |
| | | | FIFPPKPKDTLMISRTPEVTCVVVDVSQD |
| | | | DPEVQFTWYINNEQVRTARPPLREQQFNS |
| | | | TIRVVSTLPIAHQDWLRGKEFKCKVHNKA |
| | | | LPAPIEKTISKARGQPLEPKVYTMGPPRE |
| | | | ELSSRSVSLTCMINGFYPSDISVEWEKNG |
| | | | KAEDNYKTTPAVLDSDGSYFLYSKLSVPT |
| | | | SEWQRGDVFTCSVMHEALHNHYTQKSISR |
| | | | SPGK |
| 32 | 306-2 | VH | QSLEESGGDLVKPGASLTLTCTASGFDFS |
| | | | DRYYMCWVRQAPGKGLEWIGCIYVGSGDT |
| | | | YYASWAKGRFTISRTSSTTVTLQMTRLTA |
| | | | ADTATYFCARHPGTYFTLWGPGTLVTVSS |
| 33 | 306-2 | CDR-H1 | GFDFSDRYY |
| 34 | 306-2 | CDR-H2 | YVGSGDT |
| 35 | 306-2 | CDR-H3 | ARHPGTYF |
| 36 | 306-2 | Light | DVVMTQTPSSVSAAVGGTVTINCQASQNI |
| | | Chain | VNNLAWYQQRPGQPPKLLIYDTSTLASGV |
| | | | PSRFKGSGSGTEFTLTISDLECADAATYY |
| | | | CQTYYYYNKIINGFGGGTEVVVKGDPVAP |
| | | | TVLIFPPAADQVATGTVTIVCVANKYFPD |
| | | | VTVTWEVDGTTQTTGIENSKTPQNSADCT |
| | | | YNLSSTLTLTSTQYNSHKEYTCKVTQGTT |
| | | | SVVQSFNRGDC |
| 37 | 306-2 | VL | DVVMTQTPSSVSAAVGGTVTINCQASQNI |
| | | | VNNLAWYQQRPGQPPKLLIYDTSTLASGV |
| | | | PSRFKGSGSGTEFTLTISDLECADAATYY |
| | | | CQTYYYYNKIINGFGGGTEVVVK |
| 38 | 306-2 | CDR-L1 | QNIVNN |
| 39 | 306-2 | CDR-L2 | DTS |
| 40 | 306-2 | CDR-L3 | QTYYYYNKIING |
| 41 | 11-3 | Heavy | QEQLVESGGGLVQPEGSLTITCTGSGFSF |
| | | Chain | SSRFYMCWVRQAPGKGLEWIACIYGGSSG |
| | | | STYYASWAKGRFTISKTSSTTVTLQISSL |
| | | | TAADTATYFCARGGSTAAAGFNLWGPGTL |
| | | | LTVSSGQPKAPSVFPLAPCCGDTPSSTVT |
| | | | LGCLVKGYLPEPVTVTWNSGTLTNGVRTF |
| | | | PSVRQSSGLYSLSSVVSVTSSSQPVTCNV |
| | | | AHPATNTKVDKTVAPSTCSKPTCPPPELL |
| | | | GGPSVFIFPPKPKDTLMISRTPEVTCVVV |
| | | | DVSQDDPEVQFTWYINNEQVRTARPPLRE |
| | | | QQFNSTIRVVSTLPIAHQDWLRGKEFKCK |
| | | | VHNKALPAPIEKTISKARGQPLEPKVYTM |
| | | | GPPREELSSRSVSLTCMINGFYPSDISVE |
| | | | WEKNGKAEDNYKTTPAVLDSDGSYFLYSK |
| | | | LSVPTSEWQRGDVFTCSVMHEALHNHYTQ |
| | | | KSISRSPGK |
| 42 | 11-3 | VH | QEQLVESGGGLVQPEGSLTITCTGSGFSF |
| | | | SSRFYMCWVRQAPGKGLEWIACIYGGSSG |
| | | | STYYASWAKGRFTISKTSSTTVTLQISSL |
| | | | TAADTATYFCARGGSTAAAGFNLWGPGTL |
| | | | LTVSS |
| 43 | 11-3 | CDR-H1 | GFSFSSRFY |
| 44 | 11-3 | CDR-H2 | YGGSSGST |
| 45 | 11-3 | CDR-H3 | ARGGSTAAAGFNL |
| 46 | 11-3 | Light | DVVMTQTPASVSATVGGTVTIKCQASEDI |
| | | Chain | YNLLAWYQQKPGQPPKLLIYYASTLASGV |
| | | | PSRFKGSGSGTEFTLTISDLECADAATYY |
| | | | CQCNDYGGTYVPNAFGGGTEVVVKGDPVA |
| | | | PTVLIFPPAADQVATGTVTIVCVANKYFP |
| | | | DVTVTWEVDGTTQTTGIENSKTPQNSADC |
| | | | YTNLSSTLTLTSTQYNSHKEYTCKVTQGT |
| | | | TSVVQSFNRGDC |
| 47 | 11-3 | VL | DVVMTQTPASVSATVGGTVTIKCQASEDI |
| | | | YNLLAWYQQKPGQPPKLLIYYASTLASGV |
| | | | PSRFKGSGSGTEFTLTISDLECADAATYY |
| | | | CQCNDYGGTYVPNAFGGGTEVVVK |
| 48 | 11-3 | CDR-L1 | EDIYNL |
| 49 | 11-3 | CDR-L2 | YAS |
| 50 | 11-3 | CDR-L3 | QCNDYGGTYVPNA |
| 51 | 78-2 | Heavy | QQQLVESGGGLVKPGASLTLNCKASGLDF |
| | | Chain | STNSYMCWVRQAPGKGLEWIACIYVGDSS |
| | | | EIYYASWAKGRFTISKTSSTTVTLQMTSL |
| | | | TAADTATYFCARDLPSFTAPYAGYLRLWG |
| | | | PGTLVTVSSGQPKAPSVFPLAPCCGDTPS |
| | | | TSVTLGCLVKGYLPEPVTVTWNSGTLTNG |
| | | | VRTFPSVRQSSGLYSLSSVVSVTSSSQPV |
| | | | TCNVAHPATNTKVDKTVAPSTCSKPTCPP |
| | | | PELLGGPSVFIFPPKPKDTLMISRTPEVT |
| | | | CVVVDVSQDDPEVQFTWYINNEQVRTARP |
| | | | PLREQQFNSTIRVVSTLPIAHQDWLRGKE |
| | | | FKCKVHNKALPAPIEKTISKARGQPLEPK |
| | | | VYTMGPPREELSSRSVSLTCMINGFYPSD |
| | | | ISVEWEKNGKAEDNYKTTPAVLDSDGSYF |
| | | | LYSKLSVPTSEWQRGDVFTCSVMHEALHN |
| | | | HYTQKSISRSPGK |
| 52 | 78-2 | VH | QQQLVESGGGLVKPGASLTLNCKASGLDF |
| | | | STNSYMCWVRQAPGKGLEWIACIYVGDSS |
| | | | EIYYASWAKGRFTISKTSSTTVTLQMTSL |
| | | | TAADTATYFCARDLPSFTAPYAGYLRLWG |
| | | | PGTLVTVSS |
| 53 | 78-2 | CDR-H1 | GLDFSTNSY |
| 54 | 78-2 | CDR-H2 | IYVGDSSEI |
| 55 | 78-2 | CDR-H3 | ARDLPSFTAPYAGYLRL |
| 56 | 78-2 | Light | QPVLTQPPSLSASLGTTARLTCTLRTGYN |
| | | Chain | VGDYPLLWLQRVPGRPPRYLLTYHTEEFK |
| | | | HQGSGVHSRFSGSKDASENAGVLSISGLQ |
| | | | PEDEANYYCYTVHATESSLHYVFGGGTQL |
| | | | TVTGQPAVTPSVILFPPSSEELKDNKATL |
| | | | VCLINDFYPGTVKVNWKADGTPVTQGVDT |
| | | | TQPSKQSNSKYAASSFLSLSANQWKSYQS |
| | | | VTCQVTHEGHTVEKSLAPAECS |
| 57 | 78-2 | VL | QPVLTQPPSLSASLGTTARLTCTLRTGYN |
| | | | VGDYPLLWLQRVPGRPPRYLLTYHTEEFK |
| | | | HQGSGVHSRFSGSKDASENAGVLSISGLQ |
| | | | PEDEANYYCYTVHATESSLHYVFGGGTQL |
| | | | TVT |
| 58 | 78-2 | CDR-L1 | TGYNVGDYP |
| 59 | 78-2 | CDR-L2 | YHTEEFKH |
| 60 | 78-2 | CDR-L3 | YTVHATESSLHYVF |
| 85 | 270-12 | Heavy | QAQLEESGGDLVKPEGSLTLTCTASGFSF |
| | | Chain | TDRHYMCWVRQAPGKGLEWIACVYPGSSG |
| | | | STYYASWARGRFTISKTSSTTVTLRMTSL |
| | | | TAADTATYFCARSSYPDSSGYSYGMDLWG |
| | | | PGTLVTVSSGQPKAPSVFPLAPCCGDTPS |
| | | | STVTLGCLVKGYLPEPVTVTWNSGTLTNG |
| | | | VRTFPSVRQSSGLYSLSSVVSVTSSSQPV |
| | | | TCNVAHPATNTKVDKTVAPSTCSKPTCPP |
| | | | PELLGGPSVFIFPPKPKDTLMISRTPEVT |
| | | | CVVVDVSQDDPEVQFTWYINNEQVRTARP |
| | | | PLREQQFNSTIRVVSTLPIAHQDWLRGKE |
| | | | FKCKVHNKALPAPIEKTISKARGQPLEPK |
| | | | VYTMGPPREELSSRSVSLTCMINGFYPSD |
| | | | ISVEWEKNGKAEDNYKTTPAVLDSDGSYF |
| | | | LYSKLSVPTSEWQRGDVFTCSVMHEALHN |
| | | | HYTQKSISRSPGK |
| 86 | 270-12 | VH | QAQLEESGGDLVKPEGSLTLTCTASGFSF |
| | | | TDRHYMCWVRQAPGKGLEWIACVYPGSSG |
| | | | STYYASWARGRFTISKTSSTTVTLRMTSL |
| | | | TAADTATYFCARSSYPDSSGYSYGMDLWG |
| | | | PGTLVTVSS |
| 87 | 270-12 | CDR-H1 | GFSFTDRHY |
| 88 | 270-12 | CDR-H2 | YPGSSGST |
| 89 | 270-12 | CDR-H3 | ARSSYPDSSGYSYGMDL |
| 90 | 270-12 | Light | DIVMTQTPASVEAAVGGTVTIKCQASQDI |
| | | Chain | NSNLAWYQQKPGQRPKFLMYLTSKLASGV |
| | | | PSRFRGSGSGTQFTLTISDLECADAATYY |
| | | | CQTYYDISNYGYAFGGGTEVVVKGDPVAP |
| | | | TVLIFPPAADQVATGTVTIVCVANKYFPD |
| | | | VTVTWEVDGTTRTTGIENSKTPQNSADCT |
| | | | YNLSSTLTLTSTQYNSHKEYTCKVTQGTT |
| | | | SVVQSFNRGDC |
| 91 | 270-12 | VL | DIVMTQTPASVEAAVGGTVTIKCQASQDI |
| | | | NSNLAWYQQKPGQRPKFLMYLTSKLASGV |
| | | | PSRFRGSGSGTQFTLTISDLECADAATYY |
| | | | CQTYYDISNYGYAFGGGTEVVVK |
| 92 | 270-12 | CDR-L1 | QDINSN |
| 93 | 270-12 | CDR-L2 | LTS |
| 94 | 270-12 | CDR-L3 | QTYYDISNYGYA |
| 95 | 181-4 or | Heavy | QAQLEESGGDLVKPEGSLTLTCTASGFSF |
| | 329-2 | Chain | TDRHYMCWVRQAPGKGLEWIACVYPGSSG |
| | | | STYYASWARGRFTISKTSSTTVTLRMTSL |
| | | | TAADTATYFCARSSYPDSSGYSYGMDLWG |
| | | | PGTLVTVSSGQPKAPSVFPLAPCCGDTPS |
| | | | STVTLGCLVKGYLPEPVTVTWNSGTLTNG |
| | | | VRTFPSVRQSSGLYSLSSVVSVTSSSQPV |
| | | | TCNVAHPATNTKVDKTVAPSTCSKPTCPP |
| | | | PELLGGPSVFIFPPKPKDTLMISRTPEVT |
| | | | CVVVDVSQDDPEVQFTWYINNEQVRTARP |
| | | | PLREQQFNSTIRVVSTLPIAHQDWLRGKE |
| | | | FKCKVHNKALPAPIEKTISKARGQPLEPK |
| | | | VYTMGPPREELSSRSVSLTCMINGFYPSD |
| | | | ISVEWEKNGKAEDNYKTTPAVLDSDGSYF |
| | | | LYSKLSVPTSEWQRGDVFTCSVMHEALHN |
| | | | HYTQKSISRSPGK |
| 96 | 181-4 or | VH | QAQLEESGGDLVKPEGSLTLTCTASGFSF |
| | 329-2 | | TDRHYMCWVRQAPGKGLEWIACVYPGSSG |
| | | | STYYASWARGRFTISKTSSTTVTLRMTSL |
| | | | TAADTATYFCARSSYPDSSGYSYGMDLWG |
| | | | PGTLVTVSS |
| 97 | 181-4 or | CDR-H1 | GFSFTDRHY |
| | 329-2 | | |
| 98 | 181-4 or | CDR-H2 | YPGSSGST |
| | 329-2 | | |
| 99 | 181-4 or | CDR-H3 | ARSSYPDSSGYSYGMDL |
| | 329-2 | | |
| 100 | 181-4 or | Light | DIVMTQTPASVEAAVGGTVTIKCQASQNI |
| | 329-2 | Chain | NSNLAWYQQKPGQRPKFLMYLTSKLASGV |
| | | | PSRFRGSGSGTQFTLTISDLECADAATYY |
| | | | CQTYYDISNYGYAFGGGTEVVVKGDPVAP |
| | | | TVLIFPPAADQVATGTVTIVCVANKYFPD |
| | | | VTVTWEVDGTTQTTGIENSKTPQNSADCT |
| | | | YNLSSTLTLTSTQYNSHKEYTCKVTQGTT |
| | | | SVVQSFNRGDC |
| 101 | 181-4 or | VL | DIVMTQTPASVEAAVGGTVTIKCQASQNI |
| | 329-2 | | NSNLAWYQQKPGQRPKFLMYLTSKLASGV |
| | | | PSRFRGSGSGTQFTLTISDLECADAATYY |
| | | | CQTYYDISNYGYAFGGGTEVVVK |
| 102 | 181-4 or | CDR-L1 | QNINSN |
| | 329-2 | | |
| 103 | 181-4 or | CDR-L2 | LTS |
| | 329-2 | | |
| 104 | 181-4 or | CDR-L3 | QTYYDISNYGYA |
| | 329-2 | | |
| 105 | 260-3 | Heavy | QAQLEESGGDLVKPEGSLTLTCTASGFSF |
| | | Chain | DRHYMCWVRQAPGKGLEWIACVYPGSSGT |
| | | | STYYASWARGRFTISKTSSTTVTLRMTSL |
| | | | AADTATYFCARSSYPDSSGYSYGMDLWGT |
| | | | PGTLVTVPSGQPKAPSVFPLAPCCGDTPS |
| | | | STVTLGCLVKGYLPEPVTVTWNSGTLTNG |
| | | | VRTFPSVRQSSGLYSLSSVVSVTSSSQPV |
| | | | TCNVAHPATNTKVDKTVAPSTCSKPTCPP |
| | | | PELLGGPSVFIFPPKPKDTLMISRTPEVT |
| | | | CVVVDVSQDDPEVQFTWYINNEQVRTARP |
| | | | PLREQQFNSTIRVVSTLPIAHQDWLRGKE |
| | | | FKCKVHNKALPAPIEKTISKARGQPLEPK |
| | | | VYTMGPPREELSSRSVSLTCMINGFYPSD |
| | | | ISVEWEKNGKAEDNYKTTPAVLDSDGSYF |
| | | | LYSKLSVPTSEWQRGDVFTCSVMHEALHN |
| | | | HYTQKSISRSPGK |
| 106 | 260-3 | VH | QAQLEESGGDLVKPEGSLTLTCTASGFSF |
| | | | TDRHYMCWVRQAPGKGLEWIACVYPGSSG |
| | | | STYYASWARGRFTISKTSSTTVTLRMTSL |
| | | | TAADTATYFCARSSYPDSSGYSYGMDLWG |
| | | | PGTLVTVPS |
| 107 | 260-3 | CDR-H1 | GFSFTDRHY |
| 108 | 260-3 | CDR-H2 | YPGSSGST |
| 109 | 260-3 | CDR-H3 | ARSSYPDSSGYSYGMDL |
| 110 | 260-3 | Light | DIVMTQTPASVEAAVGGTVTIKCQASQNI |
| | | Chain | NSNLAWYQQKPGQRPKFLMYLTSKLASGV |
| | | | PSRFRGSGSGTQFTLTISDLECADAATYY |
| | | | CQTYYDISNYGYAFGGGTEVVVKGDPVAP |
| | | | TVLIFPPAADQVATGTVTIVCVANKYFPD |
| | | | VTVTWEVDGTTQTTGIENSKTPQNSADCT |
| | | | YNLSSTLTLTSTQYNSHKEYTCKVTQGTT |
| | | | SVVQSFNRGDC |
| 111 | 260-3 | VL | DIVMTQTPASVEAAVGGTVTIKCQASQNI |
| | | | NSNLAWYQQKPGQRPKFLMYLTSKLASGV |
| | | | PSRFRGSGSGTQFTLTISDLECADAATYY |
| | | | CQTYYDISNYGYAFGGGTEVVVK |
| 112 | 260-3 | CDR-L1 | QNINSN |
| 113 | 260-3 | CDR-L2 | LTS |
| 114 | 260-3 | CDR-L3 | QTYYDISNYGYA |
| 128 | 278-11 | Heavy | QCQSLEESGGGLVQPEGSLTLACTASEFD |
| | | Chain | SSSNAMCWVRQAPGKGLEWIARIYSGSGT |
| | | | IYYANWARGRFTISKTSSTTVTLQMTSLT |
| | | | DADTATYFCARYNTGGFYYDLWGPGTLVT |
| | | | VSSGQPKAPSVFPLAPCCGDTPSSTVTLG |
| | | | CLVKGYLPEPVTVTWNSGTLTNGVRTFPS |
| | | | VRQSSGLYSLSSVVSVTSSSQPVTCNVAH |
| | | | PATNTKVDKTVAPSTCSKPTCPPPELLGG |
| | | | PSVFIFPPKPKDTLMISRTPEVTCVVVDV |
| | | | SQDDPEVQFTWYINNEQVRTARPPLREQQ |
| | | | FNSTIRVVSTLPIAHQDWLRGKEFKCKVH |
| | | | NKALPAPIEKTISKARGQPLEPKVYTMGP |
| | | | PREELSSRSVSLTCMINGFYPSDISVEWE |
| | | | KNGKAEDNYKTTPAVLDSDGSYFLYSKLS |
| | | | VPTSEWQRGDVFTCSVMHEALHNHYTQKS |
| | | | ISRSPGK |
| 129 | 278-11 | VH | QCQSLEESGGGLVQPEGSLTLACTASEFD |
| | | | SSSNAMCWVRQAPGKGLEWIARIYSGSGT |
| | | | IYYANWARGRFTISKTSSTTVTLQMTSLT |
| | | | DADTATYFCARYNTGGFYYDLWGPGTLVT |
| | | | VSS |
| 130 | 278-11 | CDR-H1 | EFDSSSNA |
| 131 | 278-11 | CDR-H2 | IYSGSGTI |
| 132 | 278-11 | CDR-H3 | ARYNTGGFYYDL |
| 133 | 278-11 | Light | ALVMTQTPASVEAAVGGTVTINCQASQRI |
| | | Chain | GTNLAWYQQKPGQPPKVLIYKASTLASGV |
| | | | SSRFIGSGSGTDYTLTISGVECDDAATYY |
| | | | CQQGYSSNDADNTFGGGTEVVVKGDPVAP |
| | | | TVLIFPPAADQVATGTVTIVCVANKYFPD |
| | | | VTVTWEVDGTTQTTGIENSKTPQNSADCT |
| | | | YNLSSTLTLTSTQYNSHKEYTCKVTQGTT |
| | | | SVVQSFNRGDC |
| 134 | 278-11 | VL | ALVMTQTPASVEAAVGGTVTINCQASQRI |
| | | | GTNLAWYQQKPGQPPKVLIYKASTLASGV |
| | | | SSRFIGSGSGTDYTLTISGVECDDAATYY |
| | | | CQQGYSSNDADNTFGGGTEVWK |
| 135 | 278-11 | CDR-L1 | QRIGTN |
| 136 | 278-11 | CDR-L2 | KAS |
| 137 | 278-11 | CDR-L3 | QQGYSSNDADNT |

TABLE 7
Nucleic acid sequences of anti-ZIKV antibodies
102-1, 242-3, 289-3, 306-2, 11-3, 78-2, 270-12,
181-4/329-2, 260-3 and 278-11.

| SEQ ID | Clone | Protein | |
|---|---|---|---|
| NO: | ID | domain | Sequence |
| 61 | 102-1 | Heavy | ATGGAGACTGGGCTGCGCTGGCTTCTCC |
| | | Chain | TGGTCGCTGTGCTCAAAGGTGTCCAATG |
| | | | TCAGTCGTTGGAGGAGTCCGGGGGAGAC |
| | | | CTGGTCAAGCCTGGGGCATCCCTGACAC |
| | | | TCACCTCCAAAGTCTCTGGATTCTCCTT |
| | | | CAATAGCAACTATTGGATATGCTGGGTC |
| | | | CGCCAGGCTCCAGGGAAGGGGCTGGAGT |
| | | | GGATCGGATGCGTTTTTGGTGGTATTCA |
| | | | TGTCACATATTACGCGAATTGGGTGAAA |
| | | | GGCCGATTCACCATCTCCAAACCCTCGT |
| | | | CGACCACGGTGACTCTGGAAATGACCAG |
| | | | TCTGACAGCCGCGGACACGGCCACTTAT |
| | | | TTCTGTATTATTAGTACTGGCGGTAGTC |
| | | | ACAGATTTAATTTGTGGGGCCCAGGCAC |
| | | | CCTGGTCACCGTCTCCTCAGGGCAACCT |
| | | | AAGGCTCCATCAGTCTTCCCACTGGCCC |
| | | | CCTGCTGCGGGGACACACCCAGCTCCAC |
| | | | GGTGACCCTGGGCTGCCTGGTCAAAGGG |
| | | | TACCTCCCGGAGCCAGTGACCGTGACCT |
| | | | GGAACTCGGGCACCCTCACCAATGGGGT |
| | | | ACGCACCTTCCCGTCCGTCCGGCAGTCC |
| | | | TCAGGCCTCTACTCGCTGAGCAGCGTGG |
| | | | TGAGCGTGACCTCAAGCAGCCAGCCCGT |
| | | | CACCTGCAACGTGGCCCACCCAGCCACC |
| | | | AACACCAAAGTGGACAAGACCGTTGCGC |
| | | | CCTCGACATGCAGCAAGCCCACGTGCCC |
| | | | ACCCCCTGAACTCCTGGGGGGACCGTCT |
| | | | GTCTTCATCTTCCCCCCAAAACCCAAGG |
| | | | ACACCCTCATGATCTCACGCACCCCCGA |
| | | | GGTCACATGCGTGGTGGTGGACGTGAGC |
| | | | CAGGATGACCCCGAGGTGCAGTTCACAT |
| | | | GGTACATAAACAACGAGCAGGTGCGCAC |
| | | | CGCCCGGCCGCCGCTACGGGAGCAGCAG |
| | | | TTCAACAGCACGATCCGCGTGGTCAGCA |
| | | | CCCTCCCCATCGCGCACCAGGACTGGCT |
| | | | GAGGGGCAAGGAGTTCAAGTGCAAAGTC |
| | | | CACAACAAGGCACTCCCGGCCCCCATCG |
| | | | AGAAAACCATCTCCAAAGCCAGAGGGCA |
| | | | GCCCCTGGAGCCGAAGGTCTACACCATG |
| | | | GGCCCTCCCCGGGAGGAGCTGAGCAGCA |
| | | | GGTCGGTCAGCCTGACCTGCATGATCAA |
| | | | CGGCTTCTACCCTTCCGACATCTCGGTG |
| | | | GAGTGGGAGAAGAACGGGAAGGCAGAGG |
| | | | ACAACTACAAGACCACGCCGGCCGTGCT |
| | | | GGACAGCGACGGCTCCTACTTCCTCTAC |
| | | | AGCAAGCTCTCAGTGCCCACGAGTGAGT |
| | | | GGCAGCGGGGCGACGTCTTCACCTGCTC |
| | | | CGTGATGCACGAGGCCTTGCACAACCAC |
| | | | TACACGCAGAAGTCCATCTCCCGCTCTC |
| | | | CGGGTAAATGA |
| 62 | 102-1 | VH | ATGGAGACTGGGCTGCGCTGGCTTCTCC |
| | | | TGGTCGCTGTGCTCAAAGGTGTCCAATG |
| | | | TCAGTCGTTGGAGGAGTCCGGGGGAGAC |
| | | | CTGGTCAAGCCTGGGGCATCCCTGACAC |
| | | | TCACCTCCAAAGTCTCTGGATTCTCCTT |
| | | | CAATAGCAACTATTGGATATGCTGGGTC |
| | | | CGCCAGGCTCCAGGGAAGGGGCTGGAGT |
| | | | GGATCGGATGCGTTTTTGGTGGTATTCA |
| | | | TGTCACATATTACGCGAATTGGGTGAAA |
| | | | GGCCGATTCACCATCTCCAAACCCTCGT |
| | | | CGACCACGGTGACTCTGGAAATGACCAG |
| | | | TCTGACAGCCGCGGACACGGCCACTTAT |
| | | | TTCTGTATTATTAGTACTGGCGGTAGTC |
| | | | ACAGATTTAATTTGTGGGGCCCAGGCAC |
| | | | CCTGGTCACCGTCTCCTCA |
| 63 | 102-1 | Light | ATGGACACGAGGGCCCCCACTCAGCTGC |
| | | Chain | TGGGGCTCCTGCTGCTCTGGCTCCCAGG |
| | | | TGCCAGATGTGATGTTGTGATGACCCAG |
| | | | ACTCCAGCCTCCGTGGAGGCAGCTGTGG |
| | | | GAGGCACAGTCACCATCAAGTGCCAGGC |
| | | | CAGTGAGAGCATTTATACCTACTTATCC |
| | | | TGGTATCAGCAGAAACCAGGGCAGCCTC |
| | | | CCAAACTCCTGATCTATAGGGCATCCAC |
| | | | TCTGGATTCTGGGGTCCCATCGCGGTTC |
| | | | AGCGGCAGTGGATCTGGGACAGAGTTCA |
| | | | CTCTCACCATCAGCGACCTGGACTGTGC |
| | | | CGATGCTGCCAGTTATTATTGTCAGGCT |
| | | | ACTGATGTTGGTGGTAGTGGTAGGGGTG |
| | | | CTTTCGGCGGAGGGACCGAGGTGGTGGT |
| | | | CGAAGGTGATCCAGTTGCACCTACTGTC |
| | | | CTCATCTTCCCACCAGCTGCTGATCAGG |
| | | | TGGCAACTGGAACAGTCACCATCGTGTG |
| | | | TGTGGCGAATAAATACTTTCCCGATGTC |
| | | | ACCGTCACCTGGGAGGTGGATGGCACCA |
| | | | CCCAAACAACTGGCATCGAGAACAGTAA |
| | | | AACACCGCAGAATTCTGCAGATTGTACC |
| | | | TACAACCTCAGCAGCACTCTGACACTGA |
| | | | CCAGCACACAGTACAACAGCCACAAAGA |
| | | | GTACACCTGCAAGGTGACCCAGGGCACG |
| | | | ACCTCAGTCGTCCAGAGCTTCAATAGGG |
| | | | GTGACTGTTAG |
| 64 | 102-1 | VL | ATGGACACGAGGGCCCCCACTCAGCTGC |
| | | | TGGGGCTCCTGCTGCTCTGGCTCCCAGG |
| | | | TGCCAGATGTGATGTTGTGATGACCCAG |
| | | | ACTCCAGCCTCCGTGGAGGCAGCTGTGG |
| | | | GAGGCACAGTCACCATCAAGTGCCAGGC |
| | | | CAGTGAGAGCATTTATACCTACTTATCC |
| | | | TGGTATCAGCAGAAACCAGGGCAGCCTC |
| | | | CCAAACTCCTGATCTATAGGGCATCCAC |
| | | | TCTGGATTCTGGGGTCCCATCGCGGTTC |
| | | | AGCGGCAGTGGATCTGGGACAGAGTTCA |
| | | | CTCTCACCATCAGCGACCTGGACTGTGC |
| | | | CGATGCTGCCAGTTATTATTGTCAGGCT |
| | | | ACTGATGTTGGTGGTAGTGGTAGGGGTG |
| | | | CTTTCGGCGGAGGGACCGAGGTGGTGGT |
| | | | CGAA |
| 65 | 242-3 | Heavy | ATGGAGACTGGGCTGCGCTGGCTTCTCC |
| | | Chain | TGGTCGCTGTGCTCAAAGGTGTCCAGTG |
| | | | TCAGGCGCAGCTGGAGGAGTCCGGGGGA |
| | | | GACCTGGTCAAGCCTGAGGGATCCCTGA |
| | | | CACTCACCTGCACAGCCTCTGGATTCTC |
| | | | CTTCACTGACAGGCATTACATGTGCTGG |
| | | | ATCCGCCAGGCTCCGGGGAAGGGGCTGG |
| | | | AGTGGATCGCATGCGTTTATCCTGGTAG |
| | | | TAGTGGTAGCACTTACTACGCGAGCTGG |
| | | | GCGAGAGGCCGATTCACCATCTCCAAAA |
| | | | CCTCGTCGACCACGGTGACTCTGCGCAT |
| | | | GACCAGTCTGACAGCCGCGGACACGGCC |
| | | | ACCTATTTCTGTGCGAGATCCTCATATC |
| | | | CTGATAGTAGTGGCTATAGTTATGGCAT |
| | | | GGACCTCTGGGGCCCTGGGACCCTCGTC |
| | | | ACCGTCTCTTCAGGGCAACCTAAGGCTC |
| | | | CATCAGTCTTCCCACTGGCCCCCTGCTG |
| | | | CGGGGACACACCCAGCTCCACGGTGACC |
| | | | CTGGGCTGCCTGGTCAAAGGGTACCTCC |
| | | | CGGAGCCAGTGACCGTGACCTGGAACTC |
| | | | GGGCACCCTCACCAATGGGGTACGCACC |
| | | | TTCCCGTCCGTCCGGCAGTCCTCAGGCC |
| | | | TCTACTCGCTGAGCAGCGTGGTGAGCGT |
| | | | GACCTCAAGCAGCCAGCCCGTCACCTGC |
| | | | AACGTGGCCCACCCAGCCACCAACACCA |
| | | | AAGTGGACAAGACCGTTGCGCCCTCGAC |
| | | | ATGCAGCAAGCCCACGTGCCCACCCCCT |
| | | | GAACTCCTGGGGGGACCGTCTGTCTTCA |
| | | | TCTTCCCCCCAAAACCCAAGGACACCCT |
| | | | CATGATCTCACGCACCCCCGAGGTCACA |
| | | | TGCGTGGTGGTGGACGTGAGCCAGGATG |
| | | | ACCCCGAGGTGCAGTTCACATGGTACAT |
| | | | AAACAACGAGCAGGTGCGCACCGCCCGG |
| | | | CCGCCGCTACGGGAGCAGCAGTTCAACA |
| | | | GCACGATCCGCGTGGTCAGCACCCTCCC |
| | | | CATCGCGCACCAGGACTGGCTGAGGGGC |
| | | | AAGGAGTTCAAGTGCAAAGTCCACAACA |
| | | | AGGCACTCCCGGCCCCCATCGAGAAAAC |
| | | | CATCTCCAAAGCCAGAGGGCAGCCCCTG |
| | | | GAGCCGAAGGTCTACACCATGGGCCCTC |
| | | | CCCGGGAGGAGCTGAGCAGCAGGTCGGT |
| | | | CAGCCTGACCTGCATGATCAACGGCTTC |
| | | | TACCCTTCCGACATCTCGGTGGAGTGGG |
| | | | AGAAGAACGGGAAGGCAGAGGACAACTA |
| | | | CAAGACCACGCCGGCCGTGCTGGACAGC |
| | | | GACGGCTCCTACTTCCTCTACAGCAAGC |
| | | | TCTCAGTGCCCACGAGTGAGTGGCAGCG |
| | | | GGGCGACGTCTTCACCTGCTCCGTGATG |
| | | | CACGAGGCCTTGCACAACCACTACACGC |
| | | | AGAAGTCCATCTCCCGCTCTCCGGGTAA |
| | | | ATGA |
| 66 | 242-3 | VH | ATGGAGACTGGGCTGCGCTGGCTTCTCC |
| | | | TGGTCGCTGTGCTCAAAGGTGTCCAGTG |
| | | | TCAGGCGCAGCTGGAGGAGTCCGGGGGA |
| | | | GACCTGGTCAAGCCTGAGGGATCCCTGA |
| | | | CACTCACCTGCACAGCCTCTGGATTCTC |
| | | | CTTCACTGACAGGCATTACATGTGCTGG |
| | | | ATCCGCCAGGCTCCGGGGAAGGGGCTGG |
| | | | AGTGGATCGCATGCGTTTATCCTGGTAG |
| | | | TAGTGGTAGCACTTACTACGCGAGCTGG |
| | | | GCGAGAGGCCGATTCACCATCTCCAAAA |
| | | | CCTCGTCGACCACGGTGACTCTGCGCAT |
| | | | GACCAGTCTGACAGCCGCGGACACGGCC |
| | | | ACCTATTTCTGTGCGAGATCCTCATATC |
| | | | CTGATAGTAGTGGCTATAGTTATGGCAT |
| | | | GGACCTCTGGGGCCCTGGGACCCTCGTC |
| | | | ACCGTCTCTTCA |
| 67 | 242-3 | Light | ATGGACACGAGGGCCCCCACTCAGCTGC |
| | | Chain | TGGGGCTCCTGTTGCTCTGGCTCCCAGG |
| | | | TGCCAGATGTGCTGACATTGTGATGACC |
| | | | CAGACTCCAGCCTCCGTGGAGGCAGCTG |
| | | | TGGGAGGCACAGTCACCATCAAGTGCCA |
| | | | GGCCAGTCAGAACATTAACAGCAATTTA |
| | | | GCCTGGTATCAGCAGAAACCAGGGCAGC |
| | | | GTCCCAAGTTCCTGATGTATCTGACATC |
| | | | CAAACTGGCATCTGGGGTCCCATCGCGG |
| | | | TTCAGAGGCAGTGGATCTGGGACACAGT |
| | | | TCACTCTCACCATCAGCGACCTGGAGTG |
| | | | TGCCGATGCTGCCACTTACTACTGTCAA |
| | | | ACCTATTATGATATTAGTAATTATGGAT |
| | | | ATGCTTTCGGCGGAGGGACCGAGGTGGT |
| | | | GGTCAAAGGTGATCCAGTTGCACCTACT |
| | | | GTCCTCATCTTCCCACCAGCTGCTGATC |
| | | | AGGTGGCAACTGGAACAGTCACCATCGT |
| | | | GTGTGTGGCGAATAAATACTTTCCCGAT |
| | | | GTCACCGTCACCTGGGAGGTGGATGGCA |
| | | | CCACCCAAACAACTGGCATCGAGAACAG |
| | | | TAAAACACCGCAGAATTCTGCAGATTGT |
| | | | ACCTACAACCTCAGCAGCACTCTGACAC |
| | | | TGACCAGCACACAGTACAACAGCCACAA |
| | | | AGAGTACACCTGCAAGGTGACCCAGGGC |
| | | | ACGACCTCAGTCGTCCAGAGCTTCAATA |
| | | | GGGGTGACTGTTAG |
| 68 | 242-3 | VL | ATGGACACGAGGGCCCCCACTCAGCTGC |
| | | | TGGGGCTCCTGTTGCTCTGGCTCCCAGG |
| | | | TGCCAGATGTGCTGACATTGTGATGACC |
| | | | CAGACTCCAGCCTCCGTGGAGGCAGCTG |
| | | | TGGGAGGCACAGTCACCATCAAGTGCCA |
| | | | GGCCAGTCAGAACATTAACAGCAATTTA |
| | | | GCCTGGTATCAGCAGAAACCAGGGCAGC |
| | | | GTCCCAAGTTCCTGATGTATCTGACATC |
| | | | CAAACTGGCATCTGGGGTCCCATCGCGG |
| | | | TTCAGAGGCAGTGGATCTGGGACACAGT |
| | | | TCACTCTCACCATCAGCGACCTGGAGTG |
| | | | TGCCGATGCTGCCACTTACTACTGTCAA |
| | | | ACCTATTATGATATTAGTAATTATGGAT |
| | | | ATGCTTTCGGCGGAGGGACCGAGGTGGT |
| | | | GGTCAAA |
| 69 | 289-3 | Heavy | ATGGAGACTGGGCTGCGCTGGCTTCTCC |
| | | Chain | TGGTCGCTGTGCTCAAAGGTGTCCAGTG |
| | | | TCAGGAGGAGCTGGAGGAGTCCGGGGGA |
| | | | GACCTGGTCAAGCCTGGGGCATCCCTGA |
| | | | CACTCACCTGCAAAGCCTCTGGATTCTC |
| | | | CTTCAGTAGTGGCGCCTACATATGCTGG |
| | | | GTCCGCCAGGCTCCAGGGAAGGGGCCTG |
| | | | AGTGGATCGCGTGTATTTATACTGGTGA |
| | | | TGGCCGCACATACTACGCGAACTGGGCG |
| | | | AAGCGCCGCTTCACCATCTCCAGTTCCT |
| | | | CGTCGACCACGGTGGCTCTGGAAATGCC |
| | | | CAGTCTGACAGACGCGGACACGGCGACC |
| | | | TATTTCTGTGCGCGAGCAATTGCTGTTG |
| | | | GCGCTGGTTATGGTGTTGGAAACTACTT |
| | | | TACCTTGTGGGGCCCAGGCACCCTGGTC |
| | | | ACCGTCTCCTCAGGGCAACCTAAGGCTC |
| | | | CATCAGTCTTCCCACTGGCCCCCTGCTG |
| | | | CGGGGACACACCCAGCTCCACGGTGACC |
| | | | CTGGGCTGCCTGGTCAAAGGGTACCTCC |
| | | | CGGAGCCAGTGACCGTGACCTGGAACTC |
| | | | GGGCACCCTCACCAATGGGGTACGCACC |
| | | | TTCCCGTCCGTCCGGCAGTCCTCAGGCC |
| | | | TCTACTCGCTGAGCAGCGTGGTGAGCGT |
| | | | GACCTCAAGCAGCCAGCCCGTCACCTGC |
| | | | AACGTGGCCCACCCAGCCACCAACACCA |
| | | | AAGTGGACAAGACCGTTGCGCCCTCGAC |
| | | | ATGCAGCAAGCCCACGTGCCCACCCCCT |
| | | | GAACTCCTGGGGGGACCGTCTGTCTTCA |
| | | | TCTTCCCCCCAAAACCCAAGGACACCCT |
| | | | CATGATCTCACGCACCCCCGAGGTCACA |
| | | | TGCGTGGTGGTGGACGTGAGCCAGGATG |
| | | | ACCCCGAGGTGCAGTTCACATGGTACAT |
| | | | AAACAACGAGCAGGTGCGCACCGCCCGG |
| | | | CCGCCGCTACGGGAGCAGCAGTTCAACA |
| | | | GCACGATCCGCGTGGTCAGCACCCTCCC |
| | | | CATCGCGCACCAGGACTGGCTGAGGGGC |
| | | | AAGGAGTTCAAGTGCAAAGTCCACAACA |
| | | | AGGCACTCCCGGCCCCCATCGAGAAAAC |
| | | | CATCTCCAAAGCCAGAGGGCAGCCCCTG |
| | | | GAGCCGAAGGTCTACACCATGGGCCCTC |
| | | | CCCGGGAGGAGCTGAGCAGCAGGTCGGT |
| | | | CAGCCTGACCTGCATGATCAACGGCTTC |
| | | | TACCCTTCCGACATCTCGGTGGAGTGGG |
| | | | AGAAGAACGGGAAGGCAGAGGACAACTA |
| | | | CAAGACCACGCCGGCCGTGCTGGACAGC |
| | | | GACGGCTCCTACTTCCTCTACAGCAAGC |
| | | | TCTCAGTGCCCACGAGTGAGTGGCAGCG |
| | | | GGGCGACGTCTTCACCTGCTCCGTGATG |
| | | | CACGAGGCCTTGCACAACCACTACACGC |
| | | | AGAAGTCCATCTCCCGCTCTCCGGGTAA |
| | | | ATGA |
| 70 | 289-3 | VH | ATGGAGACTGGGCTGCGCTGGCTTCTCC |
| | | | TGGTCGCTGTGCTCAAAGGTGTCCAGTG |
| | | | TCAGGAGGAGCTGGAGGAGTCCGGGGGA |
| | | | GACCTGGTCAAGCCTGGGGCATCCCTGA |
| | | | CACTCACCTGCAAAGCCTCTGGATTCTC |
| | | | CTTCAGTAGTGGCGCCTACATATGCTGG |
| | | | GTCCGCCAGGCTCCAGGGAAGGGGCCTG |
| | | | AGTGGATCGCGTGTATTTATACTGGTGA |
| | | | TGGCCGCACATACTACGCGAACTGGGCG |
| | | | AAGCGCCGCTTCACCATCTCCAGTTCCT |
| | | | CGTCGACCACGGTGGCTCTGGAAATGCC |
| | | | CAGTCTGACAGACGCGGACACGGCGACC |
| | | | TATTTCTGTGCGCGAGCAATTGCTGTTG |
| | | | GCGCTGGTTATGGTGTTGGAAACTACTT |
| | | | TACCTTGTGGGGCCCAGGCACCCTGGTC |
| | | | ACCGTCTCCTCA |
| 71 | 289-3 | Light | ATGGACACGAGGGCCCCCACTCAGCTGC |
| | | Chain | TGGGGCTCCTGCTGCTCTGGCTCCCAGG |
| | | | TGCCATATGTGACATTGTGATGACCCAG |
| | | | ACTCCATCTTCCGTGTCTGCAGCTGTGG |
| | | | GAGGCACAGTCACCATCAATTGCCACGC |
| | | | CAGTGAGAACATTTACGGCTACTTATCC |
| | | | TGGTATCAGCAGAAACCAGGGCAGCCTC |
| | | | CCAAGCTCCTGATCTACAAGGCTTCCAC |
| | | | TCTGGCATCTGGGGTCCCATCGCGGTTC |
| | | | AAAGGCAGTGGAGCTGGGACACAGTTCA |
| | | | CTCTCACCATCACCGCCCTGGAGTGTGC |
| | | | CGATGCTGCCACTTACTACTGTCAAAGT |
| | | | TATTATACTAGTAGTAGTAATGCTGATG |
| | | | GTTCGGAGAATGCTTTTGGCGGAGGGAC |
| | | | CGAGGTGGTGGTCGAAGGTGATCCAGTT |
| | | | GCACCTACTGTCCTCATCTTCCCACCAG |
| | | | CTGCTGATCAGGTGGCAACTGGAACAGT |
| | | | CACCATCGTGTGTGTGGCGAATAAATAC |
| | | | TTTCCCGATGTCACCGTCACCTGGGAGG |
| | | | TGGATGGCACCACCCAAACAACTGGCAT |
| | | | CGAGAACAGTAAAACACCGCAGAATTCT |
| | | | GCAGATTGTACCTACAACCTCAGCAGCA |
| | | | CTCTGACACTGACCAGCACACAGTACAA |
| | | | CAGCCACAAAGAGTACACCTGCAAGGTG |
| | | | ACCCAGGGCACGACCTCAGTCGTCCAGA |
| | | | GCTTCAATAGGGGTGACTGTTAG |
| 72 | 289-3 | VL | ATGGACACGAGGGCCCCCACTCAGCTGC |
| | | | TGGGGCTCCTGCTGCTCTGGCTCCCAGG |
| | | | TGCCATATGTGACATTGTGATGACCCAG |
| | | | ACTCCATCTTCCGTGTCTGCAGCTGTGG |
| | | | GAGGCACAGTCACCATCAATTGCCACGC |
| | | | CAGTGAGAACATTTACGGCTACTTATCC |
| | | | TGGTATCAGCAGAAACCAGGGCAGCCTC |
| | | | CCAAGCTCCTGATCTACAAGGCTTCCAC |
| | | | TCTGGCATCTGGGGTCCCATCGCGGTTC |
| | | | AAAGGCAGTGGAGCTGGGACACAGTTCA |
| | | | CTCTCACCATCACCGCCCTGGAGTGTGC |
| | | | CGATGCTGCCACTTACTACTGTCAAAGT |
| | | | TATTATACTAGTAGTAGTAATGCTGATG |
| | | | GTTCGGAGAATGCTTTTGGCGGAGGGAC |
| | | | CGAGGTGGTGGTCGAA |
| 73 | 306-2 | Heavy | ATGGAGACTGGGCTGCGCTGGCTTCTCC |
| | | Chain | TGGTCGCTGTGCTCAAAGGTGTCCAGTG |
| | | | TCAGTCGTTGGAGGAGTCCGGGGGAGAC |
| | | | CTGGTCAAGCCTGGGGCATCCCTGACAC |
| | | | TCACCTGCACAGCCTCTGGATTCGACTT |
| | | | CAGTGACAGATATTACATGTGTTGGGTC |
| | | | CGCCAGGCTCCAGGGAAGGGGCTGGAGT |
| | | | GGATCGGATGCATTTATGTTGGTAGTGG |
| | | | TGACACTTATTATGCGAGCTGGGCGAAA |
| | | | GGCCGATTCACCATCTCCAGAACCTCGT |
| | | | CGACCACGGTGACTCTGCAAATGACCCG |
| | | | TCTGACAGCCGCGGACACGGCCACCTAT |
| | | | TTCTGTGCGAGACATCCTGGTACTTACT |
| | | | TTACTTTATGGGGCCCAGGCACCCTGGT |
| | | | CACCGTCTCCTCAGGGCAACCTAAGGCT |
| | | | CCATCAGTCTTCCCACTGGCCCCCTGCT |
| | | | GCGGGGACACACCCAGCTCCACGGTGAC |
| | | | CCTGGGCTGCCTGGTCAAAGGGTACCTC |
| | | | CCGGAGCCAGTGACCGTGACCTGGAACT |
| | | | CGGGCACCCTCACCAATGGGGTACGCAC |
| | | | CTTCCCGTCCGTCCGGCAGTCCTCAGGC |
| | | | CTCTACTCGCTGAGCAGCGTGGTGAGCG |
| | | | TGACCTCAAGCAGCCAGCCCGTCACCTG |
| | | | CAACGTGGCCCACCCAGCCACCAACACC |
| | | | AAAGTGGACAAGACCGTTGCGCCCTCGA |
| | | | CATGCAGCAAGCCCACGTGCCCACCCCC |
| | | | TGAACTCCTGGGGGGACCGTCTGTCTTC |
| | | | ATCTTCCCCCCAAAACCCAAGGACACCC |
| | | | TCATGATCTCACGCACCCCCGAGGTCAC |
| | | | ATGCGTGGTGGTGGACGTGAGCCAGGAT |
| | | | GACCCCGAGGTGCAGTTCACATGGTACA |
| | | | TAAACAACGAGCAGGTGCGCACCGCCCG |
| | | | GCCGCCGCTACGGGAGCAGCAGTTCAAC |
| | | | AGCACGATCCGCGTGGTCAGCACCCTCC |
| | | | CCATCGCGCACCAGGACTGGCTGAGGGG |
| | | | CAAGGAGTTCAAGTGCAAAGTCCACAAC |
| | | | AAGGCACTCCCGGCCCCCATCGAGAAAA |
| | | | CCATCTCCAAAGCCAGAGGGCAGCCCCT |
| | | | GGAGCCGAAGGTCTACACCATGGGCCCT |
| | | | CCCCGGGAGGAGCTGAGCAGCAGGTCGG |
| | | | TCAGCCTGACCTGCATGATCAACGGCTT |
| | | | CTACCCTTCCGACATCTCGGTGGAGTGG |
| | | | GAGAAGAACGGGAAGGCAGAGGACAACT |
| | | | ACAAGACCACGCCGGCCGTGCTGGACAG |
| | | | CGACGGCTCCTACTTCCTCTACAGCAAG |
| | | | CTCTCAGTGCCCACGAGTGAGTGGCAGC |
| | | | GGGGCGACGTCTTCACCTGCTCCGTGAT |
| | | | GCACGAGGCCTTGCACAACCACTACACG |
| | | | CAGAAGTCCATCTCCCGCTCTCCGGGTA |
| | | | AATGA |
| 74 | 306-2 | VH | ATGGAGACTGGGCTGCGCTGGCTTCTCC |
| | | | TGGTCGCTGTGCTCAAAGGTGTCCAGTG |
| | | | TCAGTCGTTGGAGGAGTCCGGGGGAGAC |
| | | | CTGGTCAAGCCTGGGGCATCCCTGACAC |
| | | | TCACCTGCACAGCCTCTGGATTCGACTT |
| | | | CAGTGACAGATATTACATGTGTTGGGTC |
| | | | CGCCAGGCTCCAGGGAAGGGGCTGGAGT |
| | | | GGATCGGATGCATTTATGTTGGTAGTGG |
| | | | TGACACTTATTATGCGAGCTGGGCGAAA |
| | | | GGCCGATTCACCATCTCCAGAACCTCGT |
| | | | CGACCACGGTGACTCTGCAAATGACCCG |
| | | | TCTGACAGCCGCGGACACGGCCACCTAT |
| | | | TTCTGTGCGAGACATCCTGGTACTTACT |
| | | | TTACTTTATGGGGCCCAGGCACCCTGGT |
| | | | CACCGTCTCCTCA |
| 75 | 306-2 | Light | ATGGACACGAGGGCCCCCACTCAGCTGC |
| | | Chain | TGGGGCTCCTGCTGCTCTGGCTCCCAGG |
| | | | TGCCAGATGTGATGTTGTGATGACCCAG |
| | | | ACTCCATCCTCCGTGTCTGCAGCTGTGG |
| | | | GAGGCACAGTCACCATCAATTGCCAGGC |
| | | | CAGTCAGAACATTGTCAACAATTTAGCC |
| | | | TGGTATCAGCAGAGACCAGGGCAGCCTC |
| | | | CCAAACTCCTGATCTATGATACATCCAC |
| | | | TCTGGCATCTGGGGTCCCATCGCGGTTC |
| | | | AAAGGCAGTGGATCTGGGACAGAGTTCA |
| | | | CTCTCACCATCAGCGACCTGGAGTGTGC |
| | | | CGATGCTGCCACTTACTACTGTCAAACC |
| | | | TATTATTATTATAATAAAATTATAAATG |
| | | | GTTTCGGCGGAGGGACCGAGGTGGTGGT |
| | | | CAAAGGTGATCCAGTTGCACCTACTGTC |
| | | | CTCATCTTCCCACCAGCTGCTGATCAGG |
| | | | TGGCAACTGGAACAGTCACCATCGTGTG |
| | | | TGTGGCGAATAAATACTTTCCCGATGTC |
| | | | ACCGTCACCTGGGAGGTGGATGGCACCA |
| | | | CCCAAACAACTGGCATCGAGAACAGTAA |
| | | | AACACCGCAGAATTCTGCAGATTGTACC |
| | | | TACAACCTCAGCAGCACTCTGACACTGA |
| | | | CCAGCACACAGTACAACAGCCACAAAGA |
| | | | GTACACCTGCAAGGTGACCCAGGGCACG |
| | | | ACCTCAGTCGTCCAGAGCTTCAATAGGG |
| | | | GTGACTGTTAG |
| 76 | 306-2 | VL | ATGGACACGAGGGCCCCCACTCAGCTGC |
| | | | TGGGGCTCCTGCTGCTCTGGCTCCCAGG |
| | | | TGCCAGATGTGATGTTGTGATGACCCAG |
| | | | ACTCCATCCTCCGTGTCTGCAGCTGTGG |
| | | | GAGGCACAGTCACCATCAATTGCCAGGC |
| | | | CAGTCAGAACATTGTCAACAATTTAGCC |
| | | | TGGTATCAGCAGAGACCAGGGCAGCCTC |
| | | | CCAAACTCCTGATCTATGATACATCCAC |
| | | | TCTGGCATCTGGGGTCCCATCGCGGTTC |
| | | | AAAGGCAGTGGATCTGGGACAGAGTTCA |
| | | | CTCTCACCATCAGCGACCTGGAGTGTGC |
| | | | CGATGCTGCCACTTACTACTGTCAAACC |
| | | | TATTATTATTATAATAAAATTATAAATG |
| | | | GTTTCGGCGGAGGGACCGAGGTGGTGGT |
| | | | CAAA |
| 77 | 11-3 | Heavy | ATGGAGACTGGGCTGCGCTGGCTTCTCC |
| | | Chain | TGGTCGCTGTGCTCAAAGGTGTCCAGTG |
| | | | TCAGGAGCAGCTGGTGGAGTCCGGGGGA |
| | | | GGCCTGGTCCAGCCTGAGGGATCCCTGA |
| | | | CAATCACCTGCACAGGTTCTGGATTCTC |
| | | | CTTCAGTAGCAGGTTCTACATGTGCTGG |
| | | | GTCCGCCAGGCTCCAGGGAAGGGGCTGG |
| | | | AGTGGATCGCATGTATTTATGGTGGTAG |
| | | | TAGTGGTAGCACTTACTACGCGAGCTGG |
| | | | GCGAAAGGCCGATTCACCATCTCCAAAA |
| | | | CCTCGTCGACCACGGTGACTCTGCAAAT |
| | | | TTCCAGTCTGACAGCCGCGGACACGGCC |
| | | | ACCTATTTCTGTGCCAGAGGTGGTAGTA |
| | | | CTGCTGCTGCGGGCTTTAACTTGTGGGG |
| | | | CCCAGGCACCCTGCTCACCGTCTCCTCA |
| | | | GGGCAACCTAAGGCTCCATCAGTCTTCC |
| | | | CACTGGCCCCCTGCTGCGGGGACACACC |
| | | | CAGCTCCACGGTGACCCTGGGCTGCCTG |
| | | | GTCAAAGGGTACCTCCCGGAGCCAGTGA |
| | | | CCGTGACCTGGAACTCGGGCACCCTCAC |
| | | | CAATGGGGTACGCACCTTCCCGTCCGTC |
| | | | CGGCAGTCCTCAGGCCTCTACTCGCTGA |
| | | | GCAGCGTGGTGAGCGTGACCTCAAGCAG |
| | | | CCAGCCCGTCACCTGCAACGTGGCCCAC |
| | | | CCAGCCACCAACACCAAAGTGGACAAGA |
| | | | CCGTTGCGCCCTCGACATGCAGCAAGCC |
| | | | CACGTGCCCACCCCCTGAACTCCTGGGG |
| | | | GGACCGTCTGTCTTCATCTTCCCCCCAA |
| | | | AACCCAAGGACACCCTCATGATCTCACG |
| | | | CACCCCCGAGGTCACATGCGTGGTGGTG |
| | | | GACGTGAGCCAGGATGACCCCGAGGTGC |
| | | | AGTTCACATGGTACATAAACAACGAGCA |
| | | | GGTGCGCACCGCCCGGCCGCCGCTACGG |
| | | | GAGCAGCAGTTCAACAGCACGATCCGCG |
| | | | TGGTCAGCACCCTCCCCATCGCGCACCA |
| | | | GGACTGGCTGAGGGGCAAGGAGTTCAAG |
| | | | TGCAAAGTCCACAACAAGGCACTCCCGG |
| | | | CCCCCATCGAGAAAACCATCTCCAAAGC |
| | | | CAGAGGGCAGCCCCTGGAGCCGAAGGTC |
| | | | TACACCATGGGCCCTCCCCGGGAGGAGC |
| | | | TGAGCAGCAGGTCGGTCAGCCTGACCTG |
| | | | CATGATCAACGGCTTCTACCCTTCCGAC |
| | | | ATCTCGGTGGAGTGGGAGAAGAACGGGA |
| | | | AGGCAGAGGACAACTACAAGACCACGCC |
| | | | GGCCGTGCTGGACAGCGACGGCTCCTAC |
| | | | TTCCTCTACAGCAAGCTCTCAGTGCCCA |
| | | | CGAGTGAGTGGCAGCGGGGCGACGTCTT |
| | | | CACCTGCTCCGTGATGCACGAGGCCTTG |
| | | | CACAACCACTACACGCAGAAGTCCATCT |
| | | | CCCGCTCTCCGGGTAAATGA |
| 78 | 11-3 | VH | ATGGAGACTGGGCTGCGCTGGCTTCTCC |
| | | | TGGTCGCTGTGCTCAAAGGTGTCCAGTG |
| | | | TCAGGAGCAGCTGGTGGAGTCCGGGGGA |
| | | | GGCCTGGTCCAGCCTGAGGGATCCCTGA |
| | | | CAATCACCTGCACAGGTTCTGGATTCTC |
| | | | CTTCAGTAGCAGGTTCTACATGTGCTGG |
| | | | GTCCGCCAGGCTCCAGGGAAGGGGCTGG |
| | | | AGTGGATCGCATGTATTTATGGTGGTAG |
| | | | TAGTGGTAGCACTTACTACGCGAGCTGG |
| | | | GCGAAAGGCCGATTCACCATCTCCAAAA |
| | | | CCTCGTCGACCACGGTGACTCTGCAAAT |
| | | | TTCCAGTCTGACAGCCGCGGACACGGCC |
| | | | ACCTATTTCTGTGCCAGAGGTGGTAGTA |
| | | | CTGCTGCTGCGGGCTTTAACTTGTGGGG |
| | | | CCCAGGCACCCTGCTCACCGTCTCCTCA |
| 79 | 11-3 | Light | ATGGACACGAGGGCCCCCACTCAGCTGC |
| | | Chain | TGGGGCTCCTGCTGCTCTGGCTCCCAGG |
| | | | TGCCAGATGTGATGTTGTGATGACCCAG |
| | | | ACTCCAGCCTCCGTGTCTGCAACTGTGG |
| | | | GAGGCACAGTCACCATCAAGTGCCAGGC |
| | | | CAGTGAGGACATTTATAACTTATTGGCC |
| | | | TGGTATCAGCAGAAACCAGGGCAGCCTC |
| | | | CCAAACTCCTGATCTATTATGCATCCAC |
| | | | TCTGGCATCTGGGGTCCCATCACGGTTC |
| | | | AAAGGCAGTGGATCTGGGACAGAGTTCA |
| | | | CTCTCACCATCAGCGACCTGGAGTGTGC |
| | | | CGATGCTGCCACTTACTACTGTCAATGT |
| | | | AATGATTATGGTGGTACTTATGTCCCCA |
| | | | ATGCTTTCGGCGGAGGGACCGAGGTGGT |
| | | | GGTCAAGGGTGATCCAGTTGCACCTACT |
| | | | GTCCTCATCTTCCCACCAGCTGCTGATC |
| | | | AGGTGGCAACTGGAACAGTCACCATCGT |
| | | | GTGTGTGGCGAATAAATACTTTCCCGAT |
| | | | GTCACCGTCACCTGGGAGGTGGATGGCA |
| | | | CCACCCAAACAACTGGCATCGAGAACAG |
| | | | TAAAACACCGCAGAATTCTGCAGATTGT |
| | | | ACCTACAACCTCAGCAGCACTCTGACAC |
| | | | TGACCAGCACACAGTACAACAGCCACAA |
| | | | AGAGTACACCTGCAAGGTGACCCAGGGC |
| | | | ACGACCTCAGTCGTCCAGAGCTTCAATA |
| | | | GGGGTGACTGTTAG |
| 80 | 11-3 | VL | ATGGACACGAGGGCCCCCACTCAGCTGC |
| | | | TGGGGCTCCTGCTGCTCTGGCTCCCAGG |
| | | | TGCCAGATGTGATGTTGTGATGACCCAG |
| | | | ACTCCAGCCTCCGTGTCTGCAACTGTGG |
| | | | GAGGCACAGTCACCATCAAGTGCCAGGC |
| | | | CAGTGAGGACATTTATAACTTATTGGCC |
| | | | TGGTATCAGCAGAAACCAGGGCAGCCTC |
| | | | CCAAACTCCTGATCTATTATGCATCCAC |
| | | | TCTGGCATCTGGGGTCCCATCACGGTTC |
| | | | AAAGGCAGTGGATCTGGGACAGAGTTCA |
| | | | CTCTCACCATCAGCGACCTGGAGTGTGC |
| | | | CGATGCTGCCACTTACTACTGTCAATGT |
| | | | AATGATTATGGTGGTACTTATGTCCCCA |
| | | | ATGCTTTCGGCGGAGGGACCGAGGTGGT |
| | | | GGTCAAG |
| 81 | 78-2 | Heavy | ATGGAGACTGGGCTGCGCTGGCTTCTCC |
| | | Chain | TGGTCGCTGTGCTCAAAGGTGTCCAGTG |
| | | | TCAGCAGCAGCTGGTGGAGTCCGGGGGA |
| | | | GGCCTGGTCAAGCCTGGGGCATCCCTGA |
| | | | CACTCAACTGCAAAGCCTCTGGACTCGA |
| | | | CTTCAGTACCAACTCCTACATGTGCTGG |
| | | | GTCCGCCAGGCTCCAGGGAAGGGCCTGG |
| | | | AGTGGATCGCATGCATTTATGTTGGTGA |
| | | | TAGTAGTGAGATTTATTACGCGAGCTGG |
| | | | GCGAAAGGCCGATTCACCATCTCCAAAA |
| | | | CCTCGTCGACCACGGTGACTCTACAAAT |
| | | | GACCAGTCTGACAGCCGCGGACACGGCC |
| | | | ACCTATTTCTGTGCGAGAGATCTCCCTT |
| | | | CCTTTACTGCTCCTTATGCTGGTTATCT |
| | | | TCGCTTGTGGGGCCCAGGCACCCTGGTC |
| | | | ACCGTCTCCTCAGGGCAACCTAAGGCTC |
| | | | CATCAGTCTTCCCACTGGCCCCCTGCTG |
| | | | CGGGGACACACCCAGCTCCACGGTGACC |
| | | | CTGGGCTGCCTGGTCAAAGGGTACCTCC |
| | | | CGGAGCCAGTGACCGTGACCTGGAACTC |
| | | | GGGCACCCTCACCAATGGGGTACGCACC |
| | | | TTCCCGTCCGTCCGGCAGTCCTCAGGCC |
| | | | TCTACTCGCTGAGCAGCGTGGTGAGCGT |
| | | | GACCTCAAGCAGCCAGCCCGTCACCTGC |
| | | | AACGTGGCCCACCCAGCCACCAACACCA |
| | | | AAGTGGACAAGACCGTTGCGCCCTCGAC |
| | | | ATGCAGCAAGCCCACGTGCCCACCCCCT |
| | | | GAACTCCTGGGGGGACCGTCTGTCTTCA |
| | | | TCTTCCCCCCAAAACCCAAGGACACCCT |
| | | | CATGATCTCACGCACCCCCGAGGTCACA |
| | | | TGCGTGGTGGTGGACGTGAGCCAGGATG |
| | | | ACCCCGAGGTGCAGTTCACATGGTACAT |
| | | | AAACAACGAGCAGGTGCGCACCGCCCGG |
| | | | CCGCCGCTACGGGAGCAGCAGTTCAACA |
| | | | GCACGATCCGCGTGGTCAGCACCCTCCC |
| | | | CATCGCGCACCAGGACTGGCTGAGGGGC |
| | | | AAGGAGTTCAAGTGCAAAGTCCACAACA |
| | | | AGGCACTCCCGGCCCCCATCGAGAAAAC |
| | | | CATCTCCAAAGCCAGAGGGCAGCCCCTG |
| | | | GAGCCGAAGGTCTACACCATGGGCCCTC |
| | | | CCCGGGAGGAGCTGAGCAGCAGGTCGGT |
| | | | CAGCCTGACCTGCATGATCAACGGCTTC |
| | | | TACCCTTCCGACATCTCGGTGGAGTGGG |
| | | | AGAAGAACGGGAAGGCAGAGGACAACTA |
| | | | CAAGACCACGCCGGCCGTGCTGGACAGC |
| | | | GACGGCTCCTACTTCCTCTACAGCAAGC |
| | | | TCTCAGTGCCCACGAGTGAGTGGCAGCG |
| | | | GGGCGACGTCTTCACCTGCTCCGTGATG |
| | | | CACGAGGCCTTGCACAACCACTACACGC |
| | | | AGAAGTCCATCTCCCGCTCTCCGGGTAA |
| | | | ATGA |
| 82 | 78-2 | VH | ATGGAGACTGGGCTGCGCTGGCTTCTCC |
| | | | TGGTCGCTGTGCTCAAAGGTGTCCAGTG |
| | | | TCAGCAGCAGCTGGTGGAGTCCGGGGGA |
| | | | GGCCTGGTCAAGCCTGGGGCATCCCTGA |
| | | | CACTCAACTGCAAAGCCTCTGGACTCGA |
| | | | CTTCAGTACCAACTCCTACATGTGCTGG |
| | | | GTCCGCCAGGCTCCAGGGAAGGGCCTGG |
| | | | AGTGGATCGCATGCATTTATGTTGGTGA |
| | | | TAGTAGTGAGATTTATTACGCGAGCTGG |
| | | | GCGAAAGGCCGATTCACCATCTCCAAAA |
| | | | CCTCGTCGACCACGGTGACTCTACAAAT |
| | | | GACCAGTCTGACAGCCGCGGACACGGCC |
| | | | ACCTATTTCTGTGCGAGAGATCTCCCTT |
| | | | CCTTTACTGCTCCTTATGCTGGTTATCT |
| | | | TCGCTTGTGGGGCCCAGGCACCCTGGTC |
| | | | ACCGTCTCCTCA |
| 83 | 78-2 | Light | ATGGCCTGGGCTCCTCTGCTCCTGCTGC |
| | | Chain | TCCTCTCCCACTGCACAGGTTCCCTCTC |
| | | | CCAGCCTGTGCTGACTCAGCCGCCCTCC |
| | | | CTGTCTGCGTCTCTGGGCACAACAGCCA |
| | | | GACTCACCTGCACCCTGAGGACTGGCTA |
| | | | CAATGTTGGCGACTACCCTTTACTGTGG |
| | | | CTCCAGCGGGTGCCAGGGAGGCCTCCCA |
| | | | GGTATCTCCTGACCTACCACACAGAAGA |
| | | | ATTTAAACATCAGGGCTCCGGGGTGCAC |
| | | | AGCCGCTTCTCTGGATCCAAAGACGCCT |
| | | | CAGAGAATGCCGGTGTCCTGAGCATCTC |
| | | | TGGGCTGCAGCCCGAGGACGAGGCCAAC |
| | | | TATTATTGTTATACTGTTCATGCTACTG |
| | | | AGAGCAGCCTCCATTATGTGTTCGGCGG |
| | | | AGGGACACAACTGACCGTCACAGGTCAG |
| | | | CCCGCGGTGACCCCCTCGGTCATTTTGT |
| | | | TCCCGCCCTCCTCTGAGGAACTCAAGGA |
| | | | CAACAAGGCCACCTTGGTGTGTCTGATC |
| | | | AATGACTTCTACCCGGGCACCGTGAAGG |
| | | | TGAACTGGAAGGCCGATGGTACTCCCGT |
| | | | CACCCAGGGCGTGGACACCACCCAGCCC |
| | | | TCCAAACAGAGCAACAGCAAGTATGCGG |
| | | | CCAGCAGCTTCCTCAGCCTGTCAGCCAA |
| | | | TCAATGGAAATCCTACCAGAGTGTCACC |
| | | | TGCCAGGTCACGCATGAGGGCCACACGG |
| | | | TGGAGAAGAGCCTGGCCCCTGCAGAATG |
| | | | CTCC |
| 84 | 78-2 | VL | ATGGCCTGGGCTCCTCTGCTCCTGCTGC |
| | | | TCCTCTCCCACTGCACAGGTTCCCTCTC |
| | | | CCAGCCTGTGCTGACTCAGCCGCCCTCC |
| | | | CTGTCTGCGTCTCTGGGCACAACAGCCA |
| | | | GACTCACCTGCACCCTGAGGACTGGCTA |
| | | | CAATGTTGGCGACTACCCTTTACTGTGG |
| | | | CTCCAGCGGGTGCCAGGGAGGCCTCCCA |
| | | | GGTATCTCCTGACCTACCACACAGAAGA |
| | | | ATTTAAACATCAGGGCTCCGGGGTGCAC |
| | | | AGCCGCTTCTCTGGATCCAAAGACGCCT |
| | | | CAGAGAATGCCGGTGTCCTGAGCATCTC |
| | | | TGGGCTGCAGCCCGAGGACGAGGCCAAC |
| | | | TATTATTGTTATACTGTTCATGCTACTG |
| | | | AGAGCAGCCTCCATTATGTGTTCGGCGG |
| | | | AGGGACACAACTGACCGTCACA |
| 115 | 270-12 | Heavy | ATGGAGACTGGGCTGCGCTGGCTTCTCC |
| | | Chain | TGGTCGCTGTGCTCAAAGGTGTCCAGTG |
| | | | TCAGGCGCAGCTGGAGGAGTCCGGGGGA |
| | | | GACCTGGTCAAGCCTGAGGGATCCCTGA |
| | | | CACTCACCTGCACAGCCTCTGGATTCTC |
| | | | CTTCACTGACAGGCATTACATGTGCTGG |
| | | | GTCCGCCAGGCTCCGGGGAAGGGGCTGG |
| | | | AGTGGATCGCATGCGTTTATCCTGGTAG |
| | | | TAGTGGTAGCACTTACTACGCGAGCTGG |
| | | | GCGAGAGGCCGATTCACCATCTCCAAAA |
| | | | CCTCGTCGACCACGGTGACTCTGCGCAT |
| | | | GACCAGTCTGACAGCCGCGGACACGGCC |
| | | | ACCTATTTCTGTGCGAGATCCTCATATC |
| | | | CTGATAGTAGTGGCTATAGTTATGGCAT |
| | | | GGACCTCTGGGGCCCTGGGACCCTCGTC |
| | | | ACCGTCTCTTCAGGGCAACCTAAGGCTC |
| | | | CATCAGTCTTCCCACTGGCCCCCTGCTG |
| | | | CGGGGACACACCCAGCTCCACGGTGACC |
| | | | CTGGGCTGCCTGGTCAAAGGGTACCTCC |
| | | | CGGAGCCAGTGACCGTGACCTGGAACTC |
| | | | GGGCACCCTCACCAATGGGGTACGCACC |
| | | | TTCCCGTCCGTCCGGCAGTCCTCAGGCC |
| | | | TCTACTCGCTGAGCAGCGTGGTGAGCGT |
| | | | GACCTCAAGCAGCCAGCCCGTCACCTGC |
| | | | AACGTGGCCCACCCAGCCACCAACACCA |
| | | | AAGTGGACAAGACCGTTGCGCCCTCGAC |
| | | | ATGCAGCAAGCCCACGTGCCCACCCCCT |
| | | | GAACTCCTGGGGGGACCGTCTGTCTTCA |
| | | | TCTTCCCCCCAAAACCCAAGGACACCCT |
| | | | CATGATCTCACGCACCCCCGAGGTCACA |
| | | | TGCGTGGTGGTGGACGTGAGCCAGGATG |
| | | | ACCCCGAGGTGCAGTTCACATGGTACAT |
| | | | AAACAACGAGCAGGTGCGCACCGCCCGG |
| | | | CCGCCGCTACGGGAGCAGCAGTTCAACA |
| | | | GCACGATCCGCGTGGTCAGCACCCTCCC |
| | | | CATCGCGCACCAGGACTGGCTGAGGGGC |
| | | | AAGGAGTTCAAGTGCAAAGTCCACAACA |
| | | | AGGCACTCCCGGCCCCCATCGAGAAAAC |
| | | | CATCTCCAAAGCCAGAGGGCAGCCCCTG |
| | | | GAGCCGAAGGTCTACACCATGGGCCCTC |
| | | | CCCGGGAGGAGCTGAGCAGCAGGTCGGT |
| | | | CAGCCTGACCTGCATGATCAACGGCTTC |
| | | | TACCCTTCCGACATCTCGGTGGAGTGGG |
| | | | AGAAGAACGGGAAGGCAGAGGACAACTA |
| | | | CAAGACCACGCCGGCCGTGCTGGACAGC |
| | | | GACGGCTCCTACTTCCTCTACAGCAAGC |
| | | | TCTCAGTGCCCACGAGTGAGTGGCAGCG |
| | | | GGGCGACGTCTTCACCTGCTCCGTGATG |
| | | | CACGAGGCCTTGCACAACCACTACACGC |
| | | | AGAAGTCCATCTCCCGCTCTCCGGGTAA |
| | | | ATGA |
| 116 | 270-12 | VH | ATGGAGACTGGGCTGCGCTGGCTTCTCC |
| | | | TGGTCGCTGTGCTCAAAGGTGTCCAGTG |
| | | | TCAGGCGCAGCTGGAGGAGTCCGGGGGA |
| | | | GACCTGGTCAAGCCTGAGGGATCCCTGA |
| | | | CACTCACCTGCACAGCCTCTGGATTCTC |
| | | | CTTCACTGACAGGCATTACATGTGCTGG |
| | | | GTCCGCCAGGCTCCGGGGAAGGGGCTGG |
| | | | AGTGGATCGCATGCGTTTATCCTGGTAG |
| | | | TAGTGGTAGCACTTACTACGCGAGCTGG |
| | | | GCGAGAGGCCGATTCACCATCTCCAAAA |
| | | | CCTCGTCGACCACGGTGACTCTGCGCAT |
| | | | GACCAGTCTGACAGCCGCGGACACGGCC |
| | | | ACCTATTTCTGTGCGAGATCCTCATATC |
| | | | CTGATAGTAGTGGCTATAGTTATGGCAT |
| | | | GGACCTCTGGGGCCCTGGGACCCTCGTC |
| | | | ACCGTCTCTTCA |
| 117 | 270-12 | Light | ATGGACACGAGGGCCCCCACTCAGCTGC |
| | | Chain | TGGGGCTCCTGTTGCTCTGGCTCCCAGG |
| | | | TGCCAGATGTGCTGACATTGTGATGACC |
| | | | CAGACTCCAGCCTCCGTGGAGGCAGCTG |
| | | | TGGGAGGCACAGTCACCATCAAGTGCCA |
| | | | GGCCAGTCAGGACATTAACAGCAATTTA |
| | | | GCCTGGTATCAGCAGAAACCAGGGCAGC |
| | | | GTCCCAAGTTCCTGATGTATCTGACATC |
| | | | CAAACTGGCATCTGGGGTCCCATCGCGG |
| | | | TTCAGAGGCAGTGGATCTGGGACACAGT |
| | | | TCACTCTCACCATCAGCGACCTGGAGTG |
| | | | TGCCGATGCTGCCACTTACTACTGTCAA |
| | | | ACCTATTATGATATTAGTAATTATGGAT |
| | | | ATGCTTTCGGCGGAGGGACCGAGGTGGT |
| | | | GGTCAAAGGTGATCCAGTTGCACCTACT |
| | | | GTCCTCATCTTCCCACCAGCTGCTGATC |
| | | | AGGTGGCAACTGGAACAGTCACCATCGT |
| | | | GTGTGTGGCGAATAAATACTTTCCCGAT |
| | | | GTCACCGTCACCTGGGAGGTGGATGGCA |
| | | | CCACCCGAACAACTGGCATCGAGAACAG |
| | | | TAAAACACCGCAGAATTCTGCAGATTGT |
| | | | ACCTACAACCTCAGCAGCACTCTGACAC |
| | | | TGACCAGCACACAGTACAACAGCCACAA |
| | | | AGAGTACACCTGCAAGGTGACCCAGGGC |
| | | | ACGACCTCAGTCGTCCAGAGCTTCAATA |
| | | | GGGGTGACTGTTAG |
| 118 | 270-12 | VL | ATGGACACGAGGGCCCCCACTCAGCTGC |
| | | | TGGGGCTCCTGTTGCTCTGGCTCCCAGG |
| | | | TGCCAGATGTGCTGACATTGTGATGACC |
| | | | CAGACTCCAGCCTCCGTGGAGGCAGCTG |
| | | | TGGGAGGCACAGTCACCATCAAGTGCCA |
| | | | GGCCAGTCAGGACATTAACAGCAATTTA |
| | | | GCCTGGTATCAGCAGAAACCAGGGCAGC |
| | | | GTCCCAAGTTCCTGATGTATCTGACATC |
| | | | CAAACTGGCATCTGGGGTCCCATCGCGG |
| | | | TTCAGAGGCAGTGGATCTGGGACACAGT |
| | | | TCACTCTCACCATCAGCGACCTGGAGTG |
| | | | TGCCGATGCTGCCACTTACTACTGTCAA |
| | | | ACCTATTATGATATTAGTAATTATGGAT |
| | | | ATGCTTTCGGCGGAGGGACCGAGGTGGT |
| | | | GGTCAA |
| 119 | 181-4 | Heavy | ATGGAGACTGGGCTGCGCTGGCTTCTCC |
| | or | Chain | TGGTCGCTGTGCTCAAAGGTGTCCAGTG |
| | 329-2 | | TCAGGCGCAGCTGGAGGAGTCCGGGGGA |
| | | | GACCTGGTCAAGCCTGAGGGATCCCTGA |
| | | | CACTCACCTGCACAGCCTCTGGATTCTC |
| | | | CTTCACTGACAGGCATTACATGTGCTGG |
| | | | GTCCGCCAGGCTCCGGGGAAGGGGCTGG |
| | | | AGTGGATCGCATGCGTTTATCCTGGTAG |
| | | | TAGTGGTAGCACTTACTACGCGAGCTGG |
| | | | GCGAGAGGCCGATTCACCATCTCCAAAA |
| | | | CCTCGTCGACCACGGTGACTCTGCGCAT |
| | | | GACCAGTCTGACAGCCGCGGACACGGCC |
| | | | ACCTATTTCTGTGCGAGATCCTCATATC |
| | | | CTGATAGTAGTGGCTATAGTTATGGCAT |
| | | | GGACCTCTGGGGCCCTGGGACCCTCGTC |
| | | | ACCGTCTCTTCAGGGCAACCTAAGGCTC |
| | | | CATCAGTCTTCCCACTGGCCCCCTGCTG |
| | | | CGGGGACACACCCAGCTCCACGGTGACC |
| | | | CTGGGCTGCCTGGTCAAAGGGTACCTCC |
| | | | CGGAGCCAGTGACCGTGACCTGGAACTC |
| | | | GGGCACCCTCACCAATGGGGTACGCACC |
| | | | TTCCCGTCCGTCCGGCAGTCCTCAGGCC |
| | | | TCTACTCGCTGAGCAGCGTGGTGAGCGT |
| | | | GACCTCAAGCAGCCAGCCCGTCACCTGC |
| | | | AACGTGGCCCACCCAGCCACCAACACCA |
| | | | AAGTGGACAAGACCGTTGCGCCCTCGAC |
| | | | ATGCAGCAAGCCCACGTGCCCACCCCCT |
| | | | GAACTCCTGGGGGGACCGTCTGTCTTCA |
| | | | TCTTCCCCCCAAAACCCAAGGACACCCT |
| | | | CATGATCTCACGCACCCCCGAGGTCACA |
| | | | TGCGTGGTGGTGGACGTGAGCCAGGATG |
| | | | ACCCCGAGGTGCAGTTCACATGGTACAT |
| | | | AAACAACGAGCAGGTGCGCACCGCCCGG |
| | | | CCGCCGCTACGGGAGCAGCAGTTCAACA |
| | | | GCACGATCCGCGTGGTCAGCACCCTCCC |
| | | | CATCGCGCACCAGGACTGGCTGAGGGGC |
| | | | AAGGAGTTCAAGTGCAAAGTCCACAACA |
| | | | AGGCACTCCCGGCCCCCATCGAGAAAAC |
| | | | CATCTCCAAAGCCAGAGGGCAGCCCCTG |
| | | | GAGCCGAAGGTCTACACCATGGGCCCTC |
| | | | CCCGGGAGGAGCTGAGCAGCAGGTCGGT |
| | | | CAGCCTGACCTGCATGATCAACGGCTTC |
| | | | TACCCTTCCGACATCTCGGTGGAGTGGG |
| | | | AGAAGAACGGGAAGGCAGAGGACAACTA |
| | | | CAAGACCACGCCGGCCGTGCTGGACAGC |
| | | | GACGGCTCCTACTTCCTCTACAGCAAGC |
| | | | TCTCAGTGCCCACGAGTGAGTGGCAGCG |
| | | | GGGCGACGTCTTCACCTGCTCCGTGATG |
| | | | CACGAGGCCTTGCACAACCACTACACGC |
| | | | AGAAGTCCATCTCCCGCTCTCCGGGTAA |
| | | | ATGA |
| 120 | 181-4 | VH | ATGGAGACTGGGCTGCGCTGGCTTCTCC |
| | or | | TGGTCGCTGTGCTCAAAGGTGTCCAGTG |
| | 329-2 | | TCAGGCGCAGCTGGAGGAGTCCGGGGGA |
| | | | GACCTGGTCAAGCCTGAGGGATCCCTGA |
| | | | CACTCACCTGCACAGCCTCTGGATTCTC |
| | | | CTTCACTGACAGGCATTACATGTGCTGG |
| | | | GTCCGCCAGGCTCCGGGGAAGGGGCTGG |
| | | | AGTGGATCGCATGCGTTTATCCTGGTAG |
| | | | TAGTGGTAGCACTTACTACGCGAGCTGG |
| | | | GCGAGAGGCCGATTCACCATCTCCAAAA |
| | | | CCTCGTCGACCACGGTGACTCTGCGCAT |
| | | | GACCAGTCTGACAGCCGCGGACACGGCC |
| | | | ACCTATTTCTGTGCGAGATCCTCATATC |
| | | | CTGATAGTAGTGGCTATAGTTATGGCAT |
| | | | GGACCTCTGGGGCCCTGGGACCCTCGTC |
| | | | ACCGTCTCTTCA |
| 121 | 181-4 | Light | ATGGACACGAGGGCCCCCACTCAGCTGC |
| | or | Chain | TGGGGCTCCTGTTGCTCTGGCTCCCAGG |
| | 329-2 | | TGCCAGATGTGCTGACATTGTGATGACC |
| | | | CAGACTCCAGCCTCCGTGGAGGCAGCTG |
| | | | TGGGAGGCACAGTCACCATCAAGTGCCA |
| | | | GGCCAGTCAGAACATTAACAGCAATTTA |
| | | | GCCTGGTATCAGCAGAAACCAGGGCAGC |
| | | | GTCCCAAGTTCCTGATGTATCTGACATC |
| | | | CAAACTGGCATCTGGGGTCCCATCGCGG |
| | | | TTCAGAGGCAGTGGATCTGGGACACAGT |
| | | | TCACTCTCACCATCAGCGACCTGGAGTG |
| | | | TGCCGATGCTGCCACTTACTACTGTCAA |
| | | | ACCTATTATGATATTAGTAATTATGGAT |
| | | | ATGCTTTCGGCGGAGGGACCGAGGTGGT |
| | | | GGTCAAAGGTGATCCAGTTGCACCTACT |
| | | | GTCCTCATCTTCCCACCAGCTGCTGATC |
| | | | AGGTGGCAACTGGAACAGTCACCATCGT |
| | | | GTGTGTGGCGAATAAATACTTTCCCGAT |
| | | | GTCACCGTCACCTGGGAGGTGGATGGCA |
| | | | CCACCCAAACAACTGGCATCGAGAACAG |
| | | | TAAAACACCGCAGAATTCTGCAGATTGT |
| | | | ACCTACAACCTCAGCAGCACTCTGACAC |
| | | | TGACCAGCACACAGTACAACAGCCACAA |
| | | | AGAGTACACCTGCAAGGTGACCCAGGGC |
| | | | ACGACCTCAGTCGTCCAGAGCTTCAATA |
| | | | GGGGTGACTGTTAG |
| 122 | 181-4 | VL | ATGGACACGAGGGCCCCCACTCAGCTGC |
| | or | | TGGGGCTCCTGTTGCTCTGGCTCCCAGG |
| | 329-2 | | TGCCAGATGTGCTGACATTGTGATGACC |
| | | | CAGACTCCAGCCTCCGTGGAGGCAGCTG |
| | | | TGGGAGGCACAGTCACCATCAAGTGCCA |
| | | | GGCCAGTCAGAACATTAACAGCAATTTA |
| | | | GCCTGGTATCAGCAGAAACCAGGGCAGC |
| | | | GTCCCAAGTTCCTGATGTATCTGACATC |
| | | | CAAACTGGCATCTGGGGTCCCATCGCGG |
| | | | TTCAGAGGCAGTGGATCTGGGACACAGT |
| | | | TCACTCTCACCATCAGCGACCTGGAGTG |
| | | | TGCCGATGCTGCCACTTACTACTGTCAA |
| | | | ACCTATTATGATATTAGTAATTATGGAT |
| | | | ATGCTTTCGGCGGAGGGACCGAGGTGGT |
| | | | GGTCAAA |
| 123 | 260-3 | Heavy | ATGGAGACTGGGCTGCGCTGGCTTCTCC |
| | | Chain | TGGTCGCTGTGCTCAAAGGTGTCCAGTG |
| | | | TCAGGCGCAGCTGGAGGAGTCCGGGGGA |
| | | | GACCTGGTCAAGCCTGAGGGATCCCTGA |
| | | | CACTCACCTGCACAGCCTCTGGATTCTC |
| | | | CTTCACTGACAGGCATTACATGTGCTGG |
| | | | GTCCGCCAGGCTCCGGGGAAGGGGCTGG |
| | | | AGTGGATCGCATGCGTTTATCCTGGTAG |
| | | | TAGTGGTAGCACTTACTACGCGAGCTGG |
| | | | GCGAGAGGCCGATTCACCATCTCCAAAA |
| | | | CCTCGTCGACCACGGTGACTCTGCGCAT |
| | | | GACCAGTCTGACAGCCGCGGACACGGCC |
| | | | ACCTATTTCTGTGCGAGATCCTCATATC |
| | | | CTGATAGTAGTGGCTATAGTTATGGCAT |
| | | | GGACCTCTGGGGCCCTGGGACCCTCGTC |
| | | | ACCGTCCCTTCAGGGCAACCTAAGGCTC |
| | | | CATCAGTCTTCCCACTGGCCCCCTGCTG |
| | | | CGGGGACACACCCAGCTCCACGGTGACC |
| | | | CTGGGCTGCCTGGTCAAAGGGTACCTCC |
| | | | CGGAGCCAGTGACCGTGACCTGGAACTC |
| | | | GGGCACCCTCACCAATGGGGTACGCACC |
| | | | TTCCCGTCCGTCCGGCAGTCCTCAGGCC |
| | | | TCTACTCGCTGAGCAGCGTGGTGAGCGT |
| | | | GACCTCAAGCAGCCAGCCCGTCACCTGC |
| | | | AACGTGGCCCACCCAGCCACCAACACCA |
| | | | AAGTGGACAAGACCGTTGCGCCCTCGAC |
| | | | ATGCAGCAAGCCCACGTGCCCACCCCCT |
| | | | GAACTCCTGGGGGGACCGTCTGTCTTCA |
| | | | TCTTCCCCCCAAAACCCAAGGACACCCT |
| | | | CATGATCTCACGCACCCCCGAGGTCACA |
| | | | TGCGTGGTGGTGGACGTGAGCCAGGATG |
| | | | ACCCCGAGGTGCAGTTCACATGGTACAT |
| | | | AAACAACGAGCAGGTGCGCACCGCCCGG |
| | | | CCGCCGCTACGGGAGCAGCAGTTCAACA |
| | | | GCACGATCCGCGTGGTCAGCACCCTCCC |
| | | | CATCGCGCACCAGGACTGGCTGAGGGGC |
| | | | AAGGAGTTCAAGTGCAAAGTCCACAACA |
| | | | AGGCACTCCCGGCCCCCATCGAGAAAAC |
| | | | CATCTCCAAAGCCAGAGGGCAGCCCCTG |
| | | | GAGCCGAAGGTCTACACCATGGGCCCTC |
| | | | CCCGGGAGGAGCTGAGCAGCAGGTCGGT |
| | | | CAGCCTGACCTGCATGATCAACGGCTTC |
| | | | TACCCTTCCGACATCTCGGTGGAGTGGG |
| | | | AGAAGAACGGGAAGGCAGAGGACAACTA |
| | | | CAAGACCACGCCGGCCGTGCTGGACAGC |
| | | | GACGGCTCCTACTTCCTCTACAGCAAGC |
| | | | TCTCAGTGCCCACGAGTGAGTGGCAGCG |
| | | | GGGCGACGTCTTCACCTGCTCCGTGATG |
| | | | CACGAGGCCTTGCACAACCACTACACGC |
| | | | AGAAGTCCATCTCCCGCTCTCCGGGTAA |
| | | | ATGA |
| 124 | 260-3 | VH | ATGGAGACTGGGCTGCGCTGGCTTCTCC |
| | | | TGGTCGCTGTGCTCAAAGGTGTCCAGTG |
| | | | TCAGGCGCAGCTGGAGGAGTCCGGGGGA |
| | | | GACCTGGTCAAGCCTGAGGGATCCCTGA |
| | | | CACTCACCTGCACAGCCTCTGGATTCTC |
| | | | CTTCACTGACAGGCATTACATGTGCTGG |
| | | | GTCCGCCAGGCTCCGGGGAAGGGGCTGG |
| | | | AGTGGATCGCATGCGTTTATCCTGGTAG |
| | | | TAGTGGTAGCACTTACTACGCGAGCTGG |
| | | | GCGAGAGGCCGATTCACCATCTCCAAAA |
| | | | CCTCGTCGACCACGGTGACTCTGCGCAT |
| | | | GACCAGTCTGACAGCCGCGGACACGGCC |
| | | | ACCTATTTCTGTGCGAGATCCTCATATC |
| | | | CTGATAGTAGTGGCTATAGTTATGGCAT |
| | | | GGACCTCTGGGGCCCTGGGACCCTCGTC |
| | | | ACCGTCCCTTCA |
| 125 | 260-3 | Light | ATGGACACGAGGGCCCCCACTCAGCTGC |
| | | Chain | TGGGGCTCCTGTTGCTCTGGCTCCCAGG |
| | | | TGCCAGATGTGCTGACATTGTGATGACC |
| | | | CAGACTCCAGCCTCCGTGGAGGCAGCTG |
| | | | TGGGAGGCACAGTCACCATCAAGTGCCA |
| | | | GGCCAGTCAGAACATTAACAGCAATTTA |
| | | | GCCTGGTATCAGCAGAAACCAGGGCAGC |
| | | | GTCCCAAGTTCCTGATGTATCTGACATC |
| | | | CAAACTGGCATCTGGGGTCCCATCGCGG |
| | | | TTCAGAGGCAGTGGATCTGGGACACAGT |
| | | | TCACTCTCACCATCAGCGACCTGGAGTG |
| | | | TGCCGATGCTGCCACTTACTACTGTCAA |
| | | | ACCTATTATGATATTAGTAATTATGGAT |
| | | | ATGCTTTCGGCGGAGGGACCGAGGTGGT |
| | | | GGTCAAAGGTGATCCAGTTGCACCTACT |
| | | | GTCCTCATCTTCCCACCAGCTGCTGATC |
| | | | AGGTGGCAACTGGAACAGTCACCATCGT |
| | | | GTGTGTGGCGAATAAATACTTTCCCGAT |
| | | | GTCACCGTCACCTGGGAGGTGGATGGCA |
| | | | CCACCCAAACAACTGGCATCGAGAACAG |
| | | | TAAAACACCGCAGAATTCTGCAGATTGT |
| | | | ACCTACAACCTCAGCAGCACTCTGACAC |
| | | | TGACCAGCACACAGTACAACAGCCACAA |
| | | | AGAGTACACCTGCAAGGTGACCCAGGGC |
| | | | ACGACCTCAGTCGTCCAGAGCTTCAATA |
| | | | GGGGTGACTGTTAG |
| 126 | 260-3 | VL | ATGGACACGAGGGCCCCCACTCAGCTGC |
| | | | TGGGGCTCCTGTTGCTCTGGCTCCCAGG |
| | | | TGCCAGATGTGCTGACATTGTGATGACC |
| | | | CAGACTCCAGCCTCCGTGGAGGCAGCTG |
| | | | TGGGAGGCACAGTCACCATCAAGTGCCA |
| | | | GGCCAGTCAGAACATTAACAGCAATTTA |
| | | | GCCTGGTATCAGCAGAAACCAGGGCAGC |
| | | | GTCCCAAGTTCCTGATGTATCTGACATC |
| | | | CAAACTGGCATCTGGGGTCCCATCGCGG |
| | | | TTCAGAGGCAGTGGATCTGGGACACAGT |
| | | | TCACTCTCACCATCAGCGACCTGGAGTG |
| | | | TGCCGATGCTGCCACTTACTACTGTCAA |
| | | | ACCTATTATGATATTAGTAATTATGGAT |
| | | | ATGCTTTCGGCGGAGGGACCGAGGTGGT |
| | | | GGTCAAA |
| 138 | 278-11 | Heavy | ATGGAGACTGGGCTGCGCTGGCTTCTCC |
| | | Chain | TGGTCGCTGTGCTCAAAGGTGTCCAGTG |
| | | | TCAGTCGTTGGAGGAGTCCGGGGGAGGC |
| | | | CTGGTCCAGCCTGAGGGATCCCTGACAC |
| | | | TCGCCTGCACAGCTTTGAATTCGACTCC |
| | | | AGTAGCAATGCAATGTGCTGGGTCCGCC |
| | | | AGGCTCCAGGGAAGGGACTGGAGTGGAT |
| | | | CGCACGCATCTATAGTGGTAGTGGTACT |
| | | | ATTTACTACGCTAACTGGGCGAGAGGCC |
| | | | GATCACCATCTCCAAGACCTCGTCGACC |
| | | | ACGGTGACTCTGCAAATGACCAGTCTGA |
| | | | CAGACGCGGACACGGCCACCTATTTCTG |
| | | | TGCGAGATACAATACTGGTGGGTTCTAT |
| | | | TATGACTTGTGGGGCCCGGCACCCTGGT |
| | | | CACCGTCTCCTCAGGGCAACCTAAGGCT |
| | | | CCATCAGTCTTCCCACTGGCCCCCTGCT |
| | | | GCGGGGACACACCCAGCTCCACGGTGAC |
| | | | CCTGGGCTGCCTGGTCAAAGGGTACCTC |
| | | | CCGGAGCCAGTGACCGTGACCTGGAACT |
| | | | GGGCACCCTCACCAATGGGGTACGCACC |
| | | | TTCCCGTCCGTCCGGCAGTCCTCAGGCC |
| | | | TCTACTCGCTGAGCAGCGTGGTGAGCGT |
| | | | GACCTCAAGCAGCCAGCCCGTCACCTGC |
| | | | AACGTGGCCCACCCACCACCAACACCAA |
| | | | AGTGGACAAGACCGTTGCGCCCTCGACA |
| | | | TGCAGCAAGCCCACGTGCCCACCCCCTG |
| | | | AACTCCTGGGGGGACCGTCTGTCTTCAT |
| | | | CTTCCCCCCAAAACCCAAGGACACCCTC |
| | | | ATATCTCACGCACCCCCGAGGTCACATG |
| | | | CGTGGTGGTGGACGTGAGCCAGGATGAC |
| | | | CCCGAGGTGCAGTTCACATGGTACATAA |
| | | | ACAACGAGCAGGTGCGCACCGCCCGGCC |
| | | | GCCGCTACGGGAGCAGCGTTCAACAGCA |
| | | | CGATCCGCGTGGTCAGCACCCTCCCCAT |
| | | | CGCGCACCAGGACTGGCTGAGGGGCAAG |
| | | | GAGTTCAAGTGCAAAGTCCACAACAAGG |
| | | | CACTCCCGGCCCCCATCGAGAAAACCAT |
| | | | CTCCAAGCCAGAGGGCAGCCCCTGGAGC |
| | | | CGAAGGTCTACACCATGGGCCCTCCCCG |
| | | | GGAGGAGCTGAGCAGCAGGTCGGTCAGC |
| | | | CTGACCTGCATGATCAACGGCTTCTACC |
| | | | CTTCCGACATCTCGGTGGATGGGAGAAG |
| | | | AACGGGAAGGCAGAGGACAACTACAAGA |
| | | | CCACGCCGGCCGTGCTGGACAGCGACGG |
| | | | CTCCTACTTCCTCTACAGCAAGCTCTCA |
| | | | GTGCCCACGAGTGAGTGGCAGCGGGGCG |
| | | | ACGTCTCACCTGCTCCGTGATGCACGAG |
| | | | GCCTTGCACAACCACTACACGCAGAAGT |
| | | | CCATCTCCCGCTCTCCGGGTAAATGA |
| 139 | 278-11 | VH | ATGGAGACTGGGCTGCGCTGGCTTCTCC |
| | | | TGGTCGCTGTGCTCAAAGGTGTCCAGTG |
| | | | TCAGTCGTTGGAGGAGTCCGGGGGAGGC |
| | | | CTGGTCCAGCCTGAGGGATCCCTGACAC |
| | | | TCGCCTGCACAGCTTTGAATTCGACTCC |
| | | | AGTAGCAATGCAATGTGCTGGGTCCGCC |
| | | | AGGCTCCAGGGAAGGGACTGGAGTGGAT |
| | | | CGCACGCATCTATAGTGGTAGTGGTACT |
| | | | ATTTACTACGCTAACTGGGCGAGAGGCC |
| | | | GATCACCATCTCCAAGACCTCGTCGACC |
| | | | ACGGTGACTCTGCAAATGACCAGTCTGA |
| | | | CAGACGCGGACACGGCCACCTATTTCTG |
| | | | TGCGAGATACAATACTGGTGGGTTCTAT |
| | | | TATGACTTGTGGGGCCCGGCACCCTGGT |
| | | | CACCGTCTCCTCA |
| 140 | 278-11 | Light | ATGGACACGAGGGCCCCCACTCAGCTGC |
| | | Chain | TGGGGCTCCTGCTGCTCTGGCTCCCAGG |
| | | | TGCCAGATGTGCCCTTGTGATGACCCAG |
| | | | ACTCCAGCCTCCGTGGAGGCAGCTGTGG |
| | | | GAGGCACAGTCACCACAATTGCCAGGCC |
| | | | AGTCAGAGAATTGGTACTAATTTAGCCT |
| | | | GGTATCAGCAGAAACCAGGGCAGCCTCC |
| | | | CAAGGTCCTGATCTACAAGGCATCCACT |
| | | | CTGGCATCTGGGGTCTCATCGCGGTTCA |
| | | | TAGCAGTGGATCTGGGACAGACTATACT |
| | | | CTCACCATCAGCGGCGTGGAGTGTGACG |
| | | | ATGCTGCCACTTACTACTGTCAACAGGG |
| | | | TTATAGTAGTAATGATGCCGATAATACT |
| | | | TTCGGCGGAGGGACCGAGTGGTGGTCAA |
| | | | AGGTGATCCAGTTGCACCTACTGTCCTC |
| | | | ATCTTCCCACCAGCTGCTGATCAGGTGG |
| | | | CAACTGGAACAGTCACCATCGTGTGTGT |
| | | | GGCGAATAAATACTTTCCCGATGTCACC |
| | | | GTCACCTGGGAGGTGGTGGCACCACCCA |
| | | | AACAACTGGCATCGAGAACAGTAAAACA |
| | | | CCGCAGAATTCTGCAGATTGTACCTACA |
| | | | ACCTCAGCAGCACTCTGACACTGACCAG |
| | | | CACACAGTACAACAGCCACAAAGAGTAC |
| | | | ACCGCAAGGTGACCCAGGGCACGACCTC |
| | | | AGTCGTCCAGAGCTTCAATAGGGGTGAC |
| | | | TGTTAG |
| 141 | 278-11 | VL | ATGGACACGAGGGCCCCCACTCAGCTGC |
| | | | TGGGGCTCCTGCTGCTCTGGCTCCCAGG |
| | | | TGCCAGATGTGCCCTTGTGATGACCCAG |
| | | | ACTCCAGCCTCCGTGGAGGCAGCTGTGG |
| | | | GAGGCACAGTCACCACAATTGCCAGGCC |
| | | | AGTCAGAGAATTGGTACTAATTTAGCCT |
| | | | GGTATCAGCAGAAACCAGGGCAGCCTCC |
| | | | CAAGGTCCTGATCTACAAGGCATCCACT |
| | | | CTGGCATCTGGGGTCTCATCGCGGTTCA |
| | | | TAGCAGTGGATCTGGGACAGACTATACT |
| | | | CTCACCATCAGCGGCGTGGAGTGTGACG |
| | | | ATGCTGCCACTTACTACTGTCAACAGGG |
| | | | TTATAGTAGTAATGATGCCGATAATACT |
| | | | TTCGGCGGAGGGACCGAGTGGTGGTCAA |
| | | | A |

Clones 181-4 and 329-2 have the same protein and nucleic acid sequences.
The underlined segment of each nucleic acid sequence in Table 7 is a leader sequence that encodes a signal peptide.

### Example 3: Characterization of Anti-ZiKV Antibody Clones 102-1, 242-3, 289-3, 306-2, 11-3, 78-2, 270-12, 181-4, 260-3 and 278-11

### Screening of Hybridoma Supernatant for Cross-Reactivity to Dengue:

To determine if antibodies present in the hybridoma supernatant are specific to ZIKV all multiclones and/or subclones were screened by indirect ELISA against inactivated dengue Virus 1 (DENV1) [West Pacific 74 (Microbix)]; Dengue Virus 2 (DENV2) [16681 (Microbix)]; Dengue Virus 3 (DENV3) [CH53489 (Microbix)]; and Dengue Virus 4 (DENV4) [TVP-360 (Microbix)]. DENV-1, 3 and 4 were inactivated with gamma irradiation and DENV2 was inactivated with formalin by the manufacturer as a part of their production process. Both ZIKV-VLP (Native Antigen) and recombinant ZIKV E-protein (Native Antigen) were used as positive control antigens.

In brief, antigens were coated on Nunc Polysorp ELISA plates at a 1 μg/mL concentration in carbonate coating buffer (pH 9.4) at 4° C. overnight prior to use. Following removal from storage, plates were washed with phosphate buffered saline plus 0.05% Tween 20 (PBST). A 5% non-fat dry milk blocking solution was added to the plates for a minimum of 1 hour at room temperature to reduce non-specific binding. Plates were washed and undiluted hybridoma supernatants were added to the plate following a predetermined layout. Plates were then incubated at 37° C. for 1-2 hours. Commercial rabbit anti-ZIKV E protein pAb (IBT) and in-house derived rabbit anti-dengue pAb were used as positive controls. In-house negative pooled rabbit serum and PBS-only were used as negative controls. These controls were performed concurrently on the same plate. Plates were again washed with PBST. Goat derived IgG (H+L) anti-rabbit horseradish peroxidase (HRP) conjugated secondary antibody (Jackson ImmunoResearch, lot Lot #L2416-X326F) was diluted 1:5,000 in 5% milk block and added to the plate. Plates were incubated at 37° C. for 1.5 hour and then washed again as indicated. 3,3′,5,5′-Tetramethylbenzidine (TMB) substrate was added and incubated for 10 minutes at room temperature. The reaction was stopped with 1N hydrochloric acid and the plates were scanned for absorbance at 450 nm and 630 nm using the Perkin Elmer EnSpire. Positive binding cutoff score was set at 0.5 A OD reading.

At first, all 311 clones were tested against DENV-2. More than 100 clones were cross-reactive to DENV-2 while the rest did not show any cross-reactivity. Finally, a subset of clones were tested against all dengue serotypes and a ZIKV E protein and a ZIKV-VLP (Table 8). Abcam data was used as a reference.

TABLE 8
Cross-reactivity examination of selected clones to all dengue serotypes. Control
clones were selected based from the DENV-2 screening.

| | | | | | | | ZIKV-VLP |
|---|---|---|---|---|---|---|---|
| Clone ID | | DEN-1 | DEN-2 | DEN-3 | DEN-4 | ZIKV_E_Abcam | Abcam |
| Test | 102-1 | 0.031 | 0.032 | 0.035 | 0.031 | 0.2 | 0 9 |
| | 242-3 | 0.04 | 0.034 | 0.04 | 0.038 | 0.33 | 0.97 |
| | 289-3 | 0.031 | 0.028 | 0.037 | 0.032 | 0.07 | 0.94 |
| | 306-2 | 0.037 | 0.034 | 0.034 | 0.048 | 0.61 | 1.15 |
| | 11-3 | 0.01 | 0.01 | 0.04 | 0.01 | 0.01 | 2.83 |
| | 78-2 | 0.91 | 1.62 | 1.00 | 1.55 | 1.13 | 2.67 |
| | 270-12 | 0.034 | 0.032 | 0.034 | 0.031 | 0.39 | 1.27 |
| | 278-11 | 0.031 | 0.03 | 0.033 | 0.035 | 0.06 | 1.19 |
| Control | 3 | 1.06 | 3.40 | 1.19 | 1.41 | 0.38 | 1.03 |
| | 6 | 1.27 | 2.53 | 1.29 | 1.46 | 0.85 | 1.19 |
| | 28 | 0.45 | 0.07 | 0.02 | 0.28 | 0.07 | 1.04 |

Hybridoma clones 11-3, 270-12, 278-11, 102-1, 242-3, 289-3 and 306-2 were highly specific to a ZIKV-VLP and did not cross react to Dengue serotypes.

### Screening of Hybridoma Supernatant for Neutralizing Activity:

The TCID50-based Micro-neutralization titers (MNT) assay was used to examine a virus neutralizing antibody titer of a hybridoma supernatant in 96-well plates. TCID50 represents tissue culture infectious dose 50%. In brief, serially diluted hybridoma supernatant was mixed with 100 TCID50/well of PRVABC59 Zika live viruses, and then added to monolayer of Vero cells and plates were observed after 5 days post-infection for presence or absence of cytopathic effect (CPE) for endpoint titer.

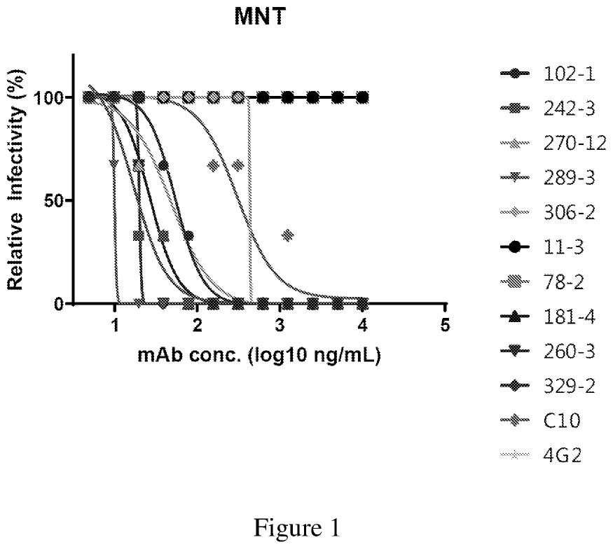

For neutralization, a subset of clones were subjected to MNT using a TCID50 based assay and PRVABC-59 ZIKV. Three clones, i.e., 102-1, 242-3, and 289-3, showed strong neutralization activities (see Table 9 and FIG. 1). Additional three clones, i.e., 270-12, 181-4/329-2, and 260-3, also showed strong neutralization activities (see FIG. 1).

TABLE 9
MNT of selected clones

| | MNT Titer (fold | |
|---|---|---|
| | of dilution) of | MNT IC₅₀ (ng/mL) of |
| Clone ID | Supernatant | Purified antibody |
| 102-1 | >64 | 54.44 |
| 242-3 | 256 | 17.04 |
| 289-3 | 16 | 9.96 |
| 306-2 | <2 | 433.10 |
| 11-3 | <2 | ND |
| 78-2 | <2 | ND |
| 270-12 | 181 | 48.30 |
| 181-4 | | 26.70 |
| 260-3 | | 19.93 |
| C10 | | 305.80 |
| 4G2 | | ND |

In summary, clones 102-1, 242-3, 289-3, 270-12, 181-4/329-2, and 260-2 showed strong neutralizing activities. Clones 306-2 and C10 showed lowest neutralizing activity (433.10 ng/mL and 305.80 ng/mL, respectively). Clones 11-3, 78-2, and 4G2 showed no neutralizing activity at starting 10,000 ng/mL.

### Screening of Hybridoma Supernatants for Antigen Binding:

Supernatants were tested by indirect ELISA for binding to a ZIKV-VLP and a ZIKV E protein. All clones showed good binding to a ZIKV-VLP (Table 10).

TABLE 10
Supernatants from Subclones for binding experiment.

| | ng/mL | VLP (OD) | E (OD) | |
|---|---|---|---|---|
| Clone ID | IgG | binding | binding | MNT titer |
| 102-1 | 1600.00 | 0.90 | 0.20 | ≥64 |
| 242-3 | 4160.00 | 0.97 | 0.33 | 256 |
| 289-3 | 880.00 | 0.94 | 0.07 | 16 |
| 306-2 | 2320.00 | 1.15 | 0.61 | <2 |
| 278-11 | 2800 | 1.19 | 0.06 | <2 |

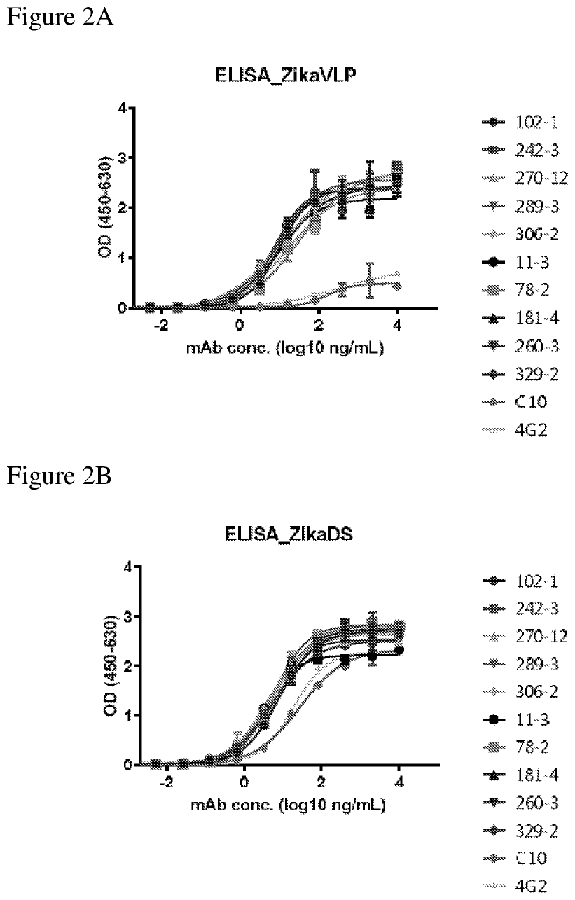

Clones 102-1, 242-3, 289-3, 306-2, 11-3, 78-2, 270-12, 181-4/329-2, and 260-3 were compared with commercially-available antibodies 4G2 and C10 (commercial anti-dengue antibodies) with the respect to the bindings to a ZIKV-VLP and a PIZV. See Table 11 and/or FIG. 2.

TABLE 11
Comparison of inhouse Mabs with 4G2 and C10 at Normalized
Concentration of purified antibodies by Indirect ELISA

| | ZIKV-VLP (IC₅₀) | PIZV (IC₅₀) |
|---|---|---|
| Clone ID | (ng/ml) | (ng/ml) |
| 102-1 | 9.98 | 7.98 |
| 242-3 | 9.47 | 6.71 |
| 289-3 | 7.24 | 5.67 |
| 306-2 | 16.81 | 4.56 |
| 11-3 | 6.57 | 2.95 |
| 78-2 | 31.89 | 7.78 |
| 270-12 | 12.76 | 4.69 |
| 181-4 | 11.41 | 5.45 |
| 260-3 | 7.93 | 5.42 |
| C10 | 162.2 | 26.83 |
| 4G2 | 329.8 | 18.22 |

10 μg/ml of each purified antibody was serially diluted and used for indirect ELISA with either ZIKV-VLP or PIZV as antigen. Absorbacnces were recorded and plotted against dilution to generate a sigmoidal curve. The sigmoidal curve was used to calculate IC₅₀ values as described (K. Stettler et al., Science 10.1126/science.aaf8505 (2016).

The binding activities of clones 102-1, 242-3, 289-3, 306-2, 11-3 and 78-2 were greater than commercially-available antibodies 4G2 and C10. These six clones had higher affinity for the PIZV than for a ZIKV-VLP.

### Screening of Hybridoma Clones for Affinity:

The dissociation rate constant (k_{off}) of a purified ZIKV-VLP (Native Antigen) with hybridoma supernatant were monitored by Bio-layer interferometry (BLI) using an Octet-96 device (Pall ForteBio). Briefly, hybridoma supernatants diluted to five times with running buffer [(0.1% Bovine Serum Albumin (BSA), PBS 0.05% Tween 20 (PBS-T)) and rabbit IgG were captured by Protein A biosensor (Pall ForteBio). The biosensors were transferred to 3.3 μg/mL of a ZIKV-VLP solution for association (10 minutes) then to running buffer for dissociation. k_{off} of each mAbs were calculated by 1:1 binding models using Octet analysis software (version 9.0 ForteBio).

To estimate the affinity of the mAbs, dissociation values (k_{off}) following binding with a ZIKV-VLP were measured. To perform this assay IgG concentration was not adjusted due to lower concentration of IgG in most of the hybridoma samples. In order to succeed in the k_{off} ranking, antibodies present in the hybridoma supernatant should saturate the protein A biosensor to get better signals. Moreover, k_{off} is not dependent on antibody concentration unlike the KD values (M). In this assay concentration of a ZIKV-VLP was kept constant. Interestingly, 190 clones of 260 clones showed lower k_{off}, less than 1×10e⁻⁷ s⁻¹. Clone nos. 102-1, 242-3, 306-2, 11-3 and 78-2 were among the 190 clones.

TABLE 12
Dissociation values (k_{off}) of selected clones

| Clone ID | K_{off} by Octet |
|---|---|
| 102-1 | <1.0E−07 |
| 242-3 | <1.0E−07 |
| 289-3 | 1.13E−04 |
| 306-2 | <1.0E−07 |
| 11-3 | <1.0E−07 |
| 78-2 | <1.0E−07 |
| 181-4 | <1.0E−07 |
| 260-3 | <1.0E−07 |

Screening of Hybridoma Clones for Epitope Binning:

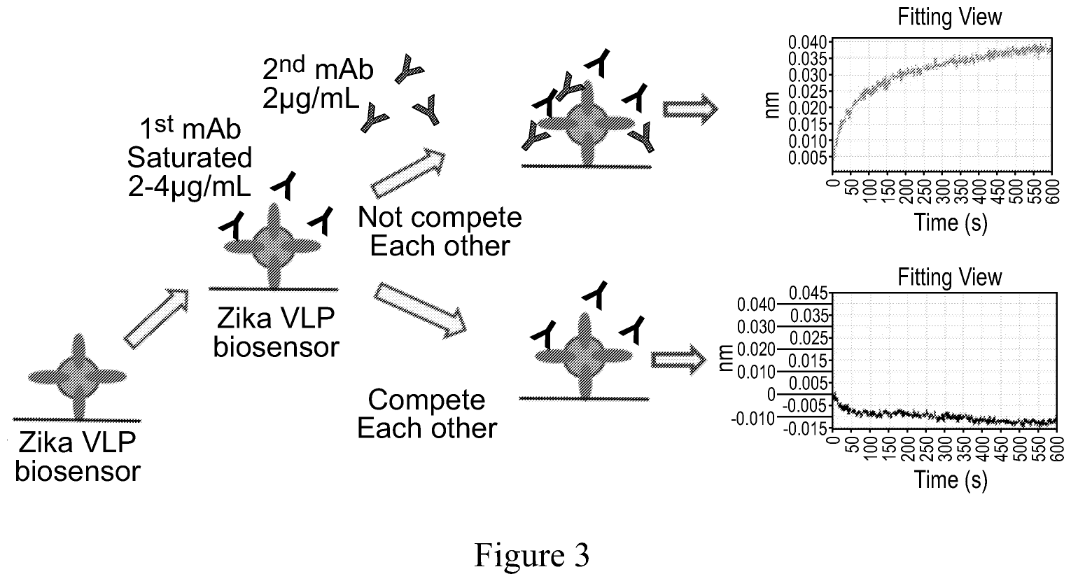

BLI technology allows to characterize and sort antibodies library into bins that bind distinct epitopes on specific antigen. In this experiment, competition assay was applied to sort the mAbs to epitope bins (FIG. 3). Briefly, ZIKV-VLPs were coated to Aminopropylsilane (APS) biosensors and blocked with 1% BSA in PBS. For each binning run, a selected primary antibody from Table 12 was bound to saturate ZIKV-VLP binding sites for 10 minutes (horizontal arrow) and then crossed bound to secondary antibodies for 10 minutes (vertical arrow). The response signal readout of secondary antibodies to each primary antibody was clustered by hierarchy clustering (Ward Method) using SAS JMP 13.1.0 (SAS, Cay, N.C., USA).

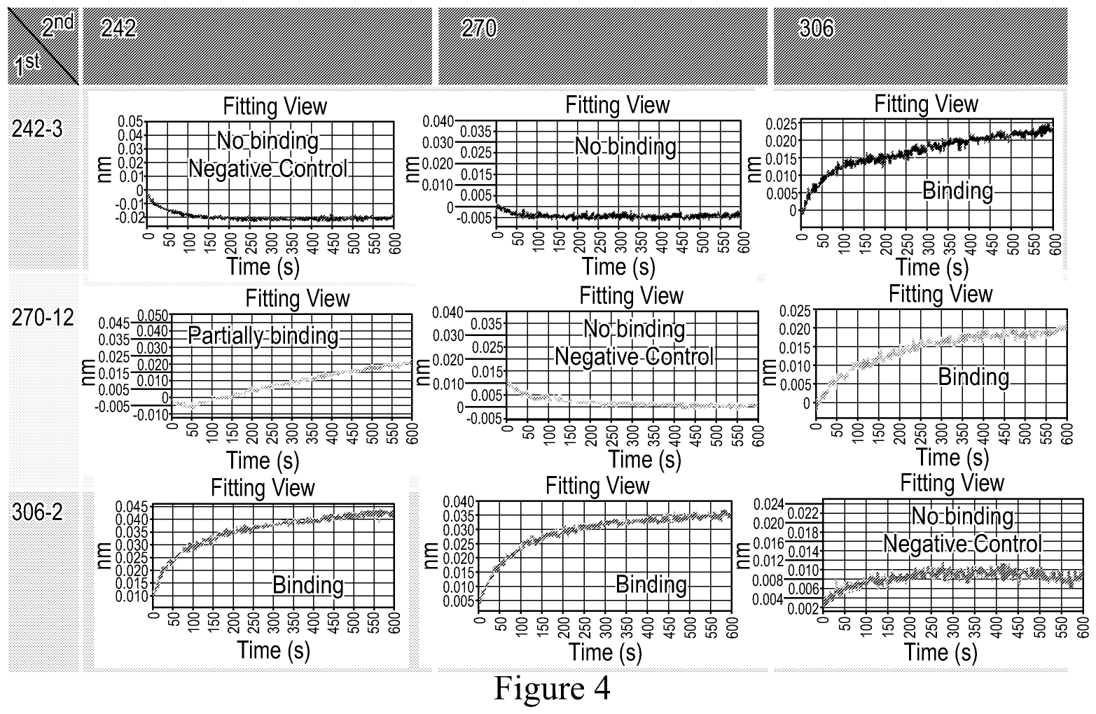

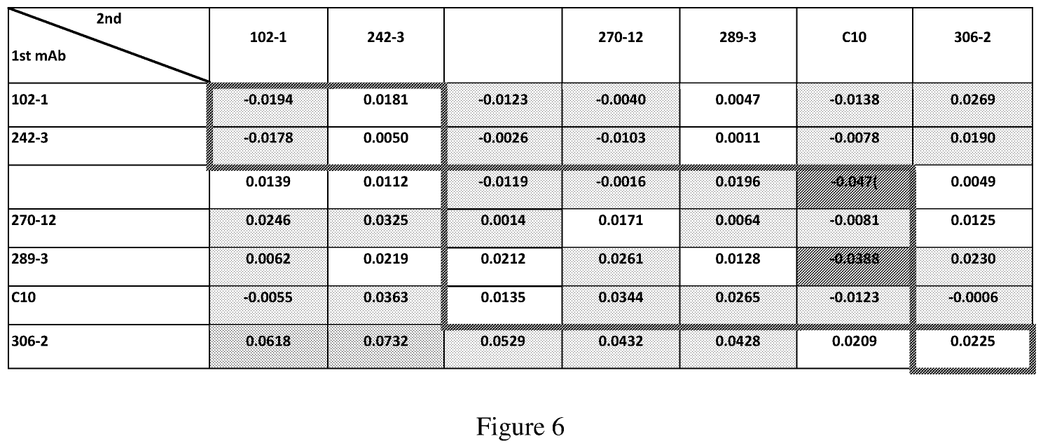

To examine the clones for sandwich ELISA pair, competition assay was performed using Octet (FIGS. 4 and 6). Clones 242-3 and 270-12 did not compete with clone 306-2.

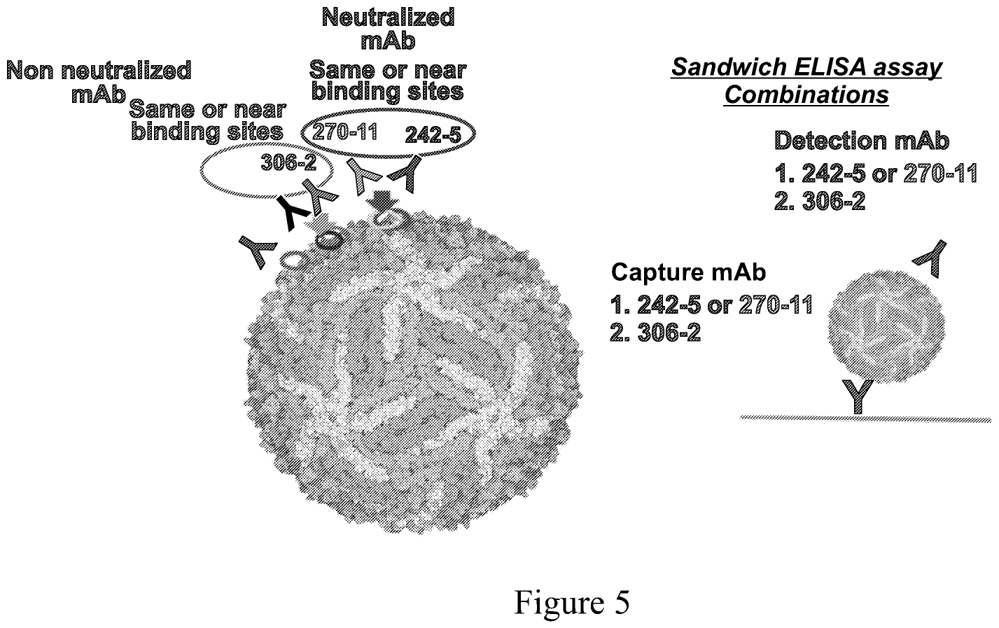

Based on the above observation, it was decided that clone 242-3 or 270-12 could be combined with 306-2 to be used for capture and detection (FIG. 5).

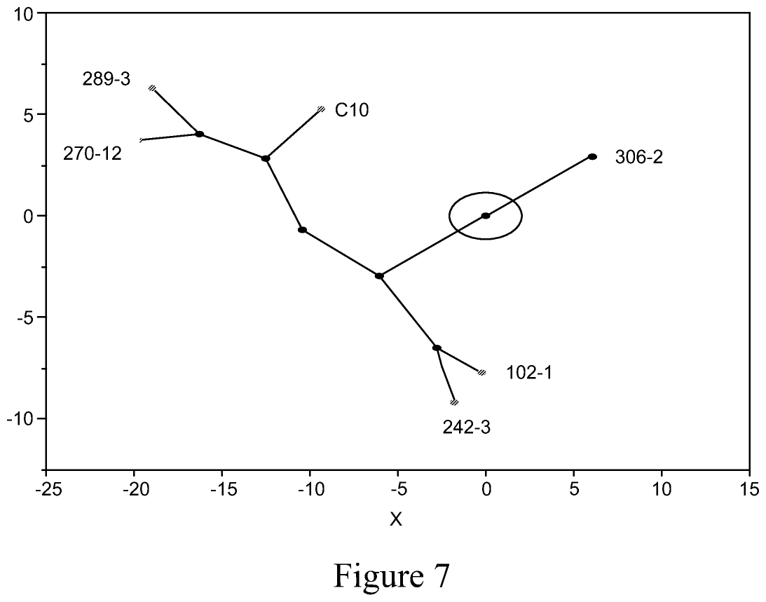

Five hybridoma clones, 102-1, 242-3, 270-12, 289-3, 306-2 and one commercial anti-dengue antibody C10 were selected for epitope binning (FIGS. 6 and 7). One cluster including 102-1/242-3 and another cluster including 270-12 and 289-3 were observed (enclosed by a broaden line box, FIG. 6). 306-2 was highly diverse from other antibodies as seen on the constellation plot (FIG. 7).

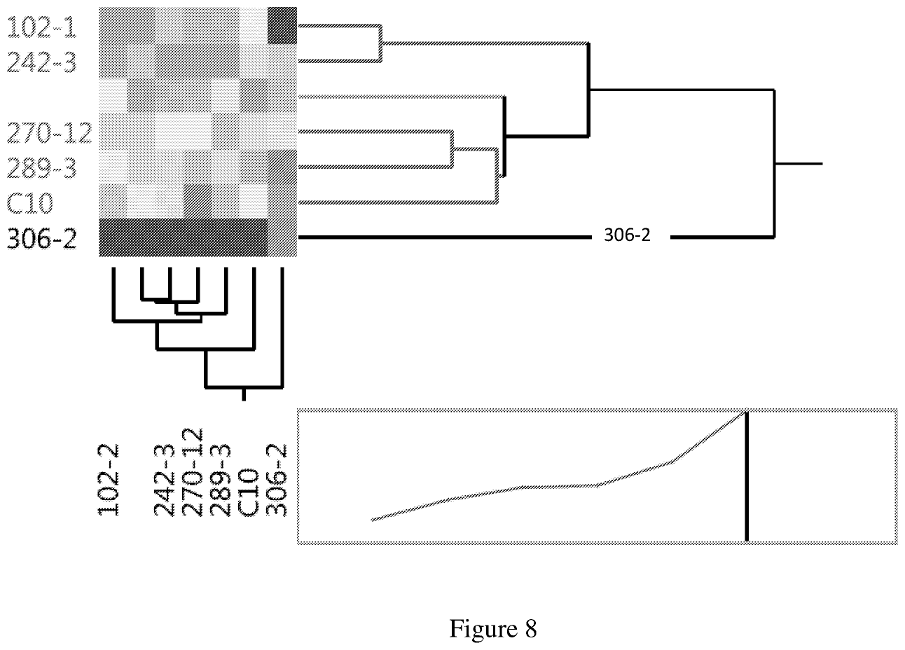

Two clear bins can be observed between low-neutralizing clone, 306-2, and high-neutralizing clones (FIG. 8). There were subtle differences between neutralizing clones and C10. From the clustering analysis by JMP, two sub-bins 102-1/242-3 and 289-3/C10 were derived. These epitope bins were correlated with its neutralization activities.

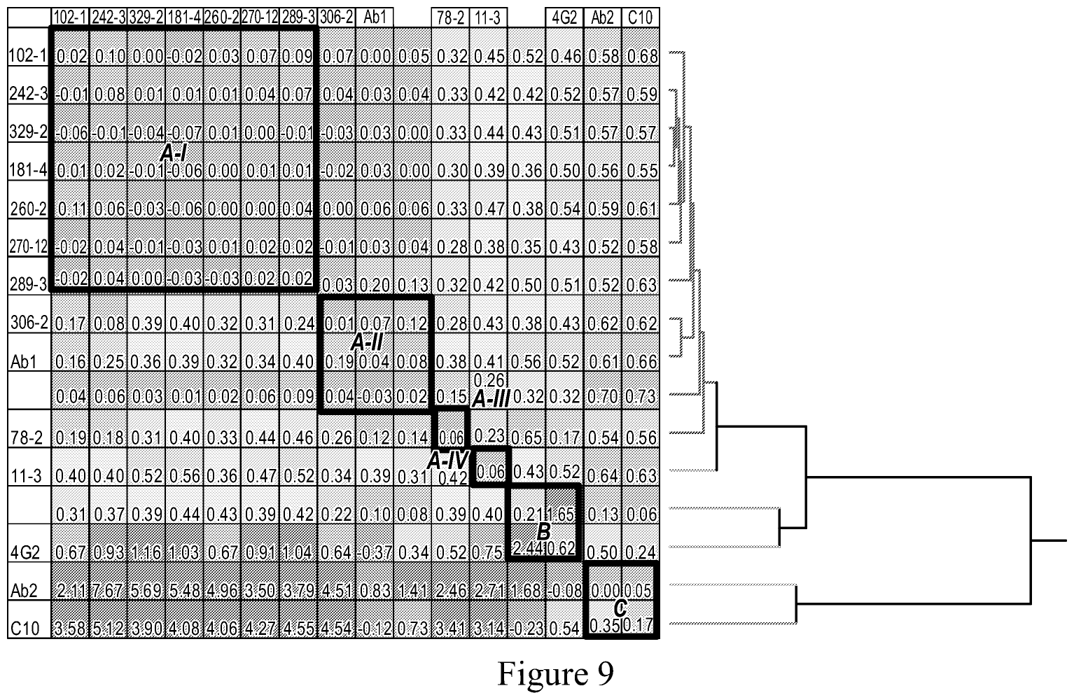

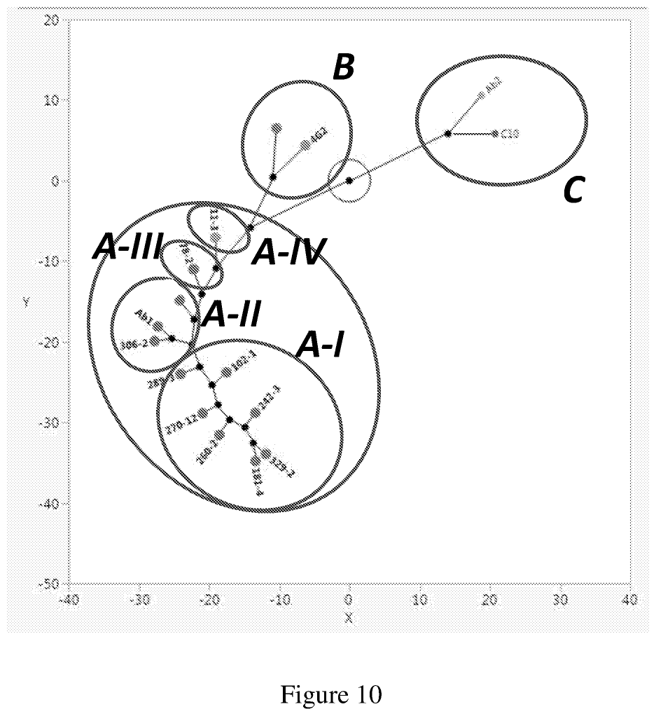

In addition, 102-1, 181-4/329-2, 242-3, 260-3, 270-12, 289-3, 306-2, 11-3 and 78-2 and two commercial anti-dengue antibodies C10 and 4G2 were selected for epitope binning (FIGS. 9 and 10). One cluster including 102-1/181-4/329-2/242-3/260-3/270-12/289-3 were observed (enclosed by a broaden line box, FIGS. 9). 306-2, 11-3, and 78-3 each were highly diverse from one another and from the other neutralizing antibodies as seen on the constellation plot (FIG. 10).

### Screening of Hybridoma Clones by Reporter Virus Particle (RVP) Assay

Selected top hybridoma clones were tested for their neutralizing activity by Zika Reporter Virus Particles (RVP) assay. In this assay, the Zika RVPs were used in place of live

Zika viruses to determine neutralizing antibody titers. The Zika RVPs retain the antigenic determinants of wild type virions including capsid, envelope, pre-membrane and membrane proteins CprME (Integral Molecular, Philadelphia, PA). The Zika RVPs express a Renilla luciferase reporter gene upon infection of permissive cells. The half maximal effective concentration EC₅₀ titer of antibodies was determined using a bioluminescent reaction which generates a glow-type luminescent signal by the interaction of the Renilla luciferase and coelenterazine substrate. The luminescent signal was measured using a luminescence enabled plate reader. Reduction in luminescent signal in the presence of serum indicates neutralization.

The selected four clones 102-1, 242-3, 289-3 and 306-2 were subjected to RVP assay. The predetermined LLOD is Log₁₀EC₅₀ 2.06. The neutralizing clones as determined by the MNT assay showed good EC₅₀ values by RVP assay. The low-neutralizing clone (Clone 306-2) did not show any EC₅₀ value by RVP assay as expected from MNT data. Positive and negative control performed as expected (See Table 14). Clones 102-1, 242-3 and 289-3 showed neutralizing activity above the negative control, whereas clone 306-2 did not show any neutralizing activity.

TABLE 14
Comparison of neutralization activity of selected Mabs to Rabbit Serum by RVP assay

| | Log₁₀ (Fold dilution | Fold dilution for | |
|---|---|---|---|
| | for 50% inhibition of | 50% inhibition of | Neutralizing |
| Clone ID | viral infectivity) | viral infectivity | ability |
| Positive Control | 3.54 | 3467.37 | Positive |
| (rabbit serum) | | | |
| Negative Control | 1.24 | 17.38 | Negative |
| (rabbit serum) | | | |
| 102-1 | 3.14 | 1380.38 | Positive |
| 242-3 | 3.24 | 1737.80 | Positive |
| 289-3 | 3.68 | 4786.30 | Positive |
| 306-2 | 1.89 | 77.62 | Negative or low |

Both MNT and RVP assays confirmed the neutralizing potential of the monoclonal antibodies.

Screening of Hybridoma Clones for Protein Specific Binding:

ProteinSimple Wes (San Jose, Calif.) is a capillary-based technology that allows the detection of specific protein from process sample using antibody of interest. To determine the binding specificity of the hybridoma supernatants to a ZIKV-VLP, selected supernatants were subjected to Wes analysis using manufacturer's instructions for the Wes system.

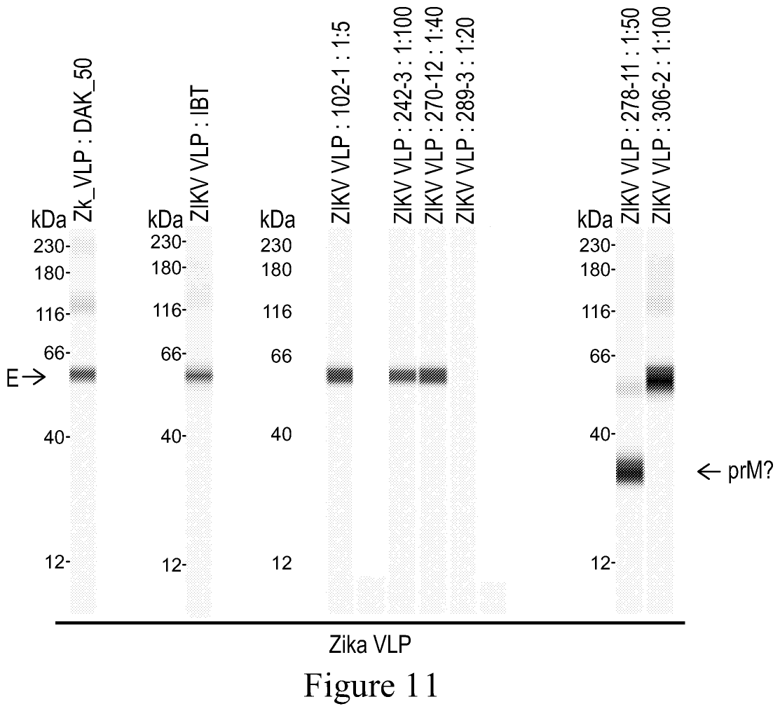

All subclones bind to the ZIKV-VLP, except clone 289-3 (FIG. 11). Additional Wes conditions (reducing and denaturing, nonreducing and denaturing) and titration of the ZIKV-VLP and antibody were run on subclone 289-3 to the ZIKV-VLP (data not shown); still, none of those additional runs show any binding to the ZIKV-VLP. However, subclone 289-3 by indirect EL1SA shows binding to the ZIKV-VLP as shown in Table 10, but not to recombinant E-protein. This could suggest that subclone 289-3 binds to a conformational structure of the virus surface.

### Example 4: Production and Characterization of Recombinant Monoclonal Antibody

### Recombinant Antibody Cloning

Hybridoma cells from selected subclones were collected and lysed for poly(A)+ mRNA isolation using a commercially available Poly(A)+RNA isolation kit. cDNA was then synthesized by RT-PCR from RNA products. The rabbit IgG variable region of the heavy chain and full-length kappa light chain were individually PCR amplified using gene specific primers. Following gel purification of PCR products, the entire kappa light chain fragment was cloned into a mammalian light chain expression vector. For the heavy chain, the variable fragment was fused in-frame to the rabbit heavy chain constant region of a heavy chain vector using standard molecular biology methods. Both heavy and light chain DNA mammalian expression plasmids were prepared for Sanger sequencing and antibody expression.

### Recombinant Antibody Expression

To express recombinant rabbit monoclonal antibody (RabMAb®), the light and heavy chain mammalian expression plasmids were co-transfected into exponential growing 293-6E cells using lipid mediated transfection reagent. The serum free culture supernatant was harvested 5 days after transfection by centrifugation.

For antibody validation, a small scale 2m1 transfection was performed. Antigen binding activity of the supernatants was confirmed by ELISA and further testing was performed prior to scale up.

For antibody production, the heavy and light chain combinations for each selected clone were scaled up for transient expression and purification.

Production and Purification of Monoclonal Antibodies from Recombinant Clones:

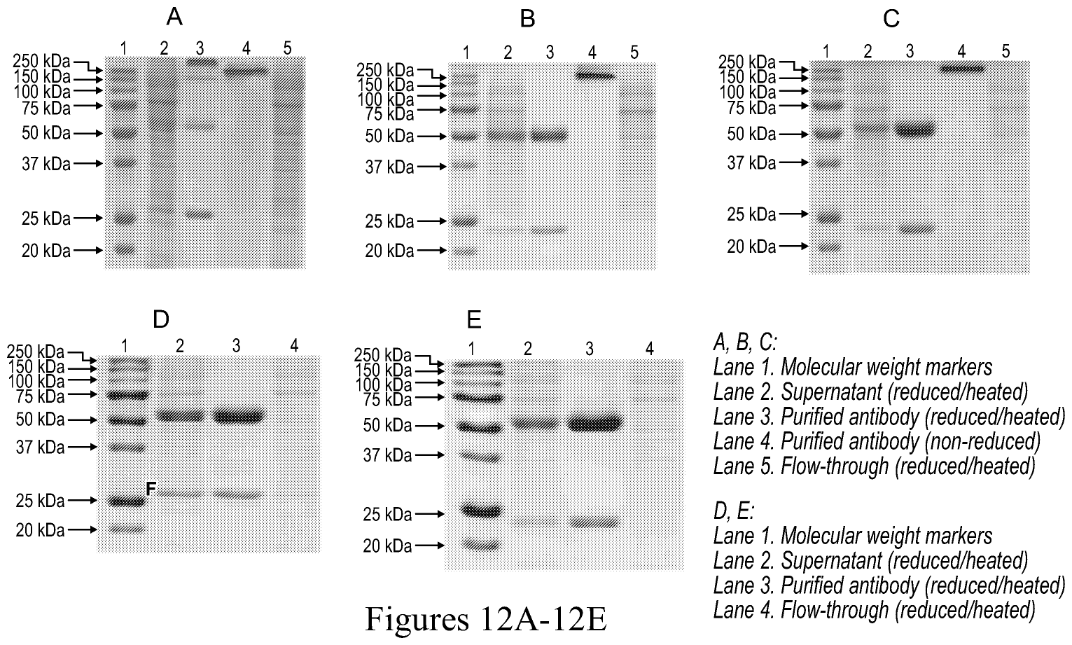

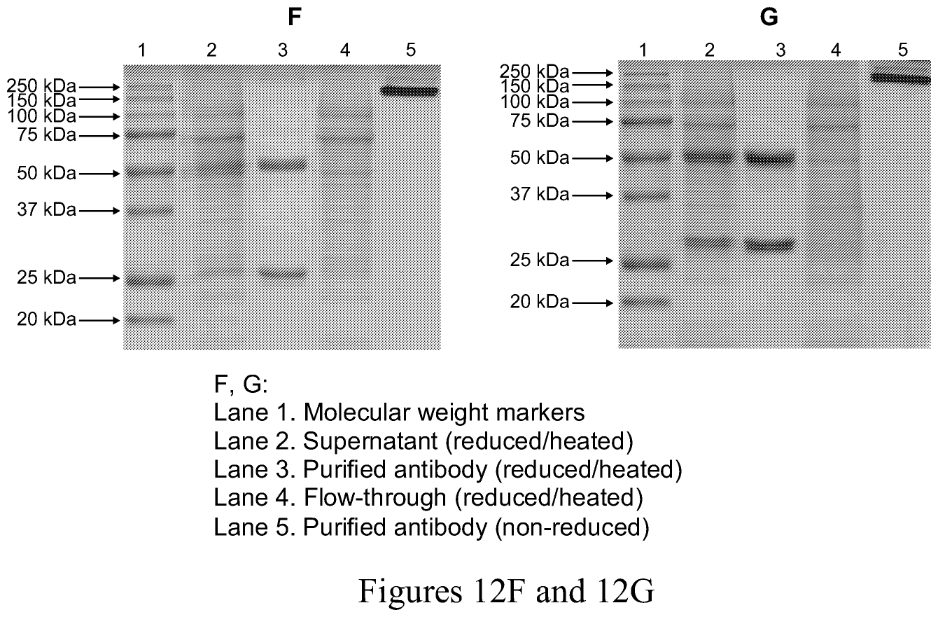

Harvested culture medium was centrifuged to remove cell debris and the clear supernatant containing secreted monoclonal antibodies was purified through MabSelect SuRe protein A column chromatography. The eluted antibody was dialyzed in PBS buffer, sterile filtered, and adjusted to pH 7.4. Purified antibody was serially diluted and tested in ELISA against a ZIKV-VLP and a recombinant ZIKV E Protein. Concentration determination was also performed by ELISA at OD405nm using goat anti-rabbit IgG. The purity and integrity of recombinant antibodies was verified by SDS-PAGE under non-reduced and reduced conditions (FIG. 12). Aliquots of 200 μl and ˜1 mg/mL concentration were prepared. A recombinant rabbit monoclonal antibody generation was achieved in approximately 3 months for all clones. The recombinant stage of RabMAb® development comprised cloning, expression, and validation prior to scale up and purification.

TABLE 15
Recombinant production and purification summary.

| | Production | Production | | Purified Antibody |
|---|---|---|---|---|
| Clone ID | Stage | Size (mL, mg) | | Yield (mg) |
| 102-1 | Phase I | 100 | mL | 16.50 |
| | Phase II | 25 | mg | 25.09 |
| 242-3 | Phase I | 100 | mL | 20.36 |
| | Phase II | 25 | mg | 25.21 |
| 289-3 | Phase I | 100 | mL | 32.40 |
| | Phase II | 25 | mg | 25.20 |
| 306-1 | Phase I | 100 | mL | 27.50 |
| | Phase II | 25 | mg | 25.11 |
| 11-3 | Phase III | 10 | ml | 11.00 |
| 78-2 | Phase III | 21 | ml | 21.84 |

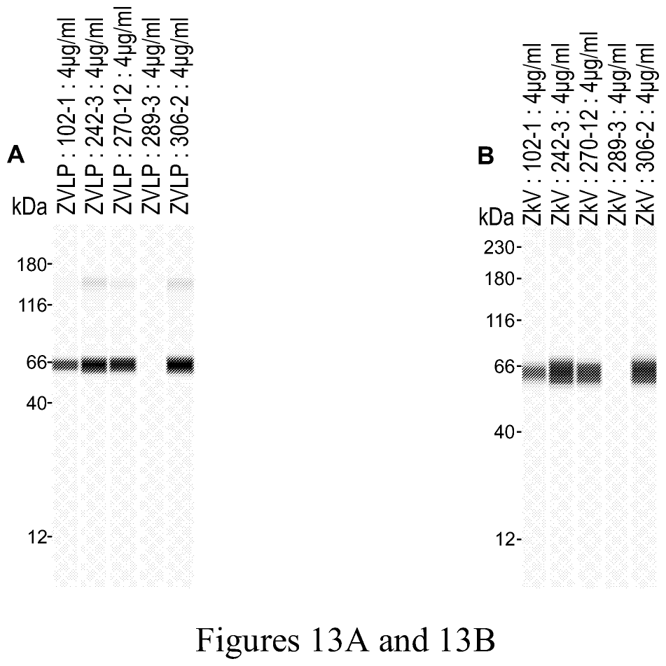

Five recombinant clones: 242-3, 306-2, 102-1, 270-12 and 289-3 were produced and purified. A total of 100mL culture was generated. Recombinant clones 11-3 (10 ml) and 78-2 (21 ml) were also produced and purified. The monoclonal antibodies were purified by affinity chromatography, tested for purity by SDS-PAGE (FIGS. 12 and 13) and quantitated by ELISA. 5-15 mg of antibodies were obtained. These antibodies were tested for neutralizing activity and cross-reactivity (Table 16).

TABLE 16
Cross-reactivity of purified mAbs by ELISA.

| | | | | ELISA | | | | | | MNT |
|---|---|---|---|---|---|---|---|---|---|---|
| Clone ID | Concentration | | Lot No. | ZVLP | ZE | DEN1 | DEN2 | DEN3 | DEN4 | Titer |
| 102-1 | 2.2 | mg/mL | H11271701 | 3.077 | 0.148 | 0.0565 | 0.0495 | 0.049 | 0.048 | 32768 |
| 242-3 | 1.77 | mg/mL | H11271702 | 3.069 | 0.7315 | 0.044 | 0.0485 | 0.049 | 0.0465 | 65536 |
| 289-3 | 2.4 | mg/mL | H11271704 | 3.2125 | 0.1025 | 0.0485 | 0.0485 | 0.0485 | 0.049 | 262144 |
| 306-2 | 2.2 | mg/mL | H11271705 | 3.3025 | 0.5465 | 0.0545 | 0.0485 | 0.0475 | 0.0475 | 8196 |
| 11-3 | 1.1 | mg/mL | H04231802 | ND | ND | ND | ND | ND | ND | ND |
| 78-2 | 1.06 | mg/ml | H04231803 | ND | ND | ND | ND | ND | ND | ND |

Western Blot Analysis of Purified mAbs Using Wes:

Western blot analysis (using Wes system) was performed using ZIKV-VLPs and Zika PRVABC59 live viruses against purified mAbs (FIG. 13). All mAbs, except for 289-3, bound to ZIKV E-protein of both ZIKV-VLP and live ZIKV. Therefore, based from the ELISA data (Table 16), 289-3 mAb binds only to a ZIKV-VLP conformationally and gives a very strong MNT titer; thus, leading us to believe that 289-3 mAb binds to a complex epitope involving two or more domains of E protein that may be sensitive to western blot reagents or conditions. To truly confirm this hypothesis, we performed epitope mapping on this mAb as stated below in this document.

### Epitope Mapping of Clones

Epitope mapping of the clones were carried out at Integral Molecular, Inc. using their alanine scanning mutagenesis library. Important amino acid residues responsible for antibody binding were determined using an alanine scanning mutant library.

Briefly, binding of each test Fab of a MAb to each mutant clone of the target protein, i.e., ZIKV E protein, in the alanine scanning library was determined, in duplicate, by high-throughput flow cytometry. For each point, background fluorescence was subtracted from the raw data, which were then normalized to Fab reactivity with WT target protein. For each mutant clone, the mean binding value was plotted as a function of expression (represented by control reactivity). To identify preliminary primary critical clones, a threshold of >70-80% WT binding to control MAb or Fab and <20-30% WT binding to test Fabs was applied. Secondary clones with slightly higher binding to Fabs as compared to the primary critical clones were identified although they did not meet the set thresholds. These secondary clones had decreased binding activity and proximity to critical residues suggesting that the mutated residue may be part of the antibody epitope.

The summary of the binding specificities is detailed in Table 17. Results indicated that most of the antibodies bound to domain III of a ZIKV E protein.

TABLE 17
Critical amino acid residues on the ZIKV E protein
important for mab binding

| Clone ID | Critical Residues | Domain |
|---|---|---|
| 102-1 | T309, T335, G337, S368 | III |
| 242-3 | T369, E370 | III |
| 270-12 | T335, S368, T369, E370 | III |
| 289-3 | T40, E162, G181, G182, K301, G302, S368 | I, III |
| 306-2 | I317, T397, H398, H399 | III |

Critical residues whose mutation gave the lowest reactivities (<10% of WT) with specific antibodies are highlighted in bold and underlined.

These represent amino acids whose side chains make the highest energetic contributions to the antibody-epitope interaction.

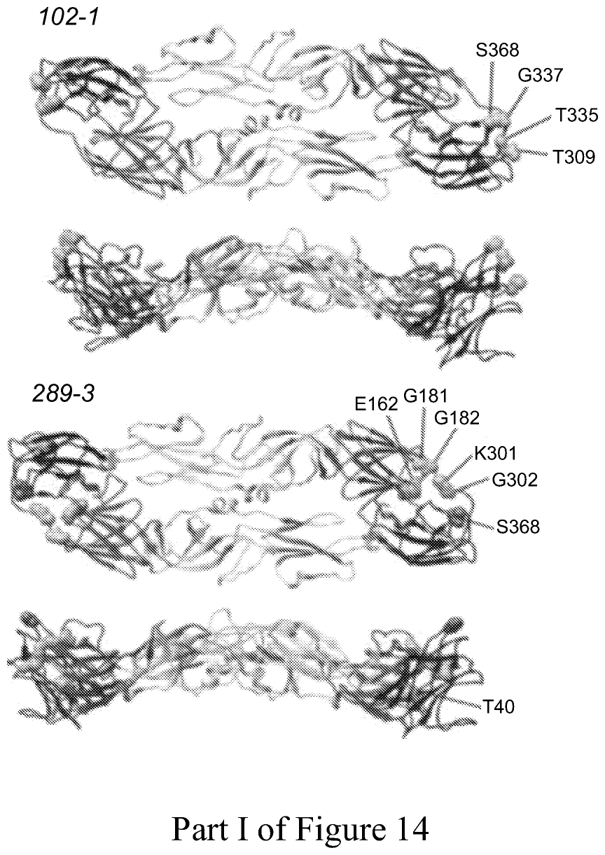

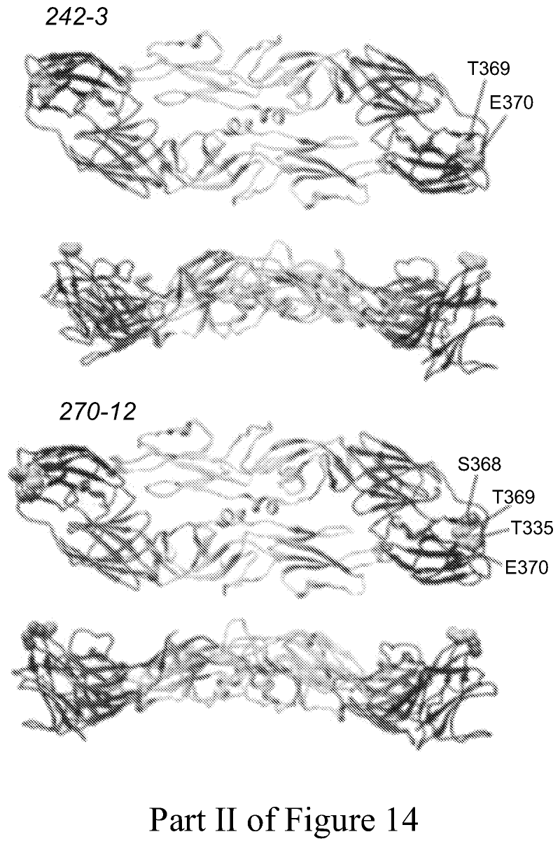

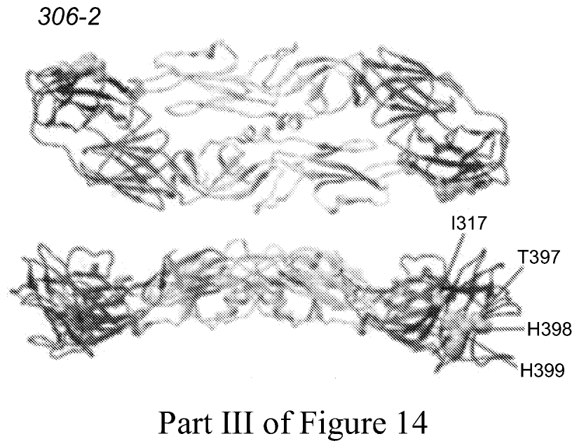

In FIG. 14, critical residues (green spheres) were visualized on a crystal structure of the target protein (PDB ID: SIRE, Sirohi et al, 2016). Secondary residues (grey spheres) that may contribute to binding are also shown. Red: Domain I, Yellow: Domain II, Blue: Domain II.

### INCORPORATION BY REFERENCE

The contents of all references, patents, pending patent applications and published patents, Sequence Listing, and Accession Numbers, cited throughout this application are hereby expressly incorporated by reference in their entireties.

### Equivalents

Those skilled in the art will recognize, or be able to ascertain using no more than routine experimentation, many equivalents to the specific embodiments of the present invention described herein. Such equivalents are intended to be encompassed by the following claims.

1. An isolated antibody, or an antigen-binding portion thereof, that binds to a ZIKV, wherein the isolated antibody, or antigen-binding portion thereof, comprises
(a) (i) a VH comprising a VH CDR1 comprising the amino acid sequence of SEQ ID NOs: 3, a VH CDR2 comprising the amino acid sequence of SEQ ID NO: 4, and a VH CDR3 comprising the amino acid sequence of SEQ ID NO: 5; and (ii) a VL comprising a VL CDR1 comprising the amino acid sequence of SEQ ID NO: 8, a VL CDR2 comprising the amino acid sequence of SEQ ID NO: 9 and a VL CDR3 comprising the amino acid sequence of SEQ ID NO: 10; or
(b) (i) a VH comprising a VH CDR1 comprising the amino acid sequence of SEQ ID NO: 13, a VH CDR2 comprising the amino acid sequence of SEQ ID NO: 14, and a VH CDR3 comprising the amino acid sequence of SEQ ID NO: 15; and (ii) a VL comprising a VL CDR1 comprising the amino acid sequence of SEQ ID NO: 18, a VL CDR2 comprising the amino acid sequence of SEQ ID NO: 19, and a VL CDR3 comprising the amino acid sequence of SEQ ID NO: 20; or
(c) (i) a VH comprising a VH CDR1 comprising the amino acid sequence of SEQ ID NO: 23, a VH CDR2 comprising the amino acid sequence of SEQ ID NO: 24, and a VH CDR3 comprising the amino acid sequence of SEQ ID NO: 25 and (ii) a VL comprising a VL CDR1 comprising the amino acid sequence of SEQ ID NO: 28, a VL CDR2 comprising the amino acid sequence of SEQ ID NO: 29, and a VL CDR3 comprising the amino acid sequence of SEQ ID NO: 30; or
(d) (i) a VH comprising a VH CDR1 comprising the amino acid sequence of SEQ ID NO: 33, a VH CDR2 comprising the amino acid sequence of SEQ ID NO: 34, and a VH CDR3 comprising the amino acid sequence of SEQ ID NO: 35; and (ii) a VL comprising a VL CDR1 comprising the amino acid sequence of SEQ ID NO: 38, a VL CDR2 comprising the amino acid sequence of SEQ ID NO: 39, and a VL CDR3 comprising the amino acid sequence of SEQ ID NO: 40; or
(e) (i) a VH comprising a VH CDR1 comprising the amino acid sequence of SEQ ID NO: 43, a VH CDR2 comprising the amino acid sequence of SEQ ID NO: 44, and a VH CDR3 comprising the amino acid sequence of SEQ ID NO: 45; and (ii) a VL comprising a VL CDR1 comprising the amino acid sequence of SEQ ID NO: 48, a VL CDR2 comprising the amino acid sequence of SEQ ID NO: 49, and a VL CDR3 comprising the amino acid sequence of SEQ ID NO: 50; or
(f) (i) a VH comprising a VH CDR1 comprising the amino acid sequence of SEQ ID NO: 53, a VH CDR2 comprising the amino acid sequence of SEQ ID NO: 54, and a VH CDR3 comprising the amino acid sequence of SEQ ID NO: 55; and (ii) a VL comprising a VL CDR1 comprising the amino acid sequence of SEQ ID NO: 58, a VL CDR2 comprising the amino acid sequence of SEQ ID NO: 59, and a VL CDR3 comprising the amino acid sequence of SEQ ID NO: 60; or
(g) (i) a VH comprising a VH CDR1 comprising the amino acid sequence of SEQ ID NO: 87, a VH CDR2 comprising the amino acid sequence of SEQ ID NO: 88, and a VH CDR3 comprising the amino acid sequence of SEQ ID NO: 89; and (ii) a VL comprising a VL CDR1 comprising the amino acid sequence of SEQ ID NO: 92, a VL CDR2 comprising the amino acid sequence of SEQ ID NO: 93, and a VL CDR3 comprising the amino acid sequence of SEQ ID NO: 94; or
(h)(i) a VH comprising a VH CDR1 comprising the amino acid sequence of SEQ ID NO: 130, a VH CDR2 comprising the amino acid sequence of SEQ ID NO: 131, and a VH CDR3 comprising the amino acid sequence of SEQ ID NO: 132; and (ii) a VL comprising a VL CDR1 comprising the amino acid sequence of SEQ ID NO: 135, a VL CDR2 comprising the amino acid sequence of SEQ ID NO: 136, and a VL CDR3 comprising the amino acid sequence of SEQ ID NO: 137.

2.-24. (canceled)

25. An isolated antibody, or an antigen-binding portion thereof, that binds to a ZIKV according to claim 1, comprising a heavy chain variable region comprising a CDR3 domain comprising the amino acid sequence of SEQ ID NO: 5, a CDR2 domain comprising the amino acid sequence of SEQ ID NO: 4, and a CDR1 domain comprising the amino acid sequence of SEQ ID NO: 3, and a light chain variable region comprising a CDR3 domain comprising the amino acid sequence of SEQ ID NO: 10, a CDR2 domain comprising the amino acid sequence of SEQ ID NO: 9, and a CDR1 domain comprising the amino acid sequence of SEQ ID NO: 8.

26. An isolated antibody, or an antigen-binding portion thereof, that binds to a ZIKV according to claim 1, comprising a heavy chain variable region comprising a CDR3 domain comprising the amino acid sequence of SEQ ID NO: 15, a CDR2 domain comprising the amino acid sequence of SEQ ID NO: 14, and a CDR1 domain comprising the amino acid sequence of SEQ ID NO: 13, and a light chain variable region comprising a CDR3 domain comprising the amino acid sequence of SEQ ID NO: 20, a CDR2 domain comprising the amino acid sequence of SEQ ID NO: 19, and a CDR1 domain comprising the amino acid sequence of SEQ ID NO: 18.

27. An isolated antibody, or an antigen-binding portion thereof, that binds to a ZIKV according to claim 1, comprising a heavy chain variable region comprising a CDR3 domain comprising the amino acid sequence of SEQ ID NO: 25, a CDR2 domain comprising the amino acid sequence of SEQ ID NO: 24, and a CDR1 domain comprising the amino acid sequence of SEQ ID NO: 23, and a light chain variable region comprising a CDR3 domain comprising the amino acid sequence of SEQ ID NO: 30, a CDR2 domain comprising the amino acid sequence of SEQ ID NO: 29, and a CDR1 domain comprising the amino acid sequence of SEQ ID NO: 28.

28. An isolated antibody, or an antigen-binding portion thereof, that binds to a ZIKV according to claim 1, comprising a heavy chain variable region comprising a CDR3 domain comprising the amino acid sequence of SEQ ID NO: 35, a CDR2 domain comprising the amino acid sequence of SEQ ID NO: 34, and a CDR1 domain comprising the amino acid sequence of SEQ ID NO: 33, and a light chain variable region comprising a CDR3 domain comprising the amino acid sequence of SEQ ID NO: 40, a CDR2 domain comprising the amino acid sequence of SEQ ID NO: 39, and a CDR1 domain comprising the amino acid sequence of SEQ ID NO: 38.

29. An isolated antibody, or an antigen-binding portion thereof, that binds to a ZIKV according to claim 1, comprising a heavy chain variable region comprising a CDR3 domain comprising the amino acid sequence of SEQ ID NO: 45, a CDR2 domain comprising the amino acid sequence of SEQ ID NO: 44, and a CDR1 domain comprising the amino acid sequence of SEQ ID NO: 43, and a light chain variable region comprising a CDR3 domain comprising the amino acid sequence of SEQ ID NO: 50, a CDR2 domain comprising the amino acid sequence of SEQ ID NO: 49, and a CDR1 domain comprising the amino acid sequence of SEQ ID NO: 48.

30. An isolated antibody, or an antigen-binding portion thereof, that binds to a ZIKV according to claim 1, comprising a heavy chain variable region comprising a CDR3 domain comprising the amino acid sequence of SEQ ID NO: 55, a CDR2 domain comprising the amino acid sequence of SEQ ID NO: 54, and a CDR1 domain comprising the amino acid sequence of SEQ ID NO: 53, and a light chain variable region comprising a CDR3 domain comprising the amino acid sequence of SEQ ID NO: 60, a CDR2 domain comprising the amino acid sequence of SEQ ID NO: 59, and a CDR1 domain comprising the amino acid sequence of SEQ ID NO: 58.

31. An isolated antibody, or an antigen-binding portion thereof, that binds to a ZIKV according to claim 1, comprising a heavy chain variable region comprising a CDR3 domain comprising the amino acid sequence of SEQ ID NO: 89, a CDR2 domain comprising the amino acid sequence of SEQ ID NO: 88, and a CDR1 domain comprising the amino acid sequence of SEQ ID NO: 87, and a light chain variable region comprising a CDR3 domain comprising the amino acid sequence of SEQ ID NO: 94, a CDR2 domain comprising the amino acid sequence of SEQ ID NO: 93, and a CDR1 domain comprising the amino acid sequence of SEQ ID NO: 92.

32. An isolated antibody, or an antigen-binding portion thereof, that binds to a ZIKV according to claim 1, comprising a heavy chain variable region comprising a CDR3 domain comprising the amino acid sequence of SEQ ID NO: 132, a CDR2 domain comprising the amino acid sequence of SEQ ID NO: 131, and a CDR1 domain comprising the amino acid sequence of SEQ ID NO: 130, and a light chain variable region comprising a CDR3 domain comprising the amino acid sequence of SEQ ID NO: 137, a CDR2 domain comprising the amino acid sequence of SEQ ID NO: 136, and a CDR1 domain comprising the amino acid sequence of SEQ ID NO: 135.

33. An isolated antibody, or an antigen-binding portion thereof, that binds to a ZIKV, comprising a heavy chain variable domain comprising an amino acid sequence set forth in SEQ ID NOs: 1 2, 11, 12, 21, 22, 31, 32, 41, 42, 51, 52, 85, 86, 95, 96, 105, 106, 128, or 129, or a sequence having at least 70%, 75%, 80%, 85%, 90%, 92%, 94%, 95%, 96%, 97%, 98%, or 99% identity to SEQ ID NOs: 1 2, 11, 12, 21, 22, 31, 32, 41, 42, 51, 52, 85, 86 , 95, 96, 105, 106, 128, or 129 and a light chain variable domain comprising an amino acid sequence set forth in SEQ ID NO: 6 7, 16, 17, 26, 27, 36, 37, 46, 47, 56, 57, 90, 91, 100, 101, 110, 111, 133, or 134, or a sequence having at least 70%, 75%, 80%, 85%, 90%, 92%, 94%, 95%, 96%, 97%, 98%, or 99% identity to SEQ ID NO: 6 7, 16, 17, 26, 27, 36, 37, 46, 47, 56, 57, 90, 91, 100, 101, 110, 111, 133, or 134.

34. An isolated antibody, or an antigen-binding portion thereof, that binds to a ZIKV according to claim 33, comprising a heavy chain variable domain comprising an amino acid sequence set forth in SEQ ID NO: 2 and a light chain variable domain comprising an amino acid sequence set forth in SEQ ID NO: 7.

35. (canceled)

36. An isolated antibody, or an antigen-binding portion thereof, that binds to a ZIKV according to claim 33, comprising a heavy chain comprising an amino acid sequence set forth in SEQ ID NO: 1 and a light chain comprising an amino acid sequence set forth in SEQ ID NO: 6.

37. (canceled)

38. An isolated antibody, or an antigen-binding portion thereof, that binds to a ZIKV according to claim 33, comprising a heavy chain variable region comprising the amino acid sequence of SEQ ID NO: 12 and a light chain variable region comprising the amino acid sequence of SEQ ID NO: 17.

39. (canceled)

40. An isolated antibody, or an antigen-binding portion thereof, that binds to a ZIKV according to claim 33, comprising a heavy chain comprising the amino acid sequence of SEQ ID NO: 11 and a light chain comprising the amino acid sequence of SEQ ID NO: 16.

41. (canceled)

42. An isolated antibody, or an antigen-binding portion thereof, that binds to a ZIKV according to claim 33, comprising a heavy chain variable region comprising the amino acid sequence of SEQ ID NO: 22 and a light chain variable region comprising the amino acid sequence of SEQ ID NO: 27.

43. (canceled)

44. An isolated antibody, or an antigen-binding portion thereof, that binds to a ZIKV according to claim 33, comprising a heavy chain comprising the amino acid sequence of SEQ ID NO: 21 and a light chain comprising the amino acid sequence of SEQ ID NO: 26.

45. (canceled)

46. An isolated antibody, or an antigen-binding portion thereof, that binds to a ZIKV according to claim 33, comprising a heavy chain variable region comprising the amino acid sequence of SEQ ID NO: 32 and a light chain variable region comprising the amino acid sequence of SEQ ID NO: 37.

47. (canceled)

48. An isolated antibody, or an antigen-binding portion thereof, that binds to a ZIKV according to claim 33, comprising a heavy chain comprising the amino acid sequence of SEQ ID NO: 31 and a light chain comprising the amino acid sequence of SEQ ID NO: 36.

49. (canceled)

50. An isolated antibody, or an antigen-binding portion thereof, that binds to a ZIKV according to claim 33, comprising a heavy chain variable region comprising the amino acid sequence of SEQ ID NO: 42 and a light chain variable region comprising the amino acid sequence of SEQ ID NO: 47.

51. (canceled)

52. An isolated antibody, or an antigen-binding portion thereof, that binds to a ZIKV according to claim 33, comprising a heavy chain comprising the amino acid sequence of SEQ ID NO: 41 and a light chain comprising the amino acid sequence of SEQ ID NO: 46.

53. (canceled)

54. An isolated antibody, or an antigen-binding portion thereof, that binds to a ZIKV according to claim 33, comprising a heavy chain variable region comprising the amino acid sequence of SEQ ID NO: 52 and a light chain variable region comprising the amino acid sequence of SEQ ID NO: 57.

55. (canceled)

56. An isolated antibody, or an antigen-binding portion thereof, that binds to a ZIKV according to claim 33, comprising a heavy chain comprising the amino acid sequence of SEQ ID NO: 51 and a light chain comprising the amino acid sequence of SEQ ID NO: 56.

57. (canceled)

58. An isolated antibody, or an antigen-binding portion thereof, that binds to a ZIKV according to claim 33, comprising a heavy chain variable domain comprising an amino acid sequence set forth in SEQ ID NO: 86 and a light chain variable domain comprising an amino acid sequence set forth in SEQ ID NO: 91.

59. (canceled)

60. An isolated antibody, or an antigen-binding portion thereof, that binds to a ZIKV according to claim 33, comprising a heavy chain comprising an amino acid sequence set forth in SEQ ID NO: 85 and a light chain comprising an amino acid sequence set forth in SEQ ID NO: 90.

61. (canceled)

62. An isolated antibody, or an antigen-binding portion thereof, that binds to a ZIKV according to claim 33, comprising a heavy chain variable domain comprising an amino acid sequence set forth in SEQ ID NO: 96 and a light chain variable domain comprising an amino acid sequence set forth in SEQ ID NO: 101.

63. (canceled)

64. An isolated antibody, or an antigen-binding portion thereof, that binds to a ZIKV according to claim 33, comprising a heavy chain comprising an amino acid sequence set forth in SEQ ID NO: 95 and a light chain comprising an amino acid sequence set forth in SEQ ID NO: 100.

65. (canceled)

66. An isolated antibody, or an antigen-binding portion thereof, that binds to a ZIKV according to claim 33, comprising a heavy chain variable domain comprising an amino acid sequence set forth in SEQ ID NO: 106 and a light chain variable domain comprising an amino acid sequence set forth in SEQ ID NO: 111.

67. (canceled)

68. An isolated antibody, or an antigen-binding portion thereof, that binds to a ZIKV according to claim 33, comprising a heavy chain comprising an amino acid sequence set forth in SEQ ID NO: 105 and a light chain comprising an amino acid sequence set forth in SEQ ID NO: 110.

69. (canceled)

70. An isolated antibody, or an antigen-binding portion thereof, that binds to a ZIKV according to claim 33, comprising a heavy chain variable domain comprising an amino acid sequence set forth in SEQ ID NO: 129 and a light chain variable domain comprising an amino acid sequence set forth in SEQ ID NO: 134.

71. (canceled)

72. An isolated antibody, or an antigen-binding portion thereof, that binds to a ZIKV according to claim 33, comprising a heavy chain comprising an amino acid sequence set forth in SEQ ID NO: 128 and a light chain comprising an amino acid sequence set forth in SEQ ID NO: 133.

73. An isolated antibody, or an antigen-binding portion thereof, that binds to an epitope on an E-protein of a ZIKV which also specifically binds to an isolated antibody or antigen binding fragment thereof of claim 1.

74. An isolated antibody, or an antigen-binding portion thereof, that binds to an epitope on an E-protein of a ZIKV according to claim 73 comprising an amino acid sequence of SEQ ID NO: 127, wherein the epitope comprises amino acids of T309, T335, G337 and 5368 present in SEQ ID NO: 127.

75. An isolated antibody, or an antigen-binding portion thereof, that binds to an epitope on an E-protein of a ZIKV according to claim 73 comprising an amino acid sequence of SEQ ID NO: 127 wherein the epitope comprises amino acids of T369 and E370 present in SEQ ID NO: 127.

76. An isolated antibody, or an antigen-binding portion thereof, that binds to an epitope on an E-protein of a ZIKV according to claim 73 comprising an amino acid sequence of SEQ ID NO: 127, wherein the epitope comprises amino acids of E162, G181, G182, K301 and G302 present in SEQ ID NO: 127.

77. An isolated antibody, or an antigen-binding portion thereof, that binds to an epitope on an E-protein of a ZIKV according to claim 73 comprising an amino acid sequence of SEQ ID NO: 127, wherein the epitope comprises amino acids of 1317, T397, H398 and H399 present in SEQ ID NO: 127.

78. An isolated antibody, or an antigen-binding portion thereof, that binds to an epitope on an E-protein of a ZIKV according to claim 73 comprising an amino acid sequence of SEQ ID NO: 127 wherein the epitope comprises amino acids of T335, 5368, T369 and E370 present in SEQ ID NO: 127.

79. The isolated antibody, or antigen-binding portion thereof of claim 1, wherein the isolated antibody, or antigen-binding portion thereof, neutralizes a viral infection caused by one or more ZIKV strains.

80. The isolated antibody, or antigen-binding portion thereof, of claim 79, wherein the isolated antibody, or antigen-binding portion thereof, binds specifically to one or more ZIKV strains are selected from the group consisting of ZikaSPH (Brazil 2015, KU321639.1), Brazil-ZKV (Brazil 2015, KU497555.1), PRVABC59 (Puerto Rico 2015, KU501215.1), Haiti1225 (Haiti 2014, KU509998.1), Natal RGN (Brazil, KU527068.1), SV0127-14 (Thailand 2014, KU681081.3), CPC-0740 (Philippine 2012, KU681082.3), SSABR1 (Brazil, KU707826.1), VE Ganxian (China, KU744693.1), MR766-NIID (Uganda, LC002520.1), MR 766 (Uganda 1947, AY632535.2), and H/PF (French Polynesia 2013, KJ776791.1).

81.-97. (canceled)

98. A pharmaceutical composition comprising at least one isolated antibody, or antigen-binding portion thereof, of claim 1, and a pharmaceutically acceptable excipient, carrier or diluent.

99.-104. (canceled)

105. An isolated nucleic acid molecule encoding the isolated antibody, or antigen-binding portion thereof, of claim 1.

106. An isolated nucleic acid molecule encoding an antibody comprising a heavy chain variable region comprising a nucleic acid sequence of SEQ ID NO: 61, 62, 65, 66, 69, 70, 73, 74, 77, 78, 81, 82, 115, 116, 119, 120, 123, 124, 138, or 139, or a sequence having at least 70%, 75%, 80%, 85%, 90%, 92%, 94%, 95%, 96%, 97%, 98%, or 99% identity to SEQ ID NO: 61, 62, 65, 66, 69, 70, 73, 74, 77, 78, 81, 82, 115, 116, 119, 120, 123, 124, 138, or 139 and a light chain variable region comprising a nucleic acid sequence of SEQ ID NO: 63, 64, 67, 68, 71, 72, 75, 76, 79, 80, 83, 84, 117, 118, 121, 122, 125, 126, 140, or 141 a sequence having at least 70%, 75%, 80%, 85%, 90%, 92%, 94%, 95%, 96%, 97%, 98%, or 99% identity to SEQ ID NO: 63, 64, 67, 68, 71, 72, 75, 76, 79, 80, 83, 84, 117, 118, 121, 122, 125, 126, 140, or 141.

107.-125. (canceled)

126. A vector comprising the nucleic acid molecule of claim 105.

127. An isolated cell expressing the nucleic acid molecule of claim 105.

128. A kit comprising an antibody, or antigen-binding portion thereof, according to claim 1 and instructions for use.

129. A method of neutralizing an infectious ZIKV, the method comprising exposing a cell infected with an infectious ZIKV to the isolated antibody, or antigen-binding portion thereof, of claim 1, wherein the exposing results in enhanced protection of the cell from viral infection, or from cell death.

130.-132. (canceled)

133. A method of preventing, treating or ameliorating at least one symptom of a ZIKV infection, or of decreasing the frequency or severity of at least one symptom of a ZIKV infection, the method comprising administering an effective amount of at least one isolated antibody, or antigen-binding portion thereof, of claim 1, to a subject in need thereof.

134. The method of claim 133, wherein the at least one symptom is selected from the group consisting of fever, headache, arthralgia, myalgia and a maculopapular rash.

135. The method of claim 133, wherein the subject in need thereof is at risk for exposure to, or for acquiring a ZIKV infection and wherein the subject is selected from the group consisting of a pregnant woman who has been exposed to a ZIKV, or who has been bitten by a mosquito suspected of harboring a ZIKV, a woman who lives in an area that has an outbreak of a ZIKV, or is visiting an area that has an outbreak of a ZIKV and who is considering conceiving a child, an immunocompromised individual, a person who is suspected of having been exposed to a person harboring a ZIKV, a person who comes into physical contact or close physical proximity with a ZIKV-infected individual, a hospital employee, a pharmaceutical researcher, maintenance personnel responsible for cleaning a hospital facility or institution where a ZIKV-infected patient has been treated, individuals who have visited or are planning to visit an area or country known to have or suspected to have an outbreak of a ZIKV, or a country known to have mosquitoes harboring a ZIKV.

136. A method of decreasing the likelihood of transmitting a ZIKV infection to the fetus of a pregnant female, and/or prevention of transmission to the male reproductive organs, the method comprising administering to a subject in need thereof an effective amount of at least one isolated antibody or antigen-binding portion of claim 1, or the pharmaceutical composition of claim 98, to a subject in need thereof.

137. The method of claim 136, wherein the isolated antibody or antigen-binding portion thereof, or the pharmaceutical composition, is administered prophylactically or therapeutically to the subject in need thereof

138.-141. (canceled)

142. The method of claim 129, wherein the isolated antibody, or antigen-binding portion thereof, is administered at a (single) dose of about 0.001 to about 60 mg/kg body weight.

143. (canceled)

144. A method of in vitro diagnosing a ZIKV infection in a subject, comprising:
(i) contacting a sample isolated from a subject with a plate coated with a ZIKV E protein, ZIKV E protein, VLP, virus or inactivated virus and incubating the sample on the plate for a period of time;
(ii) contacting the sample having been incubated on the plate in step (i) with the isolated antibody, or antigen-binding portion thereof, of claim 1, and further incubating for a period of time; and
(iii) determining inhibition of binding of the isolated antibody or antigen-binding portion thereof to the plate, wherein the inhibition of the binding of the isolated antibody or antigen-binding portion thereof to the plate reflects the presence of an anti-ZIKV antibody in the sample; and wherein the presence of the anti-ZIKV antibody in the sample indicates a ZIKV infection in the subject, thereby diagnosing a ZIKV infection in the subject.

145. The method of claim 144, wherein the sample is selected from the group consisting of blood, saliva and urine.

146.-153. (canceled)

154. A method of determining the potency/concentration of a test anti-ZIKV antibody, comprising:
(a) coating a well of a plate with a PIZV;
(b) incubating the PIZV coated on the well with one of a plurality of solutions of a test antibody, wherein the plurality of solutions of the test antibody are prepared by a serial dilution of a concentrated solution of the test antibody;
(c) incubating the test antibody, which binds to the PIZV, with a secondary antibody, wherein the binding of the secondary antibody to the isolated antibody produces a signal;
(d) detecting the signal;
(e) repeating steps (a)-(d) with the remaining of the plurality of solutions prepared in step (b); and
(f) ploting a dilution curve with the signal of the secondary antibody against the concentration of each of the plurality of solutions prepared in step (b), wherein an IC₅₀ of the dilution curve indicates the potency of the test antibody.

155. A method of determining the potency/concentration of a test PIZV, comprising:
(a) coating a well of a plate with a first anti-ZIKV antibody which binds to a first epitope on a test PIZV and captures the test PIZV;
(b) incubating the coated well with the test PIZV;
(c) further incubating the coated well with one of a plurality of solutions of a second anti-ZIKV antibody which binds to a second epitope on the test PIVZ, wherein the plurality of solutions of the second anti-ZIKV antibody are prepared by a serial dilution of a concentrated solution of the second anti-ZIKV antibody;
(d) further incubating the coated well with a secondary antibody conjugated to an enzyme or a reporter, wherein the binding of the secondary antibody to the second anti-ZIKV antibody produces a signal;
(e) detecting the signal;
(f) repeating steps (a)-(e) with the remaining of the plurality of solutions of the second anti-ZIKV antibody prepared in step (c); and
(g) ploting a dilution curve with the signal of the secondary anti-ZIKV antibody against the concentration of each of the plurality of solutions of the second anti-ZIKV antibody prepared in step (c), wherein an IC50 of the dilution curve indicates the potency of the test PIZV,
wherein at least one of the first anti-ZIKV antibody and the second anti-ZIKV antibody is the isolated antibody of claim 1 and wherein the first epitope does not overlap with the second epitope.


SEQUENCE LISTING (132 sequences)

SEQ ID NO: 1 - protein, 441 aa, Oryctolagus cuniculus
QSLEESGGDLVKPGASLTLTSKVSGFSFNSNYWICWVRQAPGKGLEWIGCVFGGIHVTYYANWVKGRFTISKPSSTTVTLEMTSLTAADTATYFCIISTGGSHRFNLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 2 - protein, 118 aa, artificial sequence
  Synthetic peptide
QSLEESGGDLVKPGASLTLTSKVSGFSFNSNYWICWVRQAPGKGLEWIGCVFGGIHVTYYANWVKGRFTISKPSSTTVTLEMTSLTAADTATYFCIISTGGSHRFNLWGPGTLVTVSS

SEQ ID NO: 3 - protein, 9 aa, artificial sequence
  Synthetic peptide
GFSFNSNYW

SEQ ID NO: 4 - protein, 7 aa, artificial sequence
  Synthetic peptide
FGGIHVT

SEQ ID NO: 5 - protein, 12 aa, artificial sequence
  Synthetic peptide
IISTGGSHRFNL

SEQ ID NO: 6 - protein, 214 aa, Oryctolagus cuniculus
DVVMTQTPASVEAAVGGTVTIKCQASESIYTYLSWYQQKPGQPPKLLIYRASTLDSGVPSRFSGSGSGTEFTLTISDLDCADAASYYCQATDVGGSGRGAFGGGTEVVVEGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 7 - protein, 110 aa, artificial sequence
  Synthetic peptide
DVVMTQTPASVEAAVGGTVTIKCQASESIYTYLSWYQQKPGQPPKLLIYRASTLDSGVPSRFSGSGSGTEFTLTISDLDCADAASYYCQATDVGGSGRGAFGGGTEVVVE

SEQ ID NO: 8 - protein, 6 aa, artificial sequence
  Synthetic peptide
ESIYTY

SEQ ID NO: 9 - protein, 12 aa, artificial sequence
  Synthetic peptide
QATDVGGSGRGA

SEQ ID NO: 10 - protein, 448 aa, Oryctolagus cuniculus
QAQLEESGGDLVKPEGSLTLTCTASGFSFTDRHYMCWIRQAPGKGLEWIACVYPGSSGSTYYASWARGRFTISKTSSTTVTLRMTSLTAADTATYFCARSSYPDSSGYSYGMDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 11 - protein, 125 aa, artificial sequence
  Synthetic peptide
QAQLEESGGDLVKPEGSLTLTCTASGFSFTDRHYMCWIRQAPGKGLEWIACVYPGSSGSTYYASWARGRFTISKTSSTTVTLRMTSLTAADTATYFCARSSYPDSSGYSYGMDLWGPGTLVTVSS

SEQ ID NO: 12 - protein, 9 aa, artificial sequence
  Synthetic peptide
GFSFTDRHY

SEQ ID NO: 13 - protein, 8 aa, artificial sequence
  Synthetic peptide
YPGSSGST

SEQ ID NO: 14 - protein, 17 aa, artificial sequence
  Synthetic peptide
ARSSYPDSSGYSYGMDL

SEQ ID NO: 15 - protein, 214 aa, Oryctolagus cuniculus
DIVMTQTPASVEAAVGGTVTIKCQASQNINSNLAWYQQKPGQRPKFLMYLTSKLASGVPSRFRGSGSGTQFTLTISDLECADAATYYCQTYYDISNYGYAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 16 - protein, 110 aa, artificial sequence
  Synthetic peptide
DIVMTQTPASVEAAVGGTVTIKCQASQNINSNLAWYQQKPGQRPKFLMYLTSKLASGVPSRFRGSGSGTQFTLTISDLECADAATYYCQTYYDISNYGYAFGGGTEVVVK

SEQ ID NO: 17 - protein, 6 aa, artificial sequence
  Synthetic peptide
QNINSN

SEQ ID NO: 18 - protein, 12 aa, artificial sequence
  Synthetic peptide
QTYYDISNYGYA

SEQ ID NO: 19 - protein, 448 aa, Oryctolagus cuniculus
QEELEESGGDLVKPGASLTLTCKASGFSFSSGAYICWVRQAPGKGPEWIACIYTGDGRTYYANWAKRRFTISSSSSTTVALEMPSLTDADTATYFCARAIAVGAGYGVGNYFTLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 20 - protein, 125 aa, artificial sequence
  Synthetic peptide
QEELEESGGDLVKPGASLTLTCKASGFSFSSGAYICWVRQAPGKGPEWIACIYTGDGRTYYANWAKRRFTISSSSSTTVALEMPSLTDADTATYFCARAIAVGAGYGVGNYFTLWGPGTLVTVSS

SEQ ID NO: 21 - protein, 9 aa, artificial sequence
  Synthetic peptide
GFSFSSGAY

SEQ ID NO: 22 - protein, 7 aa, artificial sequence
  Synthetic peptide
YTGDGRT

SEQ ID NO: 23 - protein, 18 aa, artificial sequence
  Synthetic peptide
ARAIAVGAGYGVGNYFTL

SEQ ID NO: 24 - protein, 218 aa, Oryctolagus cuniculus
DIVMTQTPSSVSAAVGGTVTINCHASENIYGYLSWYQQKPGQPPKLLIYKASTLASGVPSRFKGSGAGTQFTLTITALECADAATYYCQSYYTSSSNADGSENAFGGGTEVVVEGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 25 - protein, 114 aa, artificial sequence
  Synthetic peptide
DIVMTQTPSSVSAAVGGTVTINCHASENIYGYLSWYQQKPGQPPKLLIYKASTLASGVPSRFKGSGAGTQFTLTITALECADAATYYCQSYYTSSSNADGSENAFGGGTEVVVE

SEQ ID NO: 26 - protein, 6 aa, artificial sequence
  Synthetic peptide
ENIYGY

SEQ ID NO: 27 - protein, 16 aa, artificial sequence
  Synthetic peptide
QSYYTSSSNADGSENA

SEQ ID NO: 28 - protein, 439 aa, Oryctolagus cuniculus
QSLEESGGDLVKPGASLTLTCTASGFDFSDRYYMCWVRQAPGKGLEWIGCIYVGSGDTYYASWAKGRFTISRTSSTTVTLQMTRLTAADTATYFCARHPGTYFTLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 29 - protein, 116 aa, artificial sequence
  Synthetic peptide
QSLEESGGDLVKPGASLTLTCTASGFDFSDRYYMCWVRQAPGKGLEWIGCIYVGSGDTYYASWAKGRFTISRTSSTTVTLQMTRLTAADTATYFCARHPGTYFTLWGPGTLVTVSS

SEQ ID NO: 30 - protein, 9 aa, artificial sequence
  Synthetic peptide
GFDFSDRYY

SEQ ID NO: 31 - protein, 7 aa, artificial sequence
  Synthetic peptide
YVGSGDT

SEQ ID NO: 32 - protein, 8 aa, artificial sequence
  Synthetic peptide
ARHPGTYF

SEQ ID NO: 33 - protein, 214 aa, Oryctolagus cuniculus
DVVMTQTPSSVSAAVGGTVTINCQASQNIVNNLAWYQQRPGQPPKLLIYDTSTLASGVPSRFKGSGSGTEFTLTISDLECADAATYYCQTYYYYNKIINGFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 34 - protein, 110 aa, artificial sequence
  Synthetic peptide
DVVMTQTPSSVSAAVGGTVTINCQASQNIVNNLAWYQQRPGQPPKLLIYDTSTLASGVPSRFKGSGSGTEFTLTISDLECADAATYYCQTYYYYNKIINGFGGGTEVVVK

SEQ ID NO: 35 - protein, 6 aa, artificial sequence
  Synthetic peptide
QNIVNN

SEQ ID NO: 36 - protein, 12 aa, artificial sequence
  Synthetic peptide
QTYYYYNKIING

SEQ ID NO: 37 - protein, 444 aa, Oryctolagus cuniculus
QEQLVESGGGLVQPEGSLTITCTGSGFSFSSRFYMCWVRQAPGKGLEWIACIYGGSSGSTYYASWAKGRFTISKTSSTTVTLQISSLTAADTATYFCARGGSTAAAGFNLWGPGTLLTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 38 - protein, 121 aa, artificial sequence
  Synthetic peptide
QEQLVESGGGLVQPEGSLTITCTGSGFSFSSRFYMCWVRQAPGKGLEWIACIYGGSSGSTYYASWAKGRFTISKTSSTTVTLQISSLTAADTATYFCARGGSTAAAGFNLWGPGTLLTVSS

SEQ ID NO: 39 - protein, 9 aa, artificial sequence
  Synthetic peptide
GFSFSSRFY

SEQ ID NO: 40 - protein, 8 aa, artificial sequence
  Synthetic peptide
YGGSSGST

SEQ ID NO: 41 - protein, 13 aa, artificial sequence
  Synthetic peptide
ARGGSTAAAGFNL

SEQ ID NO: 42 - protein, 215 aa, Oryctolagus cuniculus
DVVMTQTPASVSATVGGTVTIKCQASEDIYNLLAWYQQKPGQPPKLLIYYASTLASGVPSRFKGSGSGTEFTLTISDLECADAATYYCQCNDYGGTYVPNAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 43 - protein, 111 aa, artificial sequence
  Synthetic peptide
DVVMTQTPASVSATVGGTVTIKCQASEDIYNLLAWYQQKPGQPPKLLIYYASTLASGVPSRFKGSGSGTEFTLTISDLECADAATYYCQCNDYGGTYVPNAFGGGTEVVVK

SEQ ID NO: 44 - protein, 6 aa, artificial sequence
  Synthetic peptide
EDIYNL

SEQ ID NO: 45 - protein, 13 aa, artificial sequence
  Synthetic peptide
QCNDYGGTYVPNA

SEQ ID NO: 46 - protein, 448 aa, Oryctolagus cuniculus
QQQLVESGGGLVKPGASLTLNCKASGLDFSTNSYMCWVRQAPGKGLEWIACIYVGDSSEIYYASWAKGRFTISKTSSTTVTLQMTSLTAADTATYFCARDLPSFTAPYAGYLRLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 47 - protein, 125 aa, artificial sequence
  Synthetic peptide
QQQLVESGGGLVKPGASLTLNCKASGLDFSTNSYMCWVRQAPGKGLEWIACIYVGDSSEIYYASWAKGRFTISKTSSTTVTLQMTSLTAADTATYFCARDLPSFTAPYAGYLRLWGPGTLVTVSS

SEQ ID NO: 48 - protein, 9 aa, artificial sequence
  Synthetic peptide
GLDFSTNSY

SEQ ID NO: 49 - protein, 9 aa, artificial sequence
  Synthetic peptide
IYVGDSSEI

SEQ ID NO: 50 - protein, 17 aa, artificial sequence
  Synthetic peptide
ARDLPSFTAPYAGYLRL

SEQ ID NO: 51 - protein, 225 aa, Oryctolagus cuniculus
QPVLTQPPSLSASLGTTARLTCTLRTGYNVGDYPLLWLQRVPGRPPRYLLTYHTEEFKHQGSGVHSRFSGSKDASENAGVLSISGLQPEDEANYYCYTVHATESSLHYVFGGGTQLTVTGQPAVTPSVILFPPSSEELKDNKATLVCLINDFYPGTVKVNWKADGTPVTQGVDTTQPSKQSNSKYAASSFLSLSANQWKSYQSVTCQVTHEGHTVEKSLAPAECS

SEQ ID NO: 52 - protein, 119 aa, artificial sequence
  Synthetic peptide
QPVLTQPPSLSASLGTTARLTCTLRTGYNVGDYPLLWLQRVPGRPPRYLLTYHTEEFKHQGSGVHSRFSGSKDASENAGVLSISGLQPEDEANYYCYTVHATESSLHYVFGGGTQLTVT

SEQ ID NO: 53 - protein, 9 aa, artificial sequence
  Synthetic peptide
TGYNVGDYP

SEQ ID NO: 54 - protein, 8 aa, artificial sequence
  Synthetic peptide
YHTEEFKH

SEQ ID NO: 55 - protein, 14 aa, artificial sequence
  Synthetic peptide
YTVHATESSLHYVF

SEQ ID NO: 56 - DNA, 1383 bp, Oryctolagus cuniculus
atggagactgggctgcgctggcttctcctggtcgctgtgctcaaaggtgtccaatgtcagtcgttggaggagtccgggggagacctggtcaagcctggggcatccctgacactcacctccaaagtctctggattctccttcaatagcaactattggatatgctgggtccgccaggctccagggaaggggctggagtggatcggatgcgtttttggtggtattcatgtcacatattacgcgaattgggtgaaaggccgattcaccatctccaaaccctcgtcgaccacggtgactctggaaatgaccagtctgacagccgcggacacggccacttatttctgtattattagtactggcggtagtcacagatttaatttgtggggcccaggcaccctggtcaccgtctcctcagggcaacctaaggctccatcagtcttcccactggccccctgctgcggggacacacccagctccacggtgaccctgggctgcctggtcaaagggtacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 57 - DNA, 411 bp, artificial sequence
  Synthetic nucleotide
atggagactgggctgcgctggcttctcctggtcgctgtgctcaaaggtgtccaatgtcagtcgttggaggagtccgggggagacctggtcaagcctggggcatccctgacactcacctccaaagtctctggattctccttcaatagcaactattggatatgctgggtccgccaggctccagggaaggggctggagtggatcggatgcgtttttggtggtattcatgtcacatattacgcgaattgggtgaaaggccgattcaccatctccaaaccctcgtcgaccacggtgactctggaaatgaccagtctgacagccgcggacacggccacttatttctgtattattagtactggcggtagtcacagatttaatttgtggggcccaggcaccctggtcaccgtctcctca

SEQ ID NO: 58 - DNA, 711 bp, Oryctolagus cuniculus
atggacacgagggcccccactcagctgctggggctcctgctgctctggctcccaggtgccagatgtgatgttgtgatgacccagactccagcctccgtggaggcagctgtgggaggcacagtcaccatcaagtgccaggccagtgagagcatttatacctacttatcctggtatcagcagaaaccagggcagcctcccaaactcctgatctatagggcatccactctggattctggggtcccatcgcggttcagcggcagtggatctgggacagagttcactctcaccatcagcgacctggactgtgccgatgctgccagttattattgtcaggctactgatgttggtggtagtggtaggggtgctttcggcggagggaccgaggtggtggtcgaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 59 - DNA, 396 bp, artificial sequence
  Synthetic nucleotide
atggacacgagggcccccactcagctgctggggctcctgctgctctggctcccaggtgccagatgtgatgttgtgatgacccagactccagcctccgtggaggcagctgtgggaggcacagtcaccatcaagtgccaggccagtgagagcatttatacctacttatcctggtatcagcagaaaccagggcagcctcccaaactcctgatctatagggcatccactctggattctggggtcccatcgcggttcagcggcagtggatctgggacagagttcactctcaccatcagcgacctggactgtgccgatgctgccagttattattgtcaggctactgatgttggtggtagtggtaggggtgctttcggcggagggaccgaggtggtggtcgaa

SEQ ID NO: 60 - DNA, 1404 bp, Oryctolagus cuniculus
atggagactgggctgcgctggcttctcctggtcgctgtgctcaaaggtgtccagtgtcaggcgcagctggaggagtccgggggagacctggtcaagcctgagggatccctgacactcacctgcacagcctctggattctccttcactgacaggcattacatgtgctggatccgccaggctccggggaaggggctggagtggatcgcatgcgtttatcctggtagtagtggtagcacttactacgcgagctgggcgagaggccgattcaccatctccaaaacctcgtcgaccacggtgactctgcgcatgaccagtctgacagccgcggacacggccacctatttctgtgcgagatcctcatatcctgatagtagtggctatagttatggcatggacctctggggccctgggaccctcgtcaccgtctcttcagggcaacctaaggctccatcagtcttcccactggccccctgctgcggggacacacccagctccacggtgaccctgggctgcctggtcaaagggtacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 61 - DNA, 432 bp, artificial sequence
  Synthetic nucleotide
atggagactgggctgcgctggcttctcctggtcgctgtgctcaaaggtgtccagtgtcaggcgcagctggaggagtccgggggagacctggtcaagcctgagggatccctgacactcacctgcacagcctctggattctccttcactgacaggcattacatgtgctggatccgccaggctccggggaaggggctggagtggatcgcatgcgtttatcctggtagtagtggtagcacttactacgcgagctgggcgagaggccgattcaccatctccaaaacctcgtcgaccacggtgactctgcgcatgaccagtctgacagccgcggacacggccacctatttctgtgcgagatcctcatatcctgatagtagtggctatagttatggcatggacctctggggccctgggaccctcgtcaccgtctcttca

SEQ ID NO: 62 - DNA, 714 bp, Oryctolagus cuniculus
atggacacgagggcccccactcagctgctggggctcctgttgctctggctcccaggtgccagatgtgctgacattgtgatgacccagactccagcctccgtggaggcagctgtgggaggcacagtcaccatcaagtgccaggccagtcagaacattaacagcaatttagcctggtatcagcagaaaccagggcagcgtcccaagttcctgatgtatctgacatccaaactggcatctggggtcccatcgcggttcagaggcagtggatctgggacacagttcactctcaccatcagcgacctggagtgtgccgatgctgccacttactactgtcaaacctattatgatattagtaattatggatatgctttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 63 - DNA, 399 bp, artificial sequence
  Synthetic nucleotide
atggacacgagggcccccactcagctgctggggctcctgttgctctggctcccaggtgccagatgtgctgacattgtgatgacccagactccagcctccgtggaggcagctgtgggaggcacagtcaccatcaagtgccaggccagtcagaacattaacagcaatttagcctggtatcagcagaaaccagggcagcgtcccaagttcctgatgtatctgacatccaaactggcatctggggtcccatcgcggttcagaggcagtggatctgggacacagttcactctcaccatcagcgacctggagtgtgccgatgctgccacttactactgtcaaacctattatgatattagtaattatggatatgctttcggcggagggaccgaggtggtggtcaaa

SEQ ID NO: 64 - DNA, 1404 bp, Oryctolagus cuniculus
atggagactgggctgcgctggcttctcctggtcgctgtgctcaaaggtgtccagtgtcaggaggagctggaggagtccgggggagacctggtcaagcctggggcatccctgacactcacctgcaaagcctctggattctccttcagtagtggcgcctacatatgctgggtccgccaggctccagggaaggggcctgagtggatcgcgtgtatttatactggtgatggccgcacatactacgcgaactgggcgaagcgccgcttcaccatctccagttcctcgtcgaccacggtggctctggaaatgcccagtctgacagacgcggacacggcgacctatttctgtgcgcgagcaattgctgttggcgctggttatggtgttggaaactactttaccttgtggggcccaggcaccctggtcaccgtctcctcagggcaacctaaggctccatcagtcttcccactggccccctgctgcggggacacacccagctccacggtgaccctgggctgcctggtcaaagggtacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 65 - DNA, 432 bp, artificial sequence
  Synthetic nucleotide
atggagactgggctgcgctggcttctcctggtcgctgtgctcaaaggtgtccagtgtcaggaggagctggaggagtccgggggagacctggtcaagcctggggcatccctgacactcacctgcaaagcctctggattctccttcagtagtggcgcctacatatgctgggtccgccaggctccagggaaggggcctgagtggatcgcgtgtatttatactggtgatggccgcacatactacgcgaactgggcgaagcgccgcttcaccatctccagttcctcgtcgaccacggtggctctggaaatgcccagtctgacagacgcggacacggcgacctatttctgtgcgcgagcaattgctgttggcgctggttatggtgttggaaactactttaccttgtggggcccaggcaccctggtcaccgtctcctca

SEQ ID NO: 66 - DNA, 723 bp, Oryctolagus cuniculus
atggacacgagggcccccactcagctgctggggctcctgctgctctggctcccaggtgccatatgtgacattgtgatgacccagactccatcttccgtgtctgcagctgtgggaggcacagtcaccatcaattgccacgccagtgagaacatttacggctacttatcctggtatcagcagaaaccagggcagcctcccaagctcctgatctacaaggcttccactctggcatctggggtcccatcgcggttcaaaggcagtggagctgggacacagttcactctcaccatcaccgccctggagtgtgccgatgctgccacttactactgtcaaagttattatactagtagtagtaatgctgatggttcggagaatgcttttggcggagggaccgaggtggtggtcgaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 67 - DNA, 408 bp, artificial sequence
  Synthetic nucleotide
atggacacgagggcccccactcagctgctggggctcctgctgctctggctcccaggtgccatatgtgacattgtgatgacccagactccatcttccgtgtctgcagctgtgggaggcacagtcaccatcaattgccacgccagtgagaacatttacggctacttatcctggtatcagcagaaaccagggcagcctcccaagctcctgatctacaaggcttccactctggcatctggggtcccatcgcggttcaaaggcagtggagctgggacacagttcactctcaccatcaccgccctggagtgtgccgatgctgccacttactactgtcaaagttattatactagtagtagtaatgctgatggttcggagaatgcttttggcggagggaccgaggtggtggtcgaa

SEQ ID NO: 68 - DNA, 1377 bp, Oryctolagus cuniculus
atggagactgggctgcgctggcttctcctggtcgctgtgctcaaaggtgtccagtgtcagtcgttggaggagtccgggggagacctggtcaagcctggggcatccctgacactcacctgcacagcctctggattcgacttcagtgacagatattacatgtgttgggtccgccaggctccagggaaggggctggagtggatcggatgcatttatgttggtagtggtgacacttattatgcgagctgggcgaaaggccgattcaccatctccagaacctcgtcgaccacggtgactctgcaaatgacccgtctgacagccgcggacacggccacctatttctgtgcgagacatcctggtacttactttactttatggggcccaggcaccctggtcaccgtctcctcagggcaacctaaggctccatcagtcttcccactggccccctgctgcggggacacacccagctccacggtgaccctgggctgcctggtcaaagggtacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 69 - DNA, 405 bp, artificial sequence
  Synthetic nucleotide
atggagactgggctgcgctggcttctcctggtcgctgtgctcaaaggtgtccagtgtcagtcgttggaggagtccgggggagacctggtcaagcctggggcatccctgacactcacctgcacagcctctggattcgacttcagtgacagatattacatgtgttgggtccgccaggctccagggaaggggctggagtggatcggatgcatttatgttggtagtggtgacacttattatgcgagctgggcgaaaggccgattcaccatctccagaacctcgtcgaccacggtgactctgcaaatgacccgtctgacagccgcggacacggccacctatttctgtgcgagacatcctggtacttactttactttatggggcccaggcaccctggtcaccgtctcctca

SEQ ID NO: 70 - DNA, 711 bp, Oryctolagus cuniculus
atggacacgagggcccccactcagctgctggggctcctgctgctctggctcccaggtgccagatgtgatgttgtgatgacccagactccatcctccgtgtctgcagctgtgggaggcacagtcaccatcaattgccaggccagtcagaacattgtcaacaatttagcctggtatcagcagagaccagggcagcctcccaaactcctgatctatgatacatccactctggcatctggggtcccatcgcggttcaaaggcagtggatctgggacagagttcactctcaccatcagcgacctggagtgtgccgatgctgccacttactactgtcaaacctattattattataataaaattataaatggtttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 71 - DNA, 396 bp, artificial sequence
  Synthetic nucleotide
atggacacgagggcccccactcagctgctggggctcctgctgctctggctcccaggtgccagatgtgatgttgtgatgacccagactccatcctccgtgtctgcagctgtgggaggcacagtcaccatcaattgccaggccagtcagaacattgtcaacaatttagcctggtatcagcagagaccagggcagcctcccaaactcctgatctatgatacatccactctggcatctggggtcccatcgcggttcaaaggcagtggatctgggacagagttcactctcaccatcagcgacctggagtgtgccgatgctgccacttactactgtcaaacctattattattataataaaattataaatggtttcggcggagggaccgaggtggtggtcaaa

SEQ ID NO: 72 - DNA, 1392 bp, Oryctolagus cuniculus
atggagactgggctgcgctggcttctcctggtcgctgtgctcaaaggtgtccagtgtcaggagcagctggtggagtccgggggaggcctggtccagcctgagggatccctgacaatcacctgcacaggttctggattctccttcagtagcaggttctacatgtgctgggtccgccaggctccagggaaggggctggagtggatcgcatgtatttatggtggtagtagtggtagcacttactacgcgagctgggcgaaaggccgattcaccatctccaaaacctcgtcgaccacggtgactctgcaaatttccagtctgacagccgcggacacggccacctatttctgtgccagaggtggtagtactgctgctgcgggctttaacttgtggggcccaggcaccctgctcaccgtctcctcagggcaacctaaggctccatcagtcttcccactggccccctgctgcggggacacacccagctccacggtgaccctgggctgcctggtcaaagggtacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 73 - DNA, 420 bp, artificial sequence
  Synthetic nucleotide
atggagactgggctgcgctggcttctcctggtcgctgtgctcaaaggtgtccagtgtcaggagcagctggtggagtccgggggaggcctggtccagcctgagggatccctgacaatcacctgcacaggttctggattctccttcagtagcaggttctacatgtgctgggtccgccaggctccagggaaggggctggagtggatcgcatgtatttatggtggtagtagtggtagcacttactacgcgagctgggcgaaaggccgattcaccatctccaaaacctcgtcgaccacggtgactctgcaaatttccagtctgacagccgcggacacggccacctatttctgtgccagaggtggtagtactgctgctgcgggctttaacttgtggggcccaggcaccctgctcaccgtctcctca

SEQ ID NO: 74 - DNA, 714 bp, Oryctolagus cuniculus
atggacacgagggcccccactcagctgctggggctcctgctgctctggctcccaggtgccagatgtgatgttgtgatgacccagactccagcctccgtgtctgcaactgtgggaggcacagtcaccatcaagtgccaggccagtgaggacatttataacttattggcctggtatcagcagaaaccagggcagcctcccaaactcctgatctattatgcatccactctggcatctggggtcccatcacggttcaaaggcagtggatctgggacagagttcactctcaccatcagcgacctggagtgtgccgatgctgccacttactactgtcaatgtaatgattatggtggtacttatgtccccaatgctttcggcggagggaccgaggtggtggtcaagggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 75 - DNA, 399 bp, artificial sequence
  Synthetic nucleotide
atggacacgagggcccccactcagctgctggggctcctgctgctctggctcccaggtgccagatgtgatgttgtgatgacccagactccagcctccgtgtctgcaactgtgggaggcacagtcaccatcaagtgccaggccagtgaggacatttataacttattggcctggtatcagcagaaaccagggcagcctcccaaactcctgatctattatgcatccactctggcatctggggtcccatcacggttcaaaggcagtggatctgggacagagttcactctcaccatcagcgacctggagtgtgccgatgctgccacttactactgtcaatgtaatgattatggtggtacttatgtccccaatgctttcggcggagggaccgaggtggtggtcaag

SEQ ID NO: 76 - DNA, 1404 bp, Oryctolagus cuniculus
atggagactgggctgcgctggcttctcctggtcgctgtgctcaaaggtgtccagtgtcagcagcagctggtggagtccgggggaggcctggtcaagcctggggcatccctgacactcaactgcaaagcctctggactcgacttcagtaccaactcctacatgtgctgggtccgccaggctccagggaagggcctggagtggatcgcatgcatttatgttggtgatagtagtgagatttattacgcgagctgggcgaaaggccgattcaccatctccaaaacctcgtcgaccacggtgactctacaaatgaccagtctgacagccgcggacacggccacctatttctgtgcgagagatctcccttcctttactgctccttatgctggttatcttcgcttgtggggcccaggcaccctggtcaccgtctcctcagggcaacctaaggctccatcagtcttcccactggccccctgctgcggggacacacccagctccacggtgaccctgggctgcctggtcaaagggtacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 77 - DNA, 432 bp, artificial sequence
  Synthetic nucleotide
atggagactgggctgcgctggcttctcctggtcgctgtgctcaaaggtgtccagtgtcagcagcagctggtggagtccgggggaggcctggtcaagcctggggcatccctgacactcaactgcaaagcctctggactcgacttcagtaccaactcctacatgtgctgggtccgccaggctccagggaagggcctggagtggatcgcatgcatttatgttggtgatagtagtgagatttattacgcgagctgggcgaaaggccgattcaccatctccaaaacctcgtcgaccacggtgactctacaaatgaccagtctgacagccgcggacacggccacctatttctgtgcgagagatctcccttcctttactgctccttatgctggttatcttcgcttgtggggcccaggcaccctggtcaccgtctcctca

SEQ ID NO: 78 - DNA, 732 bp, Oryctolagus cuniculus
atggcctgggctcctctgctcctgctgctcctctcccactgcacaggttccctctcccagcctgtgctgactcagccgccctccctgtctgcgtctctgggcacaacagccagactcacctgcaccctgaggactggctacaatgttggcgactaccctttactgtggctccagcgggtgccagggaggcctcccaggtatctcctgacctaccacacagaagaatttaaacatcagggctccggggtgcacagccgcttctctggatccaaagacgcctcagagaatgccggtgtcctgagcatctctgggctgcagcccgaggacgaggccaactattattgttatactgttcatgctactgagagcagcctccattatgtgttcggcggagggacacaactgaccgtcacaggtcagcccgcggtgaccccctcggtcattttgttcccgccctcctctgaggaactcaaggacaacaaggccaccttggtgtgtctgatcaatgacttctacccgggcaccgtgaaggtgaactggaaggccgatggtactcccgtcacccagggcgtggacaccacccagccctccaaacagagcaacagcaagtatgcggccagcagcttcctcagcctgtcagccaatcaatggaaatcctaccagagtgtcacctgccaggtcacgcatgagggccacacggtggagaagagcctggcccctgcagaatgctcc

SEQ ID NO: 79 - DNA, 414 bp, artificial sequence
  Synthetic nucleotide
atggcctgggctcctctgctcctgctgctcctctcccactgcacaggttccctctcccagcctgtgctgactcagccgccctccctgtctgcgtctctgggcacaacagccagactcacctgcaccctgaggactggctacaatgttggcgactaccctttactgtggctccagcgggtgccagggaggcctcccaggtatctcctgacctaccacacagaagaatttaaacatcagggctccggggtgcacagccgcttctctggatccaaagacgcctcagagaatgccggtgtcctgagcatctctgggctgcagcccgaggacgaggccaactattattgttatactgttcatgctactgagagcagcctccattatgtgttcggcggagggacacaactgaccgtcaca

SEQ ID NO: 80 - protein, 448 aa, Oryctolagus cuniculus
QAQLEESGGDLVKPEGSLTLTCTASGFSFTDRHYMCWVRQAPGKGLEWIACVYPGSSGSTYYASWARGRFTISKTSSTTVTLRMTSLTAADTATYFCARSSYPDSSGYSYGMDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 81 - protein, 125 aa, artificial sequence
  Synthetic peptide
QAQLEESGGDLVKPEGSLTLTCTASGFSFTDRHYMCWVRQAPGKGLEWIACVYPGSSGSTYYASWARGRFTISKTSSTTVTLRMTSLTAADTATYFCARSSYPDSSGYSYGMDLWGPGTLVTVSS

SEQ ID NO: 82 - protein, 9 aa, artificial sequence
  Synthetic peptide
GFSFTDRHY

SEQ ID NO: 83 - protein, 8 aa, artificial sequence
  Synthetic peptide
YPGSSGST

SEQ ID NO: 84 - protein, 17 aa, artificial sequence
  Synthetic peptide
ARSSYPDSSGYSYGMDL

SEQ ID NO: 85 - protein, 214 aa, Oryctolagus cuniculus
DIVMTQTPASVEAAVGGTVTIKCQASQDINSNLAWYQQKPGQRPKFLMYLTSKLASGVPSRFRGSGSGTQFTLTISDLECADAATYYCQTYYDISNYGYAFGGGTEVVVKGDPVAPTVLIFPPAADQVATGTVTIVCVANKYFPDVTVTWEVDGTTRTTGIENSKTPQNSADCTYNLSSTLTLTSTQYNSHKEYTCKVTQGTTSVVQSFNRGDC

SEQ ID NO: 86 - protein, 110 aa, artificial sequence
  Synthetic peptide
DIVMTQTPASVEAAVGGTVTIKCQASQDINSNLAWYQQKPGQRPKFLMYLTSKLASGVPSRFRGSGSGTQFTLTISDLECADAATYYCQTYYDISNYGYAFGGGTEVVVK

SEQ ID NO: 87 - protein, 6 aa, artificial sequence
  Synthetic peptide
QDINSN

SEQ ID NO: 88 - protein, 12 aa, artificial sequence
  Synthetic peptide
QTYYDISNYGYA

SEQ ID NO: 89 - protein, 448 aa, Oryctolagus cuniculus; identical to SEQ ID NO: 80

SEQ ID NO: 90 - protein, 125 aa, artificial sequence; identical to SEQ ID NO: 81
  Synthetic peptide

SEQ ID NO: 91 - protein, 9 aa, artificial sequence
  Synthetic peptide
GFSFTDRHY

SEQ ID NO: 92 - protein, 8 aa, artificial sequence
  Synthetic peptide
YPGSSGST

SEQ ID NO: 93 - protein, 17 aa, artificial sequence
  Synthetic peptide
ARSSYPDSSGYSYGMDL

SEQ ID NO: 94 - protein, 214 aa, Oryctolagus cuniculus; identical to SEQ ID NO: 15

SEQ ID NO: 95 - protein, 110 aa, artificial sequence; identical to SEQ ID NO: 16
  Synthetic peptide

SEQ ID NO: 96 - protein, 6 aa, artificial sequence
  Synthetic peptide
QNINSN

SEQ ID NO: 97 - protein, 12 aa, artificial sequence
  Synthetic peptide
QTYYDISNYGYA

SEQ ID NO: 98 - protein, 448 aa, Oryctolagus cuniculus
QAQLEESGGDLVKPEGSLTLTCTASGFSFTDRHYMCWVRQAPGKGLEWIACVYPGSSGSTYYASWARGRFTISKTSSTTVTLRMTSLTAADTATYFCARSSYPDSSGYSYGMDLWGPGTLVTVPSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 99 - protein, 125 aa, artificial sequence
  Synthetic peptide
QAQLEESGGDLVKPEGSLTLTCTASGFSFTDRHYMCWVRQAPGKGLEWIACVYPGSSGSTYYASWARGRFTISKTSSTTVTLRMTSLTAADTATYFCARSSYPDSSGYSYGMDLWGPGTLVTVPS

SEQ ID NO: 100 - protein, 9 aa, artificial sequence
  Synthetic peptide
GFSFTDRHY

SEQ ID NO: 101 - protein, 8 aa, artificial sequence
  Synthetic peptide
YPGSSGST

SEQ ID NO: 102 - protein, 17 aa, artificial sequence
  Synthetic peptide
ARSSYPDSSGYSYGMDL

SEQ ID NO: 103 - protein, 214 aa, Oryctolagus cuniculus; identical to SEQ ID NO: 15

SEQ ID NO: 104 - protein, 110 aa, artificial sequence; identical to SEQ ID NO: 16
  Synthetic peptide

SEQ ID NO: 105 - protein, 6 aa, artificial sequence
  Synthetic peptide
QNINSN

SEQ ID NO: 106 - protein, 12 aa, artificial sequence
  Synthetic peptide
QTYYDISNYGYA

SEQ ID NO: 107 - DNA, 1404 bp, Oryctolagus cuniculus
atggagactgggctgcgctggcttctcctggtcgctgtgctcaaaggtgtccagtgtcaggcgcagctggaggagtccgggggagacctggtcaagcctgagggatccctgacactcacctgcacagcctctggattctccttcactgacaggcattacatgtgctgggtccgccaggctccggggaaggggctggagtggatcgcatgcgtttatcctggtagtagtggtagcacttactacgcgagctgggcgagaggccgattcaccatctccaaaacctcgtcgaccacggtgactctgcgcatgaccagtctgacagccgcggacacggccacctatttctgtgcgagatcctcatatcctgatagtagtggctatagttatggcatggacctctggggccctgggaccctcgtcaccgtctcttcagggcaacctaaggctccatcagtcttcccactggccccctgctgcggggacacacccagctccacggtgaccctgggctgcctggtcaaagggtacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 108 - DNA, 432 bp, artificial sequence
  Synthetic nucleotide
atggagactgggctgcgctggcttctcctggtcgctgtgctcaaaggtgtccagtgtcaggcgcagctggaggagtccgggggagacctggtcaagcctgagggatccctgacactcacctgcacagcctctggattctccttcactgacaggcattacatgtgctgggtccgccaggctccggggaaggggctggagtggatcgcatgcgtttatcctggtagtagtggtagcacttactacgcgagctgggcgagaggccgattcaccatctccaaaacctcgtcgaccacggtgactctgcgcatgaccagtctgacagccgcggacacggccacctatttctgtgcgagatcctcatatcctgatagtagtggctatagttatggcatggacctctggggccctgggaccctcgtcaccgtctcttca

SEQ ID NO: 109 - DNA, 714 bp, Oryctolagus cuniculus
atggacacgagggcccccactcagctgctggggctcctgttgctctggctcccaggtgccagatgtgctgacattgtgatgacccagactccagcctccgtggaggcagctgtgggaggcacagtcaccatcaagtgccaggccagtcaggacattaacagcaatttagcctggtatcagcagaaaccagggcagcgtcccaagttcctgatgtatctgacatccaaactggcatctggggtcccatcgcggttcagaggcagtggatctgggacacagttcactctcaccatcagcgacctggagtgtgccgatgctgccacttactactgtcaaacctattatgatattagtaattatggatatgctttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccgaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 110 - DNA, 398 bp, artificial sequence
  Synthetic nucleotide
atggacacgagggcccccactcagctgctggggctcctgttgctctggctcccaggtgccagatgtgctgacattgtgatgacccagactccagcctccgtggaggcagctgtgggaggcacagtcaccatcaagtgccaggccagtcaggacattaacagcaatttagcctggtatcagcagaaaccagggcagcgtcccaagttcctgatgtatctgacatccaaactggcatctggggtcccatcgcggttcagaggcagtggatctgggacacagttcactctcaccatcagcgacctggagtgtgccgatgctgccacttactactgtcaaacctattatgatattagtaattatggatatgctttcggcggagggaccgaggtggtggtcaa

SEQ ID NO: 111 - DNA, 1404 bp, Oryctolagus cuniculus; identical to SEQ ID NO: 107

SEQ ID NO: 112 - DNA, 432 bp, artificial sequence; identical to SEQ ID NO: 108
  Synthetic nucleotide

SEQ ID NO: 113 - DNA, 714 bp, Oryctolagus cuniculus; identical to SEQ ID NO: 62

SEQ ID NO: 114 - DNA, 399 bp, artificial sequence; identical to SEQ ID NO: 63
  Synthetic nucleotide

SEQ ID NO: 115 - DNA, 1404 bp, Oryctolagus cuniculus
atggagactgggctgcgctggcttctcctggtcgctgtgctcaaaggtgtccagtgtcaggcgcagctggaggagtccgggggagacctggtcaagcctgagggatccctgacactcacctgcacagcctctggattctccttcactgacaggcattacatgtgctgggtccgccaggctccggggaaggggctggagtggatcgcatgcgtttatcctggtagtagtggtagcacttactacgcgagctgggcgagaggccgattcaccatctccaaaacctcgtcgaccacggtgactctgcgcatgaccagtctgacagccgcggacacggccacctatttctgtgcgagatcctcatatcctgatagtagtggctatagttatggcatggacctctggggccctgggaccctcgtcaccgtcccttcagggcaacctaaggctccatcagtcttcccactggccccctgctgcggggacacacccagctccacggtgaccctgggctgcctggtcaaagggtacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 116 - DNA, 432 bp, artificial sequence
  Synthetic nucleotide
atggagactgggctgcgctggcttctcctggtcgctgtgctcaaaggtgtccagtgtcaggcgcagctggaggagtccgggggagacctggtcaagcctgagggatccctgacactcacctgcacagcctctggattctccttcactgacaggcattacatgtgctgggtccgccaggctccggggaaggggctggagtggatcgcatgcgtttatcctggtagtagtggtagcacttactacgcgagctgggcgagaggccgattcaccatctccaaaacctcgtcgaccacggtgactctgcgcatgaccagtctgacagccgcggacacggccacctatttctgtgcgagatcctcatatcctgatagtagtggctatagttatggcatggacctctggggccctgggaccctcgtcaccgtcccttca

SEQ ID NO: 117 - DNA, 714 bp, Oryctolagus cuniculus; identical to SEQ ID NO: 62

SEQ ID NO: 118 - DNA, 399 bp, artificial sequence; identical to SEQ ID NO: 63
  Synthetic nucleotide

SEQ ID NO: 119 - protein, 504 aa, artificial sequence
  Synthetic peptide
IRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSA

SEQ ID NO: 120 - protein, 442 aa, Oryctolagus cuniculus
QCQSLEESGGGLVQPEGSLTLACTASEFDSSSNAMCWVRQAPGKGLEWIARIYSGSGTIYYANWARGRFTISKTSSTTVTLQMTSLTDADTATYFCARYNTGGFYYDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 121 - protein, 119 aa, artificial sequence
  Synthetic peptide
QCQSLEESGGGLVQPEGSLTLACTASEFDSSSNAMCWVRQAPGKGLEWIARIYSGSGTIYYANWARGRFTISKTSSTTVTLQMTSLTDADTATYFCARYNTGGFYYDLWGPGTLVTVSS

SEQ ID NO: 122 - protein, 8 aa, artificial sequence
  Synthetic peptide
EFDSSSNA

SEQ ID NO: 123 - protein, 8 aa, artificial sequence
  Synthetic peptide
IYSGSGTI

SEQ ID NO: 124 - protein, 12 aa, artificial sequence
  Synthetic peptide
ARYNTGGFYYDL

SEQ ID NO: 125 - protein, 214 aa, Oryctolagus cuniculus
ALVMTQTPASVEAAVGGTVTINCQASQRIGTNLAWYQQKPGQPPKVLIYKASTLASGVSSRFIGSGSGTDYTLTISGVECDDAATYYCQQGYSSNDADNTFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 126 - protein, 110 aa, artificial sequence
  Synthetic peptide
ALVMTQTPASVEAAVGGTVTINCQASQRIGTNLAWYQQKPGQPPKVLIYKASTLASGVSSRFIGSGSGTDYTLTISGVECDDAATYYCQQGYSSNDADNTFGGGTEVVVK

SEQ ID NO: 127 - protein, 6 aa, artificial sequence
  Synthtic peptide
QRIGTN

SEQ ID NO: 128 - protein, 12 aa, artificial sequence
  Synthetic peptide
QQGYSSNDADNT

SEQ ID NO: 129 - DNA, 1370 bp, Oryctolagus cuniculus
atggagactgggctgcgctggcttctcctggtcgctgtgctcaaaggtgtccagtgtcagtcgttggaggagtccgggggaggcctggtccagcctgagggatccctgacactcgcctgcacagctttgaattcgactccagtagcaatgcaatgtgctgggtccgccaggctccagggaagggactggagtggatcgcacgcatctatagtggtagtggtactatttactacgctaactgggcgagaggccgatcaccatctccaagacctcgtcgaccacggtgactctgcaaatgaccagtctgacagacgcggacacggccacctatttctgtgcgagatacaatactggtgggttctattatgacttgtggggcccggcaccctggtcaccgtctcctcagggcaacctaaggctccatcagtcttcccactggccccctgctgcggggacacacccagctccacggtgaccctgggctgcctggtcaaagggtacctcccggagccagtgaccgtgacctggaactgggcaccctcaccaatggggtacgcaccttcccgtccgtccggcagtcctcaggcctctactcgctgagcagcgtggtgagcgtgacctcaagcagccagcccgtcacctgcaacgtggcccacccaccaccaacaccaaagtggacaagaccgttgcgccctcgacatgcagcaagcccacgtgcccaccccctgaactcctggggggaccgtctgtcttcatcttccccccaaaacccaaggacaccctcatatctcacgcacccccgaggtcacatgcgtggtggtggacgtgagccaggatgaccccgaggtgcagttcacatggtacataaacaacgagcaggtgcgcaccgcccggccgccgctacgggagcagcgttcaacagcacgatccgcgtggtcagcaccctccccatcgcgcaccaggactggctgaggggcaaggagttcaagtgcaaagtccacaacaaggcactcccggcccccatcgagaaaaccatctccaagccagagggcagcccctggagccgaaggtctacaccatgggccctccccgggaggagctgagcagcaggtcggtcagcctgacctgcatgatcaacggcttctacccttccgacatctcggtggatgggagaagaacgggaaggcagaggacaactacaagaccacgccggccgtgctggacagcgacggctcctacttcctctacagcaagctctcagtgcccacgagtgagtggcagcggggcgacgtctcacctgctccgtgatgcacgaggccttgcacaaccactacacgcagaagtccatctcccgctctccgggtaaatga

SEQ ID NO: 130 - DNA, 405 bp, artificial sequence
  Synthetic nucleotide
atggagactgggctgcgctggcttctcctggtcgctgtgctcaaaggtgtccagtgtcagtcgttggaggagtccgggggaggcctggtccagcctgagggatccctgacactcgcctgcacagctttgaattcgactccagtagcaatgcaatgtgctgggtccgccaggctccagggaagggactggagtggatcgcacgcatctatagtggtagtggtactatttactacgctaactgggcgagaggccgatcaccatctccaagacctcgtcgaccacggtgactctgcaaatgaccagtctgacagacgcggacacggccacctatttctgtgcgagatacaatactggtgggttctattatgacttgtggggcccggcaccctggtcaccgtctcctca

SEQ ID NO: 131 - DNA, 706 bp, Oryctolagus cuniculus
atggacacgagggcccccactcagctgctggggctcctgctgctctggctcccaggtgccagatgtgcccttgtgatgacccagactccagcctccgtggaggcagctgtgggaggcacagtcaccacaattgccaggccagtcagagaattggtactaatttagcctggtatcagcagaaaccagggcagcctcccaaggtcctgatctacaaggcatccactctggcatctggggtctcatcgcggttcatagcagtggatctgggacagactatactctcaccatcagcggcgtggagtgtgacgatgctgccacttactactgtcaacagggttatagtagtaatgatgccgataatactttcggcggagggaccgagtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggtggcaccacccaaacaactggcatcgagaacagtaaaacaccgcagaattctgcagattgtacctacaacctcagcagcactctgacactgaccagcacacagtacaacagccacaaagagtacaccgcaaggtgacccagggcacgacctcagtcgtccagagcttcaataggggtgactgttag

SEQ ID NO: 132 - DNA, 393 bp, artificial sequence
  Synthetic nuleotide
atggacacgagggcccccactcagctgctggggctcctgctgctctggctcccaggtgccagatgtgcccttgtgatgacccagactccagcctccgtggaggcagctgtgggaggcacagtcaccacaattgccaggccagtcagagaattggtactaatttagcctggtatcagcagaaaccagggcagcctcccaaggtcctgatctacaaggcatccactctggcatctggggtctcatcgcggttcatagcagtggatctgggacagactatactctcaccatcagcggcgtggagtgtgacgatgctgccacttactactgtcaacagggttatagtagtaatgatgccgataatactttcggcggagggaccgagtggtggtcaaa